(19) United States
(12) Patent Application Publication
(10) Pub. No.: US 2012/0189637 A1
(43) Pub. Date: Jul. 26, 2012

(54) METHODS FOR TREATING PSORIASIS

(76) Inventors: Joaquin Mario Valdes, Mundelein, IL (US); Susan K. Paulson, Downers Grove, IL (US); Elliot K. Chartash, Baskin Ridge, NJ (US); Yanjun Bao, Buffalo Grove, IL (US); Parvez M . Mulani, Gurnee, IL (US); Murali Sundaram, Buffalo Grove, IL (US); Yihua Gu, Vernon Hills, IL (US); Michele Olds Heckaman, Gurnee, IL (US); Tom C. Harris, Gurnee, IL (US); Martin Kaul, Neustadt (DE); David Allen Williams, Lake Forest, IL (US); Richard G.B. Langley, Halifax (CA); Kenneth Gordon, Northbrook, IL (US)

(21) Appl. No.: 13/267,658

(22) Filed: Oct. 6, 2011

Related U.S. Application Data
(60) Provisional application No. 61/390,590, filed on Oct. 6, 2010.
(60) Provisional application No. 61/453,541, filed on Mar. 16, 2011.

(51) Int. Cl. A61K 39/395; A61P 17/06

(52) U.S. Cl. 424/142.1; 424/158.1

(57) ABSTRACT

The invention provides methods of treating psoriasis in a subject by administering to a subject an antibody capable of binding to the p40 subunit of IL-12 and/or IL-23.

### RELATED APPLICATIONS

This application claims priority to U.S. Provisional Patent Application Ser. No. 61/390,590 entitled “Methods For Treating Psoriasis” filed on Oct. 6, 2010, and U.S. Provisional Patent Application Ser. No. 61/453,541 entitled “Methods for Treating Psoriasis” filed on Mar. 16, 2011, the entire contents of each which are incorporated herein by reference in their entirety.

### BACKGROUND OF THE INVENTION

Psoriasis is a T cell-mediated inflammatory disease that is considered to be one of the most common autoimmune diseases, affecting approximately 2% to 3% of adults, though the global prevalence varies widely (Stem R. S., et al., J Investig Dermatol Symp Proc 2004, 9: 136-39; Davidson A and Diamond B. N Engl J Med 2001, 345: 340-50; Langley R. G. B., et al., Ann Rheum Dis 2005, 64(Suppl II): ii18-23). Psoriasis has a major impact on quality of life (de Korte J, et al., J Investig Dermatol Symp Proc 2004, 9: 140-7; Krueger G, et al., Arch Dermatol 2001, 137: 280-4; Finlay A Y and Coles E C, Br J Dermatol 1995, 132: 236-44) and is associated with a number of psychological and psychosocial problems (Kimball A B, et al., Am J Clin Dermatol 2005, 6: 383-92; Russoi P A, et al., Australas J Dermatol 2004, 45: 155-9). Many traditional psoriasis therapies have toxic adverse effects; therefore, their long-term use is limited (Lebwohl M. and Ali S., J Am Acad Dermatol 2001, 45: 487-98; Lebwohl M. and Ali S., J Am Acad Dermatol 2001, 45: 649-61). In addition, many patients with psoriasis are dissatisfied with traditional therapies (Stern R S, et al., J Investig Dermatol Symp Proc 2004, 9: 136-39; Finlay A Y and Ortonne J P, J Cutan Med Surg 2004, 8: 310-20); thus, there is a clear need for therapies that are safer and easier to use and that can be prescribed on a long-term basis.

Interleukin-12 (IL-12) and the related cytokine IL-23 are members of the IL-12 superfamily of cytokines that share a common p40 subunit (Anderson E J R, et al., Springer Semin Immunopathol 2006, 27: 425-42). Both cytokines contribute to the development of the type 1T helper cell (Th1) immune response in psoriasis, but each has a unique role (Rosmarin D and Strober B E, J Drugs Dermatol 2005, 4: 318-25; Hong K, et al., J Immunol 1999, 162: 7480-91; Yawalkar N, et al., J Invest Dermatol 1998, 111: 1053-57). IL-12 primarily stimulates differentiation of Th1 cells and subsequent secretion of interferon-gamma, whereas IL-23 preferentially stimulates differentiation of naïve T cells into effector T helper cells (Th17) that secrete IL-17, a proinflammatory mediator Rosmarin D and Strober B E, J Drugs Dermatol 2005, 4: 318-25; Harrington Le, et al., Nature Immunol 2005, 6: 1123-32; Park H, et al. Nature Immunol 2005, 6: 1132-41). The overexpression of IL-12 p40 and IL-23 p40 messenger RNA in psoriatic skin lesions suggests that the inhibition of IL-12 and IL-23 with a neutralizing antibody to the IL-12/23 p40 subunit protein may offer an effective therapeutic approach for the treatment of psoriasis (Yawalkar N, et al., J Invest Dermatol 1998, 111: 1053-57; Lee E, et al., J Exp Med 2004, 199: 125-30; Shaker O G, et al., Clin Biochem 2006, 39: 119-25; Piskin G, et al., J Immunol 2006, 176: 1908-15). Such therapeutic approaches for the treatment of psoriasis are clearly needed in the art.

### SUMMARY OF THE INVENTION

The present invention provides methods and compositions for treating psoriasis, e.g., chronic psoriasis, using an antibody, or antigen-binding portion thereof, that binds human IL-12 and/or human IL-23.

In one aspect, the invention provides methods of treating psoriasis in a subject or population of subjects, comprising administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in a Short Form 36 Health Survey domain score selected from the group consisting of a Physical Function score, a Role-Physical score, a Bodily Pain score, a General Health score, a Vitality score, a Social Function score, a Role-Emotional score, and a Mental Health score.

In one embodiment, the subject or population of subjects achieves an improvement or mean improvement in a Short Form 36 Health Survey Physical Function score of at least about 3. In one embodiment, the subject or population of subjects achieves an improvement or mean improvement in a Short Form 36 Health Survey Role-Physical score of at least about 2.5. In one embodiment, the subject or population of subjects achieves an improvement or mean improvement in a Short Form 36 Health Survey Bodily Pain score of at least about 6. In one embodiment, the subject or population of subjects achieves an improvement or mean improvement in a Short Form 36 Health Survey General Health score of at least about 2.5. In one embodiment, the subject or population of subjects achieves an improvement or mean improvement in a Short Form 36 Health Survey Vitality score of at least about 2.5. In one embodiment, the subject or population of subjects achieves an improvement or mean improvement in a Short Form 36 Health Survey Social Function score of at least about 5. In another embodiment, the subject or population of subjects achieves an improvement or mean improvement in a Short Form 36 Health Survey Role-Emotional score of at least about 4.5. In another embodiment, the subject or population of subjects achieves an improvement or mean improvement in a Short Form 36 Health Survey Mental Health score of at least about 2.5.

In another embodiment, at least 30% of the population of subjects achieves an improvement at or exceeding a minimum clinically important difference (MCID) response for a Short Form 36 Health Survey Physical Function score. In one embodiment, at least 20% of the population of subjects achieves an improvement at or exceeding a minimum clinically important difference (MCID) response for a Short Form 36 Health Survey Role-Physical score. In one embodiment, at least 40% of the population of subjects achieves an improvement at or exceeding a minimum clinically important difference (MCID) response for a Short Form 36 Health Survey Bodily Pain score. In another embodiment, at least 20% of the population of subjects achieves an improvement at or exceeding a minimum clinically important difference (MCID) response for a Short Form 36 Health Survey General Health score. In another embodiment, at least 35% of the population of subjects achieves an improvement at or exceeding a minimum clinically important difference (MCID) response for a Short Form 36 Health Survey Vitality score. In one embodiment, at least 20% of the population of subjects achieves an improvement at or exceeding a minimum clinically important difference (MCID) response for a Short Form 36 Health Survey Social Function score. In one embodiment, at least 5% of the population of subjects achieves an improvement at or exceeding a minimum clinically important difference (MCID) response for a Short Form 36 Health Survey Role Emotional score. In another embodiment, at least 40% of the population of subjects achieves an improvement at or exceeding a minimum clinically important difference (MCID) response for a Short Form 36 Health Survey Mental Health score.

In another aspect, the invention provides methods of treating psoriasis in a subject or population of subjects, comprising administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in an HRQOL outcome selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP).

In one embodiment, the subject or population of subjects achieves an improvement or mean improvement in a Dermatology Life Quality Index (DLQI) score by at least about −8. In another embodiment, the subject or population of subjects achieves an improvement or mean improvement in a psoriasis-related pain (VAS-Ps) score by at least about −25. In one embodiment, the subject or population of subjects achieves an improvement or mean improvement in a psoriatic arthritis-related pain (VAS-PsA) score by at least about −15.

In another embodiment, the subject or population of subjects achieves an improvement or mean improvement in a work productivity and activity impairment-specific health problem for psoriasis (WPAI-SHP) score by at least about −2 for % work time missed. In another embodiment, the subject or population of subjects achieves an improvement or mean improvement in a work productivity and activity impairment-specific health problem for psoriasis (WPAI-SHP) score by at least about −13 for % impairment while working. In one embodiment, the subject or population of subjects achieves an improvement or mean improvement in a work productivity and activity impairment-specific health problem for psoriasis (WPAI-SHP) score by at least about −13 for % overall work impairment. In one embodiment, the subject or population of subjects achieves an improvement or mean improvement in a work productivity and activity impairment-specific health problem for psoriasis (WPAI-SHP) score by at least about −18 for % overall activity impairment.

In another embodiment, at least about 60% of the population of subjects achieves an improvement at or exceeding a minimum clinically important difference (MCID) response for psoriasis-related pain (VAS-Ps) by about week 12 or 52. In another embodiment, at least about 50% of the population of subjects achieves an improvement at or exceeding a minimum clinically important difference (MCID) response for psoriatic arthritis-related pain (VAS-PsA) by about week 12 or 52. In one embodiment, at least about 6% of the population of subjects achieves an improvement at or exceeding a minimum clinically important difference (MCID) response for work productivity and activity impairment-specific health problem for psoriasis (WPAI-SHP) for % work time missed by about week 12 or 52. In one embodiment, at least about 35% of the population of subjects achieves an improvement at or exceeding a minimum clinically important difference (MCID) response for work productivity and activity impairment-specific health problem for psoriasis (WPAI-SHP) for % impairment while working. In one embodiment, at least about 35% of the population of subjects achieves an improvement at or exceeding a minimum clinically important difference (MCID) response for work productivity and activity impairment-specific health problem for psoriasis (WPAI-SHP) for % overall work impairment. In one embodiment, at least about 45% of the population of subjects achieves an improvement at or exceeding a minimum clinically important difference (MCID) response for work productivity and activity impairment-specific health problem for psoriasis (WPAI-SHP) for % overall activity impairment.

In another embodiment, the improvement is achieved by about week 12. In one embodiment, the improvement is achieved by about week 52.

In another aspect, the invention provides methods of treating psoriasis in a subject, comprising administering to the subject an antibody, or antigen-binding portion thereof, which is capable of binding to the p40 subunit of IL-12 and/or IL-23, wherein the subject upon treatment achieves a PGA score of 0 or 1 in less than about 60 days.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein the population of subjects upon treatment achieves a Physician's Global Assessment (PGA) score of 0 or 1 in a median time of less than about 60 days.

In another aspect, the invention provides methods of treating psoriasis in a subject, comprising administering to the subject an antibody, or antigen-binding portion thereof, which is capable of binding to the p40 subunit of IL-12 and/or IL-23, wherein the subject upon treatment achieves a Psoriasis Area and Severity Index (PASI) 75 response in less than about 70 days.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein the population of subjects upon treatment achieves a Psoriasis Area and Severity Index (PASI) 75 response in a median time of less than about 70 days.

In another aspect, the invention provides methods of treating psoriasis in a subject, comprising administering to the subject an antibody, or antigen-binding portion thereof, which is capable of binding to the p40 subunit of IL-12 and/or IL-23, wherein the subject upon treatment achieves a Dermatology Life Quality Index (DLQI) score of 0 by about week 12.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 20% of the population of subjects upon treatment achieve a Dermatology Life Quality Index (DLQI) score of 0 by about week 12.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 15% of the population of subjects upon treatment achieve at least a PGA score of 0 or 1 by about week 4.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 18% of the population of subjects upon treatment achieve a PGA score of 0 or 1 by about week 8. In one embodiment, at least about 50% of the population of subjects upon treatment achieve a PGA score of 0 or 1 by about week 8.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 20% of the population of subjects upon treatment achieve at least a PASI 75 response by about week 4.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 25% of the population of subjects upon treatment achieve at least a PASI 75 response by about week 8. In one embodiment, at least about 60% of the population of subjects upon treatment achieve at least a PASI 75 response by about week 8.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 40% of the population of subjects upon treatment achieve at least a PASI 75 response by about week 12. In one embodiment, at least about 80% of the population of subjects upon treatment achieve at least a PASI 75 response by about week 12.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 35% of the population of subjects upon treatment achieve at least a PASI 90 response by about week 8.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 15% of the population of subjects upon treatment achieve at least a PASI 90 response by about week 12. In one embodiment, at least about 50% of the population of subjects upon treatment achieve at least a PASI 90 response by about week 12.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 10% of the population of subjects upon treatment achieve a PASI 100 response by about week 8.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least about 5% of the population of subjects upon treatment achieve a PASI 100 response by about week 12. In one embodiment, at least about 25% of the population of subjects upon treatment achieve a PASI 100 response by about week 12.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein each subject was treated with a biologic prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein none of the subjects were treated with a biologic prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 65% of the population of subjects achieve a PGA score of 0 or 1 by about week 12, wherein each subject was treated with a biologic and showed no improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve a PGA score of 0 or 1 by about week 12, wherein each subject was treated with a biologic and showed improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve a PGA score of 0 or 1 by about week 52, wherein each subject was treated with a biologic and showed no improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve a PGA score of 0 or 1 by about week 52, wherein each subject was treated with a biologic and showed improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve at least a PASI 75 response by about week 12, wherein each subject was treated with a biologic and showed no improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 12, wherein each subject was treated with a biologic and showed improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 52, wherein each subject was treated with a biologic and showed no improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 52, wherein each subject was treated with a biologic and showed improvement prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein each subject has a prior history of psoriatic arthritis.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein none of the subjects has a prior history of psoriatic arthritis.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve a PGA score of 0 or 1 by about week 52, wherein each subject had a baseline weight of less than 100 kilograms prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve a PGA score of 0 or 1 by about week 52, wherein each subject had a baseline weight of greater than or equal to 100 kilograms prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein each subject had a baseline weight of less than 100 kilograms prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 52, wherein each subject had a baseline weight of greater than or equal to 100 kilograms prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve a PGA score of 0 or 1 by about week 52, wherein each subject had a baseline PASI score of less than or equal to 20 prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve a PGA score of 0 or 1 by about week 52, wherein each subject had a baseline PASI score of greater than 20 prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 52, wherein each subject had a baseline PASI score of less than or equal to 20 prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI response by about week 52, wherein each subject had a baseline PASI score of greater than 20 prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve a PGA score of 0 or 1 by about week 12, wherein each subject had less than or equal to 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve a PGA score of 0 or 1 by about week 12, wherein each subject had greater than 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve a PGA score of 0 or 1 by about week 52, wherein each subject had less than or equal to 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 70% of the population of subjects achieve a PGA score of 0 or 1 by about week 52, wherein each subject had greater than 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 80% of the population of subjects achieve at least a PASI 75 response by about week 12, wherein each subject had less than or equal to 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 12, wherein each subject had greater than 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 85% of the population of subjects achieve at least a PASI 75 response by about week 52, wherein each subject had less than or equal to 20% body surface area (BSA) affected by psoriasis prior to administration of the antibody.

In another aspect, the invention provides methods of treating psoriasis in a population of subjects, comprising administering to each subject in the population an antibody, or antigen-binding portion thereof, which is capable of binding to the p40 subunit of IL-12 and/or IL-23, wherein at least 75% of the population of subjects achieve at least a PASI 75 response by about week 52, wherein each subject had greater than 20% body surface area (BSA) affected by psoriasis prior to administration of the antibody.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PASI 75 at about week 8, wherein all of the subjects were previously exposed to a tumor necrosis factor- (TNF-) antagonist. In one embodiment, at least 80% of the population achieves a PASI 75.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 80% of the population of subjects achieves at least a PASI 75 at about week 52, wherein all of the subjects were previously exposed to a tumor necrosis factor- (TNF-) antagonist.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 80% of the population of subjects achieves at least a PASI 75 at about week 100, wherein all of the subjects were previously exposed to a tumor necrosis factor- (TNF-) antagonist. In one embodiment, at least 90% of the population achieves a PASI 75.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 50% of the population of subjects achieves a PGA score of 0 or 1 at about week 8, wherein all of the subjects were previously exposed to a tumor necrosis factor- (TNF-) antagonist. In one embodiment, at least 60% of the population achieves a PGA score of 0 or 1.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 75% of the population of subjects achieves a PGA score of 0 or 1 at about week 52, wherein all of the subjects were previously exposed to a tumor necrosis factor- (TNF-) antagonist. In one embodiment, at least 85% of the population achieves a PGA score of 0 or 1.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 75% of the population of subjects achieves a PGA score of 0 or 1 at about week 100, wherein all of the subjects were previously exposed to a tumor necrosis factor- (TNF-) antagonist. In one embodiment, at least 80% of the population achieves a PGA score of 0 or 1.

In one embodiment, the subjects of the population failed to respond to treatment with a TNF-antagonist. In another embodiment, the TNF antagonist is selected from the group consisting of anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors.

In another aspect, the invention provides methods of treating psoriasis in a subject, comprising administering to each subject in the population an antibody, or antigen binding portion thereof, which is capable of binding to the p40 subunit of IL-12 and/or IL-23, wherein the subject achieves at least a PASI 75 at about week 84.

In another aspect, the invention provides methods of treating psoriasis in a subject, comprising administering to each subject in the population an antibody, or antigen binding portion thereof, which is capable of binding to the p40 subunit of IL-12 and/or IL-23, wherein the subject achieves at least a PASI 75 at about week 124.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 90% of the population of subjects achieves at least a PASI 75 at about week 84.

In another aspect, the invention provides method of treating psoriasis in a population of subjects, comprising administering to each subject in the population an antibody, or antigen binding portion thereof, which is capable of binding to the p40 subunit of IL-12 and/or IL-23, wherein at least 90% of the population of subjects achieves at least a PASI 75 at about week 124.

In one embodiment, the subjects of the population do not suffer an adverse event during treatment with the antibody, or antigen binding portion thereof, which is capable of binding to the p40 subunit of IL-12 and/or IL-23.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 40% of the population of subjects achieves at least a PASI 90 at about week 8, wherein all of the subjects were previously exposed to a tumor necrosis factor- (TNF-) antagonist. In one embodiment, at least 50% of the population achieves a PASI 90.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 10% of the population of subjects achieves at least a PASI 100 at about week 8, wherein all of the subjects were previously exposed to a tumor necrosis factor- (TNF-) antagonist. In one embodiment, at least 20% of the population achieves a PASI 100.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PASI 90 at about week 52, wherein all of the subjects were previously exposed to a tumor necrosis factor- (TNF-) antagonist. In one embodiment, at least 80% of the population achieves a PASI 90.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 60% of the population of subjects achieves at least a PASI 100 at about week 52, wherein all of the subjects were previously exposed to a tumor necrosis factor- (TNF-) antagonist. In one embodiment, at least 70% of the population achieves a PASI 100.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PASI 90 at about week 100, wherein all of the subjects were previously exposed to a tumor necrosis factor- (TNF-) antagonist. In one embodiment, at least 80% of the population achieves a PASI 90.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 50% of the population of subjects achieves at least a PASI 100 at about week 100, wherein all of the subjects were previously exposed to a tumor necrosis factor- (TNF-) antagonist. In one embodiment, at least 60% of the population achieves a PASI 100.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 15% of the population of subjects achieves a PGA score of 0 at about week 8, wherein all of the subjects were previously exposed to a tumor necrosis factor- (TNF-) antagonist. In one embodiment, at least 25% of the population achieves a PGA score of 0.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 65% of the population of subjects achieves a PGA score of 0 at about week 52, wherein all of the subjects were previously exposed to a tumor necrosis factor- (TNF-) antagonist. In one embodiment, at least 75% of the population achieves a PGA score of 0.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 55% of the population of subjects achieves a PGA score of 0 at about week 100, wherein all of the subjects were previously exposed to a tumor necrosis factor- (TNF-) antagonist. In one embodiment, at least 65% of the population achieves a PGA score of 0 or 1.

In one embodiment, the subjects of the population failed to respond to treatment with a TNF-antagonist. In another embodiment, the TNF antagonist is selected from the group consisting of anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors.

In another aspect, the invention provides methods of treating psoriasis in a subject, comprising administering to each subject in the population an antibody, or antigen binding portion thereof, which is capable of binding to the p40 subunit of IL-12 and/or IL-23, wherein the subject achieves a PGA response rate of 0 or 1 at about week 12.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 90% of the population of subjects maintain at least a PASI 75 through about week 96.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 80% of the population of subjects maintain at least a PASI 90 through about week 96.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 65% of the population of subjects maintain at least a PASI 100 through about week 96.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 90% of the population of subjects maintain at least a PGA 0 or 1 response through about week 96.

In one embodiment, the subjects of the population do not suffer an adverse event during treatment with the antibody, or antigen binding portion thereof, which is capable of binding to the p40 subunit of IL-12 and/or IL-23.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PASI 75 at about week 28, wherein all of the subjects were previously exposed to etanercept. In one embodiment, at least 90% of the population of subjects achieves at least a PASI 75 at about week 28.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 75% of the population of subjects achieves at least a PASI 75 at about week 88, wherein all of the subjects were previously exposed to etanercept. In one embodiment, at least 85% of the population of subjects achieves at least a PASI 75 at about week 88.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 50% of the population of subjects achieves at least a PASI 90 at about week 28, wherein all of the subjects were previously exposed to etanercept. In one embodiment, at least 75% of the population of subjects achieves at least a PASI 90 at about week 28.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PASI 90 at about week 88, wherein all of the subjects were previously exposed to etanercept. In one embodiment, at least 85% of the population of subjects achieves at least a PASI 90 at about week 88.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 20% of the population of subjects achieves at least a PASI 100 at about week 28, wherein all of the subjects were previously exposed to etanercept. In one embodiment, at least 50% of the population of subjects achieves at least a PASI 100 at about week 28.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 45% of the population of subjects achieves at least a PASI 100 at about week 88, wherein all of the subjects were previously exposed to etanercept. In one embodiment, at least 55% of the population of subjects achieves at least a PASI 100 at about week 88.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 55% of the population of subjects achieves at least a PGA 0 or 1 at about week 28, wherein all of the subjects were previously exposed to etanercept. In one embodiment, at least 85% of the population of subjects achieves at least a PGA 0 or 1 at about week 28.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieves at least a PGA 0 or 1 at about week 88, wherein all of the subjects were previously exposed to etanercept. In one embodiment, at least 80% of the population of subjects achieves at least a PGA 0 or 1 at about week 88.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 20% of the population of subjects achieves at least a PGA 0 at about week 28, wherein all of the subjects were previously exposed to etanercept. In one embodiment, at least 50% of the population of subjects achieves at least a PGA 0 at about week 28.

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 45% of the population of subjects achieves at least a PGA 0 at about week 88, wherein all of the subjects were previously exposed to etanercept. In one embodiment, at least 60% of the population of subjects achieves at least a PGA 0 at about week 88.

In one embodiment, the subjects did not previously achieve a PGA response of 0 or 1. In another embodiment, the subjects previously achieved a PGA response of 0 or 1.

In one embodiment, the antibody, or antigen-binding portion thereof, is administered according to a periodicity of about once every 4 week, thereby treating psoriasis in the subjects. In another embodiment, the antibody, or antigen-binding portion thereof, is administered according to a periodicity of about once every 12 weeks, thereby treating psoriasis in the subject.

In one embodiment, the antibody, or antigen-binding portion thereof, is administered in a a) a first dose amount according to a first periodicity of about once every 4 weeks; and b) a second dose amount that is about 40-60% of the first dose amount of the antibody, or antigen-binding portion thereof, according to a second periodicity of about once every 4 weeks, thereby treating psoriasis in the subject.

In another embodiment, the antibody, or antigen-binding portion thereof, is administered in a a) a first dose amount of an antibody, or antigen-binding portion thereof, which is capable of binding to the p40 subunit of IL-12 and/or IL-23, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks, thereby treating psoriasis in the subject.

In another embodiment, the first dose amount is at least about 200 mg. In another embodiment, the second dose amount is at least about 100 mg.

In one embodiment, the antibody is a human antibody. In another embodiment, the antibody is ABT-874.

In one embodiment, the method comprises administering to the subject or to each subject in the population: a) about 200 mg of ABT-874 once every four weeks for two doses; and b) about 100 mg of ABT-874 every four weeks thereafter.

In another embodiment, the method comprises administering to the subject or to each subject in the population: a) about 200 mg of ABT-874 at weeks 0 and 4; and b) about 100 mg of ABT-874 at week 8 and every 4 weeks thereafter.

In one embodiment, the antibody is administered subcutaneously. In another embodiment, the psoriasis is moderate to severe or chronic psoriasis. In yet another embodiment, the psoriasis is plaque psoriasis.

In one aspect, the invention provides methods of treating psoriasis in a subject or population of subjects, comprising selecting a subject or population of subjects who would benefit from an improvement in a Short Form 36 Health Survey domain score (e.g., an improvement of at least about 2.5, 3, 4, 5, 6 or more, or, e.g., an improvement at or exceeding a minimum clinically important difference (MCID) response for a Short Form 36 Health Survey Physical Function score) selected from the group consisting of a Physical Function score, a Role-Physical score, a Bodily Pain score, a General Health score, a Vitality score, a Social Function score, a Role-Emotional score, and a Mental Health score, and administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in a Short Form 36 Health Survey domain score selected from the group consisting of a Physical Function score, a Role-Physical score, a Bodily Pain score, a General Health score, a Vitality score, a Social Function score, a Role-Emotional score, and a Mental Health score.

In one embodiment, the subject or population of subjects achieves an improvement or mean improvement in a Short Form 36 Health Survey Physical Function score of at least about 3, the subject or population of subjects achieves an improvement or mean improvement in a Short Form 36 Health Survey Role-Physical score of at least about 2.5, the subject or population of subjects achieves an improvement or mean improvement in a Short Form 36 Health Survey Bodily Pain score of at least about 6, the subject or population of subjects achieves an improvement or mean improvement in a Short Form 36 Health Survey General Health score of at least about 2.5, the subject or population of subjects achieves an improvement or mean improvement in a Short Form 36 Health Survey Vitality score of at least about 2.5, the subject or population of subjects achieves an improvement or mean improvement in a Short Form 36 Health Survey Social Function score of at least about 5, the subject or population of subjects achieves an improvement or mean improvement in a Short Form 36 Health Survey Role-Emotional score of at least about 4.5, and/or the subject or population of subjects achieves an improvement or mean improvement in a Short Form 36 Health Survey Mental Health score of at least about 2.5.

In another embodiment, at least 30% of the population of subjects achieves an improvement at or exceeding a minimum clinically important difference (MCID) response for a Short Form 36 Health Survey Physical Function score, at least 20% of the population of subjects achieves an improvement at or exceeding a minimum clinically important difference (MCID) response for a Short Form 36 Health Survey Role-Physical score, at least 40% of the population of subjects achieves an improvement at or exceeding a minimum clinically important difference (MCID) response for a Short Form 36 Health Survey Bodily Pain score, at least 20% of the population of subjects achieves an improvement at or exceeding a minimum clinically important difference (MCID) response for a Short Form 36 Health Survey General Health score, at least 35% of the population of subjects achieves an improvement at or exceeding a minimum clinically important difference (MCID) response for a Short Form 36 Health Survey Vitality score, at least 20% of the population of subjects achieves an improvement at or exceeding a minimum clinically important difference (MCID) response for a Short Form 36 Health Survey Social Function score, at least 5% of the population of subjects achieves an improvement at or exceeding a minimum clinically important difference (MCID) response for a Short Form 36 Health Survey Role Emotional score, and/or at least 40% of the population of subjects achieves an improvement at or exceeding a minimum clinically important difference (MCID) response for a Short Form 36 Health Survey Mental Health score.

In another aspect, the invention provides methods of treating psoriasis in a subject or population of subjects, comprising selecting a subject or population of subjects who would benefit from an improvement or mean improvement in an HRQOL outcome (e.g., an improvement or mean improvement of at least about −2, −5, −10, −15, −18, −20, −25; or e.g., an improvement at or exceeding a minimum clinically important difference (MCID) response) selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP), and administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in an HRQOL outcome selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP).

In one embodiment, the subject or population of subjects achieves an improvement or mean improvement in a Dermatology Life Quality Index (DLQI) score by at least about −8, the subject or population of subjects achieves an improvement or mean improvement in a psoriasis-related pain (VAS-Ps) score by at least about −25, and/or the subject or population of subjects achieves an improvement or mean improvement in a psoriatic arthritis-related pain (VAS-PsA) score by at least about −15.

In another embodiment, the subject or population of subjects achieves an improvement or mean improvement in a work productivity and activity impairment-specific health problem for psoriasis (WPAI-SHP) score by at least about −2 for % work time missed, the subject or population of subjects achieves an improvement or mean improvement in a work productivity and activity impairment-specific health problem for psoriasis (WPAI-SHP) score by at least about −13 for % impairment while working, the subject or population of subjects achieves an improvement or mean improvement in a work productivity and activity impairment-specific health problem for psoriasis (WPAI-SHP) score by at least about −13 for % overall work impairment, and/or the subject or population of subjects achieves an improvement or mean improvement in a work productivity and activity impairment-specific health problem for psoriasis (WPAI-SHP) score by at least about −18 for % overall activity impairment.

In yet another embodiment, at least about 60% of the population of subjects achieves an improvement at or exceeding a minimum clinically important difference (MCID) response for psoriasis-related pain (VAS-Ps) by about week 12 or 52, at least 50% of the population of subjects achieves an improvement at or exceeding a minimum clinically important difference (MCID) response for psoriatic arthritis-related pain (VAS-PsA) by about week 12 or 52, at least 6% of the population of subjects achieves an improvement at or exceeding a minimum clinically important difference (MCID) response for work productivity and activity impairment-specific health problem for psoriasis (WPAI-SHP) for % work time missed by about week 12 or 52, at least 35% of the population of subjects achieves an improvement at or exceeding a minimum clinically important difference (MCID) response for work productivity and activity impairment-specific health problem for psoriasis (WPAI-SHP) for % impairment while working, at least 35% of the population of subjects achieves an improvement at or exceeding a minimum clinically important difference (MCID) response for work productivity and activity impairment-specific health problem for psoriasis (WPAI-SHP) for % overall work impairment, and/or at least 45% of the population of subjects achieves an improvement at or exceeding a minimum clinically important difference (MCID) response for work productivity and activity impairment-specific health problem for psoriasis (WPAI-SHP) for % overall activity impairment.

In one embodiment, the improvement is achieved by about week 12. In another embodiment, the improvement is achieved by about week 52.

In another aspect, the invention provides methods of treating psoriasis in a subject or population of subjects, comprising selecting a subject or population of subjects who would benefit from an improvement in PGA score (e.g., a PGA score of 0 or 1), and administering to the subject or each subject in the population of subjects an antibody, or antigen-binding portion thereof, which is capable of binding to the p40 subunit of IL-12 and/or IL-23, wherein the subject or the population of subjects upon treatment achieves a PGA score of 0 or 1 at a time or a median time of less than about 60 days.

In another aspect, the invention provides methods of treating psoriasis in a subject or population of subjects, comprising selecting a subject or population of subjects who would benefit from an improvement in a Psoriasis Area Severity Index (PASI) score (e.g., who would benefit from a PASI 75 response), and administering to the subject or each subject in the population of subjects an antibody, or antigen-binding portion thereof, which is capable of binding to the p40 subunit of IL-12 and/or IL-23, wherein the subject or the population of subjects upon treatment achieves a Psoriasis Area and Severity Index (PASI) 75 response in a time or a median time of less than about 70 days.

In another aspect, the invention provides methods of treating psoriasis in a subject or population of subjects, comprising selecting a subject or population of subjects who would benefit from an improvement in a Dermatology Life Quality Index (DLQI) score (e.g., a DLQI score of 0), and administering to the subject or each subject in the population of subjects an antibody, or antigen-binding portion thereof, which is capable of binding to the p40 subunit of IL-12 and/or IL-23, wherein the subject or at least 20% of the population of subjects upon treatment achieves a Dermatology Life Quality Index (DLQI) score of 0 by about week 12.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), and administering to the subject or each subject in the population an antibody, or antigen-binding portion thereof, which is capable of binding to the p40 subunit of IL-12 and/or IL-23, wherein the subject or at least about 15% of the population of subjects upon treatment achieves at least a PGA score of 0 or 1 by about week 4, and/or wherein the subject or at least about 20% of the population of subjects upon treatment achieves at least a PASI 75 response by about week 4.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response, PASI 90 response or PASI 100 response), and administering to the subject or to each subject in the population an antibody, or antigen-binding portion thereof, which is capable of binding to the p40 subunit of IL-12 and/or IL-23, wherein the subject or at least about 18% of the population of subjects upon treatment achieves a PGA score of 0 or 1 by about week 8, wherein the subject or at least about 50% of the population of subjects upon treatment achieves a PGA score of 0 or 1 by about week 8, wherein the subject or at least about 25% of the population of subjects upon treatment achieves at least a PASI 75 response by about week 8, wherein the subject or at least about 60% of the population of subjects upon treatment achieves at least a PASI 75 response by about week 8, wherein the subject or at least about 35% of the population of subjects upon treatment achieves at least a PASI 90 response by about week 8, and/or wherein the subject or at least about 10% of the population of subjects upon treatment achieves a PASI 100 response by about week 8. In one embodiment, the subject or at least about 15%, 20%, 25%, 30%, 35%, 40%, 45%, 50%, 55%, 60%, 65%, 70%, 75%, or 80% of the population of subjects upon treatment achieves a PGA score of 0 or 1 by about week 8. In another embodiment, the subject or at least about 25%, 30%, 35%, 40%, 45%, 50%, 55%, 60%, 65%, 70%, 75%, or 80% of the population of subjects upon treatment achieves a PAS 75 response by about week 8.

In another aspect, the invention provides methods of treating psoriasis in a subject or a population of subjects, comprising selecting a subject or a population of subjects who would benefit from an improvement in a PASI score (e.g., who would benefit from a PASI 75 response, PASI 90 response or PASI 100 response), and administering to the subject or to each subject in the population an antibody, or antigen-binding portion thereof, which is capable of binding to the p40 subunit of IL-12 and/or IL-23, wherein the subject or at least about 40% of the population of subjects upon treatment achieves at least a PASI 75 response by about week 12, wherein the subject or at least about 80% of the population of subjects upon treatment achieves at least a PASI 75 response by about week 12, wherein the subject or at least about 15% of the population of subjects upon treatment achieves at least a PASI 90 response by about week 12, wherein the subject or at least about 50% of the population of subjects upon treatment achieves at least a PASI 90 response by about week 12, wherein the subject or at least about 5% of the population of subjects upon treatment achieves a PASI 100 response by about week 12, and/or wherein the subject or at least about 25% of the population of subjects upon treatment achieves a PASI 100 response by about week 12. In one embodiment, the subject or at least 40%, 45%, 50%, 55%, 60%, 65%, 70%, 75%, 80%, 85%, 90% or 95% of the population of subjects achieves at least a PASI 75 response by about week 12. In another embodiment, the subject or at least 15%, 20%, 25%, 30%, 35%, 40%, 45%, 50%, 55% or 60% of the population of subjects achieves at least a PASI 90 response by about week 12. In another embodiment, the subject or at least 5%, 10%, 15%, 20%, 25%, 30% or 35% of the population of subjects achieves at least a PASI 100 response by about week 12.

In another aspect, the invention provides methods of treating psoriasis in subject or a population of subjects, comprising selecting a subject or population of subjects who would benefit from an improvement in a PASI score (e.g., who would benefit from a PASI 75 response), wherein the subject or each subject in the population of subjects was treated with a biologic previously, and administering to the subject or to each subject in the population an antibody, or antigen-binding portion thereof, which is capable of binding to the p40 subunit of IL-12 and/or IL-23, wherein the subject or at least 80% of the population of subjects achieves at least a PASI 75 response by about week 52.

In another aspect, the invention provides methods of treating psoriasis in a subject or a population of subjects, comprising selecting a subject or a population of subjects who would benefit from an improvement in a PASI score (e.g., who would benefit from a PASI 75 response), wherein neither the subject nor any subject in the population of subjects was treated with a biologic previously, and administering to the subject or to each subject in the population an antibody, or antigen-binding portion thereof, which is capable of binding to the p40 subunit of IL-12 and/or IL-23, wherein the subject or at least 80% of the population of subjects achieves at least a PASI 75 response by about week 52.

In yet another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subject was treated with a biologic previously and showed no response, and administering to the subject or each subject in the population an antibody, or antigen-binding portion thereof, which is capable of binding to the p40 subunit of IL-12 and/or IL-23, wherein the subject or at least 65% of the population of subjects achieves a PGA score of 0 or 1 by about week 12, and/or wherein the subject or at least 70% of the population of subjects achieves at least a PASI 75 response by about week 12.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subject was treated with a biologic previously and showed improvement in the PGA score or PASI 75 response, and administering to the subject or each subject in the population an antibody, or antigen-binding portion thereof, which is capable of binding to the p40 subunit of IL-12 and/or IL-23, wherein the subject or at least 75% of the population of subjects achieves a PGA score of 0 or 1 by about week 12, and/or wherein the subject or at least 75% of the population of subjects achieves at least a PASI 75 response by about week 12.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subject was treated with a biologic previously and showed no response, and administering to the subject or each subject in the population an antibody, or antigen-binding portion thereof, which is capable of binding to the p40 subunit of IL-12 and/or IL-23, wherein the subject or at least 70% of the population of subjects achieves a PGA score of 0 or 1 by about week 52, and/or wherein the subject or at least 75% of the population of subjects achieves at least a PASI 75 response by about week 52.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subject was treated with a biologic previously and showed improvement in the PGA score or the PASI 75 response, and administering to the subject or each subject in the population an antibody, or antigen-binding portion thereof, which is capable of binding to the p40 subunit of IL-12 and/or IL-23, wherein the subject or at least 75% of the population of subjects achieves a PGA score of 0 or 1 by about week 52, and/or wherein the subject or at least 75% of the population of subjects achieves at least a PASI 75 response by about week 52.

In another aspect, the invention provides methods of treating psoriasis in a subject or a population of subjects, comprising selecting a subject or a population of subjects who would benefit from an improvement in a PASI score (e.g., who would benefit from a PASI 75 response), and administering to the subject or each subject in the population an antibody, or antigen-binding portion thereof, which is capable of binding to the p40 subunit of IL-12 and/or IL-23, wherein the subject or at least 80% of the population of subjects achieves at least a PASI 75 response by about week 52, wherein each subject has a prior history of psoriatic arthritis, and/or wherein the subject or at least 80% of the population of subjects achieves at least a PASI 75 response by about week 52, wherein none of the subjects has a prior history of psoriatic arthritis.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subjects had a baseline weight of less than 100 kilograms, and administering to the subject or each subject in the population an antibody, or antigen-binding portion thereof, which is capable of binding to the p40 subunit of IL-12 and/or IL-23, wherein the subject or at least 80% of the population of subjects achieves a PGA score of 0 or 1 by about week 52, and/or wherein the subject or at least 80% of the population of subjects achieves at least a PASI 75 response by about week 52.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1) or a PASI score (e.g., who would benefit from a PASI 75 response), wherein the subject or each subject in the population of subjects had a baseline weight of greater than or equal to 100 kilograms, and administering to the subject or each subject in the population an antibody, or antigen-binding portion thereof, which is capable of binding to the p40 subunit of IL-12 and/or IL-23, wherein the subject or at least 70% of the population of subjects achieves a PGA score of 0 or 1 by about week 52, and/or wherein the subject or at least 75% of the population of subjects achieves at least a PASI 75 response by about week 52.

In another aspect, the invention provides methods of treating psoriasis in a subject or a population of subjects, comprising selecting a subject or population of subjects who would benefit from an improvement in a PGA score (e.g., who would benefit from a PGA score of 0 or 1) or a PASI score (e.g., who would benefit from a PASI 75 response), wherein the subject or each subject within the population of subjects had a baseline PASI score of less than or equal to 20, and administering to each subject in the population an antibody, or antigen-binding portion thereof, which is capable of binding to the p40 subunit of IL-12 and/or IL-23, wherein the subject or at least 80% of the population of subjects achieves a PGA score of 0 or 1 by about week 52, and/or wherein the subject or at least 80% of the population of subjects achieves at least a PASI 75 response by about week 52.

In another aspect, the invention provides methods of treating psoriasis in a subject or a population of subjects, comprising selecting a subject or population of subjects who would benefit from an improvement in a PGA score (e.g., who would benefit from a PGA score of 0 or 1) or PASI score (e.g., who would benefit from a PASI 75 response), wherein the subject or each subject within the population of subjects had a baseline PASI score of greater than 20, and administering to the subject or each subject in the population an antibody, or antigen-binding portion thereof, which is capable of binding to the p40 subunit of IL-12 and/or IL-23, wherein the subject or at least 70% of the population of subjects achieves a PGA score of 0 or 1 by about week 52, and/or wherein the subject or at least 75% of the population of subjects achieves at least a PASI 75 response by about week 52.

In another aspect, the invention provides methods of treating psoriasis in a subject or a population of subjects, comprising selecting a subject or population of subjects who would benefit from an improvement in a PGA score (e.g., who would benefit from a PGA score of 0 or 1) or a PASI score (e.g., who would benefit from a PASI 75 response), wherein the subject or each subject within the population of subjects had less than or equal to 20% body surface area (BSA) affected by psoriasis, and administering to the subject or each subject in the population an antibody, or antigen-binding portion thereof, which is capable of binding to the p40 subunit of IL-12 and/or IL-23, wherein the subject or at least 80% of the population of subjects achieves a PGA score of 0 or 1 by about week 12, and/or wherein the subject or at least 80% of the population of subjects achieves at least a PASI 75 response by about week 12.

In another aspect, the invention provides methods of treating psoriasis in a subject or a population of subjects, comprising selecting a subject or population of subjects who would benefit from an improvement in a PGA score (e.g., who would benefit from a PGA score of 0 or 1) or a PASI score (e.g., who would benefit from a PASI 75 response), wherein the subject or each subject within the population of subjects had greater than 20% body surface area (BSA) affected by psoriasis, and administering to the subject or each subject in the population an antibody, or antigen-binding portion thereof, which is capable of binding to the p40 subunit of IL-12 and/or IL-23, wherein the subject or at least 70% of the population of subjects achieves a PGA score of 0 or 1 by about week 12, and/or wherein the subject or at least 75% of the population of subjects achieves at least a PASI 75 response by about week 12.

In another aspect, the invention provides methods of treating psoriasis in a subject or a population of subjects, comprising selecting a subject or population of subjects who would benefit from an improvement in a PGA score (e.g., who would benefit from a PGA score of 0 or 1) or a PASI score (e.g., who would benefit from a PASI 75 response), wherein the subject or each subject within the population of subjects had less than or equal to 20% body surface area (BSA) affected by psoriasis, and administering to the subject or each subject in the population an antibody, or antigen-binding portion thereof, which is capable of binding to the p40 subunit of IL-12 and/or IL-23, wherein the subject or at least 80% of the population of subjects achieves a PGA score of 0 or 1 by about week 52, and/or wherein the subject or at least 85% of the population of subjects achieves at least a PASI 75 response by about week 52.

In yet another aspect, the invention provides methods of treating psoriasis in a subject or a population of subjects, comprising selecting a subject or population of subjects who would benefit from an improvement in a PGA score (e.g., who would benefit from a PGA score of 0 or 1) or a PASI score (e.g., who would benefit from a PASI 75 response), wherein the subject or each subject within the population of subjects had greater than 20% body surface area (BSA) affected by psoriasis, and administering to the subject or each subject in the population an antibody, or antigen-binding portion thereof, which is capable of binding to the p40 subunit of IL-12 and/or IL-23, wherein the subject or at least 70% of the population of subjects achieves a PGA score of 0 or 1 by about week 52, and/or wherein the subject or at least 75% of the population of subjects achieves at least a PASI 75 response by about week 52.

In another aspect, the invention provides methods of treating psoriasis in a subject or a population of subjects, comprising selecting a subject or population of subjects who would benefit from an improvement in a PASI score (e.g., who would benefit from a PASI 75 response) or a PGA score (e.g., who would benefit from a PGA score of 0 or 1), wherein the subject or each subject within the population of subjects has previously been exposed to a tumor necrosis factor- (TNF-) antagonist, and administering to the subject or each subject in the population an antibody, or antigen binding portion thereof, which is capable of binding to the p40 subunit of IL-12 and/or IL-23, wherein the subject or at least 70% of the population of subjects achieves at least a PASI 75 at about week 8, wherein the subject or at least 80% of the population of subjects achieves at least a PASI 75 at about week 8, wherein the subject or at least 50% of the population of subjects achieves a PGA score of 0 or 1 at about week 8, and/or wherein the subject or at least 60% of the population achieves a PGA score of 0 or 1 at about week 8. In one embodiment, the subject or at least 70%, 75%, 80%, 85%, 90% or 95% of the population of subjects achieves at least a PASI 75 at about week 8. In another embodiment, the subject or at least 50%, 55%, 60%, 65%, 70%, 75% or 80% of the population of subjects achieves a PGA score of 0 or 1 at about week 8.

In yet another embodiment, the invention provides methods of treating psoriasis in subject or a population of subjects, comprising selecting a subject or population of subjects who would benefit from an improvement in a PASI score (e.g., who would benefit from a PASI 75 response) or a PGA score (e.g., who would benefit from a PGA score of 0 or 1), wherein the subject or each subject within the population of subjects has previously been exposed to a tumor necrosis factor- (TNF-) antagonist, and administering to the subject or each subject in the population an antibody, or antigen binding portion thereof, which is capable of binding to the p40 subunit of IL-12 and/or IL-23, wherein the subject or at least 80% of the population of subjects achieves at least a PASI 75 at about week 52, wherein the subject or at least 75% of the population of subjects achieves a PGA score of 0 or 1 at about week 52, and/or wherein the subject or at least 85% of the population of subjects achieves a PGA score of 0 or 1 by about week 52. In one embodiment, the subject or at least about 75%, 80%, 85%, 90% or 95% of the population of subjects achieves a PGA score of 0 or 1 by about week 52.

In another aspect, the invention provides methods of treating psoriasis in a subject or a population of subjects, comprising selecting a subject or population of subjects who would benefit from an improvement in a PASI score (e.g., who would benefit from a PASI 75 response) or a PGA score (e.g., who would benefit from a PGA score of 0 or 1), wherein the subject or each subject within the population of subjects has previously been exposed to a tumor necrosis factor- (TNF-) antagonist, and administering to the subject or each subject in the population an antibody, or antigen binding portion thereof, which is capable of binding to the p40 subunit of IL-12 and/or IL-23, wherein the subject or at least 80% of the population of subjects achieves at least a PASI 75 at about week 100, wherein the subject or at least 90% of the population of subjects achieves a PASI 75 at about week 100, wherein the subject or at least 75% of the population of subjects achieves a PGA score of 0 or 1 at about week 100, and/or wherein the subject or at least 80% of the population of subjects achieves a PGA score of 0 or 1 at about week 100. In one embodiment, the subject or at least 80%, 85%, 90% or 95% of the population of subjects achieves a PASI 75 at about week 100. In another embodiment, the subject or at least 75%, 76%, 77%, 78%, 79%, 80%, 85%, 90% or 95% of the population of subjects achieves a PGA score of 0 or 1 at about week 100.

In one embodiment, the subject or each subject in the population failed to respond to treatment with a TNF-antagonist. In another embodiment, the TNF antagonist is selected from the group consisting of anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors.

In another aspect, the invention provides methods of treating psoriasis in a subject or a population of subjects, comprising selecting a subject or a population of subjects who would benefit from an improvement in a PASI score (e.g., who would benefit from a PASI 75 response), and administering to the subject or each subject in the population an antibody, or antigen binding portion thereof, which is capable of binding to the p40 subunit of IL-12 and/or IL-23, wherein the subject or at least 90% of the subjects in the population maintains at least a PASI 75 through about week 84.

In another embodiment, the invention provides methods of treating psoriasis in a subject or a population of subjects, comprising selecting a subject or a population of subjects who would benefit from an improvement in a PASI score (e.g., who would benefit from a PASI 75 response), and administering to the subject or each subject in the population an antibody, or antigen binding portion thereof, which is capable of binding to the p40 subunit of IL-12 and/or IL-23, wherein the subject or at least 90% of the subjects in the population maintains at least a PASI 75 through about week 124.

In one embodiment, the subject or subjects in the population do not suffer an adverse event during treatment with the antibody, or antigen binding portion thereof, which is capable of binding to the p40 subunit of IL-12 and/or IL-23.

In another aspect, the invention provides methods of treating psoriasis in a subject or a population of subjects, comprising selecting a subject or a population of subjects who would benefit from an improvement in a PASI score (e.g., who would benefit from a PASI 90 response or a PASI 100 response), wherein the subject or all of the subjects in the population were previously exposed to a tumor necrosis factor (TNF) antagonist, and administering to the subject or each subject in the population an antibody, or antigen binding portion thereof, which is capable of binding to the p40 subunit of IL-12 and/or IL-23, wherein the subject or at least 40% of the population of subjects achieves at least a PASI 90 at about week 8, wherein the subject or at least 50% of the population of subjects achieves at least a PASI 90 at about week 8, wherein the subject or at least 10% of the population of subjects achieves at least a PASI 100 at about week 8, wherein the subject or at least 20% of the population of subjects achieves at least a PASI 100 at about week 8, wherein the subject or at least 15% of the population of subjects achieves a PGA score of 0 at about week 8, and/or wherein the subject or at least 25% of the population of subjects achieves a PGA score of 0 at about week 8. In one embodiment, the subject or at least 40%, 45%, 50%, 55%, 60%, 65% 70% or 75% of the population of subjects achieves at least a PASI 90 at about week 8.

In another embodiment, the subject or at least 10%, 15%, 20%, 25%, or 30% of the population of subjects achieves at least a PASI 100 at about week 8. In yet another embodiment, the subject or at least 15%, 20%, 25%, 30% or 35% of the population of subjects achieves a PGA score of 0 at about week 8.

In another aspect, the invention provides methods of treating psoriasis in a subject or a population of subjects, comprising selecting a subject or a population of subjects who would benefit from an improvement in a PASI score (e.g., who would benefit from a PASI 90 response or PASI 100 response), wherein the subject or all of the subjects in the population were previously exposed to a tumor necrosis factor (TNF) antagonist, and administering to the subject or each subject in the population an antibody, or antigen binding portion thereof, which is capable of binding to the p40 subunit of IL-12 and/or IL-23, wherein the subject or at least 70% of the population of subjects achieves at least a PASI 90 at about week 52, wherein the subject or at least 80% of the population of subjects achieves at least a PASI 90 at about week 52, wherein the subject or at least 60% of the population of subjects achieves at least a PASI 100 at about week 52, wherein the subject or at least 70% of the population of subjects achieves at least a PASI 100 at about week 52, wherein the subject or at least 65% of the population of subjects achieves a PGA score of 0 at about week 52, and/or wherein the subject or at least 75% of the population of subjects achieves a PGA score of 0 at about week 52. In one embodiment, the subject or at least 70%, 75%, 80% or 85% of the population of subjects achieves at least a PASI 90 at about week 52. In another embodiment, the subject or at least 60%, 65%, 70%, or 75% of the population of subjects achieves at least a PASI 100 at about week 52. In yet another embodiment, the subject or at least 65%, 70%, 75% or 80% of the population of subjects achieves a PGA score of 0 at about week 52.

In another aspect, the invention provides methods of treating psoriasis in a subject or a population of subjects, comprising selecting a subject or a population of subjects who would benefit from an improvement in a PASI score (e.g., who would benefit from a PASI 90 response or a PASI 100 response), wherein the subject or all of the subjects in the population were previously exposed to a tumor necrosis factor (TNF) antagonist, and administering to the subject or each subject in the population an antibody, or antigen binding portion thereof, which is capable of binding to the p40 subunit of IL-12 and/or IL-23, wherein the subject or at least 70% of the population of subjects achieves at least a PASI 90 at about week 100, wherein the subject or at least 80% of the population of subjects achieves at least a PASI 90 at about week 100, wherein the subject or at least 50% of the population of subjects achieves at least a PASI 100 at about week 100, wherein the subject or at least 60% of the population of subjects achieves at least a PASI 100 at about week 100, wherein the subject or at least 55% of the population of subjects achieves a PGA score of 0 at about week 100, and/or wherein the subject or at least 65% of the population of subjects achieves a PGA score of 0 or 1 at about week 100. In one embodiment, the subject or at least 70%, 75%, 80% or 85% of the population of subjects achieves at least a PASI 90 at about week 100. In another embodiment, the subject or at least 50%, 55%, 60% or 65% of the population of subjects achieves at least a PASI 100 at about week 100. In yet another embodiment, the subject or at least 55%, 60%, 65% or 70% of the population of subjects achieves a PGA score of 0 or 1 at about week 100.

In one embodiment, the subject or each of the subjects in the population failed to respond to treatment with a TNF-antagonist. In another embodiment, the TNF antagonist is selected from the group consisting of anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1), and administering to the subject or each subject in the population an antibody, or antigen binding portion thereof, which is capable of binding to the p40 subunit of IL-12 and/or IL-23, wherein the subject or each subject in the population maintains a PGA score of 0 or 1 through about week 12.

In another aspect, the invention provides methods of treating psoriasis in a subject or a population of subjects, comprising selecting a subject or a population of subjects who would benefit from an improvement in a PASI score (e.g., who would benefit from a PASI 75 response, PASI 90 response or PASI 100 response) or a PGA score (e.g., who would benefit from a PGA score of 0 or 1), and administering to the subject or each subject in the population an antibody, or antigen binding portion thereof, which is capable of binding to the p40 subunit of IL-12 and/or IL-23, wherein the subject or at least 90% of the population of subjects maintains at least a PASI 75 through about week 96, wherein the subject or at least 80% of the population of subjects maintains at least a PASI 90 through about week 96, wherein the subject or at least 65% of the population of subjects maintains at least a PASI 100 through about week 96, and/or wherein the subject or at least 90% of the population of subjects maintains at least a PGA 0 or 1 score through about week 96.

In one embodiment, the subject or each of the subjects in the population do not suffer an adverse event during treatment with the antibody, or antigen binding portion thereof, which is capable of binding to the p40 subunit of IL-12 and/or IL-23.

In another aspect, the invention provides methods of treating psoriasis in a subject or a population of subjects, comprising selecting a subject or a population of subjects who would benefit from an improvement in a PASI score (e.g., who would benefit from a PASI 75 response or a PASI 90 response), wherein the subject or each of the subjects in the population were previously exposed to etanercept, and administering to the subject or each subject in the population an antibody, or antigen binding portion thereof, which is capable of binding to the p40 subunit of IL-12 and/or IL-23, wherein the subject or at least 70% of the population of subjects achieves at least a PASI 75 response at about week 28, wherein the subject or at least 90% of the population of subjects achieves at least a PASI 75 response at about week 28, wherein the subject or at least 50% of the population of subjects achieves at least a PASI 90 at about week 28, and/or wherein the subject or at least 75% of the population of subjects achieves at least a PASI 90 at about week 28. In one embodiment, the subject or at least 70%, 75%, 80%, 85%, 90% or 95% of the population of subjects achieves at least a PASI 75 response at about week 28. In another embodiment, the subject or at least 50%, 55%, 60%, 65%, 70%, 75% or 80% of the population of subjects achieves at least a PASI 90 at about week 28.

In another aspect, the invention provides methods of treating psoriasis in a subject or a population of subjects, comprising selecting a subject or a population of subjects who would benefit from an improvement in a PASI score (e.g., who would benefit from a PASI 75 response or a PASI 90 response), wherein the subject or each of the subjects in the population were previously exposed to etanercept, and administering to the subject or each subject in the population an antibody, or antigen binding portion thereof, which is capable of binding to the p40 subunit of IL-12 and/or IL-23, wherein the subject or at least 75% of the population of subjects achieves at least a PASI 75 at about week 88, wherein the subject or at least 85% of the population of subjects achieves at least a PASI 75 at about week 88, wherein the subject or at least 70% of the population of subjects achieves at least a PASI 90 at about week 88, and/or wherein the subject or at least 85% of the population of subjects achieves at least a PASI 90 at about week 88. In one embodiment, the subject or at least 75%, 80%, 85% or 90% of the population of subjects achieves at least a PASI 75 at about week 88. In another embodiment, the subject or at least 70%, 75%, 80%, 85%, 90% or 95% of the population of subjects achieves at least a PASI 90 at about week 88.

In another aspect, the invention provides methods of treating psoriasis in a subject or a population of subjects, comprising selecting a subject or a population of subjects who would benefit from an improvement in a PASI score (e.g., who would benefit from a PASI 100 response) or a PGA score (e.g., who would benefit from a PGA score of 0), wherein the subject or each of the subjects in the population were previously exposed to etanercept, and administering to the subject or each subject in the population an antibody, or antigen binding portion thereof, which is capable of binding to the p40 subunit of IL-12 and/or IL-23, wherein the subject or at least 20% of the population of subjects achieves at least a PASI 100 at about week 28, wherein the subject or at least 50% of the population of subjects achieves at least a PASI 100 at about week 28, wherein the subject or at least 20% of the population of subjects achieves at least a PGA 0 at about week 28, and/or wherein the subject or at least 50% of the population of subjects achieves at least a PGA 0 at about week 28. In one embodiment, the subject or at least 20%, 25%, 30%, 35%, 40%, 45%, 50% or 55% of the population of subjects achieves at least a PASI 100 score at about week 28. In another embodiment, the subject or at least about 20%, 25%, 30%, 35%, 40%, 45%, 50% or 55% of the population of subjects achieves at least a PGA 0 at about week 28.

In another aspect, the invention provides methods of treating psoriasis in a subject or a population of subjects, comprising selecting a subject or a population of subjects who would benefit from an improvement in a PASI score (e.g., who would benefit from a PASI 100 response) or a PGA score (e.g., who would benefit from a PGA score of 0 or 1), wherein the subject or each of the subjects in the population were previously exposed to etanercept, and administering to the subject or each subject in the population an antibody, or antigen binding portion thereof, which is capable of binding to the p40 subunit of IL-12 and/or IL-23, wherein the subject or at least 45% of the population of subjects achieves at least a PASI 100 at about week 88, wherein the subject or at least 55% of the population of subjects achieves at least a PASI 100 at about week 88, wherein the subject or at least 70% of the population of subjects achieves at least a PGA 0 or 1 at about week 88, wherein the subject or at least 80% of the population of subjects achieves at least a PGA 0 or 1 at about week 88, wherein the subject or at least 45% of the population of subjects achieves at least a PGA 0 at about week 88, and/or wherein the subject or at least 60% of the population of subjects achieves at least a PGA 0 at about week 88. In one embodiment, the subject or at least 45%, 50%, 55% or 60% of the population of subjects achieves at least a PASI 100 at about week 88. In another embodiment, the subject or at least 70%, 75%, 80% or 85% of the population of subjects achieves at least a PGA 0 or 1 at about week 88. In yet another embodiment, the subject or at least 45%, 50%, 55%, 60% or 65% of the population of subjects achieves at least a PGA 0 at about week 88.

In another aspect, the invention provides methods of treating psoriasis in a subject or a population of subjects, comprising selecting a subject or a population of subjects who would benefit from an improvement in a PGA score (e.g., who would benefit from a PGA score of 0 or 1), wherein the subject or each of the subjects in the population were previously exposed to etanercept, and administering to the subject or each subject in the population an antibody, or antigen binding portion thereof, which is capable of binding to the p40 subunit of IL-12 and/or IL-23, wherein the subject or at least 55% of the population of subjects achieves at least a PGA 0 or 1 at about week 28, and/or wherein the subject or at least 85% of the population of subjects achieves at least a PGA 0 or 1 at about week 28. In one embodiment, the subject or at least 55%, 60%, 65%, 70%, 75%, 80%, 85% or 90% of the population of subjects achieves at least a PGA 0 or 1 at about week 28.

In one embodiment, the subject or each of the subjects in the population did not previously achieve a PGA response of 0 or 1. In another embodiment, the subject or each of the subjects in the population previously achieved a PGA response of 0 or 1.

In one embodiment, the antibody, or antigen-binding portion thereof, is administered according to a periodicity of about once every 4 week, thereby treating psoriasis in the subject or the population of subjects. In another embodiment, the antibody, or antigen-binding portion thereof, is administered according to a periodicity of about once every 12 weeks, thereby treating psoriasis in the subject or the population of subjects.

In one embodiment, the antibody, or antigen-binding portion thereof, is administered in a) a first dose amount according to a first periodicity of about once every 4 weeks; and b) a second dose amount that is about 40-60% of the first dose amount of the antibody, or antigen-binding portion thereof, according to a second periodicity of about once every 4 weeks, thereby treating psoriasis in the subject or the population of subjects.

In another embodiment, the antibody, or antigen-binding portion thereof, is administered in a a) a first dose amount of an antibody, or antigen-binding portion thereof, which is capable of binding to the p40 subunit of IL-12 and/or IL-23, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks, thereby treating psoriasis in the subject or the population of subjects.

In one embodiment, the first dose amount is at least about 200 mg. In another embodiment, the second dose amount is at least about 100 mg.

In one embodiment, the antibody is a human antibody. In another embodiment, the antibody is ABT-874.

In one embodiment, the method comprises administering to the subject or to each subjects in the population: a) about 200 mg of ABT-874 once every four weeks for two doses; and b) about 100 mg of ABT-874 every four weeks thereafter.

In another embodiment, the method comprises administering to the subject or to each subjects in the population: a) about 200 mg of ABT-874 at weeks 0 and 4; and b) about 100 mg of ABT-874 at week 8 and every 4 weeks thereafter.

In another embodiment, the antibody is administered subcutaneously.

In one embodiment, the psoriasis is moderate to severe or chronic psoriasis. In yet another embodiment, the psoriasis is plaque psoriasis.

In another aspect, the invention provides methods for treating psoriasis in difficult to treat subjects by administering antibodies, and antigen binding portions thereof, of the invention, for example, ABT-874. Difficult to treat subjects include, for example, subjects who have been previously administered other systemic therapies or treatments and, e.g., failed to respond to, or are intolerant to, other systemic therapies or treatments. Difficult to treat subjects may also include, for example, subjects who have a contraindication to other systemic therapies or treatments. Other systemic therapies or treatments may include, e.g., non-biologics or biologics, for the treatment of psoriasis.

Accordingly, in one embodiment, the invention provides methods for treating a subject who have a contraindication to another systemic therapy or treatment, e.g., non-biologics or biologics, for the treatment of psoriasis by administering antibodies, and antigen binding portions thereof, of the invention, for example, ABT-874, to the subject. Specifically, the methods involve selecting a subject who has a contraindication to another systemic therapy or treatment, e.g., non-biologics or biologics, and administering antibodies, or antigen binding portions thereof, of the invention, for example, ABT-874, to the subject.

In another embodiment, the invention provides methods for treating subjects who have been previously administered systemic therapy or treatment, e.g., non-biologics or biologics, for the treatment of psoriasis by administering antibodies, and antigen binding portions thereof, of the invention, for example, ABT-874. The subjects may be subjects that failed to respond to the prior systemic therapy or treatment, or may be subjects that were intolerant to the prior systemic therapy or treatment. Specifically, the methods involve selecting a subject who has received prior systemic therapy or treatment, e.g., non-biologics or biologics, and administering antibodies, or antigen binding portions thereof, of the invention, e.g., ABT-874, to the subject. In one embodiment, the subject failed to respond to the prior systemic therapy or treatment. In one embodiment, the subject was intolerant to the prior systemic therapy or treatment.

In another embodiment, difficult to treat subjects include subjects who have been previously exposed to non-biologics. Nonbiologics can include, for example, ciclosporin, methotrexate and PUVA. Other non-biologics that may be used to treat psoriasis and are intended to be encompassed by these methods of the invention include those described herein, as well as those commonly known in the art. The subjects may have failed to respond to the non-biologic, or the subjects may be intolerant to the non-biologic. Specifically, the methods involve selecting a subject or a population of subjects who have received a prior non-biologic treatment, and administering antibodies, or antigen binding portions thereof, of the invention, e.g., ABT-874, to the subject. In one embodiment, the subject failed to respond to the prior non-biologic. In one embodiment, the subject was intolerant to the prior non-biologic.

In another embodiment, difficult to treat subjects include subjects who have been previously exposed to biologics. The subjects may have failed to respond to the biologic, or the subjects may be intolerant to the biologic. Biologics that may be used to treat psoriasis and are intended to be encompassed by these methods of the invention include those described herein, as well as those commonly known in the art. Specifically, the methods involve selecting a subject or a population of subjects who have received a prior biologic treatment, and administering antibodies, or antigen binding portions thereof, of the invention, e.g., ABT-874, to the subject. In one embodiment, the subject failed to respond to the prior biologic. In one embodiment, the subject was intolerant to the prior biologic. In one embodiment, the subject responded to the prior biologic.

In one embodiment, difficult to treat subjects include subjects who have been previously exposed to a tumor necrosis factor- (TNF-) antagonist. As set forth in Example 7, the data demonstrates efficacy of ABT-874 in the treatment of psoriasis in a subgroup of subjects having been previously exposed to a tumor necrosis factor- (TNF-) antagonist. Accordingly, in one embodiment, the invention provides methods for treating subjects who have been previously exposed to a tumor necrosis factor- (TNF-) antagonist, for the treatment of psoriasis by administering antibodies, and antigen binding portions thereof, of the invention, for example, ABT-874. The subjects may have failed to respond to the TNF-antagonist, or the subjects may be intolerant to the TNF-antagonist. Specifically, the methods involve selecting a subject who have received a prior TNF-antagonist treatment, and administering antibodies, or antigen binding portions thereof, of the invention, e.g., ABT-874, to the subject. In one embodiment, the subject failed to respond to the prior TNF-antagonist. In one embodiment, the subject was intolerant to the prior TNF-antagonist.

In various embodiments, the TNF antagonist includes, for example, anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors. Examples of anti-TNF antibodies include adalimumab (Humira™ or D2E7, as described in U.S. Pat. No. 6,090,382), certolizumab (Cimzia™), golimumab (Simponi™), infliximab (cA2 or Remicade™), natalizumab (Tysabri™) and CDP 571, and the anti-TNF antibody fragment CDP870. Examples of soluble p55 or p75 TNF receptors and derivatives thereof (e.g., fusion proteins) include, without limitation, Etanercept (p75TNFR1gG or Enbrel™), Pegsunercept, and p55TNFR1gG (Lenercept™).

In another aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 70% of the population of subjects achieve at least a PASI 75 at about week 8, wherein all of the subjects were previously exposed to a tumor necrosis factor- (TNF-) antagonist. In certain embodiments, at least 50%, 55%, 60%, or 65% of the population achieves a PASI 75. In certain embodiments, at least 75%, 80%, or 85% of the population achieves a PASI 75.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 80% of the population of subjects achieve at least a PASI 75 at about week 52, wherein all of the subjects were previously exposed to a tumor necrosis factor- (TNF-) antagonist. In certain embodiments, at least 60%, 65%, 70%, or 75% of the population achieves a PASI 75 at about week 52. In certain embodiments, at least 85%, 90%, or 95% of the population achieves a PASI 75 at about week 52.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 80% of the population of subjects achieve at least a PASI75 at about week 100, wherein all of the subjects were previously exposed to a tumor necrosis factor- (TNF-) antagonist. In certain embodiments, at least 60%, 65%, 70%, or 75% of the population achieves a PASI 75 at about week 100. In certain embodiments, at least 85%, 90%, 93% or 95% of the population achieves a PASI 75 at about week 100.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 50% of the population of subjects achieve a PGA score of 0 or 1 at about week 8, wherein all of the subjects were previously exposed to a tumor necrosis factor- (TNF-) antagonist. In certain embodiments, at least 55%, 60%, or 65% of the population achieves a PGA score of 0 or 1 at about week 8. In certain embodiments, at least 35%, 40%, 45% of the population achieves a PGA score of 0 or 1 at bout week 8.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 75% of the population of subjects achieve a PGA score of 0 or 1 at about week 52, wherein all of the subjects were previously exposed to a tumor necrosis factor- (TNF-) antagonist. In certain embodiments, at least 60%, 65%, or 70% of the population achieves a PGA score of 0 or 1. In certain embodiments, at least 80%, 85%, or 90% of the population achieves a PGA score of 0 or 1.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 75% of the population of subjects achieve a PGA score of 0 or 1 at about week 100, wherein all of the subjects were previously exposed to a tumor necrosis factor- (TNF-) antagonist. In certain embodiments, at least 60%, 65%, or 70% of the population achieves a PGA score of 0 or 1. In certain embodiments, at least 80%, 85%, or 90% of the population achieves a PGA score of 0 or 1.

In one embodiment, the subjects of the population failed to respond to treatment with a TNF-antagonist. In one embodiment, the subjects of the population responded to treatment with a TNF-agonist.

In certain embodiments of the foregoing aspects, the TNF antagonist include, for example, anti-TNF antibodies (e.g., chimeric, humanized or human antibodies), anti-TNF antibody fragments, soluble p55 or p75 TNF receptors and derivatives thereof, soluble IL-13 receptor (sIL-13), and TNFα converting enzyme (TACE) inhibitors. Examples of anti-TNF antibodies include adalimumab (Humira™ or D2E7, as described in U.S. Pat. No. 6,090,382), certolizumab (Cimzia™), golimumab (Simponi™), infliximab (cA2 or Remicade™), natalizumab (Tysabri™) and CDP 571, and the anti-TNF antibody fragment CDP870. Examples of soluble p55 or p75 TNF receptors and derivatives thereof (e.g., fusion proteins) include, without limitation, Etanercept (p75TNFR1gG or Enbrel™), Pegsunercept, and p55TNFR1gG (Lenercept™).

In one aspect, the invention provides methods of treating psoriasis in a subject, comprising administering to each subject in the population an antibody, or antigen binding portion thereof, which is capable of binding to the p40 subunit of IL-12 and/or IL-23, wherein the subject achieves at least a PASI 75 at about week 84.

In one aspect, the invention provides methods of treating psoriasis in a subject, comprising administering to each subject in the population an antibody, or antigen binding portion thereof, which is capable of binding to the p40 subunit of IL-12 and/or IL-23, wherein the subject achieves at least a PASI 75 at about week 124.

In various embodiments, the subject achieves at least a PASI 75 at about week 84, week 86, week 88, week 90, week 92, week 94, week 96, week 98, week 100, week 102, week 104, week 106, week 108, week 110, week 112, week 114, week 116, week 118, week 120, week 122, or at week 124 of treatment. In various embodiments, the subject achieves and then maintains at least a PASI 75 through about week 84, week 86, week 88, week 90, week 92, week 94, week 96, week 98, week 100, week 102, week 104, week 106, week 108, week 110, week 112, week 114, week 116, week 118, week 120, week 122, or through week 124 of treatment.

In certain embodiments, the subject does not suffer an adverse event during treatment with the antibody, or antigen binding portion thereof, which is capable of binding to the p40 subunit of IL-12 and/or IL-23.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 90% of the population of subjects achieve at least a PASI 75 at about week 84.

In one aspect, the invention provides methods of treating psoriasis in a population of subjects, comprising administering to each subject in the population an antibody, or antigen binding portion thereof, which is capable of binding to the p40 subunit of IL-12 and/or IL-23, wherein at least 90% of the population of subjects achieve at least a PASI 75 at about week 124.

In some embodiments, at least 91%, 92%, 93%, 94%, 95%, 96%, 97%, 98% or more of the population of subjects achieves at least a PASI 75 at about week 84. In some embodiments, at least 91%, 92%, 93%, 94%, 95%, 96%, 97%, 98% or more of the population of subjects achieves at least a PASI 75 at about week 124. In various embodiments, at least 90%, 95%, 98% or more of the population of subjects achieves at least a PASI 75 at about week 84, week 86, week 88, week 90, week 92, week 94, week 96, week 98, week 100, week 102, week 104, week 106, week 108, week 110, week 112, week 114, week 116, week 118, week 120, week 122, or at week 124 of treatment. In various embodiments, at least 90%, 95%, 98% or more of the population of subjects achieves and then maintains at least a PASI 75 through about week 84, week 86, week 88, week 90, week 92, week 94, week 96, week 98, week 100, week 102, week 104, week 106, week 108, week 110, week 112, week 114, week 116, week 118, week 120, week 122, or through week 124 of treatment.

In certain embodiments, the subjects of the population do not suffer an adverse event during treatment with the antibody, or antigen binding portion thereof, which is capable of binding to the p40 subunit of IL-12 and/or IL-23.

In one embodiment, the antibody, or antigen-binding portion thereof, is administered according to a periodicity of about once every 4 week, thereby treating psoriasis in the subjects. In another embodiment, the antibody, or antigen-binding portion thereof, is administered according to a periodicity of about once every 12 weeks, thereby treating psoriasis in the subject.

In another embodiment, the antibody, or antigen-binding portion thereof, is administered in a a) a first dose amount according to a first periodicity of about once every 4 weeks; and b) a second dose amount that is about 40-60% of the first dose amount of the antibody, or antigen-binding portion thereof, according to a second periodicity of about once every 4 weeks, thereby treating psoriasis in the subject.

In another embodiment, the antibody, or antigen-binding portion thereof, is administered in a a) a first dose amount of an antibody, or antigen-binding portion thereof, which is capable of binding to the p40 subunit of IL-12 and/or IL-23, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks, thereby treating psoriasis in the subject.

In another embodiment, the first dose amount is at least about 200 mg.

In another embodiment, the second dose amount is at least about 100 mg.

In another embodiment, the antibody is a human antibody.

In another embodiment, the antibody is ABT-874.

In another embodiment, the method comprises administering to the subject or to each subject in the population: a) about 200 mg of ABT-874 once every four weeks for two doses; and b) about 100 mg of ABT-874 every four weeks thereafter.

In another embodiment, the method comprises administering to the subject or to each subject in the population: a) about 200 mg of ABT-874 at weeks 0 and 4; and b) about 100 mg of ABT-874 at week 8 and every 4 weeks thereafter.

In another embodiment, the antibody is administered subcutaneously.

In another embodiment, the psoriasis is moderate to severe or chronic psoriasis.

In a further embodiment, the psoriasis is plaque psoriasis.

In one aspect, the invention provides methods of treating psoriasis in a subject comprising administering to the subject a first dose amount of an antibody, or antigen-binding portion thereof, which is capable of binding to the p40 subunit of IL-12 and/or IL-23, according to a periodicity, and administering a second dose amount of the antibody, or antigen-binding portion thereof, at the same periodicity, thereby treating psoriasis in the subject.

In another aspect, the invention provides methods of treating psoriasis in a subject comprising administering to the subject a first dose amount of an antibody, or antigen-binding portion thereof, which is capable of binding to the p40 subunit of IL-12 and/or IL-23, according to a first periodicity, and administering a second dose amount of the antibody, or antigen-binding portion thereof, according to a second periodicity, thereby treating psoriasis in the subject.

In various embodiments, the first dose amount of the antibody, or antigen-binding portion thereof, is at least about 100 mg to about 200 mg, is at least about 100 mg, or is at least about 200 mg. In other embodiments, the first dose amount of the antibody, or antigen-binding portion thereof, is about 100 mg, 110 mg, about 120 mg, about 130 mg, about 140 mg, about 150 mg, about 160 mg, about 170 mg, about 180 mg, about 190 mg, about 200 mg.

The second dose amount of the antibody, or antigen-binding portion thereof, may be the same as the first dose amount of the antibody, or antigen-binding portion thereof, or different than the first dose amount of the antibody, or antigen-binding portion thereof. In various embodiments, the second dose amount of the antibody, or antigen-binding portion thereof, is at least about 100 mg to about 200 mg, is at least about 200 mg, or is at least about 100 mg. In other embodiment, the second dose amount of the antibody, or antigen-binding portion thereof, is about 40-60% of the first dose amount of the antibody, or antigen-binding portion thereof, or about 190-210% of the first dose amount of the antibody, or antigen-binding portion thereof. In other embodiments, the first dose amount of the antibody, or antigen-binding portion thereof, is about 100 mg, 110 mg, about 120 mg, about 130 mg, about 140 mg, about 150 mg, about 160 mg, about 170 mg, about 180 mg, about 190 mg, 200 mg.

The first and second periodicities of administration of the antibody, or antigen-binding portion thereof, may be about once a week, about once every other week, about once every four weeks. In one embodiment, the second periodicity of administration of the antibody, or antigen-binding portion thereof, is about once every 30-200 days.

The duration of the first periodicity may be about 12 weeks, about 8 weeks, about 4 weeks, about 2 weeks, or about 1 week. The duration of the first periodicity may be at least about 12 weeks, at least about 8 weeks, at least about 4 weeks, at least about 2 weeks, or at least about 1 week.

The duration of the second periodicity may be about 60 weeks, about 44 weeks, about 12 weeks, about 4 weeks, about 2 weeks, or about 1 week. The duration of the second periodicity may be at least about 60 weeks, at least about 44 weeks, at least about 12 weeks, at least about 4 weeks, at least about 2 weeks, or at least about 1 week.

In one embodiment, the second dose amount is administered to the subject upon a flare of psoriasis. In another embodiment, the second dose amount is administered to the subject prior to a flare of psoriasis.

The flare of psoriasis may be indicated by loss of a Psoriasis Area and Severity Index (PASI) 90 response, by loss of a Psoriasis Area and Severity Index (PASI) 75 response, by loss of a Psoriasis Area and Severity Index (PASI) 50 response, or by loss of a clear or minimal Physician's Global Assessment (PGA) rating.

The loss of a PASI response may be loss of PASI response of a single body region, loss of PASI response of two body regions, loss of PASI response of three body regions, or loss of PASI response of four body regions.

The body region may be trunk, lower extremities, upper extremities, or head and neck.

In another aspect, the invention provides a method of treating psoriasis in a subject comprising administering to the subject an antibody, or antigen-binding portion thereof, which is capable of binding to the p40 subunit of IL-12 and/or IL-23, according to a periodicity of about once every 4 weeks, thereby treating psoriasis in the subject.

In yet another aspect, the invention provides a method of treating psoriasis in a subject comprising administering to the subject an antibody, or antigen-binding portion thereof, which is capable of binding to the p40 subunit of IL-12 and/or IL-23, according to a periodicity of about once every 12 weeks, thereby treating psoriasis in the subject.

In a related aspect, the invention provides a method of treating psoriasis in a subject comprising administering to the subject: a) a first dose amount of an antibody, or antigen-binding portion thereof, which is capable of binding to the p40 subunit of IL-12 and/or IL-23; and b) a second dose amount that is about 40-60% of the first dose amount of the antibody, or antigen-binding portion thereof, according to a periodicity of about once every 12 weeks, thereby treating psoriasis in the subject.

In one embodiment, the subject achieves at least a PGA score of 0 or 1. In one embodiment, the subject achieves at least a PASI 75 response. In one embodiment, the subject achieves at least a PASI 90 response. In one embodiment, the subject achieves at least a PASI 100 response. In one embodiment, the subject maintains the PGA score of 0 or 1 during treatment. In one embodiment, the subject maintains the PASI 75 response during treatment. In one embodiment, the subject maintains the PASI 90 response during treatment.

In one embodiment, the first dose amount is at least about 200 mg.

In one embodiment, the second dose amount is at least about 100 mg.

In another aspect, the invention provides a method of treating psoriasis in a subject comprising administering to the subject: a) a first dose amount of an antibody, or antigen-binding portion thereof, which is capable of binding to the p40 subunit of IL-12 and/or IL-23, according to a first periodicity of about once every 4 weeks; and b) administering a second dose amount that is about 40-60% of the first dose amount of the antibody, or antigen-binding portion thereof, according to a second periodicity of about once every 4 weeks, thereby treating psoriasis in the subject.

In one embodiment, the first dose amount is at least about 200 mg.

In one embodiment, the second dose amount is at least about 100 mg.

In one embodiment, the duration of the first periodicity is at least about 8 weeks.

In one embodiment, the duration of the second periodicity is at least about 4 weeks, at least about 16 weeks, or at least about 44 weeks.

In another aspect, the invention provides a method of treating psoriasis in a subject comprising administering to the subject: a) a first dose amount of an antibody, or antigen-binding portion thereof, which is capable of binding to the p40 subunit of IL-12 and/or IL-23, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks, thereby treating psoriasis in the subject.

In one embodiment, the first dose amount is at least about 200 mg.

In one embodiment, the second dose amount is at least about 100 mg.

In one embodiment, the duration of the first periodicity is at least about 8 weeks.

In one embodiment, the duration of the second periodicity is at least about 4 weeks.

In one embodiment, the duration of the third periodicity is at least about 12 weeks or at least about 36 weeks.

In one embodiment, the subject achieves a PGA score of 0 or 1, e.g., by about week 12. In one embodiment, the subject achieves at least a PASI 75 response, e.g., by about week 12. In one embodiment, the subject achieves at least a PASI 90 response, e.g., by about week 12. In one embodiment, the subject achieves at least a PASI 100 response, e.g., by about week 12.

In one embodiment, the subject maintains the PGA score of 0 or 1 through the duration of treatment. In one embodiment, the subject maintains the PASI 75 response through the duration of treatment. In one embodiment, the subject maintains the PASI 90 response through the duration of treatment.

In another aspect, the invention provides a method of treating psoriasis in a population of subjects, comprising administering to each subject in the population an antibody, or antigen-binding portion thereof, which is capable of binding to the p40 subunit of IL-12 and/or IL-23, wherein at least 60% of the population of subjects achieve a PASI 75 response by about week 12.

In yet another aspect, the invention provides a method of treating psoriasis in a population of subjects comprising administering to each subject in the population an antibody, or antigen-binding portion thereof, which is capable of binding to the p40 subunit of IL-12 and/or IL-23, wherein at least 25% of the population of subjects achieve a PASI 90 response by about week 12.

In still another aspect, the invention provides a method of treating psoriasis in a population of subjects comprising administering to each subject in the population an antibody, or antigen-binding portion thereof, which is capable of binding to the p40 subunit of IL-12 and/or IL-23, wherein at least 10% of the population of subjects achieve a PASI 100 response by about week 12.

In one embodiment, the method comprises administering to each subject in the population: a) a first dose amount of the antibody, or antigen-binding portion thereof, according to a first periodicity of about once every 4 weeks; and b) administering a second dose amount that is about 40-60% of the first dose amount of the antibody, or antigen-binding portion thereof, according to a second periodicity of about once every 4 weeks.

In one embodiment, the method comprises administering to each subject in the population: a) a first dose amount of the antibody, or antigen-binding portion thereof, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks.

In one embodiment, the antibody is administered subcutaneously.

In one embodiment, the antibody is a human antibody. In a preferred embodiment, the antibody is ABT-874.

In one embodiment, the subject or population of subjects achieves at least a PASI 75 response by about week 24 or at least a PASI 75 response by about week 52. In another embodiment, the subject or population of subjects achieves at least a PGA score of 0 or 1 by about week 24 or at least a PGA score of 0 or 1 by about week 52.

In another aspect, the invention is directed to a method of treating psoriasis in a population of subjects, by administering to each subject in the population an antibody, or antigen-binding portion thereof, which is capable of binding to the p40 subunit of IL-12 and/or IL-23, wherein at least 41% of the population of subjects achieve at least a PASI 75 response by about week 24.

In yet another aspect, the invention is directed to a method of treating psoriasis in a population of subjects, by administering to each subject in the population an antibody, or antigen-binding portion thereof, which is capable of binding to the p40 subunit of IL-12 and/or IL-23, wherein at least 35% of the population of subjects achieve at least a PGA score of 0 or 1 by about week 24.

In a further aspect, the invention is directed to a method of treating psoriasis in a population of subjects, by administering to each subject in the population an antibody, or antigen-binding portion thereof, which is capable of binding to the p40 subunit of IL-12 and/or IL-23, wherein at least 25% of the population of subjects achieve at least a PASI 75 response by about week 52.

In another aspect, the invention is directed to a method of treating psoriasis in a population of subjects, by administering to each subject in the population an antibody, or antigen-binding portion thereof, which is capable of binding to the p40 subunit of IL-12 and/or IL-23, wherein at least 21% of the population of subjects achieve at least a PGA score of 0 or 1 by about week 52.

In certain embodiments of the foregoing aspects, the subject or population of subjects achieves (i) an improvement in a Dermatology Life Quality Index (DLQI) score or mean Dermatology Life Quality Index (DLQI) score of at least about −9; (ii) an improvement in a Short Form 36 Health Survey Physical Component Summary (PCS) score or mean Physical Component Summary (PCS) score of at least about 2; (iii) an improvement in a Short Form 36 Health Survey Mental Component Summary (MCS) score or mean Short Form 36 Health Survey Mental Component Summary (MCS) score of at least about 4; (iv) an improvement in a visual analog scale score or mean visual analog scale score for psoriasis-related pain (VAS-Ps) of at least about −25; (v) an improvement in a visual analog scale score for psoriatic arthritis-related pain (VAS-PsA) or mean visual analog scale score for psoriatic arthritis-related pain (VAS-PsA) of at least about −32; and/or (vi) a minimum clinically important difference (MCID) response rate for psoriasis-related pain (VAS-Ps) of at least about 60%.

In various aspects, the invention is directed to a method of treating psoriasis in a population of subjects comprising administering to each subject in the population an antibody, or antigen-binding portion thereof, which is capable of binding to the p40 subunit of IL-12 and/or IL-23, wherein the population of subjects achieves (i) a minimum clinically important difference (MCID) response rate for Dermatology Life Quality Index (DLQI) of at least about 70% by about week 12; (ii) a minimum clinically important difference (MCID) response rate for Dermatology Life Quality Index (DLQI) of at least about 81% by about week 52; (iii) a minimum clinically important difference (MCID) response rate for Total Activity Impairment (TAI) of at least about 45% by about week 12; and/or (iv) a minimum clinically important difference (MCID) response rate for Total Activity Impairment (TAI) of at least about 57% by about week 52. In one embodiment, the antibody, or antigen-binding portion thereof, is administered once every four weeks. In another embodiment, the antibody, or antigen-binding portion thereof, is administered once every 12 weeks.

In further aspects, the invention is directed to a method of treating psoriasis in a population of subjects, by administering to each subject in the population an antibody, or antigen-binding portion thereof, which is capable of binding to the p40 subunit of IL-12 and/or IL-23, wherein (i) at least 65% of the population of subjects achieve at least a PGA 0/1 response by about week 12, wherein each subject was treated with a biologic prior to administration of the antibody; (ii) at least 74% of the population of subjects achieve at least a PASI 75 response by about week 12, wherein each subject was treated with a biologic prior to administration of the antibody; (iii) at least 78% of the population of subjects achieve at least a PGA 0/1 response by about week 12, wherein none of the subjects were treated with a biologic prior to administration of the antibody; (iv) at least 82% of the population of subjects achieve at least a PASI 75 response by about week 12, wherein none of the subjects were treated with a biologic prior to administration of the antibody; (v) at least 78% of the population of subjects achieve at least a PGA 0/1 response by about week 52, wherein each subject was treated with a biologic prior to administration of the antibody; (vi) at least 79% of the population of subjects achieve at least a PGA 0/1 response by about week 52, wherein none of the subjects were treated with a biologic prior to administration of the antibody; (vii) at least 71% of the population of subjects achieve at least a PGA 0/1 response by about week 12, wherein each subject has a prior history of psoriatic arthritis; (viii) at least 78% of the population of subjects achieve at least a PASI 75 response by about week 12, wherein each subject has a prior history of psoriatic arthritis; (ix) at least 77% of the population of subjects achieve at least a PGA 0/1 response by about week 12, wherein none of the subjects has a prior history of psoriatic arthritis; (x) at least 81% of the population of subjects achieve at least a PASI 75 response by about week 12, wherein none of the subjects has a prior history of psoriatic arthritis; (xi) at least 77% of the population of subjects achieve at least a PGA 0/1 response by about week 52, wherein each subject has a prior history of psoriatic arthritis; and/or (xii) at least 79% of the population of subjects achieve at least a PGA 0/1 response by about week 52, wherein none of the subjects has a prior history of psoriatic arthritis.

In yet another aspect, the invention is directed to methods for decreasing the risk that a subject treated with an antibody, or antigen binding portion thereof, which is capable of binding to the p40 subunit of IL-12 and/or IL-23, will develop a Major Adverse Cardiovascular Event (MACE). The methods include (a) selecting a subject having less that 2 risk factors selected from the group consisting of (i) a body mass index (BMI) of greater than 30, (ii) a history of diabetes mellitus, (iii) blood pressure greater than 140/90, (iv) a history of myocardial infarction, (v) a history of angina requiring hospitalization, (vi) a history of coronary artery disease requiring revascularization, (vii) a history of peripheral artery disease, (viii) a history of congestive heart failure requiring hospitalization, (ix) a history of stroke or transient ischemic attack; and (b) administering the antibody, or antigen binding portion thereof to the selected subject; thereby decreasing the risk that the subject will develop a Major Adverse Cardiovascular Event. In a particular embodiment, the antibody is ABT-874 or ustekinumab.

In certain embodiments, the subject has 0 or 1 risk factor. In certain embodiments, the MACE is myocardial infarction and/or cerebrovascular stroke.

In other embodiments, the antibody, or antigen binding portion thereof, is administered to the selected subject in a first dose amount of at least about 100 mg to about 200 mg. In a further embodiment, the antibody, or antigen binding portion thereof, is administered to the selected subject in a second dose amount of at least about 100 mg to about 200 mg. In certain embodiments, the risk factors are re-evaluated prior to administration of the second dose amount to the selected subject.

In certain embodiments of the various aspects of the invention, the subject achieves at least a 50% reduction in PASI score. In one aspect the subject achieves at least a 50% reduction in PASI score by about week 4.

In other embodiments of the various aspects of the invention, the subject achieves at least an 80% reduction in PASI score. In one aspect the subject achieves at least an 80% reduction in PASI score by about week 12.

In further aspects, the invention is directed to a method of treating psoriasis in a population of subjects, comprises administering to each subject in the population an antibody, or antigen-binding portion thereof, which is capable of binding to the p40 subunit of IL-12 and/or IL-23, wherein: (i) at least 69% of the population of subjects achieve at least a PGA 0/1 response by about week 12, wherein each subject had a baseline PASI greater than 20 prior to administration of the antibody; (ii) at least 79% of the population of subjects achieve at least a PGA 0/1 response by about week 12, wherein each subject had a baseline PASI less than or equal to 20 prior to administration of the antibody; (iii) at least 79% of the population of subjects achieve at least a PASI 75 response by about week 12, wherein each subject had a baseline PASI greater than 20 prior to administration of the antibody; (iv) at least 81% of the population of subjects achieve at least a PASI 75 response by about week 12, wherein each subject had a baseline PASI less than or equal to 20 prior to administration of the antibody; (v) at least 67% of the population of subjects achieve at least a PGA 0/1 response by about week 12, wherein each subject had a baseline weight of greater than or equal to 100 kilograms prior to administration of the antibody; (vi) at least 80% of the population of subjects achieve at least a PGA 0/1 response by about week 12, wherein each subject had a baseline weight of less than 100 kilograms prior to administration of the antibody; (vii) at least 72% of the population of subjects achieve at least a PASI 75 response by about week 12, wherein each subject had a baseline weight of greater than or equal to 100 kilograms prior to administration of the antibody; and/or (viii) at least 85% of the population of subjects achieve at least a PASI 75 response by about week 12, wherein each subject had a baseline weight of less than 100 kilograms prior to administration of the antibody.

In still further aspects, the invention is directed to a method of treating psoriasis in a population of subjects comprises administering to each subject in the population an antibody, or antigen-binding portion thereof, which is capable of binding to the p40 subunit of IL-12 and/or IL-23, wherein: (i) at least 41% of the population of subjects maintains at least a PGA 0/1 response through at least week 52 of treatment; (ii) at least 79% of the population of subjects maintains at least a PGA 0/1 response through at least week 52 of treatment; (iii) at least 45% of the population of subjects maintains at least a PASI 75 response through at least week 52 of treatment; (iv) at least 82% of the population of subjects maintains at least a PASI 75 response through at least week 52 of treatment; (v) at least 23% of the population of subjects maintains at least a PASI 75 response through at least week 52 of treatment; and/or (vi) at least 63% of the population of subjects maintains at least a PASI 75 response through at least week 52 of treatment.

In certain embodiments of the various aspects of the invention, the method of treating psoriasis comprises administering to each subject in a population: a) a first dose amount of the antibody, or antigen-binding portion thereof, according to a first periodicity of about once every 4 weeks; and b) administering a second dose amount that is about 40-60% of the first dose amount of the antibody, or antigen-binding portion thereof, according to a second periodicity of about once every 4 weeks.

In other embodiments of the various aspects of the invention, the method of treating psoriasis comprises administering to each subject in a population: a) a first dose amount of the antibody, or antigen-binding portion thereof, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks.

In certain embodiments of the various aspects of the invention, a subject treated for psoriasis achieves a PGA of 0 or 1 in less than about 171 days. In some embodiments, a subject treated for psoriasis achieves a PGA of 0 or 1 in less than about 30, 40, 50, 60, 70, 80, 85, 90, 95, 100, 105, 110, 115, 120, 125, 130, 135, 140, 145, 150, 155, 160, 165, 166, 167, 168, 169 or 170 days. In certain embodiments, a patient achieves a PGA of 0 or 1 by about 69 days.

In related embodiments of the various aspects of the invention, the patient achieves a PASI 75 response in less than about 140 days. In some embodiments, a subject treated for psoriasis achieves a PASI 75 in less than about 30, 40, 50, 60, 70, 80, 85, 90, 95, 100, 105, 110, 115, 120, 125, 130, 135, 136, 137, 138 or 139 days. In certain embodiments, the patient achieves a PASI 75 by about 56 days.

In still other embodiments of the various aspects of the invention, the subject achieves at least a 60% improvement in PASI score and maintain at least a 60% improvement in PASI score, e.g., through at least week 52 of treatment.

In another aspect, the invention is directed to a method of treating psoriasis in a population of subjects, comprising administering to each subject in the population an antibody, or antigen-binding portion thereof, which is capable of binding to the p40 subunit of IL-12 and/or IL-23, wherein: (i) at least 10% of the population of subjects achieves a PGA score of 0 by week 24 of treatment; (ii) at least 5% of the population of subjects achieve at least a PASI 50 response by about week 2; (iii) at least 70% of the population of subjects achieve at least a PASI 50 response and maintain at least a PASI 50 response through at least week 52 of treatment; (iv) at least 5% of the population of subjects achieve at least a PASI 75 response by about week 4; (v) at least 40% of the population of subjects achieve at least a PASI 75 response and maintain at least a PASI 75 response through at least week 52 of treatment; (vi) at least 10% of the population of subjects achieve at least a PASI 90 response by about week 8; (vii) at least 25% of the population of subjects achieve at least a PASI 90 response and maintain at least a PASI 90 response through at least week 52 of treatment; (viii) at least 5% of the population of subjects achieve at least a PASI 100 response by about week 8; (ix) at least 10% of the population of subjects achieve at least a PASI 100 response and maintain at least a PASI 100 response through at least week 52 of treatment; (x) at least 5% of the population of subjects achieve at least a PGA score of 0 or 1 by about week 4; and/or (xi) at least 35% of the population of subjects achieve at least a PGA score of 0 or 1 and maintain at least a PGA score of 0 or 1 through at least week 52 of treatment.

In certain embodiments of the various aspects of the invention, the subject achieves a Nail Psoriasis Severity Index (NAPSI) score of about 2.1 or less. In certain embodiments, the subject achieves a Nail Psoriasis Severity Index (NAPSI) score of about 2.1 or less by about week 24. In related embodiments of the various aspects of the invention, the subject achieves a Nail Psoriasis Severity Index (NAPSI) score of about 1.2 or less. In certain embodiments, the subject achieves a Nail Psoriasis Severity Index (NAPSI) score of about 1.2 or less by about week 52.

In other embodiments of the various aspects of the invention, the subject achieves a Dermatology Life Quality Index (DLQI) score of about 0 or 1. In certain embodiments, the subject achieves a Dermatology Life Quality Index (DLQI) score of about 0 or 1 by about week 24 or by about week 52.

In certain embodiments, the subject achieves a clinically meaningful reduction in Dermatology Life Quality Index (DLQI) score. A clinically meaningful reduction in Dermatology Life Quality Index (DLQI) score may be, e.g., a decrease of greater than 5 points in DLQI score. In one embodiment, the subject achieves a clinically meaningful reduction in DLQI score by about week 24. In one embodiment, the subject achieves a clinically meaningful reduction in DLQI score by about week 52.

In certain embodiments, the subject or population of subjects achieves an improvement in Dermatology Life Quality Index (DLQI) score of at least about −7, e.g., by week 12.

In a further aspect, the invention is directed to a method of treating psoriasis in a population of subjects, comprising administering to each subject in the population an antibody, or antigen-binding portion thereof, which is capable of binding to the p40 subunit of IL-12 and/or IL-23, wherein: (i) at least 35% of the population of subjects achieves a Dermatology Life Quality Index (DLQI) score of 0 or 1 by about week 24; (ii) at least 18% of the population of subjects achieves a Dermatology Life Quality Index (DLQI) score of 0 or 1 by about week 52; (iii) at least 50% of the population of subjects achieves a clinically meaningful reduction in Dermatology Life Quality Index (DLQI) score by about week 24; and/or (iv) at least 20% of the population of subjects achieves a clinically meaningful reduction in Dermatology Life Quality Index (DLQI) score by about week 52.

In several embodiments of the various aspects of the invention, the subject achieves a minimum clinically important difference (MCID) in one or more health-related quality of life outcomes selected from the group consisting of Dermatology Life Quality Index (DLQI), Total Activity Impairment (TAI), Ps-related (VAS-Ps) pain, psoriatic arthritis-related (VAS-PsA) pain, Short Form 36 Health Survey Mental Component Summary score (MCS) and Short Form 36 Health Survey Mental Component Summary score (PCS). In various embodiments, the subject achieves a minimum clinically important difference (MCID) in two, three, four, five or all six of Dermatology Life Quality Index (DLQI), Total Activity Impairment (TAI), Ps-related (VAS-Ps) pain, psoriatic arthritis-related (VAS-PsA) pain, Short Form 36 Health Survey Mental Component Summary score (MCS) or Short Form 36 Health Survey Physical Component Summary score (PCS).

In related embodiments, the population of subjects achieves a minimum clinically important difference (MCID) response rate for one or more health-related quality of life outcomes selected from the group consisting of Dermatology Life Quality Index (DLQI), Total Activity Impairment (TAI), Ps-related (VAS-Ps) pain, psoriatic arthritis-related (VAS-PsA) pain, Short Form 36 Health Survey Mental Component Summary score (MCS) and Short Form 36 Health Survey Mental Component Summary score (PCS). In various embodiments, the population of subjects achieves a minimum clinically important difference (MCID) response rate for two, three, four, five or all six of Dermatology Life Quality Index (DLQI), Total Activity Impairment (TAI), Ps-related (VAS-Ps) pain, psoriatic arthritis-related (VAS-PsA) pain, Short Form 36 Health Survey Mental Component Summary score (MCS) or Short Form 36 Health Survey Physical Component Summary score (PCS).

In one embodiment of all of the foregoing aspects of the invention, the method comprises administering to the subject or to each subject in the population: a) a first dose amount of the antibody, or antigen-binding portion thereof, according to a first periodicity of about once every 4 weeks; and b) administering a second dose amount that is about 40-60% of the first dose amount of the antibody, or antigen-binding portion thereof, according to a second periodicity of about once every 4 weeks.

In another embodiment of all of the foregoing aspects of the invention, the method comprises administering to the subject or to each subject in the population: a) about 200 mg of ABT-874 once every four weeks for two doses; and b) about 100 mg of ABT-874 every four weeks thereafter.

In still another embodiment of all of the foregoing aspects of the invention, the method comprises administering to the subject or to each subject in the population: a) about 200 mg of ABT-874 at weeks 0 and 4; and b) about 100 mg of ABT-874 at week 8 and every 4 weeks thereafter. In one embodiment, the antibody is ABT-874 (i.e., Briakinumab™).

In a further aspect, the invention provides a method of treating psoriasis in a subject comprising administering to the subject: a) about 200 mg of an antibody, or antigen-binding portion thereof, which is capable of binding to the p40 subunit of IL-12 and/or IL-23, once every four weeks for two doses; and b) about 100 mg of the antibody, or antigen-binding portion thereof, every four weeks thereafter, thereby treating psoriasis in the subject. In one embodiment, the antibody is ABT-874. In one embodiment, the psoriasis is plaque psoriasis, e.g., chronic plaque psoriasis, such as moderate to severe chronic plaque psoriasis.

In yet a further aspect, the invention provides a method of treating psoriasis in a subject comprising administering to the subject: a) about 200 mg of an antibody, or antigen-binding portion thereof, which is capable of binding to the p40 subunit of IL-12 and/or IL-23, at weeks 0 and 4; and b) about 100 mg of the antibody, or antigen-binding portion thereof, at week 8 and every 4 weeks thereafter, thereby treating psoriasis in the subject. In one embodiment, the antibody is ABT-874. In one embodiment, the psoriasis is plaque psoriasis, e.g., chronic plaque psoriasis, such as moderate to severe chronic plaque psoriasis.

In a still further aspect, the invention provides a method of treating psoriasis in a subject comprising administering to the subject: a) about 200 mg of ABT-874 once every four weeks for two doses; and b) about 100 mg of ABT-874 every four weeks thereafter, thereby treating psoriasis in the subject. In one embodiment, the antibody is ABT-874. In one embodiment, the psoriasis is plaque psoriasis, e.g., chronic plaque psoriasis, such as moderate to severe chronic plaque psoriasis.

In a still further aspect, the invention provides a method of treating psoriasis in a subject comprising administering to the subject: a) about 200 mg of ABT-874 at weeks 0 and 4; and b) about 100 mg of ABT-874 at week 8 and every 4 weeks thereafter, thereby treating psoriasis in the subject. In one embodiment, the antibody is ABT-874. In one embodiment, the psoriasis is plaque psoriasis, e.g., chronic plaque psoriasis, such as moderate to severe chronic plaque psoriasis.

In one embodiment, the psoriasis is chronic psoriasis. In one embodiment, the psoriasis is plaque psoriasis, e.g., chronic plaque psoriasis. In another embodiment, the psoriasis is chronic psoriasis, e.g., chronic plaque psoriasis. In yet another embodiment, the psoriasis is moderate to severe psoriasis, e.g., moderate to severe plaque psoriasis, moderate to severe chronic psoriasis or moderate to severe chronic plaque psoriasis. In one embodiment, the subject has had a clinical diagnosis of psoriasis for at least 6 months. In another embodiment, the subject has had stable plaque psoriasis for at least 2 months.

In one embodiment, the antibody is administered via subcutaneous injection.

In one embodiment, the antibody, or antigen-binding portion thereof, used in the methods of the invention is capable of binding to an epitope of the p40 subunit of IL-12 and/or IL-23.

In another embodiment, the antibody, or antigen-binding portion thereof, is capable of binding to the epitope of the p40 subunit when the p40 subunit is bound to the p35 subunit of IL-12. In yet another embodiment, the antibody, or antigen-binding portion thereof, is capable of binding to the epitope of the p40 subunit when the p40 subunit is bound to a p19 subunit, i.e., the p19 subunit of IL-23. In one embodiment, the antibody, or antigen-binding portion thereof, is capable of binding to the epitope of the p40 subunit when the p40 subunit is bound to the p35 subunit of IL-12 and when the p40 subunit is bound to a p19 subunit.

In one embodiment, the antibody, or antigen binding portion thereof, binds to an epitope of the p40 subunit of IL-12 to which an antibody selected from the group consisting of Y61 and J695 binds.

In another embodiment, the antibody is further capable of binding to a first heterodimer and is also capable of binding to a second heterodimer, wherein the first heterodimer comprises the p40 subunit of Il-12 and the p35 subunit of Il-12, and wherein the second heterodimer comprises the p40 subunit of IL-12 and a p19 subunit, i.e., the p19 subunit of IL-23.

In a further embodiment, the antibody neutralizes the activity of the first heterodimer. In another embodiment, the antibody neutralizes the activity of the second heterodimer. In yet another embodiment, the antibody neutralizes the activity of the first heterodimer and the second heterodimer.

In a further embodiment, the antibody, or antigen binding portion thereof, used in the methods of the invention inhibits phytohemagglutinin blast proliferation in an in vitro PHA assay with an IC₅₀ of 1×10⁻⁹M or less, or which inhibits human IFN′ production with an IC₅₀ of 1×10⁻¹⁰ M or less.

In one embodiment, the antibody, or antigen binding portion thereof, used in the methods of the invention dissociates from the p40 subunit of IL-12 with a K_{d} of 1×10⁻¹⁰ M or less or a k_{off} rate constant of 1×10⁻³ s⁻¹ or less, as determined by surface plasmon resonance.

In one embodiment, the isolated antibody, or antigen binding portion thereof, used in the methods of the invention is a chimeric antibody, a humanized antibody or a human antibody.

In another embodiment, the antibody, or antigen binding portion thereof, used in the methods of the invention has a heavy chain CDR3 comprising the amino acid sequence of SEQ ID NO: 25 and a light chain CDR3 comprising the amino acid sequence of SEQ ID NO: 26;

In a further embodiment, the antibody, or antigen binding portion thereof, used in the methods of the invention has a heavy chain CDR2 comprising the amino acid sequence of SEQ ID NO: 27 and a light chain CDR2 comprising the amino acid sequence of SEQ ID NO: 28.

In one embodiment, the antibody, or antigen binding portion thereof, used in the methods of the invention has a heavy chain CDR1 comprising the amino acid sequence of SEQ ID NO: 29 and a light chain CDR1 comprising the amino acid sequence of SEQ ID NO: 30.

In another embodiment, the antibody, or antigen-binding portion thereof, used in the methods of the invention is capable of binding to an interleukin comprising a p40 subunit. In one embodiment, the interleukin comprises a p40 subunit and a p35 subunit, e.g., the interleukin is IL-12. In another embodiment, the interleukin comprises a p40 subunit and a p19 subunit, e.g., the interleukin is IL-23. In yet another embodiment, the antibody, or antigen binding portion thereof, neutralizes the activity of the interleukin.

In one embodiment, the antibody, or antigen binding portion thereof, binds to an epitope of the p40 subunit.

In one embodiment, the antibody, or antigen-binding portion thereof, is administered to a subject in a pharmaceutical composition comprising the antibody, or antigen binding portion thereof, and a pharmaceutically acceptable carrier. The pharmaceutical composition may also comprise an additional agent, such as a therapeutic agent, e.g., budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1β monoclonal antibodies, anti-IL-6 monoclonal antibodies, growth factors, elastase inhibitors, pyridinyl-imidazole compounds, antibodies or agonists of TNF, LT, IL-1, IL-2, IL-6, IL-7, IL-8, IL-15, IL-16, IL-18, EMAP-II, GM-CSF, FGF, and PDGF, antibodies of CD2, CD3, CD4, CD8, CD25, CD28, CD30, CD40, CD45, CD69, CD90 or their ligands, methotrexate, cyclosporin, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38, MAP kinase inhibitors, IL-1β converting enzyme inhibitors, TNFα converting enzyme inhibitors, T-cell signaling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1R1, sIL-1R11, sIL-6R, anti-inflammatory cytokines, IL-4, IL-10, IL-11, IL-13 and TGFβ.

In another embodiment, the therapeutic agent in the pharmaceutical composition administered to the subject may be selected from the group consisting of anti-TNF antibodies and antibody fragments thereof, TNFR-Ig constructs, TACE inhibitors, PDE4 inhibitors, corticosteroids, budenoside, dexamethasone, sulfasalazine, 5-aminosalicylic acid, olsalazine, IL-1β converting enzyme inhibitors, IL-1ra, tyrosine kinase inhibitors, 6-mercaptopurines and IL-11.

In another embodiment, the therapeutic agent may be selected from the group consisting of corticosteroids, prednisolone, methylprednisolone, azathioprine, cyclophosphamide, cyclosporine, methotrexate, 4-aminopyridine, tizanidine, interferon-β1a, interferon-β1b, Copolymer 1, hyperbaric oxygen, intravenous immunoglobulin, clabribine, antibodies or agonists of TNF, LT, IL-1, IL-2, IL-6, IL-7, IL-8, IL-15, IL-16, IL-18, EMAP-II, GM-CSF, FGF, PDGF, antibodies to CD2, CD3, CD4, CD8, CD25, CD28, CD30, CD40, CD45, CD69, CD80, CD86, CD90 or their ligands, methotrexate, cyclosporine, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38 or MAP kinase inhibitors, IL-1β converting enzyme inhibitors, TACE inhibitors, T-cell signaling inhibitors, kinase inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1R1, sIL-1R11, sIL-6R, sIL-13R, anti-P7s, p-selectin glycoprotein ligand (PSGL), anti-inflammatory cytokines, IL-4, IL-10, IL-13 and TGFβ.

In one embodiment, the antibody, or antigen-binding portion thereof, used in the methods of the invention binds to human IL-12 and/or human IL-23 and dissociates from human IL-12 and/or human IL-23, respectively, with a K_{d} of 1×10⁻¹⁰M or less and a k_{off} rate constant of 1×10⁻³ s⁻¹ or less, as determined by surface plasmon resonance. In one embodiment, the antibody, or antigen-binding portion thereof, dissociates from human IL-12 and/or human IL-23 with a k_{off} rate constant of 1×10⁻⁴s⁻¹ or less. In another embodiment, the antibody, or antigen-binding portion thereof, dissociates from human IL-12 and/or human IL-23 with a k_{off} rate constant of 1×10⁻⁵s⁻¹ or less.

In another embodiment, the antibody, or antigen-binding portion thereof, binds to human IL-12 and/or human IL-23 and dissociates from human IL-12 and/or human Il-23, respectively, with a k_{off} rate constant of 1×10⁻²s⁻¹ or less, as determined by surface plasmon resonance. In yet another embodiment, the antibody, or antigen-binding portion thereof, dissociates from human IL-12 and/or human IL-23 with a k_{off} rate constant of 1×10⁻³ s⁻¹ or less. In a still further another embodiment, the antibody, or antigen-binding portion thereof, dissociates from human IL-12 and/or human IL-23 with a k_{off} rate constant of 1×10⁻⁴ s⁻¹ or less. In another embodiment, the antibody, or antigen-binding portion thereof, dissociates from human IL-12 and/or human IL-23 with a k_{off} rate constant of 1×10⁻⁵s⁻¹ or less.

In still another embodiment, the antibody, or antigen-binding portion thereof, binds to human IL-12 and/or human IL-23 and dissociates from human IL-12 and/or human IL-23, respectively, with a K_{d} of 1.34×10⁻¹⁰M or less. In yet another embodiment, the antibody, or antigen-binding portion thereof, binds to human IL-12 and/or human IL-23 and dissociates from human IL-12 and/or human IL-23, respectively, with a K_{d} of 9.74×10⁻¹¹M or less. In one embodiment, the antibody, or antigen-binding portion thereof, is a recombinant antibody, or antigen-binding portion thereof.

In one embodiment, the antibody, or antigen-binding portion thereof, used in the methods of the invention is a neutralizing antibody, e.g., neutralizes the activity of human IL-12 and/or human IL-23. In one embodiment, the neutralizing antibody, or antigen-binding portion thereof, inhibits phytohemagglutinin blast proliferation in an in vitro PHA assay with an IC₅₀ of 1×10⁻⁹M or less. In another embodiment, the neutralizing antibody, or antigen-binding portion thereof, inhibits phytohemagglutinin blast proliferation in an in vitro PHA assay with an IC₅₀ of 1×10⁻¹⁰⁻M or less. In still another embodiment, the neutralizing antibody of, or antigen-binding portion thereof, inhibits phytohemagglutinin blast proliferation in an in vitro PHA assay with an IC₅₀ of 1×10⁻¹¹⁻M or less. In yet another embodiment, the neutralizing antibody, or antigen-binding portion thereof, inhibits phytohemagglutinin blast proliferation in an in vitro phytohemagglutinin blast proliferation assay (PHA assay) with an IC₅₀ of 1×10⁻⁷ M or less. In still another embodiment, the neutralizing antibody, or antigen-binding portion thereof, inhibits phytohemagglutinin blast proliferation in an in vitro PHA assay with an IC₅₀ of 1×10⁻⁸ M or less. In one embodiment, the neutralizing antibody, or antigen-binding portion thereof, inhibits human IFNγ production with an IC₅₀ of 1×10⁻¹⁰ M or less. In still another embodiment, the neutralizing antibody, or antigen-binding portion thereof, inhibits human IFNγ production with an IC₅₀ of 1×10⁻¹¹ M or less. In yet a further embodiment, the neutralizing antibody, or antigen-binding portion thereof, inhibits human IFNγ production with an IC₅₀ of 5×10⁻¹² M or less.

In one embodiment, the antibody, or an antigen-binding portion thereof, used in the methods of the invention

a) inhibits phytohemagglutinin blast proliferation in an in vitro PHA assay with an IC₅₀ of 1×10⁻⁹M or less;

b) has a heavy chain CDR3 comprising the amino acid sequence of SEQ ID NO: 25; and c) has a light chain CDR3 comprising the amino acid sequence of SEQ ID NO: 26. In one embodiment, the antibody further has a heavy chain CDR2 comprising the amino acid sequence of SEQ ID NO: 27; and a light chain CDR2 comprising the amino acid sequence of SEQ ID NO: 28. In still another embodiment, the antibody, or antigen-binding portion thereof, further has a heavy chain CDR1 comprising the amino acid sequence of SEQ ID NO: 29; and a light chain CDR1 comprising the amino acid sequence of SEQ ID NO: 30. In still another embodiment, the antibody, or antigen-binding portion thereof, further inhibits phytohemagglutinin blast proliferation in an in vitro PHA assay with an IC₅₀ of 1×10⁻¹⁰ M or less. In still another embodiment, the antibody, or antigen-binding portion thereof, further inhibits phytohemagglutinin blast proliferation in an in vitro PHA assay with an IC₅₀ of 1×10⁻¹¹ M or less.

In one embodiment, the antibody, or antigen-binding portion thereof, used in the methods of the invention has a heavy chain variable region comprising the amino acid sequence of SEQ ID NO: 31, and a light chain variable region comprising the amino acid sequence of SEQ ID NO: 32.

In one embodiment, the antibody, or antigen-binding portion thereof, used in the methods of the invention comprises a heavy chain constant region selected from the group consisting of IgG1, IgG2, IgG3, IgG4, IgM, IgA and IgE constant regions. In one embodiment, the antibody heavy chain constant region is IgG1. In another embodiment, the antibody is a Fab fragment, F(ab′)₂ fragment, or a single chain Fv fragment.

In one embodiment, the antibody, or antigen-binding portion thereof, used in the methods of the invention dissociates from human IL-12 and/or human IL-23 with a K_{d} of 1×10⁻¹⁰ M or less and binds to an epitope on the p40 subunit of human IL-12 and/or human IL-23.

In one embodiment, the antibody, or antigen-binding portion thereof, used in the methods of the invention is a human antibody, or antigen-binding portion thereof, which

a) dissociates from human IL-12 with a k_{off} rate constant of 1×10⁻³ s⁻¹ or less, as determined by surface plasmon resonance;

b) has a heavy chain CDR3 comprising the amino acid sequence of SEQ ID NO: 25; and

c) has a light chain CDR3 comprising the amino acid sequence of SEQ ID NO: 26.

In another embodiment, the antibody, or antigen-binding portion thereof, used in the methods of the invention dissociates from human IL-12 with a k_{off} rate constant of 1×10⁻⁴ s⁻¹ or less. In a further embodiment, the human antibody, or antigen-binding portion thereof, dissociates from human IL-12 with a k_{off} rate constant of 1×10⁻⁵ s⁻¹ or less.

In one embodiment, the antibody, or antigen-binding portion thereof, used in the methods of the invention is a human antibody, or antigen-binding portion thereof, that binds to human IL-12 and comprises:

a light chain CDR3 domain comprising the amino acid sequence of SEQ ID NO: 26; and

a heavy chain CDR3 domain comprising the amino acid sequence of SEQ ID NO: 25.

In one embodiment, the antibody, or antigen-binding portion thereof, has a light chain variable region (LCVR) having a CDR3 domain comprising the amino acid sequence of SEQ ID NO: 26, and has a heavy chain variable region (HCVR) having a CDR3 domain comprising the amino acid sequence of SEQ ID NO: 25. In another embodiment, the antibody, or antigen-binding portion thereof, comprises an LCVR further having a CDR2 domain comprising the amino acid sequence of SEQ ID NO: 28 and an HCVR further comprising a CDR2 domain comprising the amino acid sequence of SEQ ID NO: 27. In yet another embodiment, the LCVR further has CDR1 domain comprising the amino acid sequence of SEQ ID NO: 30 and the HCVR has a CDR1 domain comprising the amino acid sequence of SEQ ID NO: 29.

In one embodiment, the antibody, or antigen-binding portion thereof, binds human IL-12 and/or human IL-23 and is the antibody J695 (also referred to as ABT-874), or an antigen binding portion thereof.

In one embodiment, the antibody, or antigen-binding portion thereof, binds to human IL-12 and/or human IL-23 and dissociates from human IL-12 and/or human IL-23 with a K_{d} of 1.34×10⁻¹° M or less, and neutralizes human IL-12 and/or human IL-23.

In one embodiment, the antibody, or antigen-binding portion thereof, dissociates from human IL-12 and/or human IL-23 with a K_{d} of 9.74×10⁻¹¹M or less. In one embodiment, the antibody, or antigen-binding portion thereof, inhibits phytohemagglutinin blast proliferation in an in vitro PHA assay with an IC₅₀ of 1×10⁻⁷ M or less. In one embodiment, the antibody, or antigen-binding portion thereof, inhibits phytohemagglutinin blast proliferation in an in vitro PHA assay with an IC₅₀ of 1×10⁻⁸ M or less. In one embodiment, the antibody, or antigen-binding portion thereof, inhibits phytohemagglutinin blast proliferation in an in vitro PHA assay with an IC₅₀ of 1×10⁻⁹ M or less. In one embodiment, the antibody, or antigen-binding portion thereof, inhibits phytohemagglutinin blast proliferation in an in vitro PHA assay with an IC₅₀ of 1×10⁻¹⁰ M or less. In one embodiment, the antibody, or antigen-binding portion thereof, inhibits phytohemagglutinin blast proliferation in an in vitro PHA assay with an IC₅₀ of 1×10⁻¹¹ M or less. In one embodiment, the antibody, or antigen-binding portion thereof, inhibits human IFNγ production with an IC₅₀ of 1×10⁻¹⁰ M or less. In one embodiment, the antibody, or antigen-binding portion thereof, inhibits human IFNγ production with an IC₅₀ of 1×10⁻¹¹ M or less. In one embodiment, the antibody, or antigen-binding portion thereof, inhibits human IFNγ production with an IC₅₀ of 5×10⁻¹² M or less.

In one embodiment, the antibody, or antigen-binding portion thereof, used in the methods of the invention inhibits IL-12 and/or IL-23 binding to its receptor in an IL-12 or IL-23 receptor binding assay (RBA), respectively, with an IC₅₀ of 1×10⁻⁹ M or less.

In one embodiment, the antibody, or antigen-binding portion thereof, inhibits IL-12 and/or IL-23 binding to its receptor in an IL-12 or IL-23 receptor binding assay (RBA), respectively, with an IC₅₀ of 1×10⁻¹⁰ M or less. In one embodiment, the antibody, or antigen-binding portion thereof, inhibits IL-12 and/or IL-23 binding to its receptor in an IL-12 or IL-23 receptor binding assay (RBA), respectively, with an IC₅₀ of 1×10⁻¹¹ M or less.

### BRIEF DESCRIPTION OF THE DRAWINGS

FIG. 1 shows the trial design for a Phase III study conducted for ABT-874 for moderate to severe psoriasis, as exemplified in Example 1.

FIG. 2 shows the percentage of patients with improvement at Week 12 from Baseline at or exceeding the minimum clinically important difference (MCID) for each HRQOL outcome, as exemplified in Example 1. P-values for the significance testing of the comparison of MCID response rates at week 12 across treatment arms using chi-square test are indicated as follows: a. p<0.05 vs. placebo. b. p<0.05 vs. etanercept.

FIG. 3 shows the study design exemplified in Example 2. A Phase III study of patients with moderate to severe psoriasis who were treated with ABT-874, a monoclonal antibody specific for IL-12 and IL-23, or placebo. PGA, Physician's Global Assessment. Non-responders at week 12 (PGA greater than or equal to 2) or after week 12 (PGA greater than or equal to 3) may enroll in open-label study M10-016.

FIG. 4 shows the study design exemplified in Examples 3 and 4. Patients were randomized 2:2:1 to a briakinumab, etanercept, or placebo treatment arm at Week 0. A PGA of 0/1 represents a PGA of clear or minimal A PASI 75 represents a 75% reduction in PASI score from baseline. PGA, Physician's Global Assessment. PASI, Psoriasis Area Severity Index.

FIG. 5 shows patient disposition as exemplified in Example 3. 350 patients were enrolled in this study: placebo, N=72; etanercept, N=139; briakinumab, N=139. 91.7% of patients in the placebo arm, 91.4% of patients in the etanercept arm, and 94.2% of patients in the briakinumab arm completed the study. AE, adverse event.

FIG. 6 shows the proportion of patients achieving PGA 0/1 at Week 12, as exemplified in Example 3. 72.7% of patients receiving briakinumab achieved a PGA of 0/1 at Week 12, as compared with 29.5% of patients receiving etanercept and 4.2% of patients receiving placebo. *P<0.001, briakinumab vs. Placebo. ^{†}P<0.001, briakinumab vs. etanercept. NR1 used to handle missing data. NR1, non-responder imputation.

FIG. 7 shows PASI 75 response rates at Week 12, as exemplified in Example 3. 80.6% of briakinumab-treated patients achieved a PASI 75 response at Week 12, as compared with 39.6% of etanercept-treated and 6.9% of placebo-treated patients. *P<0.001, briakinumab vs. placebo. ^{†}P<0.001, briakinumab vs. etanercept. NR1 used to handle missing data. NR1, non-responder imputation.

FIG. 8 shows the proportion of patients achieving PGA 0/1 at Weeks 2, 4, 8, and 12, as exemplified in Example 3. 18.0% of briakinumab-treated patients achieved a PGA of 0/1 as compared with 4.3% of etanercept-treated and 1.4% of placebo-treated patients at Week 4, and this significant difference was maintained over the remainder of the trial (Week 8: 51.8% briakinumab, 15.8% etanercept, 2.8% placebo; Week 12: 72.7% briakinumab, 29.5% etanercept, 4.2% placebo). *P<0.001, briakinumab vs. placebo. ^{†}P<0.001, briakinumab vs. etanercept.

FIG. 9 shows PASI 75/90/100 response rates at Weeks 2, 4, 8, and 12, as exemplified in Example 3. At Weeks 4, 8, and 12, a statistically significantly greater percentage of briakinumab-treated patients achieved PASI 75 as compared with patients receiving etanercept or placebo (A). At Weeks 8 and 12, a statistically significantly greater percentage of briakinumab-treated patients achieved PASI 90 (B) or PASI 100 (C) as compared with placebo- or etanercept. ^{‡}P=0.005, briakinumab vs. etanercept. *P=0.001, briakinumab vs. placebo. ^{†}P<0.001, briakinumab vs. etanercept. §P<0.001, briakinumab vs. placebo.

FIG. 10 shows patient disposition, as exemplified in Example 4. A total of 347 patients were enrolled in the study. 92.6% of placebo-treated, 95.0% of etanercept-treated, and 92.8% of briakinumab-treated patients completed the study. A similar proportion of patients in the briakinumab treatment and etanercept treatment groups discontinued due to AE. AE, adverse event.

FIG. 11 show the proportion of patients achieving PGA 0/1 at Week 12, as exemplified in Example 4. 71.0% of briakinumab-treated patients achieved a PGA of 0/1 at Week 12, as compared with 39.7% of etanercept-treated and 2.9% of placebo-treated patients. *P<0.001, briakinumab vs. placebo. ^{†}P<0.001, briakinumab vs. etanercept. NR1 used to handle missing data. NR1, non-responder imputation.

FIG. 12 shows the proportion of patients achieving PASI 75 at Week 12, as exemplified in Example 4. 81.9% of briakinumab-treated patients achieved a PASI 75 response at Week 12, as compared with 56.0% of etanercept-treated and 7.4% of placebo-treated patients. *P<0.001, briakinumab vs. placebo. ^{†}P<0.001, briakinumab vs. etanercept. NR1 used to handle missing data. NR1, non-responder imputation.

FIG. 13 shows the proportion of patients achieving PGA 0/1 at Weeks 2, 4, 8, and 12, as exemplified in Example 4. By Week 4, 23.2% of briakinumab-treated patients achieved a achieved a PGA of 0/1 as compared with patients receiving 9.2% of etanercept-treated and 1.5% of placebo-treated patients, and a significant difference was maintained over the remainder of the trial (Week 8: 60.1% briakinumab, 22.7% etanercept, 1.5% placebo; Week 12: 71.0% briakinumab, 39.7% etanercept, 2.9% placebo). ^{‡}P=0.002, briakinumab vs. etanercept. *P<0.001, briakinumab vs. placebo. ^{†}P<0.001, briakinumab vs. etanercept.

FIG. 14 shows the proportion of patients achieving PASI 75/90/100 at Weeks 2, 4, 8, and 12, as exemplified in Example 4. At Weeks 4, 8, and 12, a statistically significantly greater percentage of briakinumab-treated patients achieved PASI 75 as compared with patients receiving etanercept or placebo (A). At Weeks 8 and 12, a statistically significantly greater percentage of briakinumab-treated patients achieved PASI 90 (B) or PASI 100 (C) as compared with placebo- or etanercept. ^{‡}P=0.002, briakinumab vs. etanercept. *P<0.001, briakinumab vs. placebo. ^{†}P<0.001, briakinumab vs. etanercept.

FIG. 15 shows the study design as exemplified in Example 5. Non-responders (PGA greater than or equal to 2 at week 12 or PGA greater than or equal to 3 after week 12) were eligible for Open-Label Extension Study. PGA=Physician's Global Assessment; PASI 75=75% improvement from baseline in Psoriasis Area and Severity Index; q4 wk=every 4 weeks; q12 wk=every 12 weeks. *Randomization stratified by treatment received in Induction Phase. ^{†}One subject in q4 wk group was re-randomized but did not receive any study drug in the Maintenance Phase.

FIG. 16 shows the primary results from Example 5. (A) The percentage of patients achieving a PGA 0/1 at week 12; (B) The percentages of patients achieving PASI 75/90/100 at week 12; (C) The percentages of patients maintaining PGAO/1 at week 52. Intention-to-treat analyses: patients with missing scores were considered non-responders. P<0.001 for all measures. Bria=Briakinumab.

FIG. 17 shows results from patients treated with/not treated with biologics prior to administration of Briakinumab, as exemplified in Example 5. Data of patients treated with biologics or with no biologics prior to administration of Briakinumab are shown. Intention to treat analysis; patients with missing data were counted as nonresponders. Represents exposure to prior biologics within 12 months prior to study enrollment. Week 52 results for the briakinumab 100 mg q 4 week dosing group vs. placebo are presented. (A) % of patients with PGA 0/1 at week 12 and at week 52 of Briakinumab treatment. (B) % of patients with PASI 75 at week 12 and at week 52 of Briakinumab treatment.

FIG. 18 shows results from patients treated with biologics prior to administration of Briakinumab, as exemplified in Example 5. Intention to treat analysis; patients with missing data were counted as nonresponders. Patients with any history of prior biologic use, including beyond 12 months prior to study enrollment were included in both groups; patients with “no lack of response” discontinued prior biologics for reasons other than lack of response. Week 52 results for the briakinumab 100 mg q 4 week dosing group vs. placebo are presented. (A) % of patients with PGA 0/1 at week 12 and week 52 are shown for Briakinumab and placebo groups; (B) % of patients with PASI 75 at week 12 and week 52 are shown for Briakinumab and placebo groups;

FIG. 19 shows the results from treating patients with a history of psoriatic arthritis, as exemplified in Example 5. Intention to treat analysis; patients with missing data were counted as nonresponders. Week 52 results for the briakinumab 100 mg q 4 week dosing group vs. placebo are presented. (A) % of patients with PGA 0/1 at week 12 and week 52 are shown for Briakinumab and placebo groups; (B) % of patients with PASI 75 at week 12 and week 52 are shown for Briakinumab and placebo groups.

FIG. 20 shows the results from treating patients who had a baseline weight of less than 100 kg or greater than or equal to 100 kg, as exemplified in Example 5. Intention to treat analysis; patients with missing data were counted as nonresponders. Week 52 results for the briakinumab 100 mg q 4 week dosing group vs. placebo are presented. (A) % of patients with PGA 0/1 at week 12 and week 52 are shown for Briakinumab and placebo groups; (B) % of patients with PASI 75 at week 12 and week 52 are shown for Briakinumab and placebo groups.

FIG. 21 shows the results from treating patients who had a baseline disease severity PASI score of less than or equal to 20 or had a baseline disease severity PASI score of greater than 20, as exemplified in Example 5. Intention to treat analysis; patients with missing data were counted as nonresponders. Week 52 results for the briakinumab 100 mg q 4 week dosing group vs. placebo are presented. (A) % of patients with PGA 0/1 at week 12 and week 52 are shown for Briakinumab and placebo groups; (B) % of patients with PASI 75 at week 12 and week 52 are shown for Briakinumab and placebo groups.

FIG. 22 shows the results from treating patients who had a baseline disease severity of less than or equal to 20% body surface area affected (BSA) by psoriasis or who had a baseline disease severity of greater than 20% body surface area affected (BSA) by psoriasis, as exemplified in Example 5. Intention to treat analysis; patients with missing data were counted as nonresponders. Week 52 results for the briakinumab 100 mg q 4 week dosing group vs. placebo are presented. (A) % of patients with PGA 0/1 at week 12 and week 52 are shown for Briakinumab and placebo groups; (B) % of patients with PASI 75 at week 12 and week 52 are shown for Briakinumab and placebo groups.

FIG. 23 shows the percentages of patients with improvement at Week 12 or exceeding the minimum clinically important difference (MCID) for each HRQOL outcome, as exemplified in Example 6. a. Treatment with ABT-874 showed significantly greater response rates compared with placebo only. b. Treatment with ABT-874 showed significantly greater response rates compared with etanercept and placebo.

FIG. 24 shows the Study Design for Example 9.

FIG. 25A shows PASI response rates at the week 8 induction phase. FIG. 25B shows PASI response rates at the week 52 maintenance phase. FIG. 25C shows PASI response rates at week 48 OLE.

FIG. 26 shows PASI 75 response rates over time.

FIG. 27A shows PGA response rates at the week 8 induction phase. FIG. 27B shows PGA response rates at the week 52 maintenance phase. FIG. 27C shows PGA response rates at the week 48 OLE.

FIG. 28 shows PGA 0 or 1 response rates over time.

FIG. 29 shows PASI 75 responses over time for the maintenance of efficacy population.

FIG. 30 shows PASI 90 responses over time for the maintenance of efficacy population.

FIG. 31 shows PASI 100 responses over time for the maintenance of efficacy population.

FIG. 32 shows PGA 0 or 1 Responses Over Time for the Maintenance of Efficacy Population.

FIG. 33 shows the study design for the phase III studies for Example 11.

FIG. 34 shows PASI 75 response rates in the OLE.

FIG. 35 shows PASI 90 response rates in the OLE.

FIG. 36 shows PASI 100 response rates in the OLE.

FIG. 37 shows PGA 0 or 1 (clear or minimal) response rates in the OLE.

FIG. 38 shows PGA 0 (clear) response rates in the OLE.

FIG. 39 shows the study design for Example 12.

### DETAILED DESCRIPTION OF THE INVENTION

In order that the present invention may be more readily understood, certain terms are first defined.

The term “activity enhancing amino acid residue” includes an amino acid residue which improves the activity of the antibody. It should be understood that the activity enhancing amino acid residue may replace an amino acid residue at a contact, hypermutation or preferred selective mutagenesis position and, further, more than one activity enhancing amino acid residue can be present within one or more CDRs. An activity enhancing amino acid residue include, an amino acid residue that improves the binding specificity/affinity of an antibody, for example anti-human IL-12 antibody binding to human IL-12. The activity enhancing amino acid residue is also intended to include an amino acid residue that improves the neutralization potency of an antibody, for example, the human IL-12 antibody which inhibits human IL-12.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In one embodiment, the antibody used in the compositions and methods of the invention is the antibody described in U.S. Pat. No. 6,914,128, incorporated by reference herein. In another embodiment, the antibody used in the compositions and methods of the invention is the antibody ABT-874 (also referred to as J695; Abbott Laboratories).

The term “antigen-binding portion” of an antibody (or “antibody portion”) includes fragments of an antibody that retain the ability to specifically bind to an antigen (e.g., hIL-12). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Still further, an antibody or antigen-binding portion thereof may be part of a larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

The term “backmutation” refers to a process in which some or all of the somatically mutated amino acids of a human antibody are replaced with the corresponding germline residues from a homologous germline antibody sequence. The heavy and light chain sequences of the human antibody of the invention are aligned separately with the germline sequences in the VBASE database to identify the sequences with the highest homology. Differences in the human antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the human antibody should not be included in the final human antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and colinear to the sequence of the human antibody of the invention for at least 10, preferably 12 amino acids, on both sides of the amino acid in question. Backmutation may occur at any stage of antibody optimization; preferably, backmutation occurs directly before or after the selective mutagenesis approach. More preferably, backmutation occurs directly before the selective mutagenesis approach.

The phrase “human interleukin 12” (abbreviated herein as hIL-12, or IL-12), as used herein, includes a human cytokine that is secreted primarily by macrophages and dendritic cells. The term includes a heterodimeric protein comprising a 35 kD subunit (p35) and a 40 kD subunit (p40) which are both linked together with a disulfide bridge. The heterodimeric protein is referred to as a “p70 subunit”. The structure of human IL-12 is described further in, for example, Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237. The term human IL-12 is intended to include recombinant human IL-12 (rh IL-12), which can be prepared by standard recombinant expression methods.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The Kabat numbering is used herein to indicate the positions of amino acid modifications made in antibodies of the invention. For example, the Y61 anti-IL-12 antibody can be mutated from serine (S) to glutamic acid (E) at position 31 of the heavy chain CDR1 (H31S→E), or glycine (G) can be mutated to tyrosine (Y) at position 94 of the light chain CDR3 (L94G→Y).

The term “human antibody” includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described herein. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds hIL-12 is substantially free of antibodies that specifically bind antigens other than hIL-12). An isolated antibody that specifically binds hIL-12 may bind IL-12 molecules from other species (discussed in further detail below). Moreover, an isolated antibody may be substantially free of other cellular material and/or chemicals.

A “neutralizing antibody” (or an “antibody that neutralized hIL-12 activity”) includes an antibody whose binding to hIL-12 results in inhibition of the biological activity of hIL-12. This inhibition of the biological activity of hIL-12 can be assessed by measuring one or more indicators of hIL-12 biological activity, such as inhibition of human phytohemagglutinin blast proliferation in a phytohemagglutinin blast proliferation assay (PHA), or inhibition of receptor binding in a human IL-12 receptor binding assay (see Example 3-Interferon-gamma Induction Assay of U.S. Pat. No. 6,914,128). These indicators of hIL-12 biological activity can be assessed by one or more of several standard in vitro or in vivo assays known in the art (see Example 3 of U.S. Pat. No. 6,914,128).

The term “activity” includes activities such as the binding specificity/affinity of an antibody for an antigen, for example, an anti-hIL-12 antibody that binds to an IL-12 antigen and/or the neutralizing potency of an antibody, for example, an anti-hIL-12 antibody whose binding to hIL-12 inhibits the biological activity of hIL-12, e.g. inhibition of PHA blast proliferation or inhibition of receptor binding in a human IL-12 receptor binding assay (see Example 3 of U.S. Pat. No. 6,914,128).

The phrase “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 of U.S. Pat. No. 6,914,128 and Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnson, B., et al. (1991) Anal. Biochem. 198:268-277.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The phrase “nucleic acid molecule” includes DNA molecules and RNA molecules. A nucleic acid molecule may be single-stranded or double-stranded, but preferably is double-stranded DNA.

The phrase “isolated nucleic acid molecule”, as used herein in reference to nucleic acids encoding antibodies or antibody portions (e.g., VH, VL, CDR3) that bind hIL-12 including “isolated antibodies”), includes a nucleic acid molecule in which the nucleotide sequences encoding the antibody or antibody portion are free of other nucleotide sequences encoding antibodies or antibody portions that bind antigens other than hIL-12, which other sequences may naturally flank the nucleic acid in human genomic DNA. Thus, for example, an isolated nucleic acid of the invention encoding a VH region of an anti-IL-12 antibody contains no other sequences encoding other VH regions that bind antigens other than IL-12. The phrase “isolated nucleic acid molecule” is also intended to include sequences encoding bivalent, bispecific antibodies, such as diabodies in which VH and VL regions contain no other sequences other than the sequences of the diabody.

The term “vector” includes a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

The phrase “contact position” includes an amino acid position of in the CDR1, CDR2 or CDR3 of the heavy chain variable region or the light chain variable region of an antibody which is occupied by an amino acid that contacts antigen in one of the twenty-six known antibody-antigen structures. If a CDR amino acid in any of the 26 known solved structures of antibody-antigen complexes contacts the antigen, then that amino acid can be considered to occupy a contact position. Contact positions have a higher probability of being occupied by an amino acid which contact antigen than non-contact positions. Preferably a contact position is a CDR position which contains an amino acid that contacts antigen in greater than 3 of the 26 structures (>11.5%). Most preferably a contact position is a CDR position which contains an amino acid that contacts antigen in greater than 8 of the 25 structures (>32%).

The term “hypermutation position” includes an amino acid residue that occupies position in the CDR1, CDR2 or CDR3 region of the heavy chain variable region or the light chain variable region of an antibody that is considered to have a high frequency or probability for somatic hypermutation during in vivo affinity maturation of the antibody. “High frequency or probability for somatic hypermutation” includes frequencies or probabilities of a 5 to about 40% chance that the residue will undergo somatic hypermutation during in vivo affinity maturation of the antibody. It should be understood that all ranges within this stated range are also intended to be part of this invention, e.g., 5 to about 30%, e.g., 5 to about 15%, e.g., 15 to about 30%.

The term “preferred selective mutagenesis position” includes an amino acid residue that occupies a position in the CDR1, CDR2 or CDR3 region of the heavy chain variable region or the light chain variable region which can be considered to be both a contact and a hypermutation position.

The phrase “selective mutagenesis approach” includes a method of improving the activity of an antibody by selecting and individually mutating CDR amino acids at least one preferred selective mutagenesis position, hypermutation, and/or contact position. A “selectively mutated” human antibody is an antibody which contains a mutation at a position selected using a selective mutagenesis approach. In another embodiment, the selective mutagenesis approach is intended to provide a method of preferentially mutating selected individual amino acid residues in the CDR1, CDR2 or CDR3 of the heavy chain variable region (hereinafter H1, H2, and H3, respectively), or the CDR1, CDR2 or CDR3 of the light chain variable region (hereinafter referred to as L1, L2, and L3, respectively) of an antibody Amino acid residues may be selected from preferred selective mutagenesis positions, contact positions, or hypermutation positions. Individual amino acids are selected based on their position in the light or heavy chain variable region. It should be understood that a hypermutation position can also be a contact position. In an embodiment, the selective mutagenesis approach is a “targeted approach”. The language “targeted approach” is intended to include a method of preferentially mutating selected individual amino acid residues in the CDR1, CDR2 or CDR3 of the heavy chain variable region or the CDR1, CDR2 or CDR3 of the light chain variable region of an antibody in a targeted manner, e.g., a “Group-wise targeted approach” or “CDR-wise targeted approach”. In the “Group-wise targeted approach”, individual amino acid residues in particular groups are targeted for selective mutations including groups I (including L3 and H3), II (including H2 and L1) and III (including L2 and H1), the groups being listed in order of preference for targeting. In the “CDR-wise targeted approach”, individual amino acid residues in particular CDRs are targeted for selective mutations with the order of preference for targeting as follows: H3, L3, H2, L1, H1 and L2. The selected amino acid residue is mutated, e.g., to at least two other amino acid residues, and the effect of the mutation on the activity of the antibody is determined. Activity is measured as a change in the binding specificity/affinity of the antibody, and/or neutralization potency of the antibody. It should be understood that the selective mutagenesis approach can be used for the optimization of any antibody derived from any source including phage display, transgenic animals with human IgG germline genes, human antibodies isolated from human B-cells. Preferably, the selective mutagenesis approach is used on antibodies which can not be optimized further using phage display technology. It should be understood that antibodies from any source including phage display, transgenic animals with human IgG germline genes, human antibodies isolated from human B-cells can be subject to backmutation prior to or after the selective mutagenesis approach.

The term “activity enhancing amino acid residue” includes an amino acid residue which improves the activity of the antibody. It should be understood that the activity enhancing amino acid residue may replace an amino acid residue at a preferred selective mutagenesis position, contact position, or a hypermutation position and, further, more than one activity enhancing amino acid residue can be present within one or more CDRs. An activity enhancing amino acid residue include, an amino acid residue that improves the binding specificity/affinity of an antibody, for example anti-human IL-12 antibody binding to human IL-12. The activity enhancing amino acid residue is also intended to include an amino acid residue that improves the neutralization potency of an antibody, for example, the human IL-12 antibody which inhibits human IL-12.

The term “Cₘₐₓ” refers to the maximum or peak serum or plasma concentration of an agent observed in a subject after its administration.

The term “Tₘₐₓ” refers to the time at which Cₘₐₓ occurred.

The term “bioavailability” or “F %” refers to a fraction or percent of a dose which is absorbed and enters the systemic circulation after administration of a given dosage form. The dose of the agent may be administered through any route, and, preferably, via intravenous or subcutaneous injection.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-IL-12, anti-IL-23 antibody and another drug. The other drug(s) may be administered concomitant with, prior to, or following the administration of an anti-IL-12, anti-IL-23 antibody.

The term “dosing”, as used herein, refers to the administration of a substance (e.g., an anti-IL-12, anti-IL-23 antibody) to achieve a therapeutic objective (e.g., treatment of psoriasis).

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered. Exemplary dose amounts, e.g., fixed dose amounts, for use in the methods of the invention include, about 100 mg, about 110 mg, about 120 mg, about 130 mg, about 140 mg, about 150 mg, about 160 mg, about 170 mg, about 180 mg, or about 190 mg, about 200 mg, about 210 mg, about 220 mg, about 230 mg, about 240 mg, about 250 mg, about 260 mg, about 270 mg, about 280 mg, about 290 mg, or about 300 mg. In one embodiment, the dose amount is about 100 to about 300 mg. In yet another embodiment, the dose amount is about 100 to about 200 mg. Ranges intermediate to the above-recited ranges are also contemplated by the invention. For example, ranges having any one of these values as the upper or lower limits are also intended to be part of the invention, e.g., about 110 mg to about 170 mg, about 150 mg to about 220 mg, etc.

As used herein, the term “periodicity” as it relates to the administration of a substance (e.g., an antibody which binds to the p40 subunit of—IL-12 and/or—IL-23) refers to a (regular) recurring cycle of administering the substance to a subject. In one embodiment, the recurring cycle of administration of the substance to the subject achieves a therapeutic objective. The periodicity of administration of the substance may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, about 160 days to about 220 days, etc.

As used herein, the phrase “periodicity of about once every 4 weeks” as it relates to the administration of a substance (e.g., an antibody which binds to the p40 subunit of—IL-12 and/or IL-23), refers to a (regular) recurring cycle of administering the substance to a subject about once every 4 weeks, about once every 28 days, or about once every month. In one embodiment, the recurring cycle of administration of the substance to the subject achieves or maintains a therapeutic objective (e.g., treating psoriasis), either alone or in conjunction with other recurring cycles (e.g., if a first periodicity, then in conjunction with a second and/or third periodicity; if a second periodicity, then in conjunction with a first and/or third periodicity; and if a third periodicity, then in conjunction with a first and second periodicity) of administering the substance. Preferably, the substance is administered once every 22-34 days, every 24-32 days, even more preferably, every 26-30 days (e.g., every 26, 27, 28, 29 or 30 days), and most preferably every 28 days.

As used herein, the phrase “periodicity of about once every 12 weeks” as it relates to the administration of a substance (e.g., an antibody which binds to the p40 subunit of—IL-12 and/or IL-23), refers to a (regular) recurring cycle of administering the substance to a subject about once every 12 weeks, about once every 84 days, or about once every 3 months. In one embodiment, the recurring cycle of administration of the substance to the subject achieves or maintains a therapeutic objective (e.g., treating psoriasis), either alone or in conjunction with other recurring cycles (e.g., if a first periodicity, then in conjunction with a second and/or third periodicity; if a second periodicity, then in conjunction with a first and/or third periodicity; and if a third periodicity, then in conjunction with a first and second periodicity) of administering the substance. Preferably, the substance is administered once every 78-90 days, every 80-88 days, even more preferably, every 82-86 days (e.g., every 82, 83, 84, 85 or 86 days), and most preferably every 84 days.

The “duration of a periodicity” refers to a time over which the recurring cycle of administration occurs.

For example, a duration of the periodicity of administration of a substance may be may about 12 weeks during which the periodicity of administration is about once every week. For example, a duration of the periodicity may be about 6 weeks during which the periodicity of administration is about once every 4 weeks, e.g., the substance is administered at week zero and at week four.

The duration of periodicity may be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 weeks, about 20 weeks, about 25 weeks, about 30 weeks, about 35 weeks, about 40 weeks, about 45 weeks, about 50 weeks, about 52 weeks, about 55 weeks, about 60 weeks, about 70 weeks, about 80 weeks, about 90 weeks, or about 100 weeks, or longer. In one embodiment, the duration of periodicity is for a length of time necessary or required to achieve a therapeutic objective, e.g., treatment, maintenance of treatment, etc. e.g., maintain a PASI 50, PASI 75, PASI 90, PASI 100 score or PGA of 0 or 1 score. Durations of a periodicity intermediate to the above-recited times are also contemplated by the invention.

The duration of periodicity may be about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, or longer. The duration of periodicity may be at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, or at least about 52 weeks.

Furthermore, the duration of periodicity may be at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 70 weeks, at least about 80 weeks, at least about 90 weeks, or at least about 100 weeks.

The term “treated,” “treating” or “treatment” includes the diminishment or alleviation of at least one symptom associated or caused by the state, disorder or disease being treated. For example, treatment can be diminishment of one or more symptoms of a disorder or complete eradication of a disorder. “Treatment” or “treating” (e.g., treating psoriasis) means achieving or maintaining a therapeutic objective. “Treatment” or “treating” can mean maintaining a response to a prior treatment (e.g., a prior response achieved following administration of a first dose amount according to a first periodicity; or achieved following administration of a first dose amount according to a first periodicity and a second dose amount according to a second periodicity; or achieved following administration of a first dose amount according to a first periodicity and a first or second dose amount according to a second periodicity, and a first, second, or third dose amount according to a third periodicity. “Treatment of” or “treating” psoriasis may mean achieving or maintaining a PGA score of 0/1 or a PASI 50, PASI 75, PASI 90, or PASI 100 response score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer). “Treatment of” or “treating” psoriasis may also mean achieving or maintaining a health-related quality of life (HRQOL) outcome.

HRQOL outcomes include Dermatology Life Quality Index (DLQI), visual analog scales for Ps-related (VAS-Ps) and psoriatic arthritis-related (VAS-PsA) pain, Short Form 36 Health Survey Mental (MCS) and Physical (PCS) Component Summary scores, a Short Form 36 Health Survey Physical Function (PF) score, a Short Form 36 Health Survey Role-Physical (RP) score, a Short Form 36 Health Survey Bodily Pain (BP) score, a Short Form 36 Health Survey General Health (GH) score, a Short Form 36 Health Survey Vitality (VT) score, a Short Form 36 Health Survey Social Function (SF) score, a Short Form 36 Health Survey Role-Emotional (RE) score, a Short Form 36 Health Survey Mental Health (MH) score, and Total Activity Impairment (TAI) scores. “Treatment of” or “treating” psoriasis may also mean achieving or maintaining a minimum clinically important difference (MCID) for any of the HRQOL outcomes provided herein, e.g., any one or combination of DLQI, VAS-Ps, VAS-PsA, MCS, PCS, TAI, PF, RP, BP, GH, VT, SF, RE, and MH. “Treatment of” or “treating” psoriasis may also mean achieving or maintaining a minimum clinically important difference (MCID) response rate for any of the HRQOL outcomes provided herein, e.g., any one or combination of DLQI, VAS-Ps, VAS-PsA, MCS, PCS, TAI, PF, RP, BP, GH, VT, SF, RE, and MH. “Treatment of” or “treating” psoriasis may also mean achieving or maintaining a clinically meaningful difference in any of the HRQOL outcomes provided herein, e.g., any one or combination of DLQI, VAS-Ps, VAS-PsA, MCS, PCS, TAI, PF, RP, BP, GH, VT, SF, RE, and MH (e.g., a clinically meaningful reduction in DLQI, VAS-Ps, and/or VAS-PsA, or a clinically meaningful increase in MCS, PCS, TAI, PF, RP, BP, GH, VT, SF, RE, and/or MH). “Treatment of” or “treating” psoriasis may also mean achieving or maintaining a Nail Psoriasis Severity Index (NAPSI) score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer). “Treatment of” or “treating” psoriasis may also mean achieving or maintaining any of the outcomes provided herein in a certain percentage of a population of subjects (e.g., in at least about 5%, 10%, 15%, 20%, 25%, 30%, 35%, 40%, 45%, 50%, 55%, 60%, 65%, 70%, 75%, 80%, 85%, 90%, 95%, 99%, or 100% of a population of subjects).

The term “kit” as used herein refers to a packaged product comprising components with which to administer the anti-IL-12, anti-IL-23 antibody of the invention for treatment of a IL-12 related disorder. The kit preferably comprises a box or container that holds the components of the kit. The box or container is affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for administering an anti-IL-12, anti-IL-23 antibody.

“Short Form 36 Health Survey” (SF-36) measures the following eight health domains: MH, Mental Health; PF, Physical Function; RE, Role-Emotional; RP, Role—Physical; SF, Social Function; VT, Vitality; BP, Bodily Pain; GH, General Health. Domain scales values range from 0 to 100. Two summary scores are derived from the domain scores: MCS, Mental Component Summary and PCS, Physical Component Summary scores. PCS and MCS values range from 0 to 100. For all SF-36 scores, positive changes in scores indicate improvement in health.

Various aspects of the invention are described in further detail in the following subsections.

I. Human Antibodies that Bind to the p40 subunit of Human IL-12/Human IL-23

This invention provides methods and compositions for using human antibodies, or antigen-binding portions thereof, that bind to human IL-12 for the treatment of psoriasis. The invention also includes methods and compositions for using an antibody which binds both IL-12 and IL-23. Preferably, the human antibodies used in the invention are recombinant, neutralizing human anti-hIL-12/IL-23 antibodies.

Antibodies that can be used in the methods of the invention include polyclonal, monoclonal, recombinant antibodies, single chain antibodies, hybrid antibodies, chimeric antibodies, humanized antibodies, or fragments thereof. Antibody-like molecules containing one or two binding sites for an antigen and a Fc-part of an immunoglobulin can also be used. Preferred antibodies used in the methods of the invention are human antibodies. In a preferred embodiment, the antibody is an isolated human recombinant antibody, or an antigen-binding portion thereof.

In one aspect, the methods of the invention utilize a human antibody that binds to an epitope of the p40 subunit of IL-12/IL-23. In one embodiment, the antibody binds to the p40 subunit when the p40 subunit is bound to the p35 subunit of IL-12. In one embodiment, the antibody binds to the p40 subunit when the p40 subunit is bound to the p19 subunit of IL-23. In one embodiment, the antibody binds to the p40 subunit when the subunit is bound to the p35 subunit of IL-12 and also when the p40 subunit is bound to the p19 subunit of Il-23. In a preferred embodiment, the antibody, or antigen-binding portion thereof, is an antibody like those described in U.S. Pat. No. 6,914,128, the entire contents of which are incorporated by reference herein. For example, in a preferred embodiment, the antibody binds to an epitope of the p40 subunit of IL-12 to which an antibody selected from the group consisting of Y61 and J695, as described in U.S. Pat. No. 6,914,128, binds. Especially preferred among the human antibodies is J695 as described in U.S. Pat. No. 6,914,128 (also referred to as ABT-874 or Briakinumab herein). Other antibodies that bind IL-12 and/or IL-23 and which can be used in the methods of the invention include the human anti-IL-12 antibody C340, as described in U.S. Pat. No. 6,902,734, the entire contents of which are incorporated by reference herein.

In one aspect, the methods of the invention utilize J695 antibodies and antibody portions, J695-related antibodies and antibody portions, and other human antibodies and antibody portions with equivalent properties to J695, such as high affinity binding to hIL-12/IL-23 with low dissociation kinetics and high neutralizing capacity. For example, in one embodiment of the invention, the formulation contains a human antibody, or antigen-binding portion thereof, that dissociates from the p40 subunit of human IL-12/IL-23 with a K_{d} of 1.34×10⁻¹⁰ M or less or with a K_{off} rate constant of 1×10⁻³ s⁻¹ or less, as determined by surface plasmon resonance. Preferably, the antibody, or antigen-binding portion thereof, dissociates from the p40 subunit of human IL-12/IL-23 with a k_{off} rate constant of 1×10⁻⁴ s⁻¹ or less, and more preferably with a k_{off} rate constant of 1×10⁻⁵s⁻¹ or less, or with a K_{d} of 1×10⁻¹⁰M or less, and more preferably with a K_{d} of 9.74×10⁻¹¹M or less.

The dissociation rate constant (K_{off})) of an IL-12/IL-23 antibody can be determined by surface plasmon resonance. Generally, surface plasmon resonance analysis measures real-time binding interactions between ligand (recombinant human IL-12 immobilized on a biosensor matrix) and analyte (antibodies in solution) by surface plasmon resonance (SPR) using the BIAcore system (Pharmacia Biosensor, Piscataway, N.J.). Surface plasmon analysis can also be performed by immobilizing the analyte (antibodies on a biosensor matrix) and presenting the ligand (recombinant IL-12/IL-23 in solution) (see, for example, assays described in Example 5 of U.S. Pat. No. 6,914,128, the contents of which are incorporated by reference herein). Neutralization activity of IL-12/IL-23 antibodies, or antigen binding portions thereof, can be assessed using one or more of several suitable in vitro assays (see for example, assays described in Example 3 of U.S. Pat. No. 6,914,128, the contents of which are incorporated by reference herein).

In another embodiment of the invention, the methods utilize a human antibody, or antigen-binding portion thereof, that neutralizes the biological activity of the p40 subunit of human IL-12/IL-23. In one embodiment, the antibody, or antigen-binding portion thereof, neutralizes the biological activity of free p40, e.g., monomer p40 or a p40 homodimer, e.g., a dimer containing two identical p40 subunits. In preferred embodiments, the antibody, or antigen-binding portion thereof, neutralizes the biological activity of the p40 subunit when the p40 subunit is bound to the p35 subunit of Il-12 and/or when the p40 subunit is bound to the p19 subunit of IL-23. In various embodiments, the antibody, or antigen-binding portion thereof, inhibits human IL-12-induced phytohemagglutinin blast proliferation in an in vitro PHA assay with an IC₅₀ of 1×10⁻⁷M or less, preferably with an IC₅₀ of 1×10⁻⁸ M or less, more preferably with an IC₅₀ of 1×10⁻⁹M or less, even more preferably with an IC₅₀ of 1×10⁻¹⁰ M or less, and most preferably with an IC₅₀ of 1×10⁻¹¹⁻M or less. In other embodiments, the antibody, or antigen-binding portion thereof, inhibits human IL-12-induced human IFN′ production with an IC₅₀ of 1×10⁻¹⁰ M or less, preferably with an IC₅₀ of 1×10⁻¹¹ M or less, and more preferably with an IC₅₀ of 5×10⁻¹² M or less.

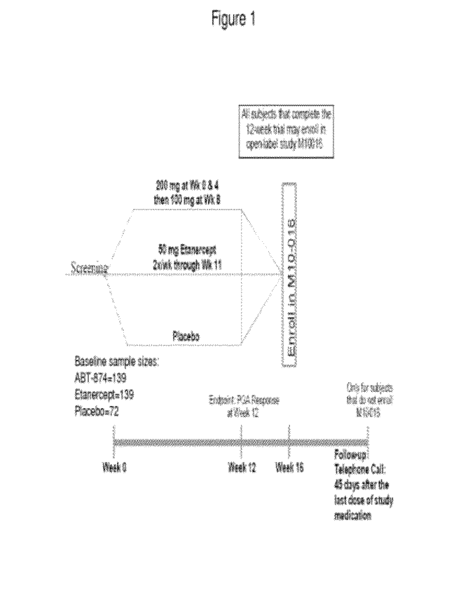

Antibodies that bind to the p40 subunit of human IL-12/IL-23 can be selected, for example, by screening one or more human V_{L} and V_{H} cDNA libraries with hIL-12, such as by phage display techniques as described in Example 1 of U.S. Pat. No. 6,914,128. Screening of human V_{L} and V_{H} cDNA libraries initially identified a series of anti-IL-12 antibodies of which one antibody, referred to herein as “Joe 9” (or “Joe 9 wild type”), was selected for further development. Joe 9 is a relatively low affinity human IL-12 antibody (e.g., a K_{off} of about 0.1 sec⁻¹), yet is useful for specifically binding and detecting hIL-12. The affinity of the Joe 9 antibody was improved by conducting mutagenesis of the heavy and light chain CDRs, producing a panel of light and heavy chain variable regions that were “mixed and matched” and further mutated, leading to numerous additional anti-hIL-12 antibodies with increased affinity for hIL-12 (see Example 1, table 2 of U.S. Pat. No. 6,914,128 and the sequence alignments of FIGS. 1A-D of U.S. Pat. No. 6,914,128.

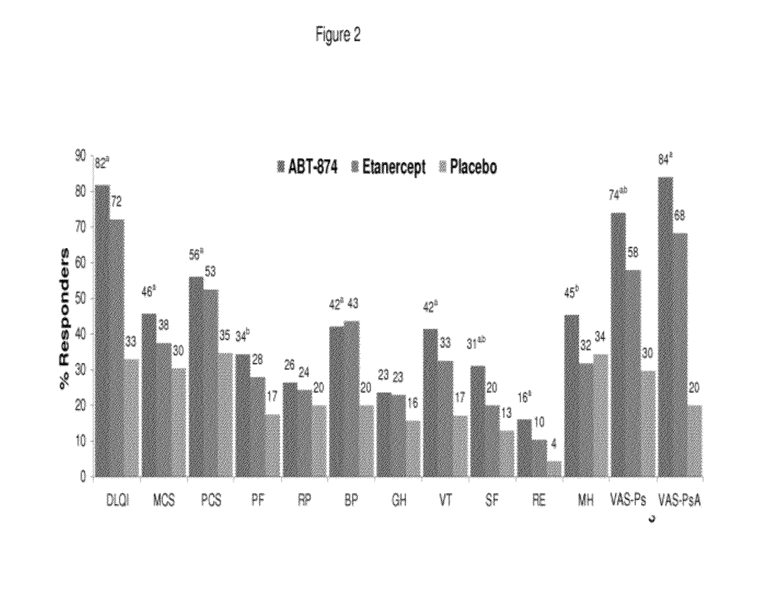

Of these antibodies, the human anti-hIL-12 antibody referred to herein as Y61 demonstrated a significant improvement in binding affinity (e.g., a K_{off} of about 2×10⁻⁴ sec⁻¹). The Y61 anti-hIL-12 antibody was selected for further affinity maturation by individually mutating specific amino acids residues within the heavy and light chain CDRs Amino acids residues of Y61 were selected for site-specific mutation (selective mutagenesis approach) based on the amino acid residue occupying a preferred selective mutagenesis position, contact and/or a hypermutation position. A summary of the substitutions at selected positions in the heavy and light chain CDRs is shown in FIGS. 2A-2H of U.S. Pat. No. 6,914,128. A preferred recombinant neutralizing antibody of the invention, referred to herein as J695 (also referred to as ABT-874 (Abbott Laboratories), resulted from a Gly to Tyr substitution at position 50 of the light chain CDR2 of Y61, and a Gly to Tyr substitution at position 94 of the light chain CDR3 of Y61.

Amino acid sequence alignments of the heavy and light chain variable regions of a panel of anti-IL-12 antibodies used in the invention, on the lineage from Joe 9 wild type to J695, are shown in FIGS. 1A-1D of U.S. Pat. No. 6,914,128. These sequence alignments allowed for the identification of consensus sequences for preferred heavy and light chain variable regions of antibodies of the invention that bind hIL-12, as well as consensus sequences for the CDR3, CDR2, and CDR1, on the lineage from Joe 9 to J695. Moreover, the Y61 mutagenesis analysis summarized in FIGS. 2A-2H of U.S. Pat. No. 6,914,128 allowed for the identification of consensus sequences for heavy and light chain variable regions that bind hIL-12, as well as consensus sequences for the CDR3, CDR2, and CDR1 that bind hIL-12 on the lineage from Y61 to J695 that encompasses sequences with modifications from Y61 yet that retain good hIL-12 binding characteristics. Preferred CDR, VH and VL sequences of the invention (including consensus sequences) as identified by sequence identifiers in the attached Sequence Listing, are summarized below.

| SEQ | | | |
|---|---|---|---|
| ID | ANTIBODY | | |
| NO: | CHAIN | REGION | SEQUENCE |
| 1 | Consensus | CDR H3 | (H/S)-G-S-(H/Y)-D- |
| | Joe 9 to | | (N/T/Y) |
| | J695 | | |
| 2 | Consensus | CDR L3 | Q-(S/T)-Y-(D/E)-(S/R/K)- |
| | Joe 9 to | | (S/G/Y)-(L/F/T/S)- |
| | J695 | | (R/S/T/W/H)-(G/P)- |
| | | | (S/T/A/L)-(R/S/M/T/L)- |
| | | | (V/I/T/M/L) |
| 3 | Consensus | CDR H2 | F I R Y D G S N K Y Y A |
| | Joe 9 to | | D-S-V-K-G |
| | J695 | | |
| 4 | Consensus | CDR L2 | (G/Y)-N-(D/S)-(Q/N)-R-P- |
| | Joe 9 to | | S |
| | J695 | | |
| 5 | Consensus | CDR H1 | F-T-F-S-(S/E)-Y-G-M-H |
| | Joe 9 to | | |
| | J695 | | |
| 6 | Consensus | CDR L1 | (S/T)-G-(G/S)-(R/S)-S-N- |
| | Joe 9 to | | I-(G/V)-(S/A)-(N/G/Y)- |
| | J695 | | (T/D)-V-(K/H) |
| 7 | Consensus | VH | (full VH sequence; see |
| | Joe 9 to | | sequence listing) |
| | J695 | | |
| 8 | Consensus | VL | (full VL sequence; see |
| | Joe 9 to | | sequence listing) |
| | J695 | | |
| 9 | Consensus | CDR H3 | H-(G/V/C/H)-(S/T)- |
| | Y61 to J695 | | (H/T/V/R/I)-(D/S)- |
| | | | (N/K/A/T/S/F/W/H) |
| 10 | Consensus | CDR L3 | Q-S-Y-(D/S)-(Xaa)- |
| | Y61 to J695 | | (G/D/Q/L/F/R/H/N/Y)-T-H- |
| | | | P-A-L-L |
| 11 | Consensus | CDR H2 | (F/T/Y)-I-(R/A)-Y- |
| | Y61 to J695 | | (D/S/E/A)-(G/R)-S-(Xaa)- |
| | | | K-(Y/E)-Y-A-D-S-V-K-G |
| 12 | Consensus | CDR L2 | (G/Y/S/T/N/Q)-N-D-Q-R-P- |
| | Y61 to J695 | | S |
| 13 | Consensus | CDR H1 | F-T-F-(Xaa)-(Xaa)-(Y/H)- |
| | Y61 to J695 | | (G/M/A/N/S)-M-H |
| 14 | Consensus | CDR L1 | S G G R S N I G |
| | Y61 to J695 | | (S/C/R/N/D/T)-(N/M/I)- |
| | | | (T/Y/D/H/K/P)-V-K |
| 15 | Consensus | VH | (full VH sequence; see |
| | Y61 to J695 | | sequence listing) |
| 16 | Consensus | VL | (full VL sequence; see |
| | Y61 to J695 | | sequence listing) |
| 17 | Y61 | CDR H3 | HGSHDN |
| 18 | Y61 | CDR L3 | QSYDRGTHPALL |
| 19 | Y61 | CDR H2 | FIRYDGSNKYYA |
| | | | D-S-V-K-G |
| 20 | Y61 | CDR L2 | G-N-D-Q-R-P-S |
| 21 | Y61 | CDR H1 | F-T-F-S-S-Y-G-M-H |
| 22 | Y61 | CDR L1 | S-G-G-R-S-N-I-G-S-N-T-V- |
| | | | K |
| 23 | Y61 | VH | (full VH sequence; see |
| | | | sequence listing) |
| 24 | Y61 | VL | (full VL sequence; see |
| | | | sequence listing) |
| 25 | J695 | CDR H3 | H-G-S-H-D-N |
| 26 | J695 | CDR L3 | Q-S-Y-D-R-Y-T-H-P-A-L-L |
| 27 | J695 | CDR H2 | F-I-R-Y-D-G-S-N-K-Y-Y-A- |
| | | | D-S-V-K-G |
| 28 | J695 | CDR L2 | Y-N-D-Q-R-P-S |
| 29 | J695 | CDR H1 | F-T-F-S-S-Y-G-M-H |
| 30 | J695 | CDR L1 | S-G-S-R-S-N-I-G-S-N-T-V- |
| | | | K |
| 31 | J695 | VH | (full VH sequence; see |
| | | | sequence listing) |
| 32 | J695 | VL | (full VL sequence; see |
| | | | sequence listing) |

Antibodies produced from affinity maturation of Joe 9 wild type were functionally characterized by surface plasmon resonance analysis to determine the K_{d} and K_{off} rate. A series of antibodies were produced having a K_{off} rate within the range of about 0.1 s⁻¹ to about 1×10⁻⁵ s⁻¹, and more preferably a K_{off} of about 1×10⁻⁴ s⁻¹ to 1×10⁻⁵ s⁻¹ or less. Antibodies were also characterized in vitro for their ability to inhibit phytohemagglutinin (PHA) blast proliferation, as described in Example 3 of U.S. Pat. No. 6,914,128. A series of antibodies were produced having an IC₅₀ value in the range of about 1×10⁻⁶ M to about 1×10⁻¹¹ M, more preferably about 1×10⁻¹⁰ M to 1×10⁻¹¹ M or less.

Accordingly, in one aspect, the invention provides methods and compositions for using an isolated human antibody, or antigen-binding portion thereof, that binds to human IL-12 and dissociates from human IL-12 with a K_{off} rate constant of 0.1 s⁻¹ or less, as determined by surface plasmon resonance, or which inhibits phytohemagglutinin blast proliferation in an in vitro phytohemagglutinin blast proliferation assay (PHA assay) with an IC₅₀ of 1×10⁻⁶ M or less. In preferred embodiments, the isolated human IL-12 antibody, or an antigen-binding portion thereof, dissociates from human IL-12 with a K_{off} rate constant of 1×10⁻² s⁻¹ or less, or inhibits phytohemagglutinin blast proliferation in an in vitro PHA assay with an IC₅₀ of 1×10⁻⁷ M or less. In more preferred embodiments, the isolated human IL-12 antibody, or an antigen-binding portion thereof, dissociates from human IL-12 with a K_{off} rate constant of 1×10⁻³ s⁻¹ or less, or inhibits phytohemagglutinin blast proliferation in an in vitro PHA assay with an IC₅₀ of 1×10⁻⁸ M or less. In more preferred embodiments, the isolated human IL-12 antibody, or an antigen-binding portion thereof, dissociates from human IL-12 with a K_{off} rate constant of 1×10⁻⁴ s⁻¹ or less, or inhibits phytohemagglutinin blast proliferation in an in vitro PHA assay with an IC₅₀ of 1×10⁻⁹ M or less. In more preferred embodiments, the isolated human IL-12 antibody, or an antigen-binding portion thereof, dissociates from human IL-12 with a K_{off} rate constant of 1×10⁻⁵ s⁻¹ or less, or inhibits phytohemagglutinin blast proliferation in an in vitro PHA assay with an IC₅₀ of 1×10⁻¹⁰ M or less. In even more preferred embodiments, the isolated human IL-12 antibody, or an antigen-binding portion thereof, dissociates from human IL-12 with a K_{off} rate constant of 1×10⁻⁵ s⁻¹ or less, or inhibits phytohemagglutinin blast proliferation in an in vitro PHA assay with an IC₅₀ of 1×10⁻¹¹ M or less.

It is well known in the art that antibody heavy and light chain CDRs play an important role in the binding specificity/affinity of an antibody for an antigen. Accordingly, the invention encompasses human antibodies having light and heavy chain CDRs of Joe 9, as well as other antibodies having CDRs that have been modified to improve the binding specificity/affinity of the antibody. As demonstrated in Example 1 of U.S. Pat. No. 6,914,128, a series of modifications to the light and heavy chain CDRs results in affinity maturation of human anti-hIL-12 antibodies. The heavy and light chain variable region amino acid sequence alignments of a series of human antibodies ranging from Joe 9 wild type to J695 that bind human IL-12 is shown in FIGS. 1A-1D of U.S. Pat. No. 6,914,128. Consensus sequence motifs for the CDRs of antibodies can be determined from the sequence alignment. For example, a consensus motif for the VH CDR3 of the lineage from Joe 9 to J695 comprises the amino acid sequence: (H/S)-G-S-(H/Y)-D-(N/T/Y) (SEQ ID NO: 1), which encompasses amino acids from position 95 to 102 of the consensus HCVR shown in SEQ ID NO: 7. A consensus motif for the VL CDR3 comprises the amino acid sequence: Q-(S/T)-Y-(D/E)-(S/R/K)-(S/G/Y)-(L/F/T/S)-(R/S/T/W/H)-(G/P)-(S/T/A/L)-(R/S/M/T/L-V/I/T/M/L) (SEQ ID NO: 2), which encompasses amino acids from position 89 to 97 of the consensus LCVR shown in SEQ ID NO: 8.

Accordingly, in another aspect, the invention provides methods and compositions comprising an isolated human antibody, or an antigen-binding portion thereof, which has the following characteristics: a) inhibits phytohemagglutinin blast proliferation in an in vitro PHA assay with an IC₅₀ of 1×10⁻⁶ M or less; b) has a heavy chain CDR3 comprising the amino acid sequence of SEQ ID NO: 1; and c) has a light chain CDR3 comprising the amino acid sequence of SEQ ID NO: 2.

In a preferred embodiment, the antibody further comprises a VH CDR2 comprising the amino acid sequence: F-1-R-Y-D-G-S-N-K-Y-Y-A-D-S-V-K-G (SEQ ID NO: 3) (which encompasses amino acids from position 50 to 65 of the consensus HCVR comprising the amino acid sequence SEQ ID NO: 7) and further comprises a VL CDR2 comprising the amino acid sequence: (G/Y)-N-(D/S)-(Q/N)-R-P-S (SEQ ID NO: 4) (which encompasses amino acids from position 50 to 56 of the consensus LCVR comprising the amino acid sequence SEQ ID NO: 8).

In another preferred embodiment, the antibody further comprises a VH CDR1 comprising the amino acid sequence: F-T-F-S-(S/E)-Y-G-M-H (SEQ ID NO: 5) (which encompasses amino acids from position 27 to 35 of the consensus HCVR comprising the amino acid sequence SEQ ID NO: 7) and further comprises a VL CDR1 comprising the amino acid sequence: (S/T)-G-(G/S)-(R/S)-S-N-1-(G/V)-(S/A)-(N/G/Y)-(T/D)-V-(K/H) (SEQ ID NO: 6) (which encompasses amino acids from position 24 to 34 of the consensus LCVR comprising the amino acid sequence SEQ ID NO: 8).

In yet another preferred embodiment, the antibody used in the invention comprises a HCVR comprising the amino acid sequence of SEQ ID NO: 7 and a LCVR comprising the amino acid sequence of SEQ ID NO: 8.

Additional consensus motifs can be determined based on the mutational analysis performed on Y61 that led to the J695 antibody (summarized in FIGS. 2A-2H of U.S. Pat. No. 6,914,128. As demonstrated by the graphs shown in FIGS. 2A-2H of U.S. Pat. No. 6,914,128, certain residues of the heavy and light chain CDRs of Y61 were amenable to substitution without significantly impairing the hIL-12 binding properties of the antibody. For example, individual substitutions at position 30 in CDR H1 with twelve different amino acid residues did not significantly reduce the K_{off} rate of the antibody, indicating that is position is amenable to substitution with a variety of different amino acid residues. Thus, based on the mutational analysis (i.e., positions within Y61 that were amenable to substitution by other amino acid residues) consensus motifs were determined. The consensus motifs for the heavy and light chain CDR3s are shown in SEQ ID NOs: 9 and 10, respectively, consensus motifs for the heavy and light chain CDR2s are shown in SEQ ID NOs: 11 and 12, respectively, and consensus motifs for the heavy and light chain CDR1s are shown in SEQ ID NOs: 13 and 14, respectively. Consensus motifs for the VH and VL regions are shown in SEQ ID NOs: 15 and 16, respectively.

Accordingly, in another aspect, the invention includes an isolated human antibody, or an antigen-binding portion thereof, which has the following characteristics: a) inhibits phytohemagglutinin blast proliferation in an in vitro PHA assay with an IC₅₀ of 1×10⁻⁹ M or less; b) has a heavy chain CDR3 comprising the amino acid sequence of SEQ ID NO: 9; and c) has a light chain CDR3 comprising the amino acid sequence of SEQ ID NO: 10.

In a preferred embodiment, the antibody further comprises a VH CDR2 comprising the amino acid sequence of SEQ ID NO: 11 and further comprises a VL CDR2 comprising the amino acid sequence of SEQ ID NO: 12.

In another preferred embodiment, the antibody further comprises a VH CDR1 comprising the amino acid sequence of SEQ ID NO: 13 and further comprises a VL CDR1 comprising the amino acid sequence of SEQ ID NO: 14.

In yet another preferred embodiment, the antibody used in the invention comprises a HCVR comprising the amino acid sequence of SEQ ID NO: 15 and a LCVR comprising the amino acid sequence of SEQ ID NO: 16.

A preferred antibody used in the invention, the human anti-hIL-12 antibody Y61, can be produced by affinity maturation of Joe 9 wild type by PCR mutagenesis of the CDR3 (as described in Example 1 of U.S. Pat. No. 6,914,128). Y61 had an improved specificity/binding affinity determined by surface plasmon resonance and by in vitro neutralization assays. The heavy and light chain CDR3s of Y61 are shown in SEQ ID NOs: 17 and 18, respectively, the heavy and light chain CDR2s of Y61 are shown in SEQ ID NOs: 19 and 20, respectively, and the heavy and light chain CDR1s of Y61 are shown in SEQ ID NOs: 21 and 22, respectively. The VH of Y61 has the amino acid sequence of SEQ ID NO: 23 and the VL of Y61 has the amino acid sequence of SEQ ID NO: 24 (these sequences are also shown in FIGS. 1A-1D of U.S. Pat. No. 6,914,128 aligned with Joe9).

Accordingly, in another aspect, the invention features use of an isolated human antibody, or an antigen-binding portion thereof, which a) inhibits phytohemagglutinin blast proliferation in an in vitro PHA assay with an IC₅₀ of 1×10⁻⁹ M or less; b) has a heavy chain CDR3 comprising the amino acid sequence of SEQ ID NO: 17; and c) has a light chain CDR3 comprising the amino acid sequence of SEQ ID NO: 18.

In a preferred embodiment, the isolated human antibody, or an antigen-binding portion thereof, used in the methods and compositions of the invention has a heavy chain CDR2 comprising the amino acid sequence of SEQ ID NO: 19 and a light chain CDR2 comprising the amino acid sequence of SEQ ID NO: 20.

In another preferred embodiment, the isolated human antibody, or an antigen-binding portion thereof, used in the methods and compositions of the invention, has a heavy chain CDR1 comprising the amino acid sequence of SEQ ID NO: 21 and a light chain CDR1 comprising the amino acid sequence of SEQ ID NO: 22.

In yet another preferred embodiment, the isolated human antibody, or an antigen-binding portion thereof, used in the methods and compositions of the invention comprising a the heavy chain variable region comprising the amino acid sequence of SEQ ID NO: 23, and a light chain variable region comprising the amino acid sequence of SEQ ID NO: 24.

In certain embodiments, the full length antibody comprises a heavy chain constant region, such as IgG1, IgG2, IgG3, IgG4, IgM, IgA and IgE constant regions, and any allotypic variant therein as described in Kabat (Kabat, E. A., et al. (1991) Sequences of Proteins of Immunological Interest, Fifth Edition, U.S. Department of Health and Human Services, NIH Publication No. 91-3242). Preferably, the antibody heavy chain constant region is an IgG1 heavy chain constant region. Alternatively, the antibody portion can be an Fab fragment, an F(ab′₂) fragment or a single chain Fv fragment.

A particularly preferred recombinant, neutralizing antibody, J695, which may be used in the invention was produced by site-directed mutagenesis of contact and hypermutation amino acids residues of antibody Y61 (see Example 2 of U.S. Pat. No. 6,914,128). J695 differs from Y61 by a Gly to Tyr substitution in Y61 at position 50 of the light chain CDR2 and by a Gly to Tyr substitution at position 94 of the light chain CDR3. The heavy and light chain CDR3s of J695 are shown in SEQ ID NOs: 25 and 26, respectively, the heavy and light chain CDR2s of J695 are shown in SEQ ID NOs: 27 and 28, respectively, and the heavy and light chain CDR1s of J695 are shown in SEQ ID NOs: 29 and 30, respectively. The VH of J695 has the amino acid sequence of SEQ ID NO: 31 and the VL of J695 has the amino acid sequence of SEQ ID NO: 32 (these sequences are also shown in FIGS. 1A-1D of U.S. Pat. No. 6,914,128, aligned with Joe9).

Accordingly, in another aspect, the invention features an isolated human antibody, or an antigen-binding portion thereof, which a) inhibits phytohemagglutinin blast proliferation in an in vitro PHA assay with an IC₅₀ of 1×10⁻⁹ M or less; b) has a heavy chain CDR3 comprising the amino acid sequence of SEQ ID NO: 25; and c) has a light chain CDR3 comprising the amino acid sequence of SEQ ID NO: 26.

In preferred embodiment, the isolated human antibody, or an antigen-binding portion thereof, used in the invention has a heavy chain CDR2 comprising the amino acid sequence of SEQ ID NO: 27, and a light chain CDR2 comprising the amino acid sequence of SEQ ID NO: 28.

In another preferred embodiment, the isolated human antibody, or an antigen-binding portion thereof, used in the invention has a heavy chain CDR1 comprising the amino acid sequence of SEQ ID NO: 29, and a light chain CDR1 comprising the amino acid sequence of SEQ ID NO: 30.

In yet another preferred embodiment, the isolated human antibody, or an antigen-binding portion thereof, used in the invention has a heavy chain variable region comprising the amino acid sequence of SEQ ID NO: 31, and a light chain variable region comprising the amino acid sequence of SEQ ID NO: 32. In certain embodiments, the full length antibody comprises a heavy chain constant region, such as IgG1, IgG2, IgG3, IgG4, IgM, IgA and IgE constant regions and any allotypic variant therein as described in Kabat (Kabat, E. A., et al. (1991) Sequences of Proteins of Immunological Interest, Fifth Edition, U.S. Department of Health and Human Services, NIH Publication No. 91-3242). Preferably, the antibody heavy chain constant region is an IgG1 heavy chain constant region. Alternatively, the antibody portion can be an Fab fragment, an F(ab′₂) fragment or a single chain Fv fragment.

Additional mutations in the preferred consensus sequences for CDR3, CDR2, and CDR1 of antibodies on the lineage from Joe 9 to J695, or from the lineage Y61 to J695, can be made to provide additional anti-IL-12 antibodies of the invention. Such methods of modification can be performed using standard molecular biology techniques, such as by PCR mutagenesis, targeting individual contact or hypermutation amino acid residues in the light chain and/or heavy chain CDRs-, followed by kinetic and functional analysis of the modified antibodies as described herein (e.g., neutralization assays described in Example 3 of U.S. Pat. No. 6,914,128, and by BIAcore analysis, as described in Example 5 of U.S. Pat. No. 6,914,128).

An ordinarily skilled artisan will also appreciate that additional mutations to the CDR regions of an antibody, for example in Y61 or in J695, can be made to provide additional anti-IL-12 antibodies of the invention. Such methods of modification can be performed using standard molecular biology techniques, as described above. The functional and kinetic analysis of the modified antibodies can be performed as described in Example 3 of U.S. Pat. No. 6,914,128 and Example 5 of U.S. Pat. No. 6,914,128, respectively. Modifications of individual residues of Y61 that led to the identification of J695 are shown in FIGS. 2A-2H of U.S. Pat. No. 6,914,128 and are described in Example 2 of U.S. Pat. No. 6,914,128. Typically, selection of antibodies with improved affinities can be carried out using phage display methods, as described in section II above and in U.S. Pat. No. 6,914,128, incorporated by reference herein.

### II. Expression of Antibodies

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To obtain a DNA fragment encoding the heavy chain variable region of Joe 9 wt or a Joe 9 wt-related antibody, antibodies specific for human IL-12 were screened from human libraries and mutated, as described in section II. Once DNA fragments encoding Joe 9 wt or Joe 9 wt-related VH and VL segments are obtained, mutagenesis of these sequences is carried out by standard methods, such as PCR site directed mutagenesis (PCR-mediated mutagenesis in which the mutated nucleotides are incorporated into the PCR primers such that the PCR product contains the mutations) or other site-directed mutagenesis methods. Human IL-12 antibodies that displayed a level of activity and binding specificity/affinity that was desirable, for example J695, were further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region and any allotypic variant therein as described in Kabat (, Kabat, E. A., et al. (1991) Sequences of Proteins of Immunological Interest, Fifth Edition, U.S. Department of Health and Human Services, NIH Publication No. 91-3242), but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions of the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the J695 or J695-related light or heavy chain sequences, the expression vector may already carry antibody constant region sequences. For example, one approach to converting the J695 or J695-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., U.S. Pat. No. 5,464,758 by Bujard et al. and U.S. Pat. No. 5,654,168 by Bujard et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hIL-12 The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hIL-12 by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Antibodies or antigen-binding portions thereof of the invention can be expressed in an animal (e.g., a mouse) that is transgenic for human immunoglobulin genes (see e.g., Taylor, L. D. et al. (1992) Nucl. Acids Res. 20: 6287-6295). Plant cells can also be modified to create transgenic plants that express the antibody or antigen binding portion thereof, of the invention.

In view of the foregoing, another aspect of the invention pertains to nucleic acid, vector and host cell compositions that can be used for recombinant expression of the antibodies and antibody portions of the invention. Preferably, the invention features isolated nucleic acids that encode CDRs of J695, or the full heavy and/or light chain variable region of J695. Accordingly, in one embodiment, the invention features an isolated nucleic acid encoding an antibody heavy chain variable region that encodes the J695 heavy chain CDR3 comprising the amino acid sequence of SEQ ID NO: 25. Preferably, the nucleic acid encoding the antibody heavy chain variable region further encodes a J695 heavy chain CDR2 which comprises the amino acid sequence of SEQ ID NO: 27. More preferably, the nucleic acid encoding the antibody heavy chain variable region further encodes a J695 heavy chain CDR1 which comprises the amino acid sequence of SEQ ID NO: 29. Even more preferably, the isolated nucleic acid encodes an antibody heavy chain variable region comprising the amino acid sequence of SEQ ID NO: 31 (the full VH region of J695).

In other embodiments, the invention features an isolated nucleic acid encoding an antibody light chain variable region that encodes the J695 light chain CDR3 comprising the amino acid sequence of SEQ ID NO: 26. Preferably, the nucleic acid encoding the antibody light chain variable region further encodes a J695 light chain CDR2 which comprises the amino acid sequence of SEQ ID NO: 28. More preferably, the nucleic acid encoding the antibody light chain variable region further encodes a J695 light chain CDR1 which comprises the amino acid sequence of SEQ ID NO: 30. Even more preferably, the isolated nucleic acid encodes an antibody light chain variable region comprising the amino acid sequence of SEQ ID NO: 32 (the full VL region of J695).

The invention also provides recombinant expression vectors encoding both an antibody heavy chain and an antibody light chain. For example, in one embodiment, the invention provides a recombinant expression vector encoding:

a) an antibody heavy chain having a variable region comprising the amino acid sequence of SEQ ID NO: 31; and

b) an antibody light chain having a variable region comprising the amino acid sequence of SEQ ID NO: 32.

The invention also provides host cells into which one or more of the recombinant expression vectors of the invention have been introduced. Preferably, the host cell is a mammalian host cell, more preferably the host cell is a CHO cell, an NS0 cell or a COS cell. Still further the invention provides a method of synthesizing a recombinant human antibody of the invention by culturing a host cell of the invention in a suitable culture medium until a recombinant human antibody of the invention is synthesized. The method can further comprise isolating the recombinant human antibody from the culture medium.

### III. Pharmaceutical Compositions and Pharmaceutical Administration

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody-portions of the invention can be incorporated into a pharmaceutical composition suitable for parenteral administration. Preferably, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In one embodiment, the invention provides a formulation comprising the antibody in combination with a polyol, a surfactant, a stabilizer, and a buffer system with a pH of about 5 to 5. In one embodiment said formulation is free of metal. In a preferred embodiment, the formulation comprises the antibody and mannitol, histidine, methionine, polysorbate 80, hydrochloric acid, and water.

In one embodiment, an aqueous formulation is prepared comprising the antibody in a pH-buffered solution. The buffer of this invention has a pH ranging from about 4 to about 8, preferably from about 4.5 to about 7.5, more preferably from about 5 to about 7, more preferably from about 5.5 to about 6.5, and most preferably has a pH of about 6.0 to about 6.2. In a particularly preferred embodiment, the buffer has a pH of about 6. Ranges intermediate to the above recited pH's are also intended to be part of this invention. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate, phosphate and other organic acid buffers. In a preferred embodiment of the invention, the formulation contains a buffer system comprising histidine. In a preferred embodiment of the invention, the buffer is histidine, e.g., L-histidine. In preferred embodiments, the formulation of the invention comprises a buffer system comprising about 1-100 mM histidine, preferably about 5-50 mM histidine, and most preferably 10 mM histidine. One of skill in the art will recognize that sodium chloride can be used to modify the toxicity of the solution, e.g., at a concentration of 1-300 mM, and optimally 150 mM for a liquid dosage form.

A polyol, which acts as a tonicifier and may stabilize the antibody, is also included in the formulation. The polyol is added to the formulation in an amount that may vary with respect to the desired isotonicity of the formulation. Preferably the aqueous formulation is isotonic. The amount of polyol added may also vary with respect to the molecular weight of the polyol. For example, a lower amount of a monosaccharide (e.g., mannitol) may be added, compared to a disaccharide (such as trehalose). In a preferred embodiment of the invention, the polyol that is used in the formulation as a tonicity agent is mannitol. In a preferred embodiment, the composition comprises about 10 to about 100 mg/ml, or about 20 to about 80, about 20 to about 70, about 30 to about 60, about 30 to about 50 mg/ml of mannitol, for example, about 10, about 20, about 30, about 40, about 50, about 60, about 70, about 80, about 90, and about 100 mg/ml of mannitol In a preferred embodiment, the formulation comprises about 40 mg/ml of mannitol (corresponding to about 4% mannitol). In a preferred embodiment, the composition comprises between about 1% to about 10% mannitol, more preferably between about 2% to about 6% mannitol, and most preferably about 4% mannitol. In another embodiment of the invention, the polyol sorbitol is included in the formulation.

A stabilizer or antioxidant is also added to the antibody formulation. A stabilizer can be used in both liquid and lyophilized dosage forms. Formulations of the invention preferably comprise the stabilizer methionine, e.g., L-Methionine. Other stabilizers useful in formulations of the invention are known to those of skill in the art and include, but are not limited to, glycine and arginine. Cryoprotectants can be included for a lyophilized dosage form, principally sucrose (e.g., 1-10% sucrose, and optimally 0.5-1.0% sucrose). Other suitable cyroprotectants include trehalose and lactose.

A detergent or surfactant is also added to the antibody formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In a preferred embodiment of the invention, the formulation includes a surfactant that is a polysorbate. In another preferred embodiment of the invention, the formulation contains the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In one preferred embodiment, the formulation contains between 0.001 to about 0.1% polysorbate 80, or between about 0.005 and 0.05% polysorbate 80, for example, about 0.001, about 0.005, about 0.01, about 0.05, or about 0.1% polysorbate 80. In a preferred embodiment, about 0.01% polysorbate 80 is found in the formulation of the invention.

In a preferred embodiment of the invention, the formulation is a 1.0 mL solution in a container containing the ingredients shown below in Table 1. In another embodiment, the formulation is a 0.8 mL solution in a container.

TABLE 1
A 1.0 mL Solution¹⁾ of J695 Formulation for Injection

| Name of Ingredient | Quantity | Function |
|---|---|---|
| Active substance: | | |
| Antibody (J695)²⁾ | 50.0 or 100.0 mg | Active substance |
| Excipients: | | |
| Mannitol | 40 mg | Tonicity agent |
| Polysorbate 80 | 0.10 mg | Detergent/Surfactant |
| Histidine | 1.55 mg | Buffer |
| Methionine | 1.49 mg | Stabilizer |
| Water for injection | To one 1 ml | Solvent |
| Hydrochloric Acid | q.s. | pH adjustment to 6.0 |

¹⁾Density of the solution: 1.0398 g/mL
²⁾Is used as concentrate

In one embodiment, the formulation is a formulation described in U.S. application Ser. No. 12/625,057, which published as U.S. 2010/0172862 A1, the entire contents of which are hereby expressly incorporated by reference.

In one embodiment, the formulation contains the above-identified agents (i.e., antibody, polyol, surfactant, stabilizer and buffer) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. One or more other pharmaceutically acceptable carriers, excipients or stabilizers such as those described in Remington's Pharmaceutical Sciences 16th edition, Osol, A. Ed. (1980) may be included in the formulation provided that they do not significantly adversely affect the desired characteristics of the formulation. Acceptable carriers, excipients or stabilizers are nontoxic to recipients at the dosages and concentrations employed and include; additional buffering agents; co-solvents; antioxidants such as ascorbic acid; chelating agents such as EDTA; metal complexes (e.g. Zn-protein complexes); biodegradable polymers such as polyesters; and/or salt-forming counterions such as sodium.

In one embodiment, the formulations of the invention have improved properties as compared to art-recognized formulations. For example, the formulations of the invention have an improved shelf life and/or stability as compared to art recognized formulations. In one embodiment, the formulations of the invention have a shelf life of at least 18 months, e.g., in a liquid state or in a solid state. In another embodiment, the formulations of the invention have a shelf life of at least 24 months, e.g., in a liquid state or in a solid state. In a preferred embodiment, the formulations of the invention have a shelf life of at least 24 months at a temperature of 2-8° C. In a preferred embodiment, the formulations of the invention have a shelf life of at least 18 months or of at least 24 months at a temperature of between about −20 and −80° C. In another embodiment, the formulations of the invention maintain stability following at least 5 freeze/thaw cycles of the formulation. In a preferred aspect, the formulations of the invention comprise, e.g., an antibody, comprising at least a portion of a lambda light chain, e.g., J695, wherein the formulation provides enhanced resistance to fragmentation of the lambda light chain, e.g., reduced cleavage of the lambda light chain, as compared to art recognized formulations.

In one embodiment, the formulations of the invention are substantially free of metal. In one embodiment, the formulations of the invention are substantially free of a metal selected from the group consisting of Fe2+, Fe3+, Ca2+ and Cu1+. In one embodiment, the formulations of the invention comprise an amount of metal that is sufficiently low to reduce or prevent cleavage of the lambda chain in the presence of histidine, e.g., the metal is present at a concentration of less than about 5,060 ppb, less than about 1,060 ppb, less than about 560 ppb, less than about 500 ppb, less than about 450 ppb, less than about 400 ppb, less than about 350 ppb, less than about 310 ppb, less than about 300 ppb, less than about 250 ppb, less than about 200 ppb, less than about 160 ppb, less than about 150 ppb, less than about 140 ppb, less than about 130 ppb, less than about 120 ppb, less than about 110 ppb, less than about 100 ppb, less than about 90 ppb, less than about 80 ppb, less than about 70 ppb, less than about 60 ppb, less than about 50 ppb, less than about 40 ppb, less than about 30 ppb, less than about 20 ppb, less than about 10 ppb, or less than about 1 ppb. In one embodiment, the metal is present at a concentration of less than about 160 ppb. In one embodiment, the metal is present at a concentration of less than about 110 ppb. In one embodiment, the metal is present at a concentration of less than about 70 ppb, e.g., a concentration of about 60 ppb. Maximum concentrations intermediate to the above recited concentrations, e.g., less than about 65 ppb, are also intended to be part of this invention. Further, ranges of values using a combination of any of the above recited values as upper and/or lower limits, e.g., concentrations between about 50 ppb and about 70 ppb, are also intended to be included.

In one embodiment, the formulations of the invention are substantially free of metal following subjection to at least one procedure that removes metal, such as filtration, buffer exchange, chromatography or resin exchange. Procedures useful to remove metal from formulations of the invention are known to one of skill in the art and are further described herein. In one embodiment, the formulations of the invention comprise a metal chelator, e.g., such that the molecule is not cleaved within the hinge region or is cleaved within the hinge region at a level which is less than the level of cleavage observed in the absence of the metal chelator. In the formulations of the invention, the metal chelator may be, for example, a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, a copper chelator, and derivatives, analogues, and combinations thereof. Metal chelators useful in formulations of the invention are known to one of skill in the art, and are further described below.

Particular siderophores useful in formulations of the invention include, but are not limited to, aerobactin, agrobactin, azotobactin, bacillibactin, N-(5-C3-L (5 aminopentyl)hydroxycarbamoyl)-propionamido)pentyl)-3(5-(N-hydroxyacetoamido)-pentyl)carbamoyl)-proprionhydroxamic acid (deferoxamine, desferrioxamine or DFO or DEF), desferrithiocin, enterobactin, erythrobactin, ferrichrome, ferrioxamine B, ferrioxamine E, fluviabactin, fusarinine C, mycobactin, parabactin, pseudobactin, vibriobactin, vulnibactin, yersiniabactin, ornibactin, and derivatives, analogues, and combinations thereof.

Aminopolycarboxylic acids useful in formulations of the invention include, but are not limited to, ethylenediaminetetraacetic acid (EDTA), nitriloacetic acid (NTA), trans-diaminocyclohexane tetraacetic acid (DCTA), diethylenetriamine pentaacetic acid (DTPA), N-2-acetamido-2-iminodiacetic acid (ADA), aspartic acid, bis(aminoethyl)glycolether N,N,N′N′-tetraacetic acid (EGTA), glutamic acid, and N,N′-bis(2-hydroxybenzyl)ethylenediamine-N,N′-diacetic acid (HBED), and derivatives, analogues, and combinations thereof.

Hydroxyaminocarboxylic acids useful in formulations of the invention include, but are not limited to, N-hydroxyethyliminodiacetic acid (HIMDA), N,N-bishydroxyethylglycine (bicine), and N-(trishydroxymethylmethyl) glycine (tricine), and derivatives, analogues, and combinations thereof. N-substituted glycines, e.g., glycylglycine, as well as derivatives, analogues, or combinations thereof, are also useful as metal chelators in formulations of the invention. The metal chelator 2-(2-amino-2-oxoethyl)aminoethane sulfonic acid (BES), and derivatives, analogues, and combinations thereof, can also be used.

Particular calixarenes useful in formulations of the invention include, but are not limited to, a macrocycle or cyclic oligomer based on a hydroxyalkylation product of a phenol and an aldehyde, and derivatives, analogues, and combinations thereof. Particular copper chelators useful in the invention include triethylenetetramine (trientine), etraethylenepentamine, D-penicillamine, ethylenediamine, bispyridine, phenantroline, bathophenanthroline, neocuproine, bathocuproine sulphonate, cuprizone, cis,cis-1,3,5,-triaminocyclohexane (TACH), tachpyr, and derivatives, analogues, and combinations thereof.

Additional metal chelators that can be employed in formulations of the invention include a hydroxypyridine-derivate, a hydrazone-derivate, and hydroxyphenyl-derivate, or a nicotinyl-derivate, such as 1,2-dimethyl-3-hydroxypyridin-4-one (Deferiprone, DFP or Ferriprox); 2-deoxy-2-(N-carbamoylmethyl-[N′-2′-methyl-3′-hydroxypyridin-4′-one])-D-glucopyranose (Feralex-G), pyridoxal isonicotinyl hydrazone (P1H); 4,5-dihydro-2-(2,4-dihydroxyphenyl)-4-methylthiazole-4-carboxylic acid (GT56-252), 4-[3,5-bis(2-hydroxyphenyl)[1,2,4]-triazol-1-yl]benzoic acid (ICL-670); N,N′-bis(o-hydroxybenzyl)ethylenediamine-N,N′-diacetic acid (HBED), 5-chloro-7-iodo-quinolin-8-ol (clioquinol), and derivatives, analogues, and combinations thereof.

It will be recognized that combinations of two or more of any of the foregoing metal chelators can be used in combination in the formulations of the invention. For example, in a particular embodiment of the invention, the formulation comprises a combination of DTPA and DEF. In another embodiment, the formulation comprises a combination of EDTA, EGTA and DEF.

The amount of antibody present in the formulation is determined, for example, by taking into account the desired dose volumes and mode(s) of administration. In one embodiment of the invention, the concentration of the antibody in the formulation is between about 0.1 to about 250 mg of antibody per ml of liquid formulation. In one embodiment of the invention, the concentration of the antibody in the formulation is between about 1 to about 200 mg of antibody per ml of liquid formulation. In various embodiments, the concentration of the antibody in the formulation is between about 30 to about 140 mg per ml, between about 40 to about 120 mg/ml, between about 50 to about 110 mg/ml, or between about 60 to about 100 mg/ml. The formulation is especially suitable for large antibody dosages of more than 15 mg/ml. In various embodiments, the concentration of the antibody in the formulation is about 1, 10, 20, 30, 40, 50, 60, 70, 80, 90, 100, 110, 120, 130, 140, 150, 160, 170, 180, 190, 200, 210, 220, 230, 240 or 250 mg/ml. In a preferred embodiment, the concentration of the antibody is 50 mg/ml. In another preferred embodiment, the concentration of the antibody is 100 mg/ml. In a preferred embodiment, the concentration of the antibody is at least about 100 mg/ml, at least about 110 mg/ml or at least about 120 mg/ml.

In various embodiments of the invention, the concentration of the antibody in the formulation is about 0.1-250 mg/ml, 0.5-220 mg/ml, 1-210 mg/ml, about 5-200 mg/ml, about 10-195 mg/ml, about 15-190 mg/ml, about 20-185 mg/ml, about 25-180 mg/ml, about 30-175 mg/ml, about 35-170 mg/ml, about 40-165 mg/ml, about 45-160 mg/ml, about 50-155 mg/ml, about 55-150 mg/ml, about 60-145 mg/ml, about 65-140 mg/ml, about 70-135 mg/ml, about 75-130 mg/ml, about 80-125 mg/ml, about 85-120 mg/ml, about 90-115 mg/ml, about 95-110 mg/ml, about 95-105 mg/ml, or about 100 mg/ml. Ranges intermediate to the above recited concentrations, e.g., about 31-174 mg/ml, are also intended to be part of this invention. For example, ranges of values using a combination of any of the above recited values as upper and/or lower limits are intended to be included.

In one, the formulation provides an effective dose of 40 mg, 50 mg, 80 mg, 100 mg, or 200 mg per injection of the active ingredient, the antibody. In another embodiment, the formulation provides an effective dose which ranges from about 0.1 to 250 mg of antibody. If desired, the effective daily dose of the pharmaceutical formulation may be administered as two, three, four, five, six or more sub-doses administered separately at appropriate intervals throughout the day, optionally, in unit dosage forms. In an embodiment of the invention, the dosage of the antibody in the formulation is between about 1 to about 200 mg. In an embodiment, the dosage of the antibody in the formulation is between about 30 and about 140 mg, between about 40 and about 120 mg, between about 50 and about 110 mg, between about 60 and about 100 mg, or between about 70 and about 90 mg. In one embodiment, the pharmaceutical composition includes the antibody at a dose of about 100 to about 200 mg. In a further embodiment, the composition includes the antibody at about 1, 10, 20, 30, 40, 50, 60, 70, 80, 90, 100, 110, 120, 130, 140, 150, 160, 170, 180, 190, 200, 210, 220, 230, 240 or 250 mg.

Ranges intermediate to the above recited dosages, e.g., about 2-139 mg, are also intended to be part of this invention. For example, ranges of values using a combination of any of the above recited values as upper and/or lower limits are intended to be included.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody, or antigen-binding fragment thereof, is administered by subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody-portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating disorders in which IL-12 activity is detrimental. For example, an anti-hIL-12 antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

Interleukin 12 plays a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, autoimmune uveitis.

Preferably, the antibodies of the invention or antigen-binding portions thereof, are used to treat rheumatoid arthritis, Crohn's disease, multiple sclerosis, insulin dependent diabetes mellitus and psoriasis, as described in more detail in section VII.

A human antibody, or antibody portion, of the invention also can be administered with one or more additional therapeutic agents useful in the treatment of autoimmune and inflammatory diseases.

Antibodies of the invention, or antigen binding portions thereof can be used alone or in combination to treat such diseases. It should be understood that the IL-12 antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function. Furthermore, additional agents described herein used in combination with an IL-12 antibody, are not limited to the disorder to which they are attributed for treatment.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the anti-IL-12 antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF (including adalimumab/HUMIRA), LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7, (U.S. application Ser. No. 08/599,226 filed Feb. 9, 1996), cA2 (Remicade™), CDP 571, anti-TNF antibody fragments (e.g., CDP870), and soluble p55 or p75 TNF receptors, derivatives thereof, (p75TNFR1gG (Enbrel™) or p55TNFR1gG (Lenercept), soluble IL-13 receptor (sIL-13), and also TNFα converting enzyme (TACE) inhibitors; similarly IL-1 inhibitors (e.g., Interleukin-1-converting enzyme inhibitors, such as Vx740, or IL-1RA etc.) may be effective for the same reason. Other preferred combinations include Interleukin 11, anti-P7s and p-selectin glycoprotein ligand (PSGL). Yet another preferred combination are other key players of the autoimmune response which may act parallel to, dependent on or in concert with IL-12 function; especially preferred are IL-18 antagonists including IL-18 antibodies or soluble IL-18 receptors, or IL-18 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

Anti-IL12 antibodies, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1R1, sIL-1R11, sIL-6R, soluble IL-13 receptor (sIL-13)) and anti-inflammatory cytokines (e.g. IL-4, IL-10, IL-11, IL-13 and TGFβ). Preferred combinations include methotrexate or leflunomide and in moderate or severe rheumatoid arthritis cases, cyclosporine.

Non-limiting examples of therapeutic agents for inflammatory bowel disease with which an anti-IL-12 antibody, or antibody portion, can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF (including adalimumab/HUMIRA), LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1R1, sIL-1R11, sIL-6R, soluble IL-13 receptor (sIL-13)) and anti-inflammatory cytokines (e.g. IL-4, IL-10, IL-11, IL-13 and TGFβ).

Preferred examples of therapeutic agents for Crohn's disease in which an antibody or an antigen binding portion can be combined include the following: TNF antagonists, for example, anti-TNF antibodies, D2E7 (adalimumab/HUMIRA), cA2 (Remicade™), CDP 571, anti-TNF antibody fragments (e.g., CDP870), TNFR-Ig constructs (p75TNFRIgG (Enbrel™) and p55TNFRIgG (Lenercept)), anti-P7s, p-selectin glycoprotein ligand (PSGL), soluble IL-13 receptor (sIL-13), and PDE4 inhibitors. Antibodies of the invention or antigen binding portions thereof, can be combined with corticosteroids, for example, budenoside and dexamethasone. Antibodies may also be combined with agents such as sulfasalazine, 5-aminosalicylic acid and olsalazine, and agents which interfere with synthesis or action of proinflammatory cytokines such as IL-1, for example, IL-1β converting enzyme inhibitors (e.g., Vx740) and IL-1ra. Antibodies or antigen binding portion thereof may also be used with T cell signaling inhibitors, for example, tyrosine kinase inhibitors 6-mercaptopurines. Antibodies or antigen binding portions thereof, can be combined with IL-11.

Non-limiting examples of therapeutic agents for multiple sclerosis with which an antibody, or antibody portion, can be combined include the following: corticosteroids; prednisolone; methylprednisolone; azathioprine; cyclophosphamide; cyclosporine; methotrexate; 4-aminopyridine; tizanidine; interferon-β1a (Avonex; Biogen); interferon-β1b (Betaseron; Chiron/Berlex); Copolymer 1 (Cop-1; Copaxone; Teva Pharmaceutical Industries, Inc.); hyperbaric oxygen; intravenous immunoglobulin; clabribine; antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80, CD86, CD90 or their ligands. The antibodies of the invention, or antigen binding portions thereof, may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-10 converting enzyme inhibitors (e.g., Vx740), anti-P7s, p-selectin glycoprotein ligand (PSGL), TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1R1, sIL-1R11, sIL-6R, soluble IL-13 receptor (sIL-13)) and anti-inflammatory cytokines (e.g. IL-4, IL-10, IL-13 and TGFβ).

Preferred examples of therapeutic agents for multiple sclerosis in which the antibody or antigen binding portion thereof can be combined to include interferon-0, for example, IFNβ1a and IFNβ1b; copaxone, corticosteroids, IL-1 inhibitors, TNF inhibitors, and antibodies to CD40 ligand and CD80.

An antibody, antibody portion, may be used in combination with other agents to treat skin conditions. For example, an antibody, antibody portion, or other IL-12 inhibitor of the invention is combined with PUVA therapy. PUVA is a combination of psoralen (P) and long-wave ultraviolet radiation (UVA) that is used to treat many different skin conditions. The antibodies, antibody portions, or other IL-12 inhibitors of the invention can also be combined with pimecrolimus. In another embodiment, the antibodies of the invention are used to treat psoriasis, wherein the antibodies are administered in combination with tacrolimus. In a further embodiment, tacrolimus and IL-12 inhibitors are administered in combination with methotrexate and/or cyclosporine. In still another embodiment, the IL-12 inhibitor of the invention is administered with excimer laser treatment for treating psoriasis.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

The present invention provides various methods of treating psoriasis, as described herein. Related methods of treating psoriasis are described in U.S. application Ser. No. 12/881,902, filed Sep. 14, 2010, the entire contents of which are expressly incorporated herein by reference.

Treatment of psoriasis may be achieved by administration of a single dose amount (or more than one sub-doses totaling the dose amount) of a substance according to a single periodicity.

In one embodiment, a method of treating psoriasis in a subject comprises administering to the subject an antibody, or antigen-binding portion thereof, which is capable of binding to the p40 subunit of IL-12 and/or IL-23, according to a periodicity of about once every 4 weeks, thereby treating psoriasis in the subject.

In another embodiment, a method of treating psoriasis in a subject comprises administering to the subject an antibody, or antigen-binding portion thereof, which is capable of binding to the p40 subunit of IL-12 and/or IL-23, according to a periodicity of about once every 12 weeks, thereby treating psoriasis in the subject.

Thus, a single periodicity may be employed in a single treatment regimen. Alternatively, multiple periodicities may be employed in a single treatment regimen. For example, a first dose amount may be administered according to a first periodicity, and then the first dose amount or a second dose amount may be administered according to a second periodicity. Furthermore, the first dose amount or second dose amount administered according to a second periodicity may optionally be followed by a first, second, or third dose amount administered according to a third periodicity.

In one embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount at the same periodicity.

In another embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount according to a second periodicity.

In one embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount according to a second periodicity, and is further administered to the subject as a first, second, or third dose amount according to a third periodicity.

The first dose amount of the antibody, or antigen-binding portion thereof, may be at least about 100 mg to about 200 mg, is at least about 100 mg, or is at least about 200 mg. The first dose amount of the antibody, or antigen-binding portion thereof, may be about 100 mg, about 110 mg, about 120 mg, about 130 mg, about 140 mg, about 150 mg, about 160 mg, about 170 mg, about 180 mg, about 190 mg, or about 200 mg. In one embodiment, the first dose amount is about 180-220 mg, 185-215 mg, 190-210 mg, or 195-205 mg. In one embodiment, the first dose amount is 200 mg. In one embodiment, the first dose amount is about 80-120 mg, 85-115 mg, 90-110 mg or 95-105 mg. In one embodiment, the first dose amount is 100 mg. It should be noted that doses intermediate to the above specified doses are also included herein, e.g., 105 mg, 127 mg, etc.

The second dose amount of the antibody, or antigen-binding portion thereof, may be the same as the first dose amount of the antibody, or antigen-binding portion thereof, or different than the first dose amount of the antibody, or antigen-binding portion thereof. The second dose amount of the antibody, or antigen-binding portion thereof, may be at least about 100 mg to about 200 mg, is at least about 200 mg, or is at least about 100 mg. Alternatively, the second dose amount of the antibody, or antigen-binding portion thereof, is about 40-60% (e.g., 40, 41, 42, 43, 44, 45, 46, 47, 48, 49, 50, 51, 52, 53, 54, 55, 56, 57, 58, 59 or 60%), e.g., about 50%, of the first dose amount of the antibody, or antigen-binding portion thereof, or antigen-binding portion thereof, or about 190-210% (e.g., 190, 191, 192, 193, 194, 195, 196, 197, 198, 199, 200, 201, 202, 203, 204, 205, 206, 207, 208, 209, 210%), e.g., about 200%, of the first dose amount of the antibody, or antigen-binding portion thereof. The second dose amount of the antibody, or antigen-binding portion thereof, may be about 100 mg, about 110 mg, about 120 mg, about 130 mg, about 140 mg, about 150 mg, about 160 mg, about 170 mg, about 180 mg, about 190 mg, or about 200 mg. In one embodiment, the second dose amount is about 80-120 mg, 85-115 mg, 90-110 mg or 95-105 mg. In one embodiment, the second dose amount is 100 mg. In another embodiment, the second dose amount is about 180-220 mg, 185-215 mg, 190-210 mg, or 195-205 mg. In one embodiment, the second dose amount is 200 mg. It should be noted that doses intermediate to the above specified doses are also included herein, e.g., 105 mg, 127 mg, etc.

The first and second periodicities of administration of the antibody, or antigen-binding portion thereof, may be about once a week, about once every other week, about once every four weeks. The second periodicity of administration of the antibody, or antigen-binding portion thereof, may be about once every 30-200 days.

The duration of the first periodicity may be about 12 weeks, about 8 weeks, about 4 weeks, about 2 weeks, or about 1 week. The duration of the second periodicity may be about 60 weeks, about 44 weeks, about 12 weeks, about 4 weeks, about 2 weeks, or about 1 week.

The duration of a third periodicity may be, for example, about 4 weeks, about 12 weeks, about 24 weeks, about 36 weeks, about 48 weeks or about 60 weeks.

Thus, in one aspect, a method of treating psoriasis in a subject comprises administering to the subject a first dose amount of an antibody, or antigen-binding portion thereof, which is capable of binding to the p40 subunit of IL-12 and/or IL-23; and a second dose amount that is about 40-60% of the first dose amount of the antibody, or antigen-binding portion thereof, according to a periodicity of about once every 12 weeks, thereby treating psoriasis in the subject.

In another aspect, a method of treating psoriasis in a subject comprises administering to the subject a first dose amount of an antibody, or antigen-binding portion thereof, which is capable of binding to the p40 subunit of IL-12 and/or IL-23, according to a first periodicity of about once every 4 weeks; and administering a second dose amount that is about 40-60% of the first dose amount of the antibody, or antigen-binding portion thereof, according to a second periodicity of about once every 4 weeks, thereby treating psoriasis in the subject.

In another aspect, a method of treating psoriasis in a subject comprises administering to the subject a first dose amount of an antibody, or antigen-binding portion thereof, which is capable of binding to the p40 subunit of IL-12 and/or IL-23, according to a first periodicity of about once every 4 weeks; and a second dose amount that is about 40-60% of the first dose amount of the antibody, or antigen-binding portion thereof, according to a second periodicity of about once every 4 weeks; and the second dose amount of the antibody, or antigen-binding portion thereof, according to a third periodicity of about once every 12 weeks, thereby treating psoriasis in the subject.

In one embodiment, the second dose amount is administered to the subject upon a flare of psoriasis. In another embodiment, the second dose amount is administered to the subject prior to a flare of psoriasis.

The flare of psoriasis may be monitored by determining a subject's Psoriasis Area and Severity Index (PASI), e.g., PASI 100 response, PASI 90 response, PASI 75 response, PASI 50 response, the PASI response of a single body region, two body regions, three body regions, or four body regions, e.g., trunk, lower extremities, upper extremities, or head and neck. Alternatively, the flare of psoriasis may be monitored by determining a subject's Physician's Global Assessment (PGA) rating.

In one embodiment, the subject achieves or maintains a specific response to treatment. In one embodiment, the subject achieves or maintains at least a PASI 50 response. In one embodiment, the subject achieves or maintains at least a PASI 75 response. In one embodiment, the subject achieves or maintains at least a PASI 90 response. In one embodiment, the subject achieves or maintains at least a PASI 100 response. In one embodiment the PASI 50, 75, 90, or 100 response is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one embodiment, the PASI 50, 75, 90, or 100 response is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PASI 50, 75, 90 or 100 response is maintained, once achieved, throughout the duration of treatment.

In one embodiment, the subject achieves a PGA score of 0 or 1. In one embodiment the PGA score of 0 or 1 is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one embodiment, the PGA score of 0 or 1 is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PGA score of 0 or 1 is maintained, once achieved, throughout the duration of treatment.

In one embodiment, the subject achieves a PGA score of 0, i.e., total clearance. In one embodiment the PGA score of 0 is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one embodiment, the PGA score of 0 is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PGA score of 0 is maintained, once achieved, throughout the duration of treatment.

A method of treating psoriasis in a population of subjects may comprise administering to each subject in the population an antibody, or antigen-binding portion thereof, which is capable of binding to the p40 subunit of IL-12 and/or IL-23, wherein at least 60% of the population of subjects achieve a PASI 75 response, e.g., by about week 12.

A method of treating psoriasis in a population of subjects may comprise administering to each subject in the population an antibody, or antigen-binding portion thereof, which is capable of binding to the p40 subunit of IL-12 and/or IL-23, wherein at least 25% of the population of subjects achieve a PASI 90 response, e.g., by about week 12.

A method of treating psoriasis in a population of subjects may comprise administering to each subject in the population an antibody, or antigen-binding portion thereof, which is capable of binding to the p40 subunit of IL-12 and/or IL-23, wherein at least 10% of the population of subjects achieve a PASI 100 response, e.g., by about week 12.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 50 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 75 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 90 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 100 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PGA score of 0 or 1 by about week 12, 24, 36, 48, 52, or 60.

In one aspect, the subject or population of subjects treated achieves an improvement in a Dermatology Life Quality Index (DLQI) score or mean Dermatology Life Quality Index (DLQI) score of at least about −6.8, −6.9, −7.0, −8.0, −8.5, −9, −10, −10.5, −11, −12, −13, −14, −15, −16, −17, −18, −19, −20 or lower. An improvement in DLQI is a reduction in DLQI score, e.g., a reduction by at least about 6.8, 6.9, 7.0, 8.0, 8.5, 9, 10, 10.5, 11, 12, 13, 14, 15, 16, 17, 18, 19, 20 or more. Dermatology Life Quality Index (DLQI) is a patient-reported measure of the extent to which psoriasis impacts health-related quality of life. The DLQI yields a score ranging from 0 to 30, with a lower score indicating lower impact.

In certain embodiments, the subject achieves a clinically meaningful reduction in Dermatology Life Quality Index (DLQI) score. A clinically meaningful reduction in Dermatology Life Quality Index (DLQI) score may be, e.g., a decrease of greater than 5, 6, 7, or 8 points in DLQI score.

In another aspect, the subject or population of subjects treated achieves an improvement in a Short Form 36 Health Survey Physical Component Summary (PCS) score or mean Physical Component Summary (PCS) score of at least about 2, 3, 4, 5, 6, or more. An improvement in PCS is an increase in PCS score, e.g., an increase by at least about 2, 3, 4, 5, 6, or more.

In another aspect, the subject or population of subjects treated achieves an improvement in a Short Form 36 Health Survey Mental Component Summary (MCS) score or mean Mental Component Summary (MCS) score of at least about 3.5, 4, 4.5, 6, 6.5, 7, or more. An improvement in PCS is an increase in MCS score, e.g., an increase by at least about 3.5, 4, 4.5, 6, 6.5, 7, or more.

In another aspect, the subject or population of subjects treated achieves an improvement in a visual analog scale score or a mean visual analog scale score for psoriasis-related pain (VAS-Ps) of at least about −22, −23, −24, −25, −26, −27, −28, −29, −30, −31, −32, −33, −34, −35, −40, −45, −50, or less. An improvement in VAS-Ps is a reduction in VAS-Ps score, e.g., a reduction by at least about 22, 23, 24, 25, 26, 27, 28, 29, 30, 31, 32, 33, 34, 35, 40, 45, 50, or more.

In another aspect, the subject or population of subjects treated achieves an improvement in a visual analog scale score for psoriatic arthritis-related pain (VAS-PsA) or a mean visual analog scale score for psoriatic arthritis-related pain (VAS-PsA) of at least about −16, −18, −20, −25, −26, −27, −28, −29, −30, −31, −32, −33, −34, −35, −40, −45, −50, or less. An improvement in VAS-PsA is a reduction in VAS-Ps score, e.g., a reduction by at least about 16, 18, 20, 25, 26, 27, 28, 29, 30, 31, 32, 33, 34, 35, 40, 45, 50, or more.

In another aspect, the population of subjects treated achieves a minimum clinically important difference (MCID) response rate in any one or more HRQOL outcomes including, e.g., DLQI, TAI, VAS-Ps, Vas-PsA, MCS and PCS of at least about 40%, 45%, 50%, 55%, 60%, 65%, 70%, 75%, 80%, 85%, 90%.

In another aspect, the population of subjects treated achieves a minimum clinically important difference (MCID) response rate for psoriasis-related pain (VAS-Ps) of at least about 55%, 57%, 60%, 65%, 70%, 75%, or more, e.g., by about week 12 or by about week 52.

In another aspect, the population of subjects treated achieves a minimum clinically important difference (MCID) response rate for Dermatology Life Quality Index (DLQI) of at least about 70%, 75%, 80%, 82% or more by about week 12.

In another aspect, the population of subjects treated achieves a minimum clinically important difference (MCID) response rate for Dermatology Life Quality Index (DLQI) of at least about 75%, 80%, 85%, 90%, or more by about week 52.

In another aspect, the population of subjects treated achieves a minimum clinically important difference (MCID) response rate for Total Activity Impairment (TAI) of at least about 45%, 50%, 55%, 60%, 70%, or more by about week 12.

In another aspect, the population of subjects treated achieves a minimum clinically important difference (MCID) response rate for Total Activity Impairment (TAI) of at least about 50%, 55%, 57%, 60%, 65% or more by about week 52.

In another aspect, efficacy may be assessed by Nail Psoriasis Severity Index (NAPSI) scores, which range from 0 (no nail psoriasis) to 80 (psoriasis in all 10 fingernails). In certain embodiments, the subject achieves a Nail Psoriasis Severity Index (NAPSI) score of about 40, 35, 30, 25, 20, 15, 10, 9, 8, 7, 6, 5, 4, 3, 2, 1 or less. In certain embodiments, the subject achieves a Nail Psoriasis Severity Index (NAPSI) score of about 2.1 or less. In certain embodiments, the subject achieves a Nail Psoriasis Severity Index (NAPSI) score of about 2.1 or less by about week 24. In certain embodiments, the subject achieves a Nail Psoriasis Severity Index (NAPSI) score of about 1.2 or less. In certain embodiments, the subject achieves a Nail Psoriasis Severity Index (NAPSI) score of about 1.2 or less by about week 52.

In another aspect at least 40%, 45%, 50%, 55%, 60%, 65%, or more of the population of subjects treated achieve at least a PGA 0/1 response by about week 12, wherein each subject was treated with a biologic prior to administration of the antibody.

In another aspect, at least 50%, 55%, 60%, 65%, 70%, 75% of the population of subjects treated achieve at least a PASI 75 response by about week 12, wherein each subject was treated with a biologic prior to administration of the antibody.

In another aspect, at least 60%, 65%, 70%, 75%, 78%, or more of the population of subjects treated achieve at least a PGA 0/1 response by about week 12, wherein none of the subjects were treated with a biologic prior to administration of the antibody.

In another aspect, at least 60%, 65%, 70%, 75%, 80%, 82% or more of the population of subjects achieve at least a PASI 75 response by about week 12, wherein none of the subjects were treated with a biologic prior to administration of the antibody.

In another aspect, at least 60%, 65%, 70%, 75%, 78%, or more of the population of subjects treated achieve at least a PGA 0/1 response by about week 52, wherein each subject was treated with a biologic prior to administration of the antibody.

In another aspect at least 60%, 65%, 70%, 75%, 79%, 80%, 82% or more of the population of subjects treated achieve at least a PGA 0/1 response by about week 52, wherein none of the subjects were treated with a biologic prior to administration of the antibody.

In another aspect, at least 50%, 55%, 60%, 65%, 70%, 71%, or more of the population of subjects treated achieve at least a PGA 0/1 response by about week 12, wherein each subject treated has a prior history of psoriatic arthritis.

In another aspect, at least 60%, 65%, 70%, 75%, 78%, or more of the population of subjects treated achieve at least a PASI 75 response by about week 12, wherein each subject treated has a prior history of psoriatic arthritis.

In another aspect, at least 60%, 65%, 70%, 75%, 77%, or more of the population of subjects treated achieve at least a PGA 0/1 response by about week 12, wherein none of the subjects treated has a prior history of psoriatic arthritis.

In another aspect, at least 60%, 65%, 70%, 75%, 81%, or more of the population of subjects treated achieve at least a PASI 75 response by about week 12, wherein none of the subjects treated has a prior history of psoriatic arthritis.

In another aspect, at least 60%, 65%, 70%, 75%, 77%, or more of the population of subjects treated achieve at least a PGA 0/1 response by about week 52, wherein each subject treated has a prior history of psoriatic arthritis.

In another aspect, at least 60%, 65%, 70%, 75%, 79%, or more of the population of subjects treated achieve at least a PGA 0/1 response by about week 52, wherein none of the subjects treated has a prior history of psoriatic arthritis.

In another aspect, at least 50%, 55%, 60%, 65%, 69%, or more of the population of subjects achieve at least a PGA 0/1 response by about week 12, wherein each subject had a baseline PASI greater than 20 prior to administration of the antibody.

In another aspect, at least 60%, 65%, 70%, 75%, 79%, or more of the population of subjects achieve at least a PGA 0/1 response by about week 12, wherein each subject had a baseline PASI less than or equal to 20 prior to administration of the antibody.

In another aspect, at least 60%, 65%, 70%, 75%, 79%, or more of the population of subjects achieve at least a PASI 75 response by about week 12, wherein each subject had a baseline PASI greater than 20 prior to administration of the antibody.

In another aspect, at least 60%, 65%, 70%, 75%, 80%, 81%, or more of the population of subjects achieve at least a PASI 75 response by about week 12, wherein each subject had a baseline PASI less than or equal to 20 prior to administration of the antibody.

In another aspect, at least 50%, 55%, 60%, 65%, 67%, or more of the population of subjects achieve at least a PGA 0/1 response by about week 12, wherein each subject had a baseline weight of greater than or equal to 100 kilograms prior to administration of the antibody.

In another aspect, at least 60%, 65%, 70%, 75%, 80%, or more of the population of subjects achieve at least a PGA 0/1 response by about week 12, wherein each subject had a baseline weight of less than 100 kilograms prior to administration of the antibody.

In another aspect, at least 50%, 55%, 60%, 65%, 70%, 72% or more of the population of subjects achieve at least a PASI 75 response by about week 12, wherein each subject had a baseline weight of greater than or equal to 100 kilograms prior to administration of the antibody.

In another aspect, at least 60%, 65%, 70%, 75%, 80%, 85%, or more of the population of subjects achieve at least a PASI 75 response by about week 12, wherein each subject had a baseline weight of less than 100 kilograms prior to administration of the antibody.

In another aspect, a method of treating psoriasis in a subject or population of subjects comprises administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in a Short Form 36 Health Survey domain score selected from the group consisting of a Physical Function score, a Role-Physical score, a Bodily Pain score, a General Health score, a Vitality score, a Social Function score, a Role-Emotional score, and a Mental Health score. In one embodiment, the subject or population of subjects achieves an improvement or mean improvement in a Short Form 36 Health Survey Physical Function score of at least about 2, 2.5, 3, 3.5, or 4. In another embodiment, the subject or population of subjects achieves an improvement or mean improvement in a Short Form 36 Health Survey Role-Physical score of at least about 2, 2.5, 3, or 3.5. In another embodiment, the subject or population of subjects achieves an improvement or mean improvement in a Short Form 36 Health Survey Bodily Pain score of at least about 5, 5.5, 6, 6.5, or 7. In another embodiment, the subject or population of subjects achieves an improvement or mean improvement in a Short Form 36 Health Survey General Health score of at least about 2, 2.5, 3, 3.5, or 4. In another embodiment, the subject or population of subjects achieves an improvement or mean improvement in a Short Form 36 Health Survey Vitality score of at least about 2, 2.5, 3 or 3.5. In another embodiment, the subject or population of subjects achieves an improvement or mean improvement in a Short Form 36 Health Survey Social Function score of at least about 3, 3.5, 4, 4.5, 5, 5.5, or 6. In another embodiment, the subject or population of subjects achieves an improvement or mean improvement in a Short Form 36 Health Survey Role-Emotional score of at least about 4, 4.5, 5, 5.5, or 6. In another embodiment, the subject or population of subjects achieves an improvement or mean improvement in a Short Form 36 Health Survey Mental Health score of at least about 2, 2.5, 3, or 3.5.

In another embodiment, at least 20%, 25%, 30%, 34%, or 35% of the population of subjects achieves an improvement at or exceeding a minimum clinically important difference (MCID) response for a Short Form 36 Health Survey Physical Function score. In another embodiment, at least 20%, 25%, 26% or 30% of the population of subjects achieves an improvement at or exceeding a minimum clinically important difference (MCID) response for a Short Form 36 Health Survey Role-Physical score. In another embodiment, at least 25%, 30%, 35%, 40%, 42% or 45% of the population of subjects achieves an improvement at or exceeding a minimum clinically important difference (MCID) response for a Short Form 36 Health Survey Bodily Pain score. In another embodiment, at least 16%, 20%, 23%, or 25% of the population of subjects achieves an improvement at or exceeding a minimum clinically important difference (MCID) response for a Short Form 36 Health Survey General Health score. In another embodiment, at least 20%, 25%, 30%, 35%, 40%, 42%, or 45% of the population of subjects achieves an improvement at or exceeding a minimum clinically important difference (MCID) response for a Short Form 36 Health Survey Vitality score. In another embodiment, at least 5%, 10%, 15%, 20%, 25%, 30%, 31%, or 35% of the population of subjects achieves an improvement at or exceeding a minimum clinically important difference (MCID) response for a Short Form 36 Health Survey Social Function score. In another embodiment, at least 5%, 10%, 15%, 16%, or 20% of the population of subjects achieves an improvement at or exceeding a minimum clinically important difference (MCID) response for a Short Form 36 Health Survey Role Emotional score. In another embodiment, at least 35%, 40%, or 45% of the population of subjects achieves an improvement at or exceeding a minimum clinically important difference (MCID) response for a Short Form 36 Health Survey Mental Health score.

In another aspect, a method of treating psoriasis in a subject or population of subjects comprises administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in an HRQOL outcome selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP). In one embodiment, the subject or population of subjects achieves an improvement or mean improvement in a Dermatology Life Quality Index (DLQI) score by at least about −8, −10, −11, −12, −13, −14, or −15. In another embodiment, the subject or population of subjects achieves an improvement or mean improvement in a psoriasis-related pain (VAS-Ps) score by at least about −25, −30, −35, or −40. In another embodiment, the subject or population of subjects achieves an improvement or mean improvement in a psoriatic arthritis-related pain (VAS-PsA) score by at least about −15, −20, −25, or −30. In another embodiment, the subject or population of subjects achieves an improvement or mean improvement in a work productivity and activity impairment-specific health problem for psoriasis (WPAI-SHP) score by at least about −2, −3, or −3.5 for % work time missed. In another embodiment, the subject or population of subjects achieves an improvement or mean improvement in a work productivity and activity impairment-specific health problem for psoriasis (WPAI-SHP) score by at least about −13, −14, −15, −16, −17, −18, or −19 for % impairment while working. In another embodiment, the subject or population of subjects achieves an improvement or mean improvement in a work productivity and activity impairment-specific health problem for psoriasis (WPAI-SHP) score by at least about −13, −14, −15, −16, −17, −18, −19, or −20 for % overall work impairment. In another embodiment, the subject or population of subjects achieves an improvement or mean improvement in a work productivity and activity impairment-specific health problem for psoriasis (WPAI-SHP) score by at least about −18, −20, −22, or −25 for % overall activity impairment. In another embodiment, at least about 60%, 65%, 68%, or 70% of the population of subjects achieves an improvement at or exceeding a minimum clinically important difference (MCID) response for psoriasis-related pain (VAS-Ps) by about week 12 or 52. In another embodiment, at least about 50%, 55%, 59%, or 60% of the population of subjects achieves an improvement at or exceeding a minimum clinically important difference (MCID) response for psoriatic arthritis-related pain (VAS-PsA) by about week 12 or 52. In another embodiment, at least about 6%, 7%, 8%, or 8.4% of the population of subjects achieves an improvement at or exceeding a minimum clinically important difference (MCID) response for work productivity and activity impairment-specific health problem for psoriasis (WPAI-SHP) for % work time missed by about week 12 or 52. In another embodiment, at least about 35%, 40%, 41%, 42%, or 45% of the population of subjects achieves an improvement at or exceeding a minimum clinically important difference (MCID) response for work productivity and activity impairment-specific health problem for psoriasis (WPAI-SHP) for % impairment while working. In another embodiment, at least about 35%, 40%, 41%, 42%, or 45% of the population of subjects achieves an improvement at or exceeding a minimum clinically important difference (MCID) response for work productivity and activity impairment-specific health problem for psoriasis (WPAI-SHP) for % overall work impairment. In another embodiment, at least about 45%, 50%, 55%, 56%, or 58% of the population of subjects achieves an improvement at or exceeding a minimum clinically important difference (MCID) response for work productivity and activity impairment-specific health problem for psoriasis (WPAI-SHP) for % overall activity impairment.

In one aspect, the invention provides methods of treating psoriasis in a subject or population of subjects, comprising administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in a EQ-5D score or in a EQ-5D-VAS score. In a related aspect, the invention provides methods of treating psoriasis in a subject or population of subjects, comprising selecting a subject or population of subjects who would benefit from an improvement in a Health Related Quality of Life score, e.g., a EQ-5D score or EQ-5D-VAS score, and administering to the subject or population of subjects an antibody, or antigen-binding portion thereof, which is capable of binding to the p40 subunit of IL-12 and/or IL-23, wherein the subject or population of subjects upon treatment achieves an improvement or mean improvement in a EQ-5D score or in a EQ-5 D-VAS score.

The EQ-5D scores (also referred to herein as EQ-5D_{index} scores) are based on the EQ-5D Descriptive System Health Questionnaire (EQ-5D_{index} land include five dimensions of HRQOL: anxiety/depression, mobility, self care, usual activities and pain/discomfort. The scoring algorithm is based on the social preferences of the UK population, and scores range from −0.594 to 1.0, with 1.0 being the best possible score.

In one embodiment, the subject or population of subjects achieves an improvement or mean improvement in a EQ-5D score of at least about 0.15, e.g., at week 12. In one embodiment, the subject or population of subjects achieves an improvement or mean improvement in a EQ-5D score of at least about 0.20, e.g., at week 12.

In one embodiment, the subject or population of subjects achieves an improvement or mean improvement in a EQ-5D score of at least about 0.16, e.g., at week 24. In one embodiment, the subject or population of subjects achieves an improvement or mean improvement in a EQ-5D score of at least about 0.17, 0.18, 0.19 or 0.20, e.g., at week 24. In one embodiment, the subject or population of subjects achieves an improvement or mean improvement in a EQ-5D-VAS score of at least about 12.0, e.g., at week 24. In one embodiment, the subject or population of subjects achieves an improvement or mean improvement in a EQ-5D-VAS score of at least about 13, 14, 15, 16, 17, 18, 19 or 19.49, e.g., at week 24.

In one embodiment, the subject or population of subjects achieves an improvement or mean improvement in a EQ-5D score of at least about 0.16, e.g., at week 52. In one embodiment, the subject or population of subjects achieves an improvement or mean improvement in a EQ-5D score of at least about 0.24, e.g., at week 52. In one embodiment, the subject or population of subjects achieves an improvement or mean improvement in a EQ-5D-VAS score of at least about 12.3, e.g., at week 52. In one embodiment, the subject or population of subjects achieves an improvement or mean improvement in a EQ-5D-VAS score of at least about 13, 14, 15, 16, 17, 18, 19, 20 or 21, e.g., at week 52.

In another embodiment, at least 44% of the population of subjects achieves an improvement at or exceeding a minimum clinically important difference (MCID) response (i.e., a MCID response rate) for a EQ-5D score, e.g., at week 12. In one embodiment, at least 50%, or 57% of the population of subjects achieves an improvement at or exceeding a minimum clinically important difference (MOD) response for a EQ-5D score, e.g., at week 12.

In another embodiment, at least 50% of the population of subjects achieves an improvement at or exceeding a minimum clinically important difference (MCID) response (i.e., a MOD response rate) for a EQ-5D score, e.g., at week 24. In one embodiment, at least 51, 52, 53, 54, 55, 56, 57, 58, 59, 60, 61 or 61.6% of the population of subjects achieves an improvement at or exceeding a minimum clinically important difference (MCID) response for a EQ-5D score, e.g., at week 24.

In another embodiment, at least 57% of the population of subjects achieves an improvement at or exceeding a minimum clinically important difference (MCID) response (i.e., a MCID response rate) for a EQ-5D-VAS score, e.g., at week 24. In one embodiment, at least 58, 59, 60, 61, 62, 63, 65, 66, 67, 68, 69, 70, 71 or 71.6% of the population of subjects achieves an improvement at or exceeding a minimum clinically important difference (MCID) response for a EQ-5D-VAS score, e.g., at week 24.

In another embodiment, at least 17.5% of the population of subjects achieves an improvement at or exceeding a minimum clinically important difference (MCID) response (i.e., a MCID response rate) for a EQ-5D score, e.g., at week 52. In one embodiment, at least 20%, 25%, 30%, 35%, 40%, 45%, 48% or 49% of the population of subjects achieves an improvement at or exceeding a minimum clinically important difference (MCID) response for a EQ-5D score, e.g., at week 52.

In one embodiment of the various methods of the invention, the improvement described herein is achieved by about week 12. In another embodiment, the improvement described herein is achieved by about week 52.

In yet another aspect of the invention, a method of treating psoriasis in a subject comprises administering to the subject an antibody, or antigen-binding portion thereof, which is capable of binding to the p40 subunit of IL-12 and/or IL-23, wherein the subject upon treatment achieves a PGA score of 0 or 1 in less than about 54, 55, 56, 57, 58, 59, or 60 days. In another aspect, a method of treating psoriasis in a population of subjects comprises administering to each subject in the population an antibody, or antigen-binding portion thereof, which is capable of binding to the p40 subunit of IL-12 and/or IL-23, wherein the population of subjects upon treatment achieves a Physician's Global Assessment (PGA) score of 0 or 1 in a median time of less than about 54, 55, 56, 57, 58, 59, or 60 days.

In another aspect, a method of treating psoriasis in a subject comprises administering to the subject an antibody, or antigen-binding portion thereof, which is capable of binding to the p40 subunit of IL-12 and/or IL-23, wherein the subject upon treatment achieves a Psoriasis Area and Severity Index (PASI) 75 response in less than about 57, 58, 59, 60, 65, 70, 75, 80 or 85 days. In another aspect, a method of treating psoriasis in a population of subjects comprises administering to each subject in the population an antibody, or antigen-binding portion thereof, which is capable of binding to the p40 subunit of IL-12 and/or IL-23, wherein the population of subjects upon treatment achieves a Psoriasis Area and Severity Index (PASI) 75 response in a median time of less than about 57, 58, 59, 60, 65, 70, 75, 80 or 85 days.

In another aspect, a method of treating psoriasis in a subject comprises administering to the subject an antibody, or antigen-binding portion thereof, which is capable of binding to the p40 subunit of IL-12 and/or IL-23, wherein the subject upon treatment achieves a Dermatology Life Quality Index (DLQI) score of 0 by about week 12. In another aspect, a method of treating psoriasis in a population of subjects comprises administering to each subject in the population an antibody, or antigen-binding portion thereof, which is capable of binding to the p40 subunit of IL-12 and/or IL-23, wherein at least about 20%, 25%, 30%, 35%, 40%, 45%, 49%, or 50% of the population of subjects upon treatment achieve a Dermatology Life Quality Index (DLQI) score of 0 by about week 12.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 5%, 10%, 15%, or 20% of the population of subjects upon treatment achieve at least a PGA score of 0 or 1 by about week 4.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 18%, 20%, 25%, 30%, 35%, 40%, 45%, or 50% of the population of subjects upon treatment achieve a PGA score of 0 or 1 by about week 8.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 5%, 10%, 15%, or 20% of the population of subjects upon treatment achieve at least a PASI 75 response by about week 4.

In another aspect, a method of treating psoriasis in a population of subjects, comprising administering to each subject in the population an antibody, or antigen-binding portion thereof, which is capable of binding to the p40 subunit of IL-12 and/or IL-23, wherein at least about 25%, 30%, 35%, 40%, 45%, 50%, 55%, or 60% of the population of subjects upon treatment achieve at least a PASI 75 response by about week 8.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 40%, 40%, 45%, 50%, 55%, 60%, 65%, 70%, 75%, or 80% of the population of subjects upon treatment achieve at least a PASI 75 response by about week 12.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 10%, 15%, 20%, 25%, 30%, or 35% of the population of subjects upon treatment achieve at least a PASI 90 response by about week 8.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 15%, 20%, 25%, 30%, 35%, 40%, 45%, or 50% of the population of subjects upon treatment achieve at least a PASI 90 response by about week 12.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 10%, 15%, 20%, 25%, 30%, 40%, or 50% of the population of subjects upon treatment achieve a PASI 100 response by about week 8.

In another aspect, a method of treating psoriasis in a population of subjects comprises administering to each subject in the population an antibody, or antigen-binding portion thereof, which is capable of binding to the p40 subunit of IL-12 and/or IL-23, wherein at least about 5%, 10%, 20%, 25%, or 30% of the population of subjects upon treatment achieve a PASI 100 response by about week 12.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 80%, or 85% of the population of subjects achieve at least a PASI 75 response by about week 52, wherein each subject was treated with a biologic prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 80%, 82% or 85% of the population of subjects achieve at least a PASI 75 response by about week 52, wherein none of the subjects were treated with a biologic prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 60%, 65%, or 70% of the population of subjects achieve a PGA score of 0 or 1 by about week 12, wherein each subject was treated with a biologic and showed no improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 65%, 70%, 75%, or 80% of the population of subjects achieve a PGA score of 0 or 1 by about week 12, wherein each subject was treated with a biologic and showed improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 65%, 70%, 72%, 75%, or 80% of the population of subjects achieve a PGA score of 0 or 1 by about week 52, wherein each subject was treated with a biologic and showed no improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 65%, 70%, 72%, 75%, 76% or 80% of the population of subjects achieve a PGA score of 0 or 1 by about week 52, wherein each subject was treated with a biologic and showed improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 70%, 73%, 75%, or 78% of the population of subjects achieve at least a PASI 75 response by about week 12, wherein each subject was treated with a biologic and showed no improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 79%, 80%, or 85% of the population of subjects achieve at least a PASI 75 response by about week 12, wherein each subject was treated with a biologic and showed improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 70%, 75%, 77%, or 80% of the population of subjects achieve at least a PASI 75 response by about week 52, wherein each subject was treated with a biologic and showed no improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 78%, 80%, or 85% of the population of subjects achieve at least a PASI 75 response by about week 52, wherein each subject was treated with a biologic and showed improvement prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 78%, 80%, 82%, or 85% of the population of subjects achieve at least a PASI 75 response by about week 52, wherein each subject has a prior history of psoriatic arthritis.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 78%, 80%, 82%, or 85% of the population of subjects achieve at least a PASI 75 response by about week 52, wherein none of the subjects has a prior history of psoriatic arthritis.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 78%, 80%, 82%, or 85% of the population of subjects achieve a PGA score of 0 or 1 by about week 52, wherein each subject had a baseline weight of less than 100 kilograms prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 70%, 73%, 75%, or 78% of the population of subjects achieve a PGA score of 0 or 1 by about week 52, wherein each subject had a baseline weight of greater than or equal to 100 kilograms prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 80%, 83%, 84%, or 85% of the population of subjects achieve at least a PASI 75 response by about week 52, wherein each subject had a baseline weight of less than 100 kilograms prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 79%, 80%, or 85% of the population of subjects achieve at least a PASI 75 response by about week 52, wherein each subject had a baseline weight of greater than or equal to 100 kilograms prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 79%, 80%, 81%, or 85% of the population of subjects achieve a PGA score of 0 or 1 by about week 52, wherein each subject had a baseline PASI score of less than or equal to 20 prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 70%, 73%, or 75% of the population of subjects achieve a PGA score of 0 or 1 by about week 52, wherein each subject had a baseline PASI score of greater than 20 prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 80%, 84%, 85%, or 90% of the population of subjects achieve at least a PASI 75 response by about week 52, wherein each subject had a baseline PASI score of less than or equal to 20 prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 78%, or 80% of the population of subjects achieve at least a PASI response by about week 52, wherein each subject had a baseline PASI score of greater than 20 prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 80%, or 85% of the population of subjects achieve a PGA score of 0 or 1 by about week 12, wherein each subject had less than or equal to 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 65%, 70%, 75%, or 80% of the population of subjects achieve a PGA score of 0 or 1 by about week 12, wherein each subject had greater than 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 80%, 85%, 87%, 90%, or 95% of the population of subjects achieve a PGA score of 0 or 1 by about week 52, wherein each subject had less than or equal to 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 65%, 70%, 75%, or 80% of the population of subjects achieve a PGA score of 0 or 1 by about week 52, wherein each subject had greater than 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 80%, 83%, 85%, or 90% of the population of subjects achieve at least a PASI 75 response by about week 12, wherein each subject had less than or equal to 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 75%, 77%, 80%, or 85% of the population of subjects achieve at least a PASI 75 response by about week 12, wherein each subject had greater than 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 80%, 85%, 86%, or 90% of the population of subjects achieve at least a PASI 75 response by about week 52, wherein each subject had less than or equal to 20% body surface area (BSA) affected by psoriasis prior to administration of the antibody.

In another aspect, a method of treating psoriasis in a population of subjects comprises administering to each subject in the population an antibody, or antigen-binding portion thereof, which is capable of binding to the p40 subunit of IL-12 and/or IL-23, wherein at least 70%, 75%, 76%, 80%, or 85% of the population of subjects achieve at least a PASI 75 response by about week 52, wherein each subject had greater than 20% body surface area (BSA) affected by psoriasis prior to administration of the antibody.

Unless otherwise stated, in any embodiment described herein, the antibody, or antigen-binding portion thereof, may be administered according to a periodicity of about once every 4 week, thereby treating psoriasis in the subjects.

Unless otherwise stated, in any embodiment described herein, the antibody, or antigen-binding portion thereof, may be administered according to a periodicity of about once every 12 weeks, thereby treating psoriasis in the subject.

Unless otherwise stated, in any embodiment described herein, the antibody, or antigen-binding portion thereof, may be administered in a) a first dose amount according to a first periodicity of about once every 4 weeks; and b) a second dose amount that is about 40-60% of the first dose amount of the antibody, or antigen-binding portion thereof, according to a second periodicity of about once every 4 weeks, thereby treating psoriasis in the subject.

Unless otherwise stated, in any embodiment described herein, the antibody, or antigen-binding portion thereof, may be administered in a) a first dose amount of an antibody, or antigen-binding portion thereof, which is capable of binding to the p40 subunit of IL-12 and/or IL-23, according to a first periodicity of about once every 4 weeks; and b) a second dose amount that is about 40-60% of the first dose amount of the antibody, or antigen-binding portion thereof, according to a second periodicity of about once every 4 weeks; and c) the second dose amount of the antibody, or antigen-binding portion thereof, according to a third periodicity of about once every 12 weeks, thereby treating psoriasis in the subject.

Unless otherwise stated, in any embodiment described herein, the first dose amount may be at least about 200 mg. Unless otherwise stated, in any embodiment described herein, the second dose amount may be at least about 100 mg. Unless otherwise stated, in any embodiment described herein the antibody may be a human antibody. Unless otherwise stated, in any embodiment described herein, the antibody may be ABT-874.

Unless otherwise stated, in any embodiment described herein, any method of the invention may comprise administering to the subject or to each subject in the population: a) about 200 mg of ABT-874 once every four weeks for two doses; and b) about 100 mg of ABT-874 every four weeks thereafter.

Unless otherwise stated, in any embodiment described herein, any method of the invention may comprise administering to the subject or to each subject in the population: a) about 200 mg of ABT-874 at weeks 0 and 4; and b) about 100 mg of ABT-874 at week 8 and every 4 weeks thereafter.

Unless otherwise stated, in any embodiment described herein, the antibody may be administered subcutaneously.

Unless otherwise stated, in any embodiment described herein, the psoriasis to be treated may be moderate to severe psoriasis, chronic psoriasis, or plaque psoriasis.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### III. Uses of the Invention

The invention provides methods for inhibiting IL-12 activity in a subject suffering from a disorder in which IL-12 activity is detrimental.

IL-12 has been implicated in the pathophysiology of a wide variety of disorders (Windhagen et al., (1995) J. Exp. Med. 182: 1985-1996; Morita et al. (1998) Arthritis and Rheumatism. 41: 306-314; Bucht et al., (1996) Clin. Exp. Immunol. 103: 347-367; Fais et al. (1994) J. Interferon Res. 14:235-238; Parronchi et al., (1997) Am. J. Path. 150:823-832; Monteleone et al., (1997) Gastroenterology. 112:1169-1178, and Berrebi et al., (1998) Am. J. Path 152:667-672; Parronchi et al (1997) Am. J. Path. 150:823-832). The invention provides methods for inhibiting IL-12 activity in a subject suffering from such a disorder, which method comprises administering to the subject an antibody or antibody portion of the invention such that IL-12 activity in the subject is inhibited. Preferably, the IL-12 is human IL-12 and the subject is a human subject. Alternatively, the subject can be a mammal expressing a IL-12 with which an antibody of the invention cross-reacts. Still further the subject can be a mammal into which has been introduced hIL-12 (e.g., by administration of hIL-12 or by expression of an hIL-12 transgene). An antibody of the invention can be administered to a human subject for therapeutic purposes (discussed further below). Moreover, an antibody of the invention can be administered to a non-human mammal expressing a IL-12 with which the antibody cross-reacts for veterinary purposes or as an animal model of human disease. Regarding the latter, such animal models may be useful for evaluating the therapeutic efficacy of antibodies of the invention (e.g., testing of dosages and time courses of administration).

As used herein, the phrase “a disorder in which IL-12 activity is detrimental” is intended to include diseases and other disorders in which the presence of IL-12 in a subject suffering from the disorder has been shown to be or is suspected of being either responsible for the pathophysiology of the disorder or a factor that contributes to a worsening of the disorder. Accordingly, a disorder in which IL-12 activity is detrimental is a disorder in which inhibition of IL-12 activity is expected to alleviate the symptoms and/or progression of the disorder. Such disorders may be evidenced, for example, by an increase in the concentration of IL-12 in a biological fluid of a subject suffering from the disorder (e.g., an increase in the concentration of IL-12 in serum, plasma, synovial fluid, etc. of the subject), which can be detected, for example, using an anti-IL-12 antibody as described above. There are numerous examples of disorders in which IL-12 activity is detrimental. In one embodiment, the antibodies or antigen binding portions thereof, can be used in therapy to treat the diseases or disorders described herein. In another embodiment, the antibodies or antigen binding portions thereof, can be used for the manufacture of a medicine for treating the diseases or disorders described herein. The use of the antibodies and antibody portions of the invention in the treatment of a few non-limiting specific disorders is discussed further below:

### A. Rheumatoid Arthritis:

Interleukin-12 has been implicated in playing a role in inflammatory diseases such as rheumatoid arthritis. Inducible IL-12p40 message has been detected in synovia from rheumatoid arthritis patients and IL-12 has been shown to be present in the synovial fluids from patients with rheumatoid arthritis (see e.g., Morita et al., (1998) Arthritis and Rheumatism 41: 306-314). IL-12 positive cells have been found to be present in the sublining layer of the rheumatoid arthritis synovium. The human antibodies, and antibody portions of the invention can be used to treat, for example, rheumatoid arthritis, juvenile rheumatoid arthritis, Lyme arthritis, rheumatoid spondylitis, osteoarthritis and gouty arthritis. Typically, the antibody, or antibody portion, is administered systemically, although for certain disorders, local administration of the antibody or antibody portion may be beneficial. An antibody, or antibody portion, of the invention also can be administered with one or more additional therapeutic agents useful in the treatment of autoimmune diseases.

In the collagen induced arthritis (CIA) murine model for rheumatoid arthritis, treatment of mice with an anti-IL-12 mAb (rat anti-mouse IL-12 monoclonal antibody, C17.15) prior to arthritis profoundly suppressed the onset, and reduced the incidence and severity of disease. Treatment with the anti-IL-12 mAb early after onset of arthritis reduced severity, but later treatment of the mice with the anti-IL-12 mAb after the onset of disease had minimal effect on disease severity.

### B. Crohn's Disease

Interleukin-12 also plays a role in the inflammatory bowel disease, Crohn's disease. Increased expression of IFN-7 and IL-12 occurs in the intestinal mucosa of patients with Crohn's disease (see e.g., Fais et al., (1994) J. Interferon Res. 14: 235-238; Parronchi et al., (1997) Amer. J. Pathol. 150: 823-832; Monteleone et al., (1997) Gastroenterology 112: 1169-1178; Berrebi et al., (1998) Amer. J. Pathol. 152: 667-672). Anti-IL-12 antibodies have been shown to suppress disease in mouse models of colitis, e.g., TNBS induced colitis IL-2 knockout mice, and recently in IL-10 knock-out mice. Accordingly, the antibodies, and antibody portions, of the invention, can be used in the treatment of inflammatory bowel diseases.

### C. Multiple Sclerosis

Interleukin-12 has been implicated as a key mediator of multiple sclerosis. Expression of the inducible IL-12 p40 message or IL-12 itself can be demonstrated in lesions of patients with multiple sclerosis (Windhagen et al., (1995) J. Exp. Med. 182: 1985-1996, Drulovic et al., (1997) J. Neurol. Sci. 147: 145-150). Chronic progressive patients with multiple sclerosis have elevated circulating levels of IL-12. Investigations with T-cells and antigen presenting cells (APCs) from patients with multiple sclerosis revealed a self-perpetuating series of immune interactions as the basis of progressive multiple sclerosis leading to a Th1-type immune response. Increased secretion of IFN-γ from the T cells led to increased IL-12 production by APCs, which perpetuated the cycle leading to a chronic state of a Th1-type immune activation and disease (Balashov et al., (1997) Proc. Natl. Acad. Sci. 94: 599-603). The role of IL-12 in multiple sclerosis has been investigated using mouse and rat experimental allergic encephalomyelitis (EAE) models of multiple sclerosis. In a relapsing-remitting EAE model of multiple sclerosis in mice, pretreatment with anti-IL-12 mAb delayed paralysis and reduced clinical scores.

Treatment with anti-IL-12 mAb at the peak of paralysis or during the subsequent remission period reduced clinical scores. Accordingly, the antibodies or antigen binding portions thereof of the invention may serve to alleviate symptoms associated with multiple sclerosis in humans.

### D. Insulin-Dependent Diabetes Mellitus

Interleukin-12 has been implicated as an important mediator of insulin-dependent diabetes mellitus (IDDM). IDDM was induced in NOD mice by administration of IL-12, and anti-IL-12 antibodies were protective in an adoptive transfer model of IDDM. Early onset IDDM patients often experience a so-called “honeymoon period” during which some residual islet cell function is maintained. These residual islet cells produce insulin and regulate blood glucose levels better than administered insulin. Treatment of these early onset patients with an anti-IL-12 antibody may prevent further destruction of islet cells, thereby maintaining an endogenous source of insulin.

### E. Psoriasis

Interleukin-12 (IL-12) and the related cytokine IL-23 have been implicated as key mediators in psoriasis. Psoriasis involves acute and chronic skin lesions that are associated with a TH1-type cytokine expression profile (Hamid et al. (1996) J. Allergy Clin. Immunol. 1:225-231; Turka et al. (1995) Mol. Med. 1:690-699). Both IL-12 and IL-23 contribute to the development of the type 1T helper cell (Th1) immune response in psoriasis. Moreover, the IL-12 p40 and IL-23 p40 messenger RNA is overexpressed in psoriatic skin lesions. Accordingly, the antibodies or antigen binding portions thereof of the invention may serve to alleviate chronic skin disorders such psoriasis.

In one embodiment, the invention provides a method for treating psoriasis. Treatment for psoriasis often includes a topical corticosteroids, vitamin D analogs, and topical or oral retinoids, or combinations thereof. In one embodiment, an IL-12 and/or IL-23 antibody is administered in combination with or the presence of one of these common treatments. Additional therapeutic agents which can be combined with the IL-12 and/or IL-23 antibody for treatment of psoriasis are described in more detail below.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

Improvements in psoriasis in a subject can be monitored by the subject's Psoriasis Area and Severity Index Score (PASI). The method for determining the PASI has been described in Fredriksson and Pettersson (1978) Dermatologica 157:238 and Marks et al. (1989) Arch Dermatol 125:235. Briefly, the index is based on evaluation of four anatomic sites, including the head, upper extremities, trunk, and lower extremities, for erythema, induration, and desquamation using a 5 point scale (0=no symptoms; 1=slight; 2=moderate; 3=marked; 4=very marked). Based on the extent of lesions in a given anatomic site, the area affected is assigned a numerical value (0=0; 1=<10%; 2=10-29%; 3=30-49%; 4=50-69%; 5=70=89%; 6=90-100%). The PASI score is then calculated, wherein the possible range of PASI score is 0.0 to 72.0 with the highest score representing complete erythroderma of the severest degree.

In one embodiment of the invention, an IL-12 and/or IL-23 antibody is used for the treatment of psoriasis, including plaque psoriasis, e.g., chronic plaque psoriasis, moderate plaque psoriasis, and severe plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA). In another embodiment, an IL-12 and/or IL-23 antibody, such as J695/ABT-874, is used to treat subjects who have psoriasis in combination with PsA. In one embodiment of the invention, an IL-12 and/or IL-23 antibody is used for the treatment of nail psoriasis.

In one aspect, the invention provides methods for treating psoriasis in difficult to treat subjects by administering antibodies, and antigen binding portions thereof, of the invention, for example, ABT-874. Difficult to treat subjects may include, for example, subjects who have been previously administered biologics for the treatment of psoriasis, subjects who have had a history of psoriatic arthritis, subjects who have psoriasis and weigh greater than 100 kg, and subjects who have a baseline PASI greater than 20. Accordingly, in one aspect, the invention provides methods for treating subjects who have been previously administered biologics for the treatment of psoriasis by administering antibodies, and antigen binding portions thereof, of the invention, for example, ABT-874. Specifically, the methods involve selecting subjects who have received prior biologic treatment and administering antibodies of the invention. As set forth in Example 19, the data demonstrates efficacy of ABT-874 in the treatment of psoriasis in this subgroup of subjects. In another aspect, the invention provides methods for treating subjects who have had a history of psoriatic arthritis by administering antibodies, and antigen binding portions thereof, of the invention, for example, ABT-874. Specifically, the methods involve selecting subjects who have had a history of psoriatic arthritis and administering antibodies of the invention. In another aspect, the invention provides methods for treating subjects who weigh greater than 100 kg by administering antibodies, and antigen binding portions thereof, of the invention, for example, ABT-874. Specifically, the methods involve selecting subjects who weigh greater than 100 kg and administering antibodies of the invention. In yet another aspect, the invention provides methods for treating subjects who had a baseline PASI greater than 20 by administering antibodies, and antigen binding portions thereof, of the invention, for example, ABT-874. Specifically, the methods involve selecting subjects who have a baseline PASI greater than 20 prior to administration of the antibody and administering antibodies of the invention.

Specific types of psoriasis included in the treatment methods of the invention are described in detail below:

a. Chronic Plaque Psoriasis

Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

b. Guttate Psoriasis

Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

c. Inverse Psoriasis

Inverse psoriasis is a form of psoriasis in which the patient has smooth, usually moist areas of skin that are red and inflammed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irriate the affected areas.

d. Pustular Psoriasis

Pustular psoriasis, also referred to as palmar plantar psoriasis, is a form of psoriasis that causes pus-filled blisters that vary in size and location, but often occur on the hands and feet. The blisters may be localized, or spread over large areas of the body. Pustular psoriasis can be both tender and painful, can cause fevers.

e. Other Psoriasis Disorders

Other examples of psoriatic disorders which can be treated with the IL-12 and/or IL-23 antibody include erythrodermic psoriasis, vulgaris, psoriasis associated with IBD, and psoriasis associated with arthritis, including rheumatoid arthritis.

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the tables of Appendix A of U.S. Pat. No. 6,914,128, as well as the entire contents of U.S. Pat. No. 6,914,128 are incorporated herein by reference.

### EXAMPLES

### Example 1

### Effects of ABT-874 Versus Etanercept or Placebo on Health-Related Quality of Life in Patients With Moderate to Severe Psoriasis

### Methods

A Phase III study was conducted with the trial design shown in FIG. 1 for ABT-874 for moderate to severe psoriasis. (Revicki D A, et al. J Dermatolog Treat. 2007, 18:341-50 and Shikiar R, et al. Health Qual Life Outcomes. 2006, 4:71, the entire contents of each of which are expressly incorporated herein by reference.)

DLQI, VAS-Ps, VAS-PsA, SF-36 (subscales and summaries) were measured. MCID Response Rates were determined as the percentage of patients having improvement at or exceeding the minimum clinically important difference (MCID). MCID Criteria is shown in Table 1.

TABLE 1
MCID Criteria

| DLQI¹ | VAS Score¹ | SF-36 Scores² | |
|---|---|---|---|
| ≧5 points | ≧½ SD | MCS ≧5 points | PCS ≧3 points |
| | from BL | RP ≧10.51 points | BP ≧9.05 points |
| | | GH ≧4.97 points | VT ≧6.54 points |
| | | SF ≧13.62 points | RE ≧24.71 points |
| | | MH ≧4.90 points | PF ≧1/2 SD |

Abbreviations used herein are as follows: MCID, minimum clinically important difference; DLQI, Dermatology Life Quality Index; HRQOL, health-related quality of life; PGA, Physician's Global Assessment; SF-36, Short Form 36 Health Survey; MCS, Mental Component Summary; MH, Mental Health; PCS, Physical Component Summary; PF, Physical Function; RE, Role—Emotional; RP, Role—Physical; SF, Social Function; VT, Vitality; BP, Bodily Pain; GH, General Health; VAS-Ps, visual analog scale for Ps pain; VAS-PsA, VAS for psoriatic arthritis pain.

### Results

No significant differences were observed in baseline HRQOL scores among the 3 treatment arms, as shown in Table 2.

TABLE 2
Baseline HRQOL Scores

| | Treatment Group | | | |
|---|---|---|---|---|
| HRQOL Measurements | ABT-874 | Etanercept | Placebo | |
| (Mean ± SD) | (Mean ± SD) | (Mean ± SD) | (Mean ± SD) | P-Value^{a} |
| DLQI | 13.78 ± 7.24 | 13.77 ± 7.82 | 13.58 ± 6.85 | 0.9800 |
| SF-36 summary scores | | | | |
| MCS | 45.28 ± 12.13 | 46.09 ± 12.05 | 46.34 ± 11.64 | 0.6222 |
| PCS | 47.89 ± 9.90 | 48.24 ± 9.03 | 47.81 ± 9.88 | 0.9801 |
| VAS scores | | | | |
| VAS-Ps | 46.01 ± 29.67 | 44.08 ± 30.57 | 44.67 ± 29.79 | 0.9056 |
| VAS-PsA | 58.55 ± 27.13 | 59.57 ± 27.00 | 62.80 ± 32.23 | 0.7444 |
| SF-36 domain scores | | | | |
| Physical Function | 47.32 ± 11.06 | 47.91 ± 10.87 | 48.53 ± 10.55 | 0.6075 |
| Role—Physical | 47.07 ± 11.32 | 47.39 ± 10.93 | 47.11 ± 11.10 | 0.9908 |
| Bodily Pain | 45.05 ± 12.13 | 45.36 ± 10.86 | 44.95 ± 11.95 | 0.9937 |
| General Health | 49.1 ± 9.42 | 48.74 ± 8.46 | 48.42 ± 8.92 | 0.6661 |
| Vitality | 48.75 ± 9.95 | 49.15 ± 10.20 | 49.65 ± 10.23 | 0.7733 |
| Social Function | 43.25 ± 12.92 | 45.19 ± 12.71 | 44.62 ± 12.11 | 0.3800 |
| Role—Emotional | 45.87 ± 13.06 | 46.03 ± 12.99 | 46.41 ± 11.95 | 0.9820 |
| Mental Health | 45.27 ± 11.87 | 45.93 ± 11.46 | 46.44 ± 10.83 | 0.8679 |

^{a}Bonferroni method for multiple testing was used.
Patients with non-missing values were included for the baseline characteristics analysis.

ABT-874 was associated with significantly greater mean improvement in all HRQOL outcomes as compared to placebo (p<0.05). ABT-874 was associated with significantly greater mean improvement in DLQI, VAS-Ps, MCS, and several SF-36 domain scores (Vitality, Role—Emotional, Mental Health) as compared to etanercept.

TABLE 3
Mean Change in HRQOL By Week 12

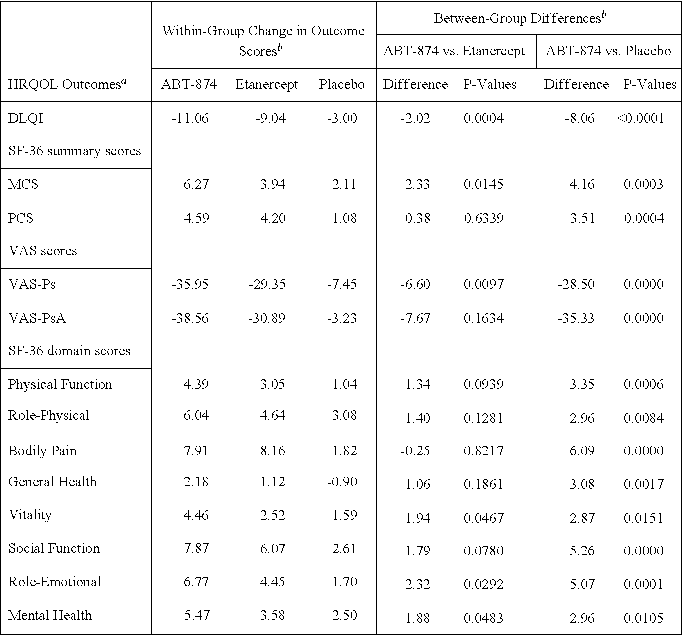

^{a}Increased scores signifies improvement for all HRQOL outcomes except DLQI, VAS-Ps, and VAS-PsA. Missing values at Week 12 were imputed using the last-observation-carried-forward method.
^{b}Least squares means were reported for within group and between group differences. Highlighted cells indicate 5% statistical significance based on the analysis of covariance, adjusting for baseline score and treatment.

The percentages of patients with improvement at or exceeding the MCID were significantly greater in ABT-874-treated patients at Week 12 than those in the placebo group for the following HRQOL outcomes: DLQI, SF-36 PCS and MCS, VAS-Ps and SF-36 domain scores (Bodily Pain, Vitality, Social Function, Role—Emotional) (FIG. 2). The percentages of patients with improvement at or exceeding the MCID were significantly greater in ABT-874-treated patients at Week 12 for Physical Function, Social Function and Mental Health compared with those in the etanercept group (FIG. 2).

### Conclusions

ABT-874 demonstrated significantly greater improvements in all HRQOL outcome measurements vs. placebo. ABT-874 demonstrated significantly greater improvements in DLQI, VAS-Ps, MCS and several SF-36 domain scores vs. etanercept. A significantly greater proportion of ABT-874 treated patients achieved clinically meaningful improvement at Week 12 as compared to placebo in DLQI, SF-36 PCS and MCS, VAS pain score for Ps and several SF-36 domain scores, and as compared to etanercept in Social Function and Mental Health domains of SF-36. These results further enhance the treatment benefits of ABT-874 on patients' lives beyond the previously described clinical efficacy in significantly reducing Ps symptoms vs. placebo and etanercept.

### Example 2

### Psoriasis Treatment With ABT-874: Effects on Health-Related Quality of Life and Work Productivity and Activity Impairment

### Methods

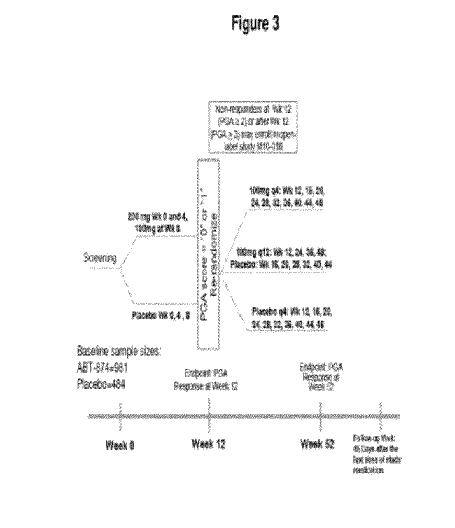

A Phase III study of patients with moderate to severe psoriasis who were treated with ABT-874, a monoclonal antibody specific for IL-12 and IL-23, or placebo. Study design is shown in FIG. 3. (Revicki D A, et al. J Dermatolog Treat. 2007; 18:341-50.)

Measure included DLQI, VAS-Ps, VAS-PsA, and WPAI:SHP for psoriasis.

Mean improvement in HRQOL and WPAI outcomes from baseline (BL) to Weeks 12 and 52 were compared. Percentages of patients achieving improvement≧MCID in outcomes (MCID response rates) at Weeks 12 and 52 were compared. (DLQI, Dermatology Life Quality Index; HRQOL, health-related quality of life; MCID, minimum clinically important difference; PGA, Physician's Global Assessment; VAS-Ps, visual analog scale for Ps pain; VAS-PsA, VAS for psoriatic arthritis pain; WPAI:SHP for Ps, Work Productivity and Activity Impairment Questionnaire: Specific Health Problem for Ps)

TABLE 4
MCID criteria

| DLQI¹ | VAS Score¹ | WPAI Scores |
|---|---|---|
| ≧5 points | ≧½ SD from BL | ≧½ SD from BL |

### Results

Baseline characteristics were similar in both arms, with no significant differences observed (Table 5).

Compared with placebo, ABT-874 was associated with significantly greater mean improvement in all HRQOL outcomes at Week 12 (p<0.008)

Following re-randomization at week 12, both ABT-874 treatment groups achieved significantly greater mean improvements in DLQI, VAS-Ps, and Ps related impairment while working and overall activity impairment compared to the placebo group at week 52. Compared to ABT-874 every 12 weeks dosing group, ABT-874 every 4 weeks dosing group achieved significantly greater mean improvements in DLQI, VAS-Ps, VAS-PsA, and all WPAI outcomes at Week 52 except for Ps related work time missed (all p<0.05). (Table 6)

Compared with placebo, significantly greater percentages of ABT-874-treated patients achieved clinically meaningful improvement in all outcomes at Week 12 (p<0.001). (Table 7) Following re-randomization at week 12, both ABT-874 treatment groups had significantly greater percentages of patients achieving clinically meaningful improvement in DLQI, VAS-Ps, VAS-PsA, and Ps related impairment while working and overall activity impairment compared to the placebo group at Week 52. Compared to ABT-874 every 12 weeks dosing group, ABT-874 every 4 weeks dosing group had significantly more patients achieving clinically meaningful improvement in DLQI, VAS-Ps, and Ps-related overall activity impairment (all p<0.05). (Table 7)

TABLE 5
Baseline Characteristics

| | Treatment Group | | |
|---|---|---|---|
| | ABT-874 | Placebo | P- |
| Baseline HRQOL Outcomes | Mean ± SD | Mean ± SD | Value^{a} |
| DLQI | 12.83 ± 7.02 | 12.69 ± 6.89 | 0.7459 |
| VAS scores | | | |
| VAS-Ps | 43.14 ± 29.63 | 41.67 ± 29.15 | 0.3763 |
| VAS-PsA | 48.80 ± 28.21 | 47.82 ± 31.56 | 0.7849 |
| WPAI: SHP for Ps score | | | |
| % Work time missed | 23.11 ± 26.92 | 21.99 ± 25.82 | 0.3814 |
| % Impairment while working | 21.07 ± 24.32 | 20.07 ± 24.03 | 0.2865 |
| % Overall work impairment | 4.85 ± 16.99 | 3.46 ± 13.18 | 0.3873 |
| % Overall activity impairment | 30.45 ± 28.85 | 28.22 ± 28.62 | 0.1210 |

^{a}One-way ANOVA was used to compare continuous variables.
Patients with non-missing values were included for the baseline characteristics analysis.

TABLE 6
Mean Change in HRQOL

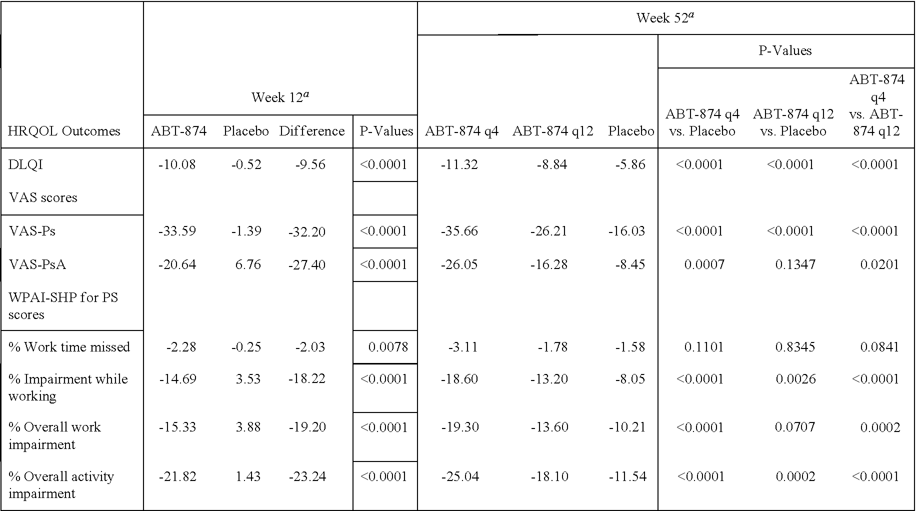

^{a}Least square means were reported for within group and between group analyses. Highlighted cells indicate a statistically significant difference at 5% level based on analysis of covariance, adjusting for treatment and baseline score. Missing data at Week-12 and Week-52 were imputed by Last-observation-carried-forward.
Decreased scores (i.e. values less than zero) signify improvement.

TABLE 7
HRQOL MCID Response Rates

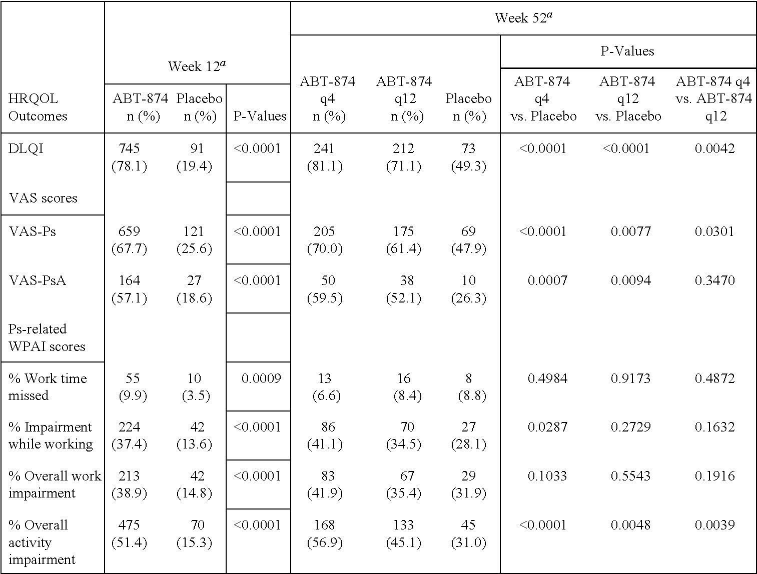

^{a}Highlighted cells indicate a statistically significant difference at 5% level based on chi-square test using last-observation-carried-forward approach (Week-12 data) or non-responder imputation (Week-52 data).

### Conclusions

ABT-874 demonstrated significantly greater improvements in almost all HRQOL outcome measurements vs. placebo at both Week 12 and Week 52, with significantly greater percentage of patients achieving clinically meaningful improvements. Following re-randomization at week 12, ABT-874 every-4-week dosing group achieved significantly greater mean improvements in DLQI, VAS-Ps, VAS-PsA, and Ps related impairment while working and overall activity impairment, with significantly more patients achieving clinically meaningful improvement, compared to the every-12-week dosing group. These results added to the treatment benefits of ABT-874 on patient lives beyond already-shown clinical efficacy in significantly reducing Ps symptoms vs. placebo in both the induction and maintenance phases.

### Example 3

### Efficacy and Safety Results from a Phase III, Randomized Controlled Trial Comparing the Safety and Efficacy of Briakinumab to Etanercept and Placebo in Patients with Moderate to Severe Chronic Plaque Psoriasis

### Introduction

Chronic plaque psoriasis is a common, immunologic skin disease characterized by thick, red, scaly cutaneous lesions that can be pruritic and painful, and cause profound effects on quality of life. Rapp S R, Feldman S R, Exum M L et al. Psoriasis Causes as Much Disability as Other Major Medical Diseases. J Am Acad Dermatol (1999) 41: 401-7). For moderate to severe psoriasis, systemic therapies such as methotrexate and cyclosporine have proven effective; however, long-term use with these agents may lead to cumulative toxicities (Thaci D. Long-Term Data in the Treatment of Psoriasis. Br J Dermatol (2008) 159 Suppl 2: 18-240). More recently, biologics have emerged as promising alternatives for the treatment of psoriasis. The proinflammatory cytokine tumour necrosis factor-alpha (TNF-α) has been implicated in the pathogenesis of psoriasis, and clinical trials repeatedly have demonstrated the efficacy of TNF-α antagonists as psoriasis therapy (Reich K, Nestle F O, Papp K et al. Infliximab Induction and Maintenance Therapy for Moderate-to-Severe Psoriasis: A Phase III, Multicentre, Double-Blind Trial. Lancet (2005) 366: 1367-74; Gottlieb A B, Matheson R T, Lowe N et al. A Randomized Trial of Etanercept as Monotherapy for Psoriasis. Arch Dermatol (2003) 139: 1627-32, discussion 32; Leonardi C L, Powers J L, Matheson R T et al. Etanercept as Monotherapy in Patients with Psoriasis. N Engl J Med (2003) 349: 2014-22; Menter A, Tyring S K, Gordon K et al. Adalimumab Therapy for Moderate to Severe Psoriasis: A Randomized, Controlled Phase III Trial. J Am Acad Dermatol (2008) 58: 106-15; Papp K A, Tyring S, Lahfa Metal. A Global Phase III Randomized Controlled Trial of Etanercept in Psoriasis: Safety, Efficacy, and Effect of Dose Reduction. Br J Dermatol (2005) 152: 1304-12; Saurat J H, Stingl G, Dubertret L et al. Efficacy and Safety Results from the Randomized Controlled Comparative Study of Adalimumab vs. Methotrexate vs. Placebo in Patients with Psoriasis (CHAMPION). Br J Dermatol (2008) 158: 558-66). Etanercept is a recombinant human fusion protein consisting of the extracellular domain of the p75 TNF-α receptor and the constant fragment of IgG1. It competitively antagonizes TNF-α by interfering with its interaction with cell-surface TNF receptors. Studies with 50 mg etanercept twice weekly have demonstrated efficacy, with 49% of moderate to severe psoriasis patients achieving a PASI 75 following 12 weeks of treatment (Leonardi C L, Powers J L, Matheson R T et al. Etanercept as Monotherapy in Patients with Psoriasis. N Engl J Med (2003) 349: 2014-22; Papp K A, Tyring S, Lahfa M et al. A Global Phase III Randomized Controlled Trial of Etanercept in Psoriasis: Safety, Efficacy, and Effect of Dose Reduction. Br J Dermatol (2005) 152: 1304-12).

Despite the efficacy results seen with etanercept use, a need remains for additional treatment options. Not all patients with moderate to severe psoriasis achieve success with TNF-α antagonists, and a subset of patients that initially respond to anti-TNF-α therapy lose response with long-term use (Tyring S, Gordon K B, Poulin Y et al. Long-Term Safety and Efficacy of 50 Mg of Etanercept Twice Weekly in Patients with Psoriasis. Arch Dermatol (2007) 143: 719-26). Interleukins (IL) 12 and 23 are thought to play a key role in the immune response underlying psoriasis, and their shared p40 subunit has become a potential target in the treatment of psoriasis (Koutruba N, Emer J, Lebwohl M. Review of Ustekinumab, an Interleukin-12 and Interleukin-23 Inhibitor Used for the Treatment of Plaque Psoriasis. Ther Clin Risk Manag; 6: 123-41; Nestle F O, Conrad C. The IL-12 Family Member P40 Chain as a Master Switch and Novel Therapeutic Target in Psoriasis. J Invest Dermatol (2004) 123: xiv-xv; Nestle F O, Kaplan D H, Barker J. Psoriasis. N Engl J Med (2009) 361: 496-509). Blockade of IL-12 and IL-23 results in the suppression of Th1 and Th17 cells and the inflammatory cellular cascade that results from activation of these immune cells (Nestle F O, Conrad C. The IL-12 Family Member P40 Chain as a Master Switch and Novel Therapeutic Target in Psoriasis. J Invest Dermatol (2004) 123: xiv-xv). Two fully human monoclonal antibodies directed against IL-12/23p40 have been developed: ustekinumab and briakinumab. Several clinical trials have demonstrated the efficacy of ustekinumab as psoriasis therapy, with >60% of patients achieving PASI 75 after 12 weeks of treatment (Kauffman C L, Aria N, Toichi E et al. A Phase I Study Evaluating the Safety, Pharmacokinetics, and Clinical Response of a Human IL-12 p40 Antibody in Subjects with Plaque Psoriasis. J Invest Dermatol (2004) 123: 1037-44; Krueger G G, Langley R G, Leonardi C et al. A Human Interleukin-12/23 Monoclonal Antibody for the Treatment of Psoriasis. N Engl J Med (2007) 356: 580-92; Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (PHOENIX 1). Lancet (2008) 371: 1665-74; Papp K A, Langley R G, Lebwohl M et al. Efficacy and Safety of Ustekinumab, a Human Interleukin-12/23 Monoclonal Antibody, in Patients with Psoriasis: 52-Week Results from a Randomised, Double-Blind, Placebo-Controlled Trial (PHOENIX 2). Lancet (2008) 371: 1675-84). In addition, this efficacy has been shown to be maintained for up to a year (Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (PHOENIX 1). Lancet (2008) 371: 1665-74). Similar results were observed in a 12-week, dose-ranging, phase II trial with briakinumab in which >90% of patients receiving multiple doses of briakinumab achieved a PASI 75 (Kimball A B, Gordon K B, Langley R G et al. Safety and Efficacy of Abt-874, a Fully Human Interleukin 12/23 Monoclonal Antibody, in the Treatment of Moderate to Severe Chronic Plaque Psoriasis: Results of a Randomized, Placebo-Controlled, Phase 2 Trial. Arch Dermatol (2008) 144: 200-7). A long-term extension of this trial revealed that patients were able to maintain good efficacy for a sustained period, even following withdrawal of briakinumab (Kimball A B, Gordon K, Langley R G et al. Efficacy and Safety of ABT-874, a Monoclonal Anti-Interleukin 12/23, for the Treatment of Chronic Plaque Psoriasis: 36-Week Observation/Retreatment and 60-Week Open-Label Extension Phases of a Randomized Phase 2 Trial J Am Acad Dermatol; in press).

Although these data suggest that the IL-12/23 antagonists may be a beneficial addition to dermatologists' armamentarium, head to head trials comparing the efficacy and safety of IL 12/23 inhibitors versus TNF-α antagonists would assist in the consideration of this new class of drug as a viable alternative to the TNF-α antagonists. In addition to the comparative efficacy data, it is critical to develop a more definitive safety profile for the IL 12/23 inhibitors. To this end, the recent 12 week trial, ACCEPT, demonstrated superior efficacy of ustekinumab over etanercept; similar safety was seen for both treatments (Griffiths C E, Strober B E, van de Kerkhof P et al. Comparison of Ustekinumab and Etanercept for Moderate-to-Severe Psoriasis. N Engl J Med (2010) 362: 118-28).

To increase knowledge regarding the use of IL-12/23 antagonists as psoriasis treatment, the phase III trial described in this Example was designed to compare the efficacy and safety of briakinumab to both etanercept and placebo in patients with moderate-to-severe chronic plaque psoriasis at 12 weeks. A parallel study with identical design was concurrently conducted.

Briefly, 350 patients were enrolled in this Phase III, 12-week study (M10-315) and randomized in a 2:2:1 ratio as follows: 139 patients received 200 mg briakinumab at Weeks 0 and 4 followed by 100 mg briakinumab at Week 8; 139 patients received 50 mg of etanercept twice weekly 3-4 days apart at Weeks 0-11 (n=141); 72 patients received placebo injections matching active treatment. The co-primary efficacy endpoints were the proportion of patients achieving a PGA of 0/1 at Week 12, and the proportion of patients achieving a Psoriasis Area Severity Index (PASI) 75 response at Week 12.

The results showed that 72.7% of patients treated with briakinumab achieved a PGA of 0/1 at Week 12 as compared with 29.5% of etanercept-treated patients and 4.2% of placebo-treated patients (P<0.001 for both comparisons). 80.6% of briakinumab-treated patients achieved a PASI 75 response at Week 12 as compared with 39.6% of etanercept-treated and 6.9% of placebo-treated patients (P<0.001 for both comparisons). Conclusions: In moderate to severe psoriasis patients, briakinumab had superior efficacy to both placebo and etanercept at 12 weeks as administered in this study.

### Methods

### Patients

This Phase III, 12-week double-blind, double dummy, multicenter, randomized study was performed at 41 sites in the United States. Eligible patients were: ≧18 years old with a clinical diagnosis of chronic plaque psoriasis for at least 6 months; had stable plaque Ps for at least 2 months before Screening and at Baseline (Week 0) visits; affected body surface area (BSA)≧10%; Physician's Global Assessment (PGA) of at least moderate (≧3); and Psoriasis Area and Severity Index (PASI) score of ≧12 at the Baseline (Week 0) visit. Exclusion criteria included: previous exposure to systemic anti-IL-12/23p40 therapy, including briakinumab; previous exposure to etanercept or known hypersensitivity to etanercept; or inability to discontinue topical therapies, phototherapies, or systemic therapies.

An independent ethics committee or institutional review board at each study site approved the protocol; each patient provided written informed consent.

### Study Design

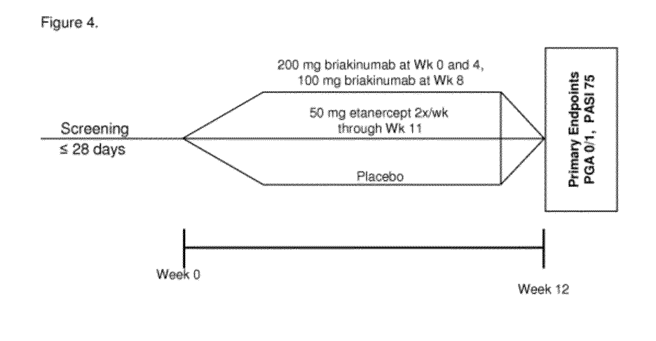

Patients were randomized 2:2:1 to a briakinumab, etanercept, or placebo treatment arm at Week 0 (FIG. 4). Briakinumab-treated patients received 200 mg briakinumab subcutaneously (SC) at Weeks 0 and 4 followed by 100 mg briakinumab SC at Week 8. Etanercept-treated patients received 50 mg of etanercept SC twice weekly 3-4 days apart at Weeks 0-11. Patients enrolled in the placebo arm received SC injections matching active treatment. To maintain the blind, all patients received two SC injections at Weeks 0 and 4 and one SC injection at Week 8, consisting of either briakinumab or matching placebo, depending on the treatment arm. In addition, each patient also received two SC injections biweekly, 3 days apart Week 0 through Week 11, consisting of either etanercept or matching placebo, depending on the treatment arm.

### Efficacy and Safety Measures

The co-primary efficacy endpoints were: 1) the proportion of patients achieving a Physician's Global Assessment (PGA) of 0/1, which is defined as a PGA score of “clear” or “minimal” at Week 12; 2) the proportion of patients achieving a ≧Psoriasis Area Severity Index (PASI) 75 response, defined as at least a 75% reduction in PASI score at Week 12 relative to Baseline PASI score.

Secondary efficacy measures included median time to achieve PASI 75 and PGA 0/1, proportion of patients achieving PASI 90 and 100 responses over 12 weeks, and proportion of patients achieving a DLQI score of 0 at Week 12.

Adverse events, laboratory parameters, and vital signs were monitored throughout the study. Adverse events occurring up to 45 days after study drug administration were included in the analyses.

### Statistical Methods

350 patients were planned to be randomized in a 2:2:1 ratio to receive briakinumab (140 subjects); etanercept (140 subjects), and placebo (70 subjects). Assuming that 70% of briakinumab-treated, 50% of etanercept-treated, and 4% of placebo-treated patients achieved a PGA 0/1 at Week 12, this sample size would provide 90% power to determine the superiority of briakinumab relative to etanercept using a Chi-square test and more than 90% power to show that briakinumab treatment was superior to placebo.

The primary efficacy analysis consisted of four comparisons performed in the intent-to-treat (ITT) population (ie, all randomized subjects), which were tested in a fixed sequence at the alpha=0.05 significance level in order to address the issues of multiplicity as follows: 1) PGA 0/1 response rates for briakinumab vs. placebo at Week 12; 2) PASI 75 response rates for briakinumab vs. placebo at Week 12; 3) PGA 0/1 response rates for briakinumab vs. etanercept at Week 12; 4) PASI 75 response rates for briakinumab vs. etanercept at Week 12.

The primary efficacy analyses were performed using a Cochran-Mantel-Haenszel test stratified by pooled centre. A Chi-Square test, or Fisher's exact test as appropriate, was used to compare proportions of patients in each treatment group achieving PASI 75, 90 or 100 at Weeks 2, 4, 8, and 12, as well as DLQI score of 0 at Week 12. Non-responder imputation (NR1) was used to handle missing data. Median time to achieve PASI 75 and PGA 0/1 were calculated using the Kaplan Meier method for each treatment group, and treatment comparison was performed using the Log-rank test. Patients not achieving the response on or before Week 12 were censored at the date of the last PASI or PGA evaluation. Statistical comparisons were made for briakinumab vs. etanercept and briakinumab vs. placebo. All statistical tests were two-sided with the significance level of 0.05.

The safety analyses were conducted for all subjects who received at least one dose of study drug; safety endpoints were summarized by treatment group.

### Results

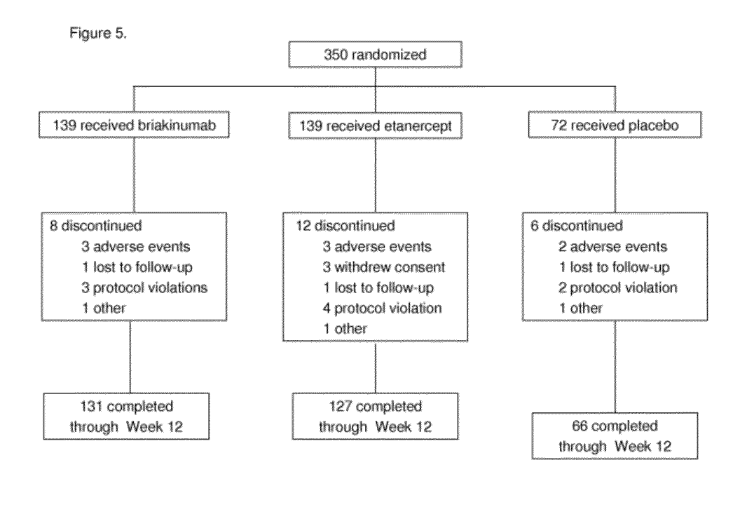

A total of 350 patients were enrolled in this study: placebo, N=72; etanercept, N=139; briakinumab, N=139. Sixty-six (91.7%) patients receiving placebo, 127 (91.4%) patients receiving etanercept, and 131 (94.2%) patients receiving briakinumab completed the study (FIG. 5). Baseline demographics and clinical characteristics were similar across treatment groups and comparable to those seen in a typical moderate to severe psoriasis patient population (Table 8). A similar percentage of patients in each treatment group had a PGA of moderate or severe at baseline and there was no significant difference across treatment groups for PASI score at baseline. At baseline, a similar percentage of patients across treatment groups had ≧1, ≧2 or ≧3 of the following cardiovascular risk factors: ≧45 years of age for males or ≧55 years of age for females; BMI≧30, current cigarette smoking; history of diabetes or baseline glucose≧126 mg/dL; history of hypertension or baseline SBP≧140 or DBP≧90; history of cardiovascular disease (angina, coronary artery disease, myocardial infarction, cerebrovascular accident, cerebral haemorrhage, transient ischemic attack, congestive heart failure, and peripheral vascular disease-arterial) (placebo, 83.3%, 59.7%, 34.7%; etanercept, 90.6%, 64.0%, 30.9%; briakinumab, 88.5%, 53.2%, 26.6%; ≧1, ≧2 or ≧3 risk factors, respectively). 28.8% of patients in the briakinumab arm, 19.4% of patients in the etanercept arm, and 16.7% of patients in the placebo arm received prior phototherapy 10.8% of briakinumab-treated patients, 7.9% of etanercept-treated patients, and 4.2% of placebo-treated patients received prior systemic biologic therapy. Specifically, 5.8% of etanercept and briakinumab-treated patients received prior adalimumab therapy as compared with 1.4% of placebo-treated patients; there was no difference across groups for any other prior TNF-α antagonist therapy (prior infliximab therapy: placebo group, 1.4%; etanercept group, 0%; briakinumab group, 2.2%).

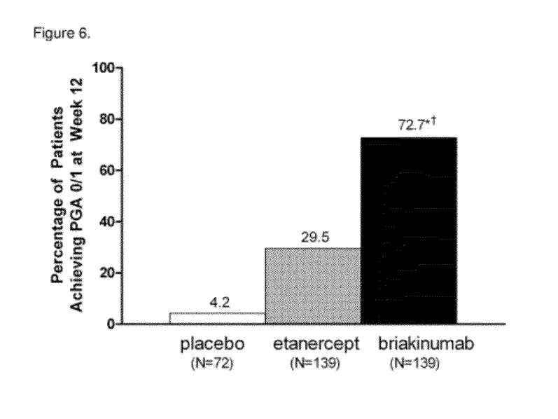

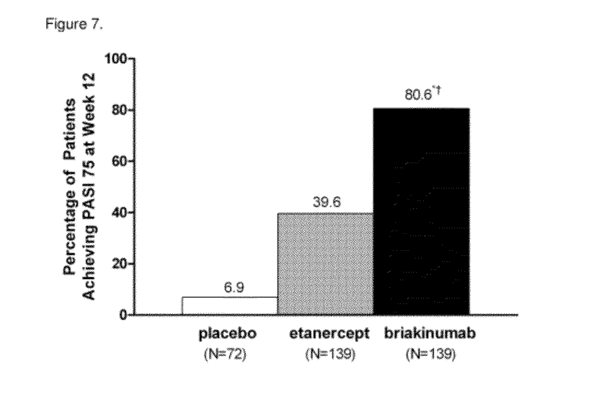

A statistically significant greater percentage of patients receiving briakinumab treatment (72.7%) achieved the co-primary endpoint of PGA of 0/1 at Week 12, as compared with patients receiving etanercept (29.5%) or placebo (4.2%; P<0.001, for both comparisons; FIG. 6). A statistically significantly greater percentage of patients in the briakinumab treatment group (80.6%) also achieved the co-primary endpoint, a PASI 75 response at Week 12, as compared with patients in the etanercept (39.6%) and placebo treatment groups (6.9%; P<0.001, for both comparisons; FIG. 7).

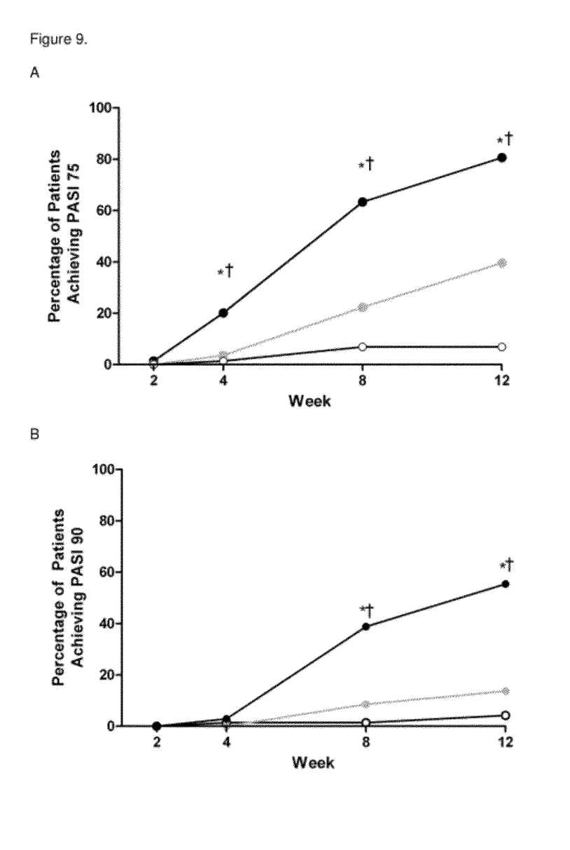

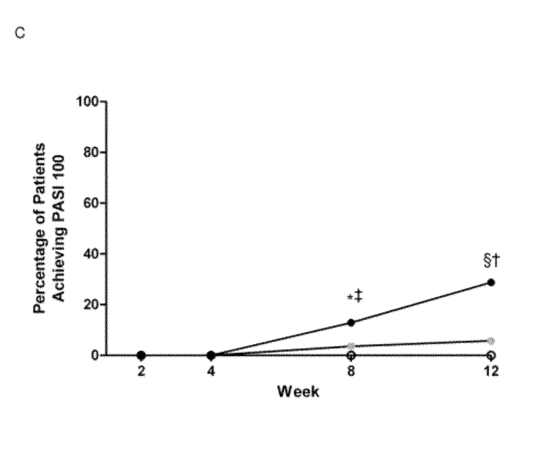

PASI 90 and PASI 100 response rates were statistically significantly greater for patients receiving briakinumab as compared with patients receiving etanercept or placebo at Week 12 (PASI 90/PASI 100: placebo, 4.2%/0%; etanercept, 13.7%/5.8; briakinumab, 55.4%/28.8% (P<0.001, for both comparisons for both endpoints; FIG. 9).

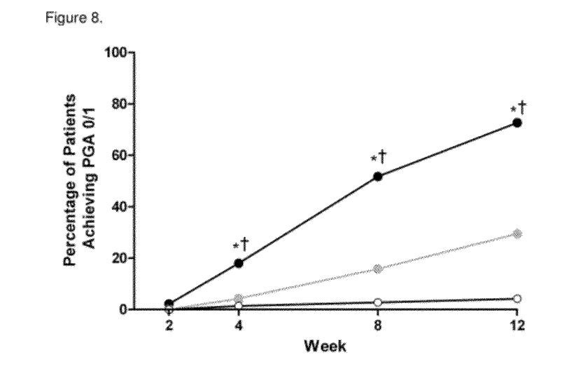

The median time to achieve a PGA 0/1 was 58 days for briakinumab-treated patients; too few etanercept- and placebo-treated patients achieved a PGA 0/1 during the trial to compute a median (P<0.001, for both comparisons; Table 9). The median time to achieve PASI 75 was 57 days for briakinumab-treated patients and 86 days for etanercept-treated patients; again, too few placebo-treated patients achieved a PASI 75 during the trial to compute a median (P<0.001, for both comparisons; Table 9). Superior efficacy of briakinumab versus etanercept was demonstrated for secondary efficacy variables prior to Week 12, including the proportion of patients achieving PASI 75/90/100 responses and a PGA score of 0/1 at all time points measured (FIGS. 8 and 9).

Further, 30.2% of briakinumab-treated patients achieved a DLQI score of 0 as compared with 15.1% of etanercept-treated patients and 2.8% of placebo-treated patients (P<0.003, for both comparisons; Table 9).

No deaths occurred during this study (Table 10). 50.4% of briakinumab-, 49.6% of etanercept-, and 44.4% of placebo-treated patients experienced adverse events. 2.9% of patients in the briakinumab and etanercept arms and 2.8% of placebo-treated patients discontinued due to adverse events. Serious adverse events were reported in 2 (1.4%) briakinumab-treated patients (colon cancer, convulsion), 1 (0.7%) etanercept-treated patient (breast cancer in situ), and 2 (2.8%) placebo-treated patients (coronary artery disease, psoriasis). No serious infections were reported. One patient in the briakinumab arm was diagnosed with colon cancer on day 66 of the study and underwent hemicolectomy, splenectomy, and distal pancreatectomy. One briakinumab-treated patient was diagnosed with lip neoplasm, malignant stage unspecified, on study day 92 and underwent excision of the lesion. One patient receiving briakinumab was diagnosed with a basal cell carcinoma on study day 41 and underwent excision of lesion. Two patients in the etanercept group were diagnosed with basal cell carcinoma, on study days 29 and 60, respectively; both patients underwent lesion excision. One etanercept-treated patient was diagnosed with squamous cell carcinoma on study day 28 and underwent biopsy. One etanercept-treated patient was diagnosed with breast cancer in situ on study day 110 and underwent biopsy. One patient placebo-treated patient was diagnosed with malignant melanoma on study day 30; study drug was discontinued and the patient was scheduled to have Mohs surgery. No major adverse cardiac events (MACE), defined as nonfatal myocardial infarction, nonfatal stroke, or cardiovascular death, were reported in any of the treatment groups. Two patients in the briakinumab group, 4 patients in the etanercept group, and 2 patients in the placebo group reported ischemic heart disease adverse events. There was one event of coronary artery disease in a placebo patient; the remainder of events were increased creatine phosphokinase (CPK). Elevated CPK was one of the broad search terms used for the “ischemic heart disease” Standardised MedDRA term Query (SMQ) search; however, as these elevated CPK values were not fractionated, a direct link cannot be inferred. The most frequently reported treatment-emergent adverse events occurring in at least 5% of patients in both the briakinumab and etanercept treatment groups were upper respiratory tract infection (7.2% and 11.5%, respectively) and nasopharyngitis (7.2% and 7.9%, respectively); these were reported more frequently for etanercept than briakinumab. The most frequently reported adverse event for placebo patients was nasopharyngitis (8.3%; Table 11) which was reported more frequently than for either etanercept or briakinumab. No clinically meaningful changes in laboratory values or vital signs across treatment groups were observed.

### Discussion

The results of the current study, along with those from the parallel M10-114 trial (Gottlieb A, Leonardi C, Kerdel F et al. Efficacy and Safety Results of Briakinumab Versus Etanercept and Placebo in Patients with Moderate to Severe Chronic Plaque Psoriasis. Br J. Dermatol. (2011) 165:652-60), provide support for the benefit of an IL-12/23 antagonist for the treatment of moderate to severe psoriasis. Statistically significantly greater percentages of patients receiving briakinumab as compared with etanercept or placebo achieved the co-primary endpoints of a PGA 0/1 and PASI 75, as well as other ranked and non-ranked secondary endpoints (including PASI 90 and 100 responses, and DLQI score of 0) at week 12, demonstrating the superiority of briakinumab over etanercept and placebo. Comparable rates of adverse events were seen across treatment groups; no important safety concerns were observed during the trial.

Although the overall results of the current study are very similar to those obtained during the M10-114 trial, the magnitude of the etanercept efficacy effect over placebo was smaller in the current study than in M10-114 and prior etanercept trials (Gottlieb A B, Matheson R T, Lowe N et al. A Randomized Trial of Etanercept as Monotherapy for Psoriasis. Arch Dermatol (2003) 139: 1627-32, discussion 32; Leonardi C L, Powers J L, Matheson R T et al. Etanercept as Monotherapy in Patients with Psoriasis. N Engl J Med (2003) 349: 2014-22; Tyring S, Gordon K B, Poulin Y et al. Long-Term Safety and Efficacy of 50 Mg of Etanercept Twice Weekly in Patients with Psoriasis. Arch Dermatol (2007) 143: 719-26). At Week 12 of M10-114, 39.7% of etanercept-treated patients achieved a PGA 0/1 and 56.0% achieved a PASI 75. In the current study, 29.5% of etanercept-treated patients achieved a PGA 0/1 at Week 12; 39.6% achieved a PASI 75 at Week 12. Interestingly, comparable proportions of briakinumab-treated patients achieved both primary endpoints in both studies. It is possible the differential etanercept efficacy benefit arose from a difference in the patient population enrolled in each study. The current study was comprised of patients with more severe disease than those enrolled in M10-114; 47.1% and 5.4% of patients in the current study had a PGA of severe or very severe, respectively, as compared with 40.6% and 4.3% of patients enrolled in M10-114. It has been shown that among patients treated with etanercept still achieving a PGA of moderate, marked, or severe after 12 weeks of therapy, >40% were able to achieve PASI 75 and a PGA of clear or mild after switching to ustekinumab (Griffiths C E, Strober B E, van de Kerkhof P et al. Comparison of Ustekinumab and Etanercept for Moderate-to-Severe Psoriasis. N Engl J Med (2010) 362: 118-28). Thus, it is possible that the higher average severity of psoriasis in the current patient population resulted in less robust efficacy effects with etanercept therapy, but comparable effects with briakinumab treatment.

These data contribute to a growing body of evidence indicating the utility of anti-IL-12/23 monoclonal antibodies as alternatives to anti-TNF-α treatment for moderate to severe psoriasis. In addition to the phase II and phase III studies demonstrating the robust efficacy of briakinumab and ustekinumab as treatment for psoriasis, ACCEPT demonstrated the superior efficacy of ustekinumab over etanercept (Krueger G G, Langley R G, Leonardi C et al. A Human Interleukin-12/23 Monoclonal Antibody for the Treatment of Psoriasis. N Engl Med (2007) 356: 580-92; Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (PHOENIX 1). Lancet (2008) 371: 1665-74; Kimball A B, Gordon K B, Langley R G et al. Safety and Efficacy of Abt-874, a Fully Human Interleukin 12/23 Monoclonal Antibody, in the Treatment of Moderate to Severe Chronic Plaque Psoriasis: Results of a Randomized, Placebo-Controlled, Phase 2 Trial. Arch Dermatol (2008) 144: 200-7; Griffiths C E, Strober B E, van de Kerkhof P et al. Comparison of Ustekinumab and Etanercept for Moderate-to-Severe Psoriasis. N Engl J Med (2010) 362: 118-28). In the current study, M10-114, and ACCEPT, anti-IL-12/23 treatment resulted in significantly higher efficacy responses over etanercept. Moreover, when therapy was withdrawn in the ACCEPT trial, the time to disease recurrence was much longer for ustekinumab compared with etanercept. Another key finding from ACCEPT, as noted above, was the substantial improvement observed in patients switched from etanercept to ustekinumab, suggesting that anti-IL-12/23 therapy may be a viable alternative treatment option for patients not achieving success with etanercept.

Despite these promising results regarding the efficacy of anti-IL-12/23 therapy for psoriasis, a need still exists for a clear long-term safety profile. Substantial data exists supporting the long-term safety of TNF-α antagonists for psoriasis treatment (Tyring S, Gordon K B, Poulin Y et al. Long-Term Safety and Efficacy of 50 Mg of Etanercept Twice Weekly in Patients with Psoriasis. Arch Dermatol (2007) 143: 719-26; Burmester G R, Mease P, Dijkmans B A et al. Adalimumab Safety and Mortality Rates from Global Clinical Trials of Six Immune-Mediated Inflammatory Diseases. Ann Rheum Dis (2009) 68: 1863-9; Gordon K B, Langley R G, Leonardi C et al. Clinical Response to Adalimumab Treatment in Patients with Moderate to Severe Psoriasis: Double-Blind, Randomized Controlled Trial and Open-Label Extension Study. J Am Acad Dermatol (2006) 55: 598-606). Of particular concern with TNF-α antagonist use is the potential for serious infections and cardiac disorders. During both the current trial and M10-114, a similar safety profile was observed for briakinumab and etanercept. Overall adverse event rates were not different between briakinumab and etanercept patients enrolled in either study. During the current trial, a higher percentage of briakinumab-treated (1.4%) and placebo-treated (2.8%) patients experienced serious adverse events compared with etanercept patients (0.7%). Similar rates of serious infections and skin cancers were reported for all treatment groups across both trials. No MACE were reported either the current trial or M10-114. Of note, 3 MACE were reported during ACCEPT, all in patients receiving ustekinumab at some point during the study and 7 MACE were reported during a phase 3, 52-week briakinumab trial (Gordon K, Langley R G, Gottlieb A B et al. Efficacy and Safety Results from a Phase III, Randomized Controlled Trial Comparing Two Dosing Regimens of ABT-874 to Placebo in Patients with Moderate to Severe Psoriasis. Presented at the 3rd International Congress of Psoriasis: Paris, France; Jul. 1-4, 2010. Abstract number 68). Although several patients in M10-114 and the current trial experienced ischemic heart disease events, all events in etanercept and briakinumab treatment groups were due to increased creatine phosphokinase; one case of coronary artery disease was reported for a placebo-treated patient in the current trial. Results from a phase II extension study with briakinumab and from ACCEPT offer some evidence for safety of IL-12/23 antagonists beyond the short term. Both trials collected safety data through at least 60 weeks and reported low rates of adverse events during this extended treatment period; there were no MACE reported during the phase II briakinumab trial (Kimball A B, Gordon K, Langley R G et al. Efficacy and Safety of ABT-874, a Monoclonal Anti-Interleukin 12/23, for the Treatment of Chronic Plaque Psoriasis: 36-Week Observation/Retreatment and 60-Week Open-Label Extension Phases of a Randomized Phase 2 Trial, J Am Acad Dermatol (2011) 64:263-74).

The results of this study, along with those seen during M10-114, strongly suggest that briakinumab is a valuable therapeutic tool for the management of moderate to severe psoriasis. These results provide further confirmation of the benefit of targeting the IL-12/23 pathway during psoriasis, and offer physicians a much needed alternative to TNF-α antagonist therapy. Future studies will address the benefits of briakinumab over the long-term, as well as develop a more complete safety profile of this new class of drug.

TABLE 8
Baseline Demographics and Clinical Characteristics

| | Treatment Group | | | |
|---|---|---|---|---|
| | Placebo | Etanercept | Briakinumab | Total |
| Characteristic | (N = 72) | (N = 139) | (N = 139) | (N = 350) |
| Age (yrs)^{a} | 45.0 (13.9) | 45.2 (14.8) | 44.9 (12.9) | 45.1 (13.8) |
| Male, n (%) | 46 (63.9) | 85 (61.2) | 93 (66.9) | 224 (64.0) |
| Caucasian, n (%) | 67 (93.1) | 127 (91.4) | 122 (87.8) | 316 (90.3) |
| Duration of psoriasis (yrs)^{a} | 15.5 (11.7) | 15.2 (12.1) | 16.3 (12.0) | 15.7 (11.9) |
| Body weight (kg)^{a} | 92.9 (25.2) | 96.9 (24.9) | 96.1 (24.5) | 95.8 (24.8) |
| BMI, n (%) | | | | |
| <25 (normal) | 11 (15.3) | 20 (14.4) | 24 (17.3) | 55 (15.7) |
| 25-<30 (overweight) | 22 (30.6) | 41 (29.5) | 42 (30.2) | 105 (30.0) |
| ≧30 (obese) | 39 (54.2) | 78 (56.1) | 73 (52.5) | 190 (54.3) |
| % BSA affected^{a} | 22.1 (13.4) | 24.7 (13.9) | 24.9 (17.8) | 24.2 (15.5) |
| PGA, n (%) | | | | |
| Moderate | 34 (47.2) | 69 (49.6) | 63 (45.3) | 166 (47.4) |
| Severe | 35 (48.6) | 63 (45.3) | 67 (48.2) | 165 (47.1) |
| Very severe | 3 (4.2) | 7 (5.0) | 9 (6.5) | 19 (5.4) |
| PASI score^{a} | 18.3 (6.4) | 18.5 (6.0) | 19.4 (7.9) | 18.8 (6.9) |
| History of PsA, n (%) | 15 (20.8) | 46 (33.1) | 33 (23.7) | 94 (26.9) |
| Previous medical history, n (%) | | | | |
| Any CV^{b} | 26 (36.1) | 56 (40.3) | 52 (37.4) | 134 (38.3) |
| Hyperlipidemia | 6 (8.3) | 8 (5.8) | 15 (10.8) | 29 (8.3) |
| Diabetes mellitus | 7 (9.7) | 13 (9.4) | 9 (6.5) | 29 (8.3) |
| Cardiovascular risk factors, n (%) ^{c} | | | | |
| 0 | 12 (16.7) | 13 (9.4) | 16 (11.5) | 41 (11.7) |
| 1+ | 60 (83.3) | 126 (90.6) | 123 (88.5) | 309 (88.3) |
| 2+ | 43 (59.7) | 89 (64.0) | 74 (53.2) | 206 (58.9) |
| 3+ | 25 (34.7) | 43 (30.9) | 37 (26.6) | 105 (30.0) |
| Previous psoriasis treatment, n (%) | | | | |
| Topical therapy | 70 (97.2) | 132 (95.0) | 128 (92.1) | 330 (94.3) |
| Phototherapy | 12 (16.7) | 27 (19.4) | 40 (28.8) | 79 (22.6) |
| Systemic non-biologic | 20 (27.8) | 44 (31.7) | 41 (29.5) | 105 (30.0) |
| Systemic biologic | 3 (4.2) | 11 (7.9) | 15 (10.8) | 29 (8.3) |

BMI, body mass index; BSA, body surface area; PGA, Physician's Global Assessment; PASI, Psoriasis Area Severity Index; PsA, psoriatic arthritis; CV, cardiovascular body system.
^{a}Mean (SD).
^{b}A patient who reported 2 or more diagnoses in the cardiovascular body system were only counted once for “Any CV”.
^{c}Risk factors defined as: ≧45 years of age for males or ≧55 years of age for females; BMI ≧30, current cigarette smoking; history of diabetes or baseline glucose ≧126 mg/dL; history of hypertension or baseline SBP ≧140 or DBP ≧90; history of cardiovascular disease (angina, coronary artery disease, myocardial infarction, cerebrovascular accident, cerebral haemorrhage, transient ischemic attack, congestive heart failure, and peripheral vascular disease-arterial)

TABLE 9
Secondary Clinical Measures at Week 12

| | Treatment Group | | |
|---|---|---|---|
| | Placebo | Etanercept | Briakinumab |
| | (N = 72) | (N = 139) | (N = 139) |
| Time to achieve PGA 0/1, | — | — | 58 |
| Median (days) | | | |
| Time to achieve PASI 75, | — | 86 | 57 |
| Median (days) | | | |
| Patients with DLQI score | 2 (2.8) | 21 (15.1) | 42 (30.2) |
| of 0 at Week 12, n (%) | | | |

PGA, Physician's Global Assessment; PASI, Psoriasis Area Severity Index; DLQI, Dermatology Life Quality Index;
— indicates median can not be estimated due to too few patients achieving PGA 0/1 or PASI 75 during trial.

TABLE 10
Overview of adverse events

| | Treatment Group, n (%) | | |
|---|---|---|---|
| | Placebo | Etanercept | Briakinumab |
| | (N = 72) | (N = 139) | (N = 139) |
| Any AE | 32 (44.4) | 69 (49.6) | 70 (50.4) |
| Any severe AE | 2 (2.8) | 1 (0.7) | 2 (1.4) |
| Any serious AE | 2 (2.8) | 1 (0.7) | 2 (1.4) |
| Any AE leading to discontinuation | 2 (2.8) | 4 (2.9) | 4 (2.9) |
| of study drug | | | |
| Deaths | 0 | 0 | 0 |
| Any infection | 10 (13.9) | 39 (28.1) | 34 (24.5) |
| Any serious infection | 0 | 0 | 0 |
| Any malignancy | 1 (1.4)^{a} | 4 (2.9)^{b} | 3 (2.2)^{c} |
| Squamous cell carcinoma | 0 | 1 (0.7) | 0 |
| Basal cell carcinoma | 0 | 2 (1.4) | 1 (0.7) |
| Other | 1 (1.4) | 1 (0.7) | 2 (1.4) |
| MACE^{d} | 0 | 0 | 0 |

^{a}Patient was diagnosed with malignant melanoma on study day 30.
^{b}Two patients diagnosed with basal cell carcinoma on study days 29 and 60, one patient each was diagnosed with squamous cell carcinoma on study day 28 and breast cancer in situ on study day 110.
^{c}One patient each was diagnosed with colon cancer (study day 66), lip neoplasm, malignant stage unspecified (study day 92), and basal cell carcinoma (study day 41).
^{d}MACE events were defined as non-fatal myocardial infarction, non-fatal stroke, or cardiovascular death.

TABLE 11
Adverse events occurring in ≧5%
of patients in any treatment group

| | Treatment Group, n (%) | | |
|---|---|---|---|
| | Placebo | Etanercept | Briakinumab |
| | (N = 72) | (N = 139) | (N = 139) |
| Upper respiratory tract infection | 0 | 16 (11.5) | 10 (7.2)* |
| Nasopharyngitis | 6 (8.3) | 11 (7.9) | 10 (7.2) |

*P = 0.017, briakinumab vs. placebo

### Example 4

### Efficacy and Safety Results of Briakinumab Versus Etanercept and Placebo in Patients with Moderate to Severe Chronic Plaque Psoriasis

### Introduction

Psoriasis is a chronic, immunologic skin disease affecting 1% to 3% of the general population (Greaves M W, Weinstein G D. Treatment of Psoriasis. N Engl J Med (1995) 332: 581-8). Much progress has been made in elucidating the pathogenesis of psoriasis, and it is now recognized as one of the most common T-cell-mediated disorders. Following antigen-induced activation, a cellular cascade is initiated that results in proliferation and differentiation of cutaneous T cells, producing keratinocyte alterations and consequent psoriatic plaques (Krueger J G. The Immunologic Basis for the Treatment of Psoriasis with New Biologic Agents. J Am Acad Dermatol (2002) 46: 1-23; quiz −6). As part of this cellular cascade, many proinflammatory cytokines are released, including tumour necrosis factor α (TNF-α) (Nestle F O, Kaplan D H, Barker J. Psoriasis. N Engl J Med (2009) 361: 496-509). TNF-α is highly expressed in psoriatic plaques, and there is evidence supporting a role for TNF-α as a master cytokine regulating psoriasis (Boyman O, Hefti H P, Conrad C et al. Spontaneous Development of Psoriasis in a New Animal Model Shows an Essential Role for Resident T Cells and Tumor Necrosis Factor-Alpha. J Exp Med (2004) 199: 731-6; Nestle F O, Conrad C. The IL-12 Family Member P40 Chain as a Master Switch and Novel Therapeutic Target in Psoriasis. J Invest Dermatol (2004) 123: xiv-xv). Serum and lesional concentrations of TNF-α correlate with disease severity, and decrease following effective therapy (Ameglio F, Bonifati C, Pietravalle M et al. Interleukin-6 and Tumour Necrosis Factor Levels Decrease in the Suction Blister Fluids of Psoriatic Patients During Effective Therapy. Dermatology (1994) 189: 359-63; Mussi A, Bonifati C, Carducci M et al. Serum TNF-Alpha Levels Correlate with Disease Severity and Are Reduced by Effective Therapy in Plaque-Type Psoriasis. J Biol Regul Homeost Agents (1997) 11: 115-8; Olaniran A K, Baker B S, Paige D G et al. Cytokine Expression in Psoriatic Skin Lesions During PUVA Therapy. Arch Dermatol Res (1996) 288: 421-5). In addition, a recent study showed that TNF blockade potently interferes with the multiple aspects of the inflammatory response underlying psoriasis (Gottlieb A B, Chamian F, Masud S et al. TNF Inhibition Rapidly Down-Regulates Multiple Proinflammatory Pathways in Psoriasis Plaques. J Immunol (2005) 175: 2721-9). The critical role of TNF-α in the pathogenesis of psoriasis is further underscored by the use of biological therapies targeted against this cytokine; several clinical trials have demonstrated the effectiveness of TNF-α antagonists as psoriasis treatment (Gottlieb A B, Matheson R T, Lowe N et al. A Randomized Trial of Etanercept as Monotherapy for Psoriasis. Arch Dermatol (2003) 139: 1627-32; Leonardi C L, Powers J L, Matheson R T et al. Etanercept as Monotherapy in Patients with Psoriasis. N Engl J Med (2003) 349: 2014-22; Menter A, Tyring S K, Gordon K et al. Adalimumab Therapy for Moderate to Severe Psoriasis: A Randomized, Controlled Phase III Trial. J Am Acad Dermatol (2008) 58: 106-15; Papp K A, Tyring S, Lahfa M et al. A Global Phase III Randomized Controlled Trial of Etanercept in Psoriasis: Safety, Efficacy, and Effect of Dose Reduction. Br J Dermatol (2005) 152: 1304-12; Saurat J H, Stingl G, Dubertret L et al. Efficacy and Safety Results from the Randomized Controlled Comparative Study of Adalimumab Vs. Methotrexate Vs. Placebo in Patients with Psoriasis (CHAMPION). Br J Dermatol (2008) 158: 558-66). Etanercept is a dimeric fusion protein consisting of the extracellular ligand-binding portion of the human 75 kilodalton (p75) TNF receptor linked to the Fc portion of the human IgG1 that inhibits TNF by interfering with its interaction with cell-surface receptors. Studies administering etanercept, at a dose of 50 mg subcutaneously (SC) twice weekly, have demonstrated efficacy, with 49% of patients achieving a PASI 75 following 12 weeks of treatment (Leonardi C L, Powers J L, Matheson R T et al. Etanercept as Monotherapy in Patients with Psoriasis. N Engl J Med (2003) 349: 2014-22; Papp K A, Tyring S, Lahfa M et al. A Global Phase III Randomized Controlled Trial of Etanercept in Psoriasis: Safety, Efficacy, and Effect of Dose Reduction. Br J Dermatol (2005) 152: 1304-12).

More recently, the structurally related cytokines interleukin (IL) 12 and 23 have emerged as attractive targets for psoriasis therapy (Nestle F O, Conrad C. The IL-12 Family Member P40 Chain as a Master Switch and Novel Therapeutic Target in Psoriasis. J Invest Dermatol (2004) 123: xiv-xv). IL-12 and IL-23 share the common subunit IL-12p40, and are thought to be integrally involved in controlling differentiation of Th1 and Th17 cells, respectively, during the psoriasis immune response (Torti D C, Feldman S R. Interleukin-12, Interleukin-23, and Psoriasis: Current Prospects. J Am Acad Dermatol (2007) 57: 1059-68). Following differentiation, Th1 cells produce interferon-γ; Th17 cells produce the proinflammatory mediators IL-17 and IL-22.³ IL-12p40 is upregulated in human psoriasis plaques, and blocking it abrogates lesions in a mouse model of psoriasis (Hong K, Chu A, Ludviksson B R et al. IL-12, Independently of IFN-Gamma, Plays a Crucial Role in the Pathogenesis of a Murine Psoriasis-Like Skin Disorder. J Immunol (1999) 162: 7480-91; Lee E, Trepicchio W L, Oestreicher J L et al. Increased Expression of Interleukin 23 P19 and P40 in Lesional Skin of Patients with Psoriasis Vulgaris. J Exp Med (2004) 199: 125-30). In addition, IL-12/23 expression is reduced following psoriasis treatment (Gottlieb A B, Chamian F, Masud S et al. TNF Inhibition Rapidly Down-Regulates Multiple Proinflammatory Pathways in Psoriasis Plaques. J Immunol (2005) 175: 2721-9; Piskin G, Tursen U, Sylva-Steenland R M et al. Clinical Improvement in Chronic Plaque-Type Psoriasis Lesions after Narrow-Band UVB Therapy Is Accompanied by a Decrease in the Expression of IFN-Gamma Inducers—IL-12, IL-18 and IL-23. Exp Dermatol (2004) 13: 764-72; Chamian F, Lowes M A, Lin S L et al. Alefacept Reduces Infiltrating T Cells, Activated Dendritic Cells, and Inflammatory Genes in Psoriasis Vulgaris. Proc Natl Acad Sci USA (2005) 102: 2075-80). Further support for the critical role of IL-12/23 in the pathogenesis of psoriasis comes from clinical trials demonstrating the efficacy of anti-IL-12/23p40 monoclonal antibodies as psoriasis therapy (Gottlieb A B, Cooper K D, McCormick T S et al. A Phase 1, Double-Blind, Placebo-Controlled Study Evaluating Single Subcutaneous Administrations of a Human Interleukin-12/23 Monoclonal Antibody in Subjects with Plaque Psoriasis. Curr Med Res Opin (2007) 23: 1081-92; Kauffman C L, Aria N, Toichi E et al. A Phase I Study Evaluating the Safety, Pharmacokinetics, and Clinical Response of a Human Il-12 P40 Antibody in Subjects with Plaque Psoriasis. J Invest Dermatol (2004) 123: 1037-44; Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (Phoenix 1). Lancet (2008) 371: 1665-74; Papp K A, Langley R G, Lebwohl M et al. Efficacy and Safety of Ustekinumab, a Human Interleukin-12/23 Monoclonal Antibody, in Patients with Psoriasis: 52-Week Results from a Randomised, Double-Blind, Placebo-Controlled Trial (Phoenix 2). Lancet (2008) 371: 1675-84). Briakinumab, a fully human, anti-IL-12/23p40 monoclonal antibody, was shown to be highly effective and well tolerated in the treatment of psoriasis in a 12-week, Phase II trial (Kimball A B, Gordon K B, Langley R G et al. Safety and Efficacy of ABT-874, a Fully Human Interleukin 12/23 Monoclonal Antibody, in the Treatment of Moderate to Severe Chronic Plaque Psoriasis: Results of a Randomized, Placebo-Controlled, Phase 2 Trial. Arch Dermatol (2008) 144: 200-7).

In light of the demonstrated efficacy of both the TNF-α and IL-12/23 antagonist classes of drugs as psoriasis therapy, the study described in this Example sought to determine the efficacy, safety, and tolerability of briakinumab compared with etanercept and placebo for the treatment of moderate to severe chronic plaque psoriasis at 12 weeks. A companion study with identical design was concurrently conducted (Strober B E, Crowley J J, Yamauchi P S et al. Efficacy and Safety Results from a Phase III, Randomized Controlled Trial Comparing the Safety and Efficacy of Briakinumab to Etanercept and Placebo in Patients with Moderate to Severe Chronic Plaque Psoriasis. Br J Dermatol (2011) 165:661-8).

Briefly, in this Phase III, 12-week study, 347 patients were randomized in a 2:2:1 ratio to receive 200 mg briakinumab at Weeks 0 and 4 followed by 100 mg briakinumab at Week 8 (n=138); 50 mg of etanercept twice weekly 3-4 days apart at Weeks 0-11 (n=141); or placebo injections matching active treatment (n=68). The co-primary efficacy endpoints were the proportion of patients achieving a PGA of 0/1 at Week 12, and the proportion of patients achieving a Psoriasis Area Severity Index (PASI) 75 response at Week 12.

The results showed that 71.0% of briakinumab-treated patients achieved a PGA of 0/1 at Week 12 as compared with 39.7% of etanercept-treated patients and 2.9% of placebo-treated patients (P<0.001, for both comparisons). 81.9% of briakinumab-treated patients achieved a PASI 75 response at Week 12 as compared with 56.0% of etanercept-treated and 7.4% of placebo-treated patients (P<0.001, comparisons). Serious adverse event rates were reported in 4 (2.9%) patients receiving briakinumab, 1 (0.7%) patient receiving etanercept, and 1 (1.5%) placebo-treated patient. In summary, in patients with moderate to severe psoriasis, briakinumab had superior efficacy to both placebo and etanercept at 12 weeks as administered in this study.

### Methods

### Patients

This Phase III, 12-week double-blind, double dummy, multicenter, randomized study took place at 33 sites in the United States (M10-114). Patients were eligible for enrolment if they were ≧18 years of age with a clinical diagnosis of chronic plaque psoriasis for at least 6 months; had stable plaque Ps for at least 2 months before Screening and at Baseline (Week 0) visits; affected body surface area (BSA)≧10%; Physician's Global Assessment (PGA) of at least moderate (≧3); and Psoriasis Area and Severity Index (PASI) score of ≧12 at the Baseline (Week 0) visit. Patients were ineligible for this trial if they had previous exposure to systemic anti-IL-12/23p40 therapy, including briakinumab; previous exposure to etanercept or known hypersensitivity to etanercept; or inability to discontinue topical therapies, phototherapies, or systemic therapies.

The study protocol was approved by an independent ethics committee or institutional review board at each study site, and each patient provided written informed consent.

### Study Design

At Week 0, patients were randomized 2:2:1 to a briakinumab, etanercept, or placebo treatment arm (FIG. 4). Patients in the briakinumab arm received 200 mg briakinumab subcutaneously (SC) at Weeks 0 and 4 followed by 100 mg briakinumab SC at Week 8. Etanercept-treated patients received 50 mg of etanercept SC twice weekly 3-4 days apart at Weeks 0-11. Patients enrolled in the placebo arm received SC injections matching active treatment to maintain the blind. To maintain the blind, all patients received two SC injections at Weeks 0 and 4 and one SC injection at Week 8, consisting of either briakinumab or matching placebo, depending on the treatment arm. In addition, each patient also received two SC injections biweekly, 3 days apart Week 0 through Week 11, consisting of either etanercept or matching placebo, depending on the treatment arm.

### Efficacy and Safety Measures

The co-primary efficacy endpoints were the proportion of patients achieving a Physician's Global Assessment (PGA) of 0/1, defined as a PGA score of “clear” or “minimal” at Week 12, and the proportion of patients achieving ≧Psoriasis Area Severity Index (PASI) 75 response, defined as at least a 75% reduction in PASI score at Week 12 relative to Baseline PASI score.

Secondary efficacy measures included median time to achieve PASI 75 and PGA 0/1, proportion of patients achieving PASI 90 and 100 responses over 12 weeks, and proportion of patients with a DLQI score of 0 at Week 12.

Adverse events, labs, and vital signs were assessed over the entire trial. Adverse events occurring up to 45 days after study drug administration were included in the analyses.

### Statistical Methods

Approximately 350 subjects were to be randomized in a 2:2:1 ratio to receive briakinumab (140 subjects); etanercept (140 subjects), and placebo (70 subjects). Assuming that the clinical response (PGA 0/1) rates at Week 12 were 70% in the briakinumab group, 50% in the etanercept group, and 4% in the placebo group, this sample size would have provided 90% power to determine the superiority of briakinumab relative to etanercept using a Chi-square test and more than 90% power to show that briakinumab treatment was superior to placebo.

The primary efficacy analysis consisted of four comparisons performed in the intent-to-treat (ITT) population (ie, all randomized subjects), which were tested in a fixed sequence at the alpha=0.05 significance level in order to address the issues of multiplicity as follows: 1) PGA 0/1 response rates for briakinumab vs. placebo at Week 12; 2) PASI 75 response rates for briakinumab vs. placebo at Week 12; 3) PGA 0/1 response rates for briakinumab vs. etanercept at Week 12; 4) PASI 75 response rates for briakinumab vs. etanercept at Week 12.

The primary analyses were performed using a Cochran-Mantel-Haenszel tests stratified by pooled centre. A Chi-Square test, or Fisher's exact test as appropriate, was used to compare proportions of patients in each treatment group achieving PASI 75, 90 or 100 at Weeks 2, 4, 8, and 12, as well as DLQI score of 0 at Week 12. Non-responder imputation (NR1) was used to handle missing data. Median time to achieve PASI 75 and PGA 0/1 were calculated using the Kaplan Meier method for each treatment group, and treatment comparison was performed using the Log-rank test. Patients not achieving the response on or before Week 12 were censored at the date of the last PASI or PGA evaluation. Statistical comparisons were made for briakinumab vs. etanercept and briakinumab vs. placebo. All statistical tests were two-sided with the significance level of 0.05.

The safety analyses were conducted for all subjects who received at least one dose of study drug; safety endpoints were summarized by treatment group.

### Results

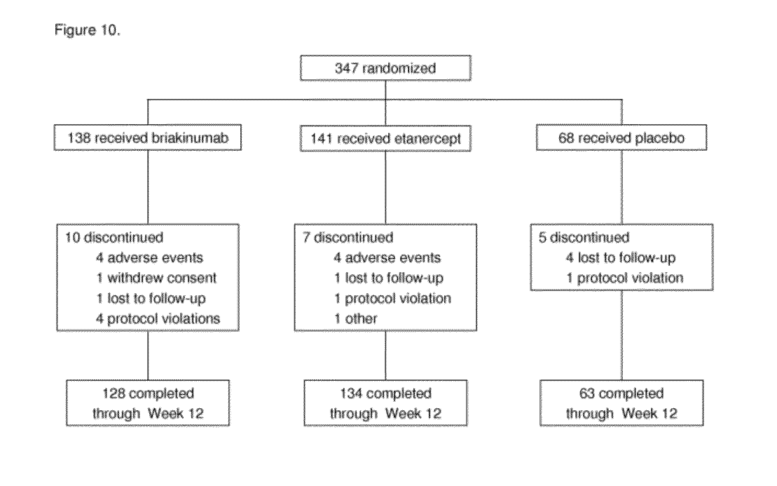

A total of 347 patients were enrolled in the study (placebo, n=68; etanercept, n=141; briakinumab, n=138). Sixty-three (92.6%) placebo-treated, 134 (95.0%) etanercept-treated, and 128 (92.8%) briakinumab-treated patients completed the study (FIG. 10). Four patients each (2.8%) in the briakinumab and etanercept treatment arms discontinued the study due to adverse events; no patients in the placebo arm discontinued due to adverse events. Baseline demographic and clinical characteristics were similar across treatment groups, and reflective of a moderate to severe psoriasis patient population (Table 12). The majority of patients across all treatment groups had a PGA of moderate at baseline, and mean PASI scores ranged from 18.4 to 19.4. At baseline, 85.3% of placebo patients, 88.7% of etanercept-treated patients, and 88.4% of briakinumab-treated patients had 1 or more of the following cardiovascular risk factors: ≧45 years of age for males or ≧55 years of age for females; BMI≧30, current cigarette smoking; history of diabetes or baseline glucose≧126 mg/dL; history of hypertension or baseline SBP≧140 or DBP≧90; history of cardiovascular disease (angina, coronary artery disease, myocardial infarction, cerebrovascular accident, cerebral haemorrhage, transient ischemic attack, congestive heart failure, and peripheral vascular disease-arterial). A greater percentage of patients in the placebo treatment group had ≧2 or ≧3 of these cardiovascular risk factors than patients in the other treatment groups (placebo, 67.6% and 33.8%; etanercept, 57.4% and 24.8%; briakinumab, 58.0% and 26.8%; ≧2 and ≧3 factors, respectively). Similar percentages of patients across treatment groups received prior phototherapy or systemic non-biologic therapy as psoriasis treatment. In addition, there was no difference in the proportion of patients across treatment groups reporting any prior systemic biologic psoriasis treatment, including therapy with TNF-αantagonists.

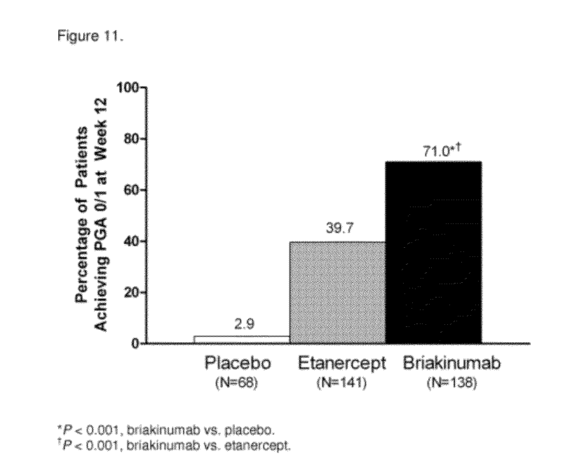

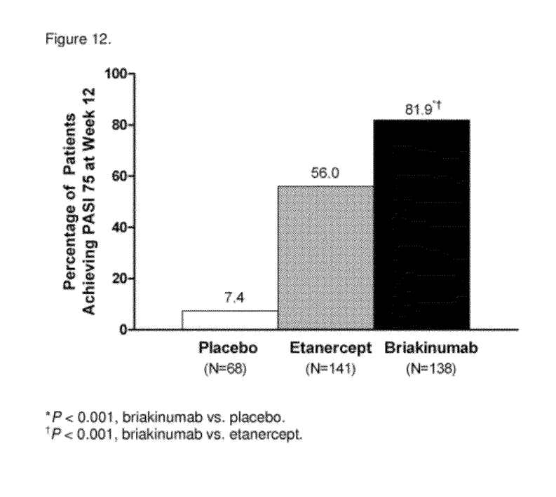

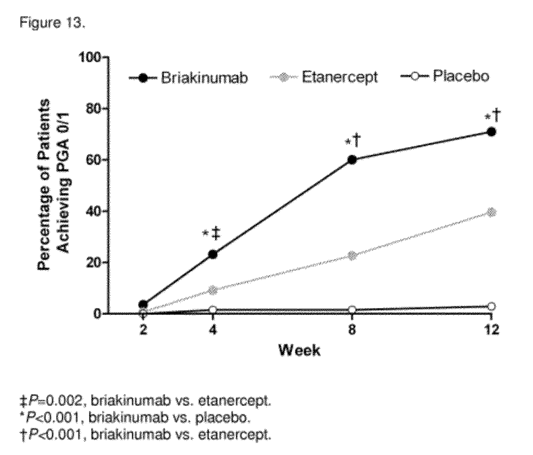

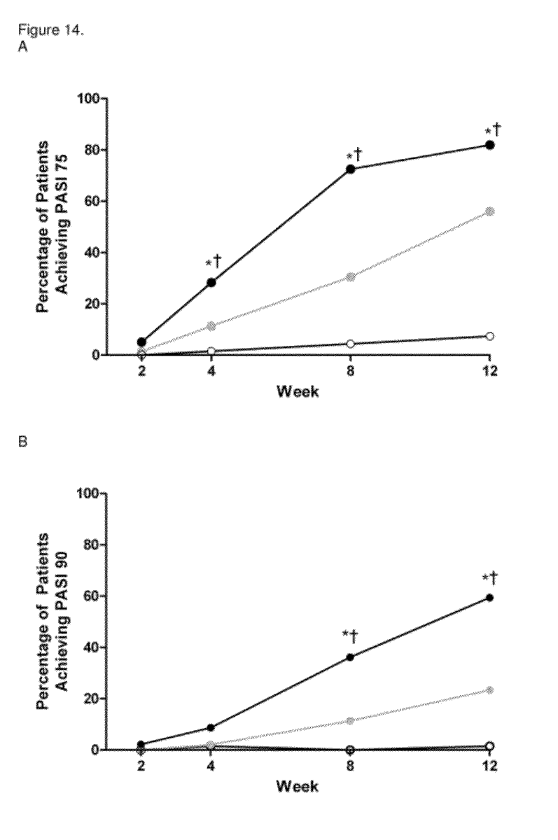

At Week 12, a statistically significantly greater percentage of patients in the briakinumab treatment group (71.0%) achieved the first co-primary endpoint, a PGA of 0/1, as compared with patients receiving etanercept (39.7%) or placebo (2.9%; P<0.001, for both comparisons; FIG. 11). A statistically significantly greater percentage of patients in the briakinumab treatment group (81.9%) also achieved the second co-primary endpoint, a PASI 75 response at Week 12, as compared with patients receiving etanercept (56.0%) or placebo (7.4%; P<0.001, for both comparisons; FIG. 12).

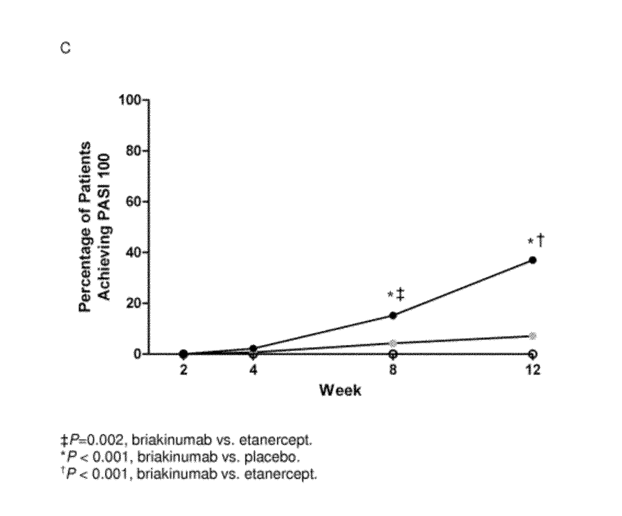

At Week 12, a statistically significantly greater percentage of briakinumab-treated patients achieved PASI 90 or PASI 100 as compared with placebo- or etanercept-treated patients (P≦0.002, for both comparisons for both endpoints; FIG. 14). Median time to achieve a PGA 0/1 was 57 days for patients in the briakinumab treatment group, as compared with 87 days for etanercept-treated patients; too few placebo-treated patients achieved a PGA 0/1 to compute a median (P<0.001, for both comparisons; Table 13). In addition, median time to achieve PASI 75 was statistically significantly shorter for briakinumab-treated patients (57 days) compared with etanercept- or placebo-treated patients (85 and 96 days, respectively) (P<0.001, for both comparisons; Table 13). Analysis of secondary efficacy variables, including the proportion of patients achieving PASI 75/90/100 responses and a PGA score of 0/1 at all time points measured, demonstrated superior efficacy of briakinumab versus etanercept prior to Week 12 (FIGS. 5 and 6).

A statistically significantly greater proportion of patients in the briakinumab treatment group (35.5%) achieved a DLQI score of 0 at Week 12 as compared with patients in either the etanercept (21.3%) or placebo (2.9%) treatment groups (P<0.008, for both comparisons; Table 13).

No deaths occurred during this study (Table 14). A higher percentage of patients receiving etanercept (53.9%) or briakinumab (49.3%) experienced adverse events as compared with patients receiving placebo (45.6%); however, the safety profile for the 2 active treatments was similar. Serious adverse events were reported in 4 (2.9%) patients receiving briakinumab (viral infection requiring hospitalization for dehydration, malignant melanoma in situ, anxiety/pain, and lumbar vertebral fracture), 1 (0.7%) patient receiving etanercept (skin infection involving right shoulder), and 1 (1.5%) placebo patient (hip fracture). The percentage of patients with serious infections was comparable between etanercept and briakinumab (placebo, 0%; etanercept, 0.7%; briakinumab, 0.7%). One malignancy each was reported in the etanercept and briakinumab groups. One patient receiving etanercept was diagnosed with a basal cell carcinoma on study day 84; the patient underwent surgical removal of the lesion. One briakinumab-treated patient was diagnosed with malignant melanoma in situ on study Day 29; study drug was discontinued. No major adverse cardiac events (MACE), defined as non-fatal myocardial infarction, non-fatal stroke, or cardiovascular death, were reported in any of the treatment groups. Three patients in the etanercept group and 4 patients in the briakinumab group reported an ischemic heart disease AE; all 7 events were associated with increased creatine phosphokinase (CPK). Elevated CPK was one of the broad search terms used for the “ischemic heart disease” Standardised MedDRA term Query (SMQ) search; however, as these elevated CPK values were not fractionated, a direct link cannot be inferred. The most common adverse events occurring in ≧5% of patients receiving briakinumab or etanercept were nasopharyngitis, upper respiratory tract infection, injection site reaction, and headache; the adverse events reported more frequently for etanercept patients than briakinumab patients were nasopharyngitis and injection site reactions. The most frequently reported adverse event for placebo patients was upper respiratory tract infection (Table 15). No clinically meaningful changes in laboratory values or vital signs across treatment groups were observed.

### Discussion

This 12-week, double-blind, double-dummy, randomized trial demonstrated the superiority of briakinumab over etanercept and placebo for the treatment of moderate to severe psoriasis. Superiority was demonstrated by a statistically significantly greater percentage of briakinumab-treated patients compared with etanercept- or placebo-treated patients achieving the co-primary endpoints of a PGA 0/1 and PASI 75 at Week 12, as well as PASI 90 and 100, and all other ranked and non-ranked secondary endpoints, including a DLQI score of 0 at Week 12. In addition, patients receiving briakinumab achieved these endpoints significantly faster than their etanercept- and placebo-treated counterparts, and a significantly higher percentage of briakinumab-treated patients reported a DLQI score of 0 at Week 12 compared with patients in the etanercept or placebo treatment groups. Although a higher percentage of briakinumab- and etanercept-treated patients experienced adverse events as compared with patients receiving placebo, there was no difference between active treatment groups, and no clinically important safety trends were identified during the trial.

The efficacy and safety results obtained with etanercept treatment during this trial were generally consistent with those seen in previous etanercept clinical trials using the same dosing regimen (Gottlieb A B, Matheson R T, Lowe N et al. A Randomized Trial of Etanercept as Monotherapy for Psoriasis. Arch Dermatol (2003) 139: 1627-32; Leonardi C L, Powers J L, Matheson R T et al. Etanercept as Monotherapy in Patients with Psoriasis. N Engl J Med (2003) 349: 2014-22; Papp K A, Tyring S, Lahfa M et al. A Global Phase III Randomized Controlled Trial of Etanercept in Psoriasis: Safety, Efficacy, and Effect of Dose Reduction. Br J Dermatol (2005) 152: 1304-12; Tyring S, Gordon K B, Poulin Y et al. Long-Term Safety and Efficacy of 50 Mg of Etanercept Twice Weekly in Patients with Psoriasis. Arch Dermatol (2007) 143: 719-26). In addition, the results of the current study support previous findings demonstrating the therapeutic benefits of targeting the IL-12/23 pathway as psoriasis therapy. A recent phase II trial in which briakinumab was administered found that the majority of briakinumab-treated patients were able to achieve a PASI 75 in 12 weeks (Kimball A B, Gordon K B, Langley R G et al. Safety and Efficacy of ABT-874, a Fully Human Interleukin 12/23 Monoclonal Antibody, in the Treatment of Moderate to Severe Chronic Plaque Psoriasis: Results of a Randomized, Placebo-Controlled, Phase 2 Trial. Arch Dermatol (2008) 144: 200-7). In addition, several trials have shown that another IL-12/23 inhibitor, ustekinumab, is efficacious for the treatment of moderate to severe psoriasis. Similar to the results seen with briakinumab treatment, the majority of ustekinumab-treated patients achieved a PASI 75 within 12 weeks (Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (Phoenix 1). Lancet (2008) 371: 1665-74; Papp K A, Langley R G, Lebwohl M et al. Efficacy and Safety of Ustekinumab, a Human Interleukin-12/23 Monoclonal Antibody, in Patients with Psoriasis: 52-Week Results from a Randomised, Double-Blind, Placebo-Controlled Trial (Phoenix 2). Lancet (2008) 371: 1675-84). Furthermore, the ustekinumab-treated patients were able to maintain their PASI 75 for up to 40 weeks (Leonardi C L, Kimball A B, Papp K A et al. Efficacy and Safety of Ustekinumab, a Human Interleukin-12/23 Monoclonal Antibody, in Patients with Psoriasis: 76-Week Results from a Randomised, Double-Blind, Placebo-Controlled Trial (Phoenix 1). Lancet (2008) 371: 1665-74).

More recently, the safety and efficacy of ustekinumab was directly compared to that of etanercept in a 12 week trial, ACCEPT (Griffiths C E, Strober B E, van de Kerkhof P et al. Comparison of Ustekinumab and Etanercept for Moderate-to-Severe Psoriasis. N Engl J Med (2010) 362: 118-28). The efficacy results for briakinumab from the current study minor both those seen during ACCEPT, as well as during a parallel trial comparing the efficacy and safety of briakinumab and etanercept, M10-315 (Strober B E, Crowley J J, Yamauchi P S et al. Efficacy and Safety Results from a Phase III, Randomized Controlled Trial Comparing the Safety and Efficacy of Briakinumab to Etanercept and Placebo in Patients with Moderate to Severe Chronic Plaque Psoriasis. Br J Dermatol (2011) 165:661-8). Although interpretation of the safety data from ACCEPT, M10-315, and the current trial is limited given that the trials were of too short a duration and statistically underpowered to detect safety differences between etanercept and briakinumab or ustekinumab, a generally similar safety profile was observed for briakinumab, ustekinumab, and etanercept. Overall observed adverse events rates were not different between briakinumab and etanercept patients enrolled in the current study. During the current trial, a higher percentage of briakinumab-treated patients (2.9%) experienced serious adverse events compared with etanercept patients (0.7%) or placebo-treated patients (1.5%). Similar rates of serious infections and skin cancers were reported in the current trial and M10-315. No major adverse cardiac events (MACE) were reported in the current trial or M10-315. One MACE each were reported in patients receiving 45 mg and 90 mg ustekinumab during ACCEPT; however, the ACCEPT investigators did not report when these events occurred, thus it's possible these events occurred after the initial 12-week phase of the trial. Of note, during the first 12-weeks of a 52-week, phase III trial comparing the efficacy and safety of briakinumab versus placebo, 5 MACE were reported, all in the briakinumab treatment arm.

Although both the TNF-α and IL-12/23 pathways have been shown to be critically involved in the pathogenesis of psoriasis, the results of the current trial, M10-315, and ACCEPT suggest that the IL-12/23 antagonists are more efficacious than etanercept for the treatment of psoriasis. IL12B, the gene that encodes IL-12p40, and IL23R, the gene that encodes the receptor of IL-23, have been indentified as psoriasis susceptibility genes, providing additional support for an integral role of these cytokines in psoriasis (Cargill M, Schrodi S J, Chang M et al. A Large-Scale Genetic Association Study Confirms Il12b and Leads to the Identification of IL23r as Psoriasis-Risk Genes. Am J Hum Genet (2007) 80: 273-90). Interestingly, following successful psoriasis treatment with ustekinumab or etanercept in patients with moderate to severe disease, a downregulation was seen in the cell products necessary for activation of Th1 and Th17 cells (Toichi E, Torres G, McCormick T S et al. An Anti-IL-12p40 Antibody Down-Regulates Type 1 Cytokines, Chemokines, and IL-12/IL-23 in Psoriasis. J Immunol (2006) 177: 4917-26; Zaba L C, Cardinale I, Gilleaudeau P et al. Amelioration of Epidermal Hyperplasia by TNF Inhibition Is Associated with Reduced Th17 Responses. J Exp Med (2007) 204: 3183-94). More recently, the genomic response to ustekinumab and etanercept treatment during ACCEPT was assessed, and it was shown that a similar set of genes were downregulated in both etanercept- and ustekinumab-treated patients achieving PASI 75 at Week 12 (Krueger J, Brodmerkel C, Li K et al. The Molecular Profile of Psoriatic Skin in Responders to Ustekinumab or Etanercept after 12 Weeks of Treatment: Results from the ACCEPT Trial. Am Acad Dermatol (2010) 62: AB13). A more complete elucidation of the mechanism of action underlying the effects of etanercept, briakinumab, and ustekinumab will provide a greater understanding of the efficacy differences seen with etanercept and IL-12/23 antagonist treatment in psoriasis.

Taken together, the results of this 12-week, head-to-head trial, demonstrate the superior efficacy of briakinumab over etanercept and placebo for the treatment of moderate to severe psoriasis. These results highlight the potential advantage of using briakinumab as alternative therapy to etanercept, offering dermatologists a wider range of options when treating the moderate to severe psoriasis patient population.

TABLE 12
Baseline Demographics and Clinical Characteristics

| | Treatment Group | | | |
|---|---|---|---|---|
| | Placebo | Etanercept | Briakinumab | Total |
| Characteristic | (N = 68) | (N = 141) | (N = 138) | (N = 347) |
| Age (yrs)^{a} | 44.0 (13.6) | 43.1 (12.5) | 43.6 (14.3) | 43.4 (13.4) |
| Male, n (%) | 47 (69.1) | 98 (69.5) | 89 (64.5) | 234 (67.4) |
| Caucasian, n (%) | 65 (95.6) | 127 (90.1) | 126 (91.3) | 318 (91.6) |
| Duration of psoriasis (yrs)^{a} | 19.1 (13.2) | 17.0 (12.7) | 16.1 (12.5) | 17.0 (12.7) |
| Body weight (kg)^{a} | 96.5 (27.2) | 94.5 (20.4) | 93.2 (22.9) | 94.3 (22.8) |
| BMI, n (%) | | | | |
| <25 (normal) | | | | |
| 25-<30 (overweight) | | | | |
| ≧30 (obese) | | | | |
| % BSA affected^{a} | 23.8 (15.5) | 24.1 (15.0) | 23.6 (16.6) | 23.8 (15.7) |
| PGA, n (%) | | | | |
| Moderate | 42 (61.8) | 72 (51.1) | 77 (55.8) | 191 (55.0) |
| Severe | 24 (35.3) | 60 (42.6) | 57 (41.3) | 141 (40.6) |
| Very severe | 2 (2.9) | 9 (6.4) | 4 (2.9) | 15 (4.3) |
| PASI score^{a} | 18.5 (6.9) | 19.4 (8.0) | 18.4 (7.2) | 18.8 (7.5) |
| History of PsA, n (%) | 14 (20.6) | 32 (22.7) | 27 (19.6) | 73 (21.0) |
| Previous medical history, n (%) | | | | |
| Any CV^{b} | 28 (41.2) | 37 (26.2) | 49 (35.5) | 114 (32.9) |
| Hyperlipidemia | 5 (7.4) | 11 (7.8) | 15 (10.9) | 31 (8.9) |
| Diabetes mellitus | 4 (5.9) | 6 (4.3) | 6 (4.3) | 16 (4.6) |
| Cardiac risk factors, n (%) ^{c} | | | | |
| 0 | 10 (14.7) | 16 (11.3) | 16 (11.6) | 42 (12.1) |
| 1+ | 58 (85.3) | 125 (88.7) | 122 (88.4) | 305 (87.9) |
| 2+ | 46 (67.6) | 81 (57.4) | 80 (58.0) | 207 (59.7) |
| 3+ | 23 (33.8) | 35 (24.8) | 37 (26.8) | 95 (27.4) |
| Previous psoriasis treatment, n (%) | | | | |
| Topical therapy | 62 (91.2) | 130 (92.2) | 123 (89.1) | 315 (90.8) |
| Phototherapy | 20 (29.4) | 33 (23.4) | 35 (25.4) | 88 (25.4) |
| Systemic non-biologic | 19 (27.9) | 37 (26.2) | 39 (28.3) | 95 (27.4) |
| Systemic biologic | 10 (14.7) | 20 (14.2) | 15 (10.9) | 45 (13.0) |

BMI, body mass index; BSA, body surface area; PGA, Physician's Global Assessment; PASI, Psoriasis Area Severity Index; PsA, psoriatic arthritis; CV, cardiovascular body system.
^{a}Mean (SD).
^{b}A patient who reported 2 or more diagnoses in the cardiovascular body system was only counted once for “Any CV”.
^{c}Cardiovascular risk factors: ≧45 years of age for males or ≧55 years of age for females; BMI ≧30, current cigarette smoking; history of diabetes or baseline glucose ≧126 mg/dL; history of hypertension or baseline SBP ≧140 or DBP ≧90; history of cardiovascular disease (angina, coronary artery disease, myocardial infarction, cerebrovascular accident, cerebral haemorrhage, transient ischemic attack, congestive heart failure, and peripheral vascular disease-arterial)

TABLE 13
Secondary Clinical Measures at Week 12

| | Treatment Group | | |
|---|---|---|---|
| | Placebo | Etanercept | Briakinumab |
| | (N = 68) | (N = 141) | (N = 138) |
| Time to achieve PGA 0/1, | — | 87 | 57 |
| Median (days) | | | |
| Time to achieve PASI 75, | 96 | 85 | 57 |
| Median (days) | | | |
| Patients with DLQI score | 2 (2.9) | 30 (21.3) | 49 (35.5) |
| of 0 at Week 12, n (%) | | | |

PGA, Physician's Global Assessment; PASI, Psoriasis Area Severity Index; DLQI, Dermatology Life Quality Index;
— indicates median can not be estimated due to too few patients achieving PGA 0/1 during trial.

TABLE 14
Overview of adverse events

| | Treatment Group, n (%) | | |
|---|---|---|---|
| | Placebo | Etanercept | Briakinumab |
| | (N = 68) | (N = 141) | (N = 138) |
| Any AE | 31 (45.6) | 76 (53.9) | 68 (49.3) |
| Any severe AE | 1 (1.5) | 3 (2.1) | 6 (4.3) |
| Any serious AE | 1 (1.5) | 1 (0.7) | 4 (2.9) |
| Any AE leading to discontinuation | 0 | 4 (2.8) | 4 (2.9) |
| of study drug | | | |
| Deaths | 0 | 0 | 0 |
| Any infection | 13 (19.1) | 34 (24.1) | 31 (22.5) |
| Any serious infection | 0 | 1 (0.7)^{a} | 1 (0.7)^{b} |
| Any malignancy | 0 | 1 (0.7)^{c} | 1 (0.7)^{d} |
| Squamous cell carcinoma | 0 | 1 (0.7) | 0 |
| Basal cell carcinoma | 0 | 0 | 0 |
| Other | 0 | 0 | 1 (0.7) |
| MACE^{e} | 0 | 0 | 0 |

^{a}Skin infection.
^{b}Viral infection.
^{c}Patient was diagnosed with basal cell carcinoma on study day 84.
^{d}Patient was diagnosed with malignant melanoma in situ on study day 29.
^{e}MACE events were defined as non-fatal myocardial infarction, non-fatal stroke, or cardiovascular death.

TABLE 15
Adverse events occurring in ≧5%
of patients in any treatment group.

| | Treatment Group, n (%) | | |
|---|---|---|---|
| | Placebo | Etanercept | Briakinumab |
| | (N = 68) | (N = 141) | (N = 138) |
| Nasopharyngitis | 2 (2.9) | 11 (7.8) | 10 (7.2) |
| Upper respiratory tract infection | 6 (8.8) | 8 (5.7) | 9 (6.5) |
| Injection site reaction | 3 (4.4) | 13 (9.2) | 8 (5.8) |
| Headache | 2 (2.9) | 7 (5.0) | 2 (1.4) |

### Example 5

### Responses to Briakinumab Across Subgroups of Patients with Moderate to Severe Psoriasis

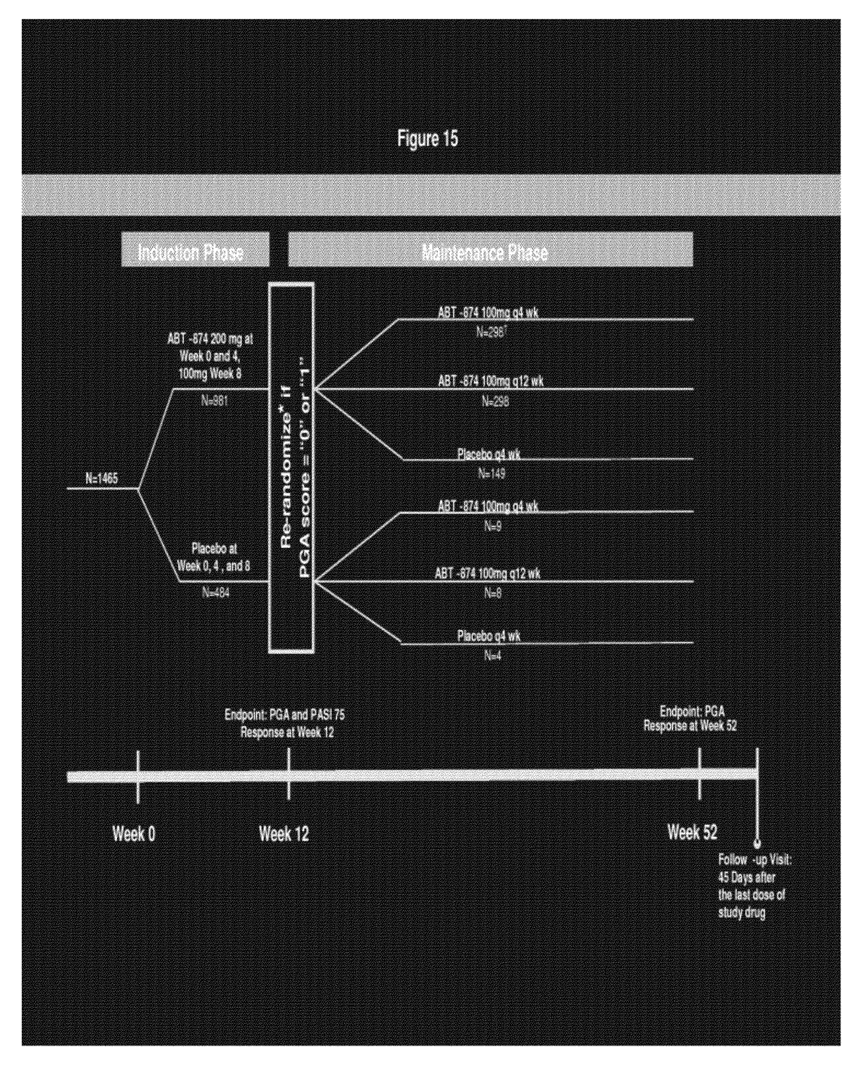

Briakinumab is a fully human monoclonal antibody targeting interleukins 12 and 23. Patients with psoriatic arthritis, more severe psoriasis, higher weight, and prior failure of systemic psoriasis therapies have previously been identified as having suboptimal response to anti-IL-12/23 treatment. M06-890 was a phase 3, randomized, placebo-controlled trial that assessed the efficacy and safety of briakinumab in moderate to severe psoriasis. A subanalysis of M06-890 was conducted to determine efficacy responses across subgroups of patients is described in this Example. The study design is shown in FIG. 15. Baseline characteristics for the study are shown in Table 16.

TABLE 16
Baseline Characteristics

| | | | Maintenance Phase* | | |
|---|---|---|---|---|---|
| | Induction Phase | | Briakinumab | Briakinumab | |
| | Briakinumab | Placebo | q4 weeks | q12 weeks | Placebo |
| | (N = 981) | (N = 484) | (N = 297)^{†} | (N = 298) | (N = 149) |
| Age (years) | 46 (13.2) | 45 (13.5) | 45 (13.3) | 46 (12.6) | 45 (13.4) |
| Sex, male | 666 (67.9%) | 343 (70.9%) | 206 (69.1%) | 201 (67.4%) | 100 (67.1%) |
| Weight (kg) | 94 (23.6) | 93 (23.0) | 91 (21.3) | 94 (23.4) | 89 (24.6) |
| Duration of | 19 (12.3) | 19 (11.9) | 19 (12.1) | 19 (12.1) | 19 (12.4) |
| Ps^{‡} (years) | | | | | |
| PsA | 290 (29.6%) | 150 (31.0%) | 86 (28.9%) | 79 (26.5%) | 42 (28.2%) |
| BSA (%) | 25 (16.3) | 26 (16.9) | 24 (14.8) | 22 (13.5) | 25 (17.1) |
| PASI | 19 (7.5) | 19 (7.3) | 18 (6.5) | 18 (6.2) | 19 (8.2) |
| PGA | | | | | |
| Moderate | 514 (52.4%) | 242 (50.0%) | 170 (57.0) | 164 (55.0%) | 85 (57.0%) |
| Severe | 408 (41.6%) | 218 (45.0%) | 113 (37.9%) | 124 (41.6%) | 53 (35.6%) |
| Very severe | 59 (6.0%) | 24 (5.0%) | 15 (5.0%) | 10 (3.4%) | 11 (7.4%) |

Data are mean (SD) or n (%). Ps = psoriasis; PsA = psoriatic arthritis; BSA = body surface area affected by Ps; PASI = Psoriasis Area and Severity Index; PGA = Physician's Global Assessment.
*Patients randomized to briakinumab during Induction Phase.
^{†}One patient re-randomized to briakinumab q4 weeks did not receive any study drug during the Maintenance Phase.
^{‡}Missing for several patients in the briakinumab and placebo groups during Induction Phase (N = 977 and N = 482, respectively); and in the briakinumab q12 week group during the Maintenance Phase (N = 296).

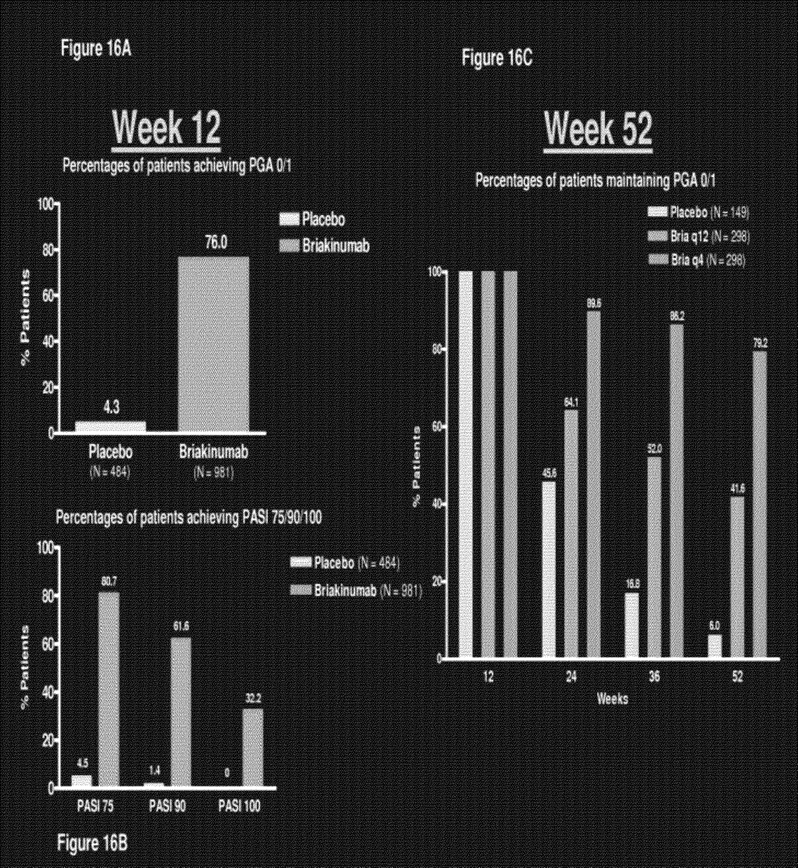

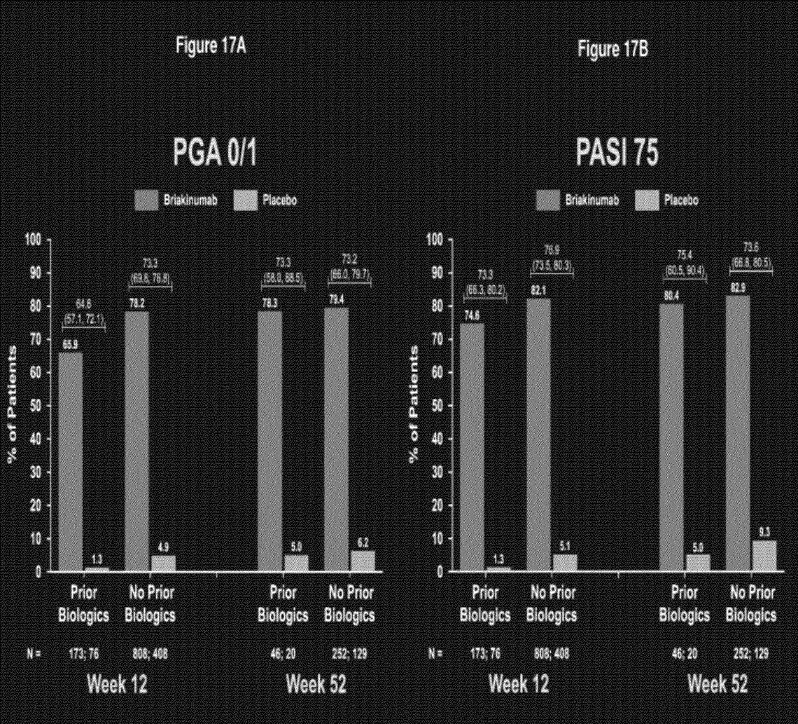

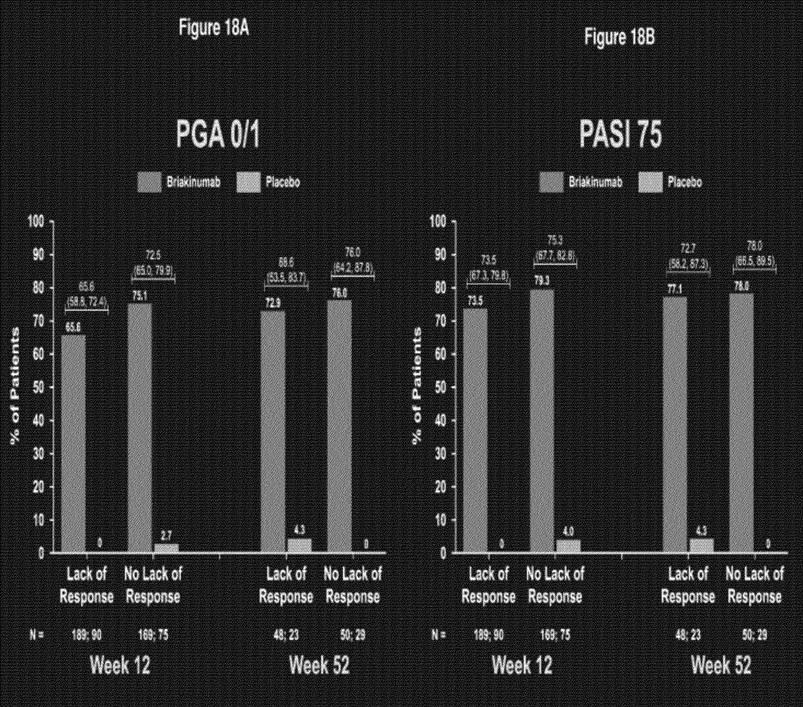

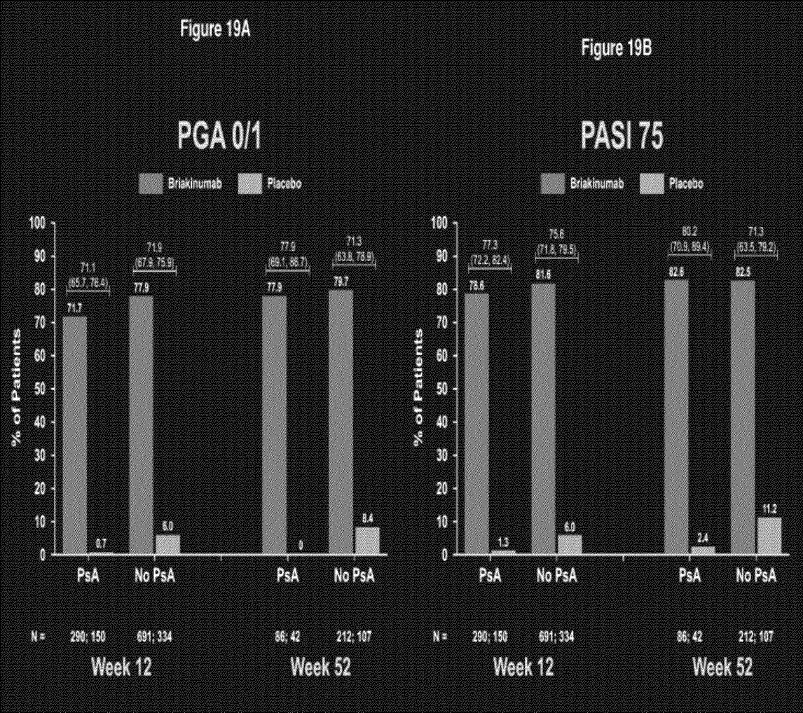

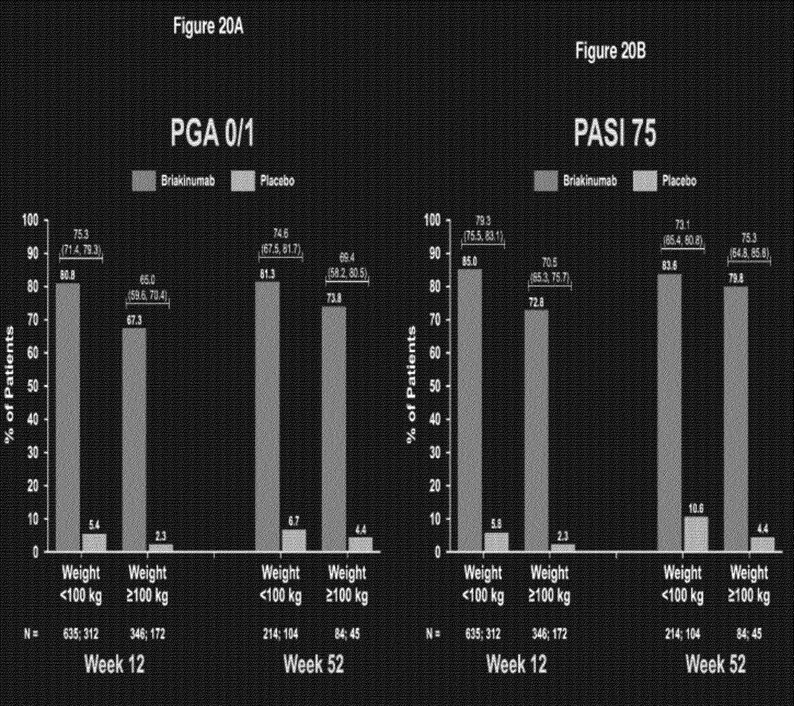

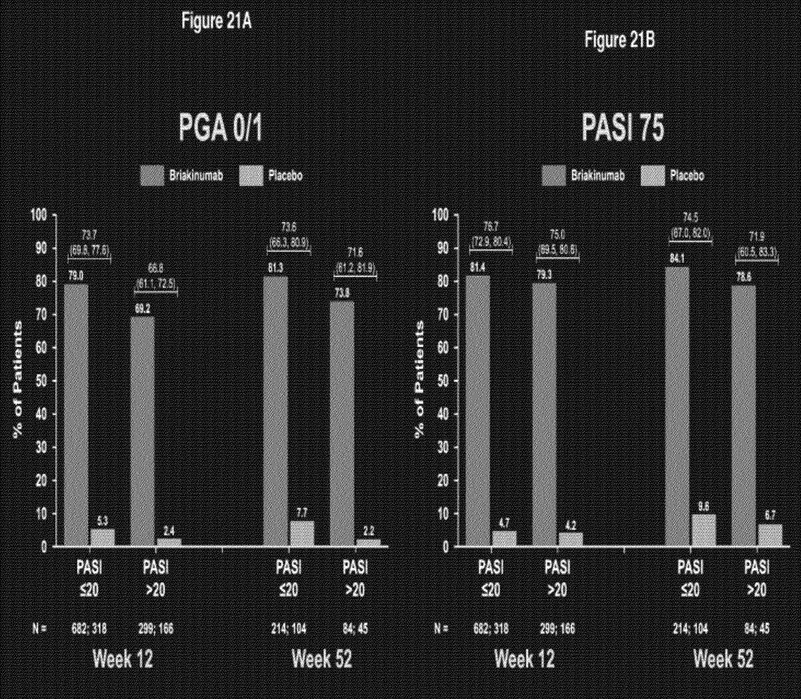

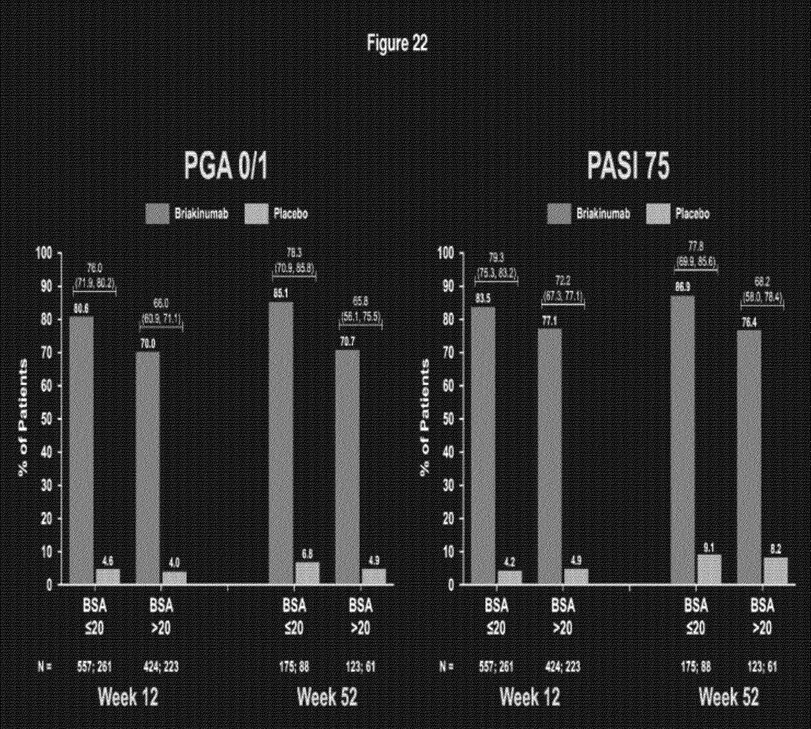

Primary results of the study are shown in FIG. 16. FIG. 17 shows results from patients treated with or not treated with biologics prior to administration of Briakinumab. FIG. 18 shows results from patients treated with biologics prior to administration of Briakinumab and who showed lack of response and no lack of response from the prior biologic treatment. FIG. 19 shows the results from treating patients with a history of psoriatic arthritis. FIG. 20 shows the results from treating patients who had a baseline weight of less than 100 kg or greater than or equal to 100 kg. FIG. 21 shows the results from treating patients who had a baseline disease severity PASI score of less than or equal to 20 or had a baseline disease severity PASI score of greater than 20. FIG. 22 shows the results from treating patients who had a baseline disease severity of less than or equal to 20% body surface area affected (BSA) by psoriasis or who had a baseline disease severity of greater than 20% body surface area affected (BSA) by psoriasis. Safety data are shown in Table 17.

TABLE 17
Safety

| | | | Maintenance Phase* | | |
|---|---|---|---|---|---|
| | Induction Phase | | Briakinumab | Briakinumab | |
| | Briakinumab | Placebo | q4 weeks | q12 weeks | Placebo |
| | (N = 981) | (N = 484) | (N = 297) | (N = 298) | (N = 149) |
| Any AE | 517 (52.7) | 229 (47.3) | 215 (72.4) | 183 (61.4) | 86 (57.7) |
| Any AE leading | 17 (1.7) | 4 (0.8) | 3 (1.0) | 6 (2.0) | 1 (0.7) |
| to discontinuation | | | | | |
| Serious AE | 20 (2.0) | 6 (1.2) | 4 (1.3) | 9 (3.0) | 2 (1.3) |
| Deaths | 1 (0.1)^{†} | 0 | 0 | 0^{‡} | 0 |
| AEs of special | | | | | |
| interest: | | | | | |
| Any infection | 219 (22.3) | 96 (19.8) | 132 (44.4) | 107 (35.9) | 41 (27.5) |
| Serious | 5 (0.5) | 1 (0.2) | 0 | 2 (0.7) | 1 (0.7) |
| infection | | | | | |
| Opportunistic | 0 | 2 (0.4) | 1 (0.3) | 0 | 0 |
| infection | | | | | |
| Malignancy | 6 (0.6) | 0 | 3 (1.0) | 5 (1.7) | 0 |
| SCC | 4 (0.4) | 0 | 0 | 2 (0.7) | 0 |
| BCC | 0 | 0 | 2 (0.7) | 2 (0.7) | 0 |
| Other | 2 (0.2) | 0 | 1 (0.3) | 1 (0.3) | 0 |
| Major adverse | 5 (0.5) | 0 | 1 (0.3) | 1 (0.3) | 0 |
| cardiovascular | | | | | |
| events | | | | | |
| Cardiac arrest | 1 (0.1) | 0 | 0 | 0 | 0 |
| Myocardial | 3 (0.3) | 0 | 0 | 1 (0.3) | 0 |
| infarction | | | | | |
| Stroke | 1 (0.1) | 0 | 0 | 0 | 0 |
| Acute | 0 | 0 | 1 (0.3)^{¶} | 0 | 0 |
| coronary | | | | | |
| syndrome | | | | | |

Data are number of patients (%).
*Patients randomized to briakinumab during Induction Phase (one patient in q4 week dosing group did not receive a dose of study drug during the Maintenance Phase).
^{†}One patient with cardiac arrest resulting in death.
^{‡}One death occurred >45 days after the last dose of study drug, in a patient who had a myocardial infarction prior to study discontinuation.
^{¶}Event was adjudicated to non-fatal myocardial infarction.

### Conclusions

Patients with prior biologic treatment, including those with previous biologic failure, had high efficacy responses as measured by PGA and PASI. Large numbers of patients achieved PGA 0/1 and PASI 75 at weeks 12 and 52, regardless of PsA history, higher weight, or more severe disease at baseline. More infections, malignancies and MACE occurred in briakinumab vs placebo-treated patients, indicating the importance of close surveillance for these events.

### Example 6

### ABT-874 Versus Etanercept or Placebo Treatment for Moderate to Severe Psoriasis Health-Related Quality of Life Outcomes

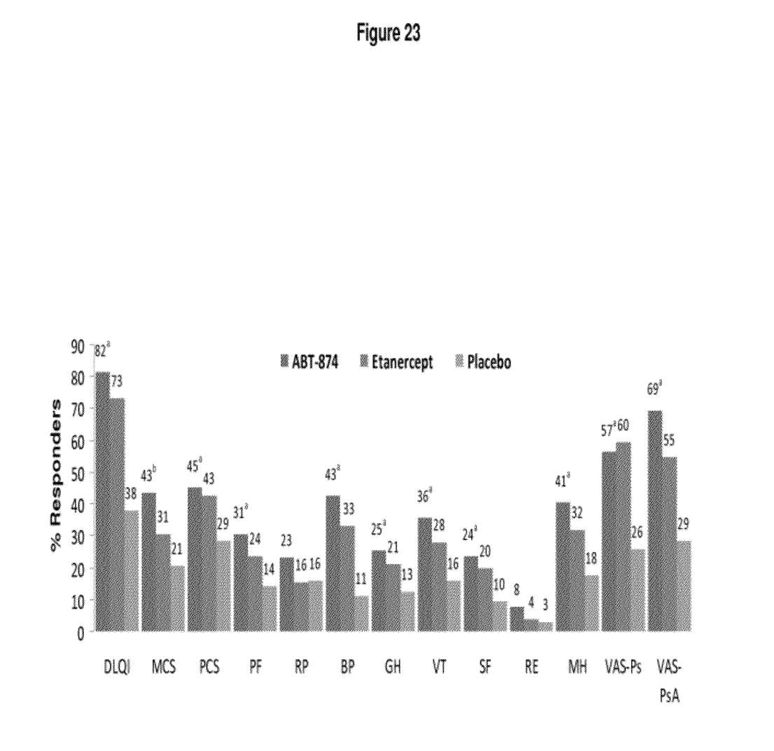

The results presented in this Example and, in particular, shown in Table 18 and FIG. 23, were obtained in a clinical trial conducted concurrently with and using the same protocol as the trial described in Example 1 above.

TABLE 18
Mean change in HRQOL by Week 12

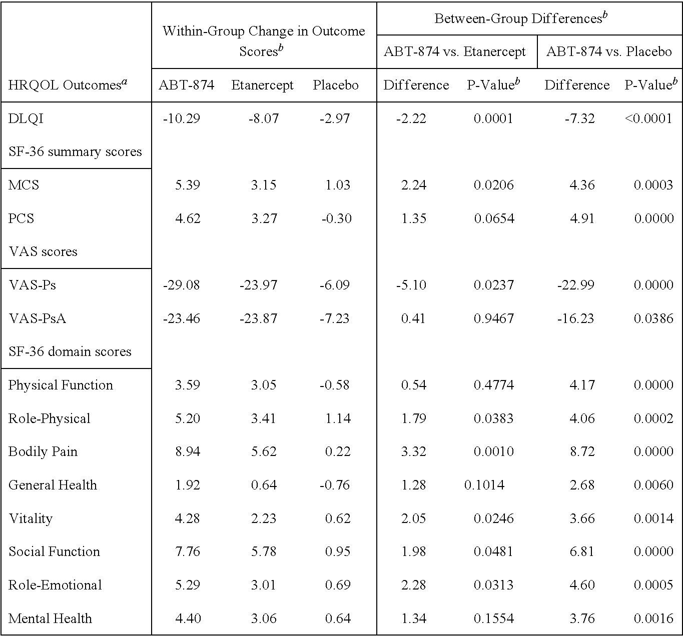

^{a}Increased scores signifies improvement for all HRQOL outcomes except DLQI, VAS-Ps, and VAS-PsA. Missing values at Week 12 were imputed using the last-observation-carried-forward method.
^{b}Least squares means were reported for within group and between group differences. Highlighted cells indicate 5% statistical significane based on the analysis or covariance, adjusting for baseline score and treatment.

ABT-874 was associated with significantly greater mean improvement in all HRQOL outcomes as compared to placebo (p<0.05). ABT-874 was associated with significantly greater mean improvement in DLQI, VAS-Ps, MCS, and several SF-36 domain scores (Role-Physical, Bodily Pain, Vitality, Social Function, and Role—Emotional, Mental Health) as compared to etanercept (Table 18).

As shown in FIG. 23, significantly greater percentages of ABT-874 treated patients achieved clinically meaningful improvement compared with placebo for the following outcomes: DLQI, SF-36 summary scores and domain scores (except for Role—Emotional and Role—Physical), VAS scores for Ps pain and for PsA pain. Patients treated with ABT-874 had significantly greater SF-36 Mental Component Score compared with etanercept.

### Conclusions:

ABT-874 demonstrated significantly greater improvements in all HRQOL measurements vs. placebo and in DLQI, VAS-Ps, SF-36 Mental Component Score (MCS) and the majority of the domain scores vs. etanercept. Significantly greater percentages of patients achieved improvements that were clinically meaningful with ABT-874 treatment in all HRQOL measurements vs. placebo in SF-36 MCS vs. etanercept. These results further enhanced the treatment benefits of ABT-874 on patients' lives beyond the previously described clinical efficacy in significantly reducing Ps symptoms vs. placebo and etanercept.

### Example 7

### Short- and Long-Term Briakinumab Efficacy in Moderate to Severe Psoriasis Patients with Prior TNF Antagonist Exposure: Subanalysis of a 52-Week Phase III Trial and Open-Label Extension

Results from a subset of subjects enrolled in a 52-week double-blind trial that continued into an open label extension trial were evaluated to determine the impact of prior anti-TNF use on short- and long-term efficacy of briakinumab in moderate to severe psoriasis patients.

In the 52-week, Phase III, double-blind trial patients were randomized to briakinumab (200 mg Weeks 0 and 4, 100 mg Week 8) or placebo. If at Week 12 PGA 0 or 1 was achieved, patients were re-randomized to briakinumab 100 mg every 4 weeks (q4wk), every 12 weeks (q12wk), or placebo up to Week 52. OLE enrollment (q4wk dosing) was permitted upon response loss or trial completion.

For patients completing the trial and receiving briakinumab q4wk during all 3 periods (week 0-12, week 13-52, and through the OLE 48 week extension period, weeks 53-100), PASI 75 response rates and proportion of patients achieving PGA 0 or 1 were analyzed by prior anti-TNF exposure. NR1 was used for missing data.

Two hundred fifty two (252) patients achieving Week 12 PGA 0 or 1 were analyzed (anti-TNF naïve, n=190; prior anti-TNF exposure, n=62). For anti-TNF naïve vs. prior anti-TNF exposed patients, respectively, Week 8/Week 52 PASI 75 response rates were 77.4%/96.8% vs. 83.9%/95.2%; PGA of 0 or 1 was achieved by 73.2%/93.2% vs. 66.1%/90.3%. At OLE Week 48, for anti-TNF naïve vs. prior anti-TNF exposed patients, PASI 75 response rates were 93.7% vs. 93.5%; 87.9% vs. 85.5% achieved PGA of 0 or 1. Serious adverse event rates through OLE Week 48 were 4.2% vs. 3.2% for anti-TNF naïve vs. prior anti-TNF exposed patients.

These data demonstrate that regardless of prior anti-TNF exposure, a high percentage of briakinumab-treated patients achieved PASI 75 and a PGA of 0 or 1 at Weeks 8 and 52; this level of response was maintained through OLE Week 48. Serious adverse event rates were low and similar across groups.

### Example 8

### Interim Results from an Open-Label Extension Study of Briakinumab for the Treatment of Moderate to Severe Psoriasis

An interim safety and efficacy results were determined from an ongoing, open-label extension study (OLE) of the anti-IL-12/23 agent, briakinumab, in moderate to severe psoriasis (NCT00626002). Patients from briakinumab phase 2/3 psoriasis trials could elect to enroll in an OLE upon loss of response or study completion, and receive 100 mg briakinumab q4 weeks. The phase 2 and phase 3 trials were 12 or 52 weeks in length. Adverse events (AEs) from first briakinumab dose in any study and up to 45 days following last dose in OLE are collected (malignancies collected anytime following last dose). Maintenance of efficacy is determined in patients with ≧1 dose in preceding study and OLE, and PGA “clear/minimal” prior to first OLE dose (analysis by LOCF). Interim cutoff was set arbitrarily.

Two thousand five hundred twenty (2520) patients (4703.8 PYs drug exposure) received ≧1 dose briakinumab during the interim period. At OLE week 72, 98.7% (623/627) of evaluable patients had PASI 75. 5.6% of OLE patients withdrew due to AEs. Infectious AEs occurred in 54.8% (serious infections 1.3%; opportunistic infections 0.6%), and malignancies in 2.6% (NMSCs 1.7% [BCC, N=25; SCC, N=21]). Twenty (20) major adverse cardiovascular events (MACE) were observed during OLE, in addition to 7 from one run-in study (27 total events [incidence=0.57 events/100 PY]; 19 non-fatal MIs, 3 non-fatal strokes, and 5 cardiovascular deaths). Using a composite of 4 defined cardiovascular risk factors, retrospective analysis revealed that MACE occurred at a rate of 0.27 events/100 PY in patients with ≦1 risk factor compared to 1.61 events/100 PY in patients with ≧2 risk factors.

These results show sustained response of subjects with psoriasis treated with briakinumab through at least 84 weeks, and up to 124 weeks or more.

### Example 9

### Short- and Long-term Briakinumab Efficacy in Moderate to Severe Psoriasis Patients with Prior TNF Antagonist Exposure: Subanalysis of a 52-week Phase III Trial and Open-Label Extension

### Introduction

Psoriasis is a chronic, auto-immune, inflammatory cell-mediated disease that can be physically and socially disabling for patients. Therapeutic efficacy has been demonstrated with anti-tumor necrosis factor (TNF) therapies (such as adalimumab, infl iximab, and etanercept). Briakinumab is a fully human monoclonal antibody targeting the shared IL-12 and -23 p40 subunit, and has recently been shown to be efficacious in the treatment of moderate to severe psoriasis. (See Gottlieb et al., Br. J. Dermatol., DOI 10.1111/j.1365-2133.2011.10418.x; Kimball et al., Arch. Dermatol., 2008, 144:200-207; Kimball et al., J. Am. Acad. Dermatol., 2011; 64:263-274; and Strober et al., Br. J. Dermatol., DOI: 10.1111/j.1365-2133.2011.10419.x). The aim of this study was to evaluate the impact of prior anti-TNF use on short- and long-term efficacy of briakinumab in moderate to severe psoriasis patients.

### Study Design

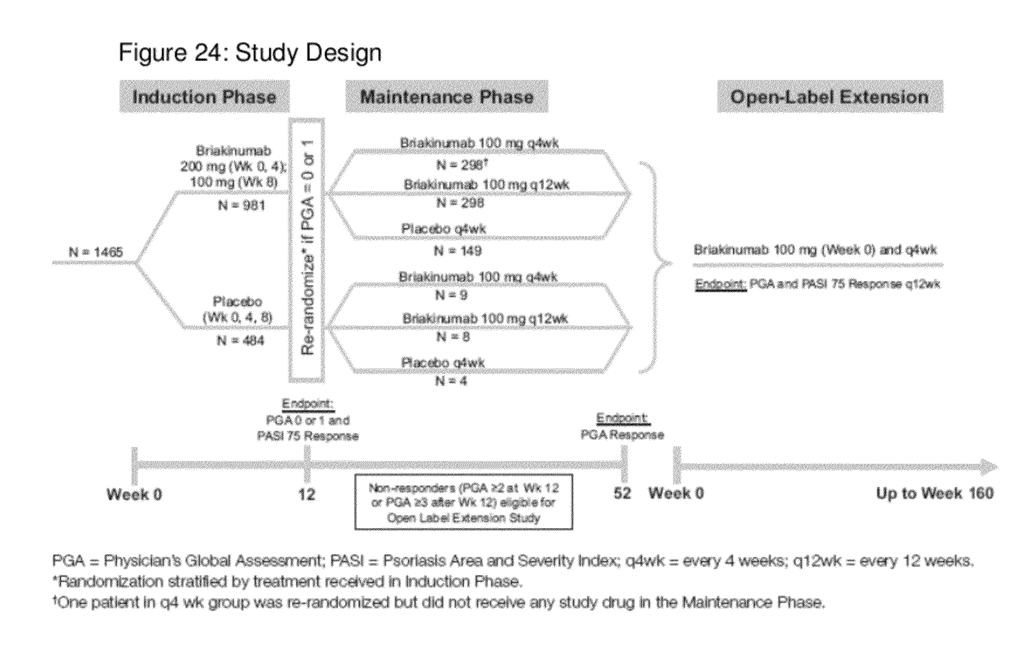

Short- and long-term efficacy of briakinumab was determined in adults who had completed a Phase III study, and then enrolled in an open-label extension study (FIG. 24); patients were analyzed post hoc by prior exposure to anti-TNF therapy at baseline—Phase III, 52-week, double-blind, placebo-controlled, multi-center clinical trial with 2 phases (Induction and Maintenance) (NCT00570986)

Induction Phase:
Patients were randomized 2:1 and received 1 of 2 treatments:
   Briakinumab, 200 mg at Weeks 0 and 4, followed by 100 mg at Week 8
   Placebo

Maintenance Phase:
Patients who achieved a Physicians Global Assessment (PGA) score of
“clear” or “minimal” (PGA 0 or 1) at Week 12 in the Induction Phase were re-randomized 2:2:1 (stratified by treatment received in Induction Phase) to 1 of 3 treatment arms:
   Briakinumab, 100 mg every 4 weeks (q4wk)
   Briakinumab, 100 mg every 12 weeks (q12wk)
   Placebo (q4wk)

Open-Label Extension:
OLE enrollment (q4wk) permitted upon response loss or trial completion (in preceding Phase III study)

Planned duration of 160 weeks

For q4wk patients (all 3 periods), Psoriasis Area and Severity Index (PASI) and PGA response rates were analyzed by prior anti-TNF exposure at baseline

### Patients

Key inclusion criteria:
Adult patients with chronic plaque psoriasis for at least 6 months (and stable for at least 2 months) prior to baseline
Moderate to severe psoriasis defined by the following at baseline:
   Affected body surface area (BSA)≧10%
   PGA at least “moderate” (defined as ≧3)
   PASI≧12

Key exclusion criteria:
Previous exposure to anti-interleukin 12 therapy, including briakinumab
Other forms of psoriasis (other than plaque psoriasis)
Treatment with any of the following:
   Topical treatments (ie, corticosteroids, vitamin D analogs, or retinoids) or UVB phototherapy within 2 weeks of baseline
   PUVA phototherapy or systemic treatments for psoriasis within 4 weeks of baseline
   Biologic treatments within 12 weeks of baseline

### Efficacy and Safety Measures

Efficacy was measured using a 6-point PGA scale and PASI at Weeks 0, 1, 4, and 8 in the Induction Phase, and every month during the Maintenance Phase (Weeks 12 to 52) and every 12 weeks in the OLE. Patients were assessed for adverse events throughout the study, and up to 45 days following the last dose of study medication.

### Statistical Methods

Patients achieving a PGA of 0 or 1 at Week 12 and receiving briakinumab q4wk during all 3 periods (Induction, Maintenance, and Open Label) were included in the efficacy analyses. Missing values were dealt with using non-responder imputation (NR1).

### Results

252 patients achieving Week 12 PGA 0 or 1 were analyzed (anti-TNF naïve, n=190; prior anti-TNF exposure, n=62). Baseline demographics and clinical characteristics were generally similar between the 2 groups with the exception of disease severity; a greater proportion of prior anti-TNF exposed patients had severe or very severe disease at baseline as compared with anti-TNF naïve patients (Table 19).

TABLE 19
Baseline Demographic and Clinical Characteristics

| | Anti-TNF | Prior Anti-TNF |
|---|---|---|
| | Naïve | Use |
| Variable | N = 190 | N = 62 |
| Age (years), n (%) | | |
| <40 | 71 (37.4) | 19 (30.6) |
| 40 to <60 | 103 (54.2) | 40 (64.5) |
| ≧60 | 16 (8.4) | 3 (4.8) |
| Male, n (%) | 134 (70.5) | 39 (62.9) |
| White, n (%) | 172 (90.5) | 58 (93.5) |
| Weight, kg | 89.6 ± 21.6 | 93.2 ± 19.9 |
| PASI | 17.7 ± 6.0 | 19.9 ± 7.7 |
| Affected BSA (%) | 22.2 ± 13.6 | 26.8 ± 18.7 |
| Duration of plaque psoriasis (years) | 17.5 ± 11.9 | 23.8 ± 12.5 |
| Duration of PsA (years)^{a} | 8.7 ± 9.8 | 11.6 ± 10.4 |
| Currently have tender/stiff joints | 42 (22.1) | 22 (35.5) |
| Family history of psoriasis | 93 (48.9) | 33 (53.2) |
| Physician's Gobal Assesment | | |
| Clear, Minimal or Mild | 0 | 0 |
| Moderate | 118 (62.1) | 31 (50.0) |
| Severe | 62 (32.6) | 27 (43.5) |
| Very Severe | 10 (5.3) | 4 (6.5) |

Data are Mean ± SD except where indicated as n (%).
^{a}N = 49 for anti-TNF naive patients; N = 23 for prior anti-TNF use patients.
PASI = psoriasis area and severity index; BSA = body surface area; PsA = psoriatic arthritis.

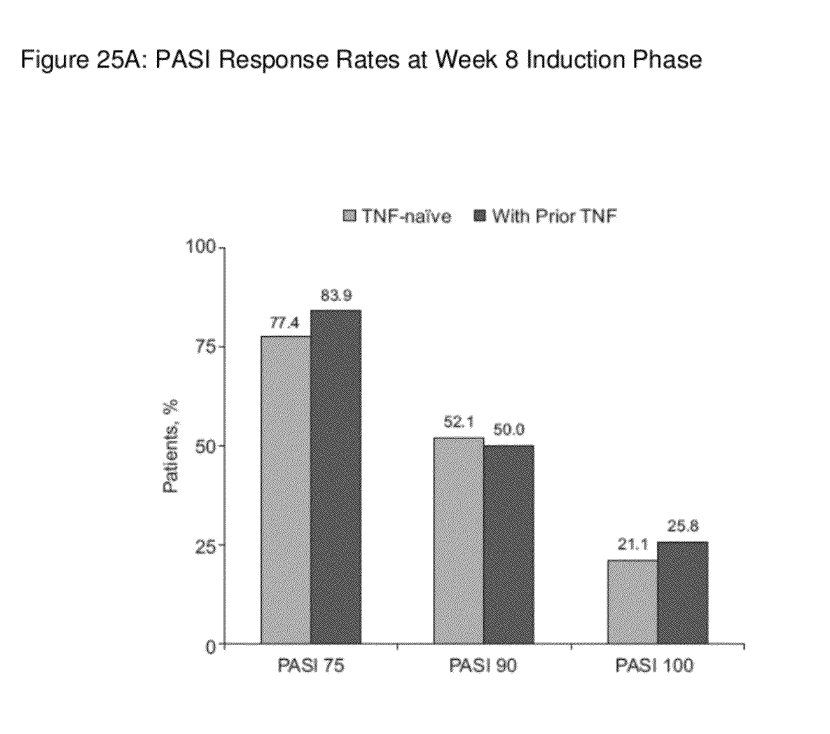

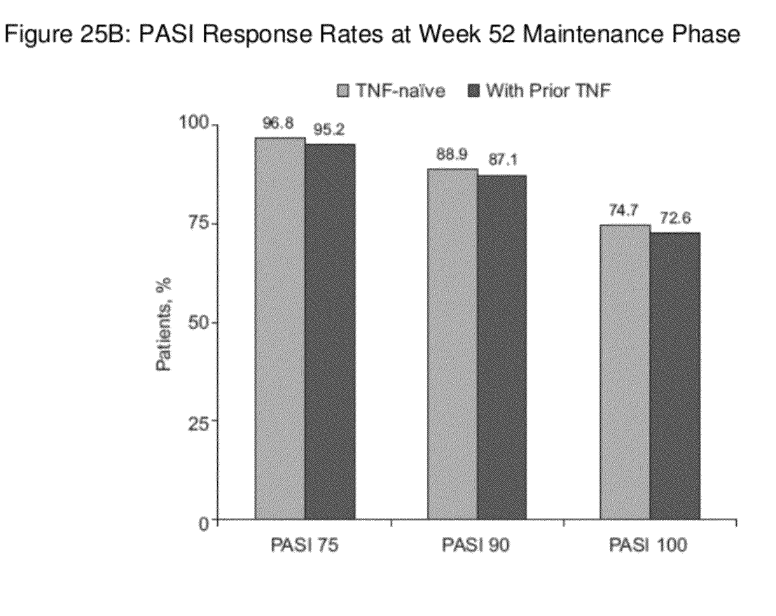

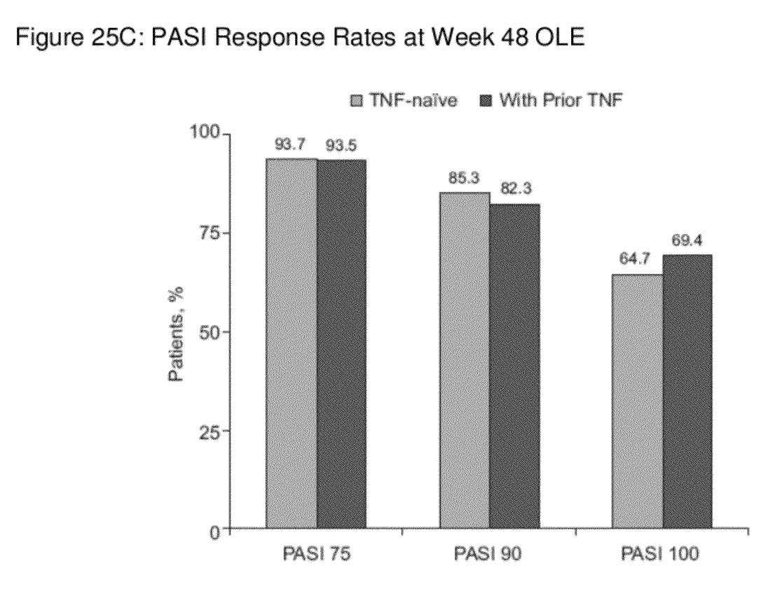

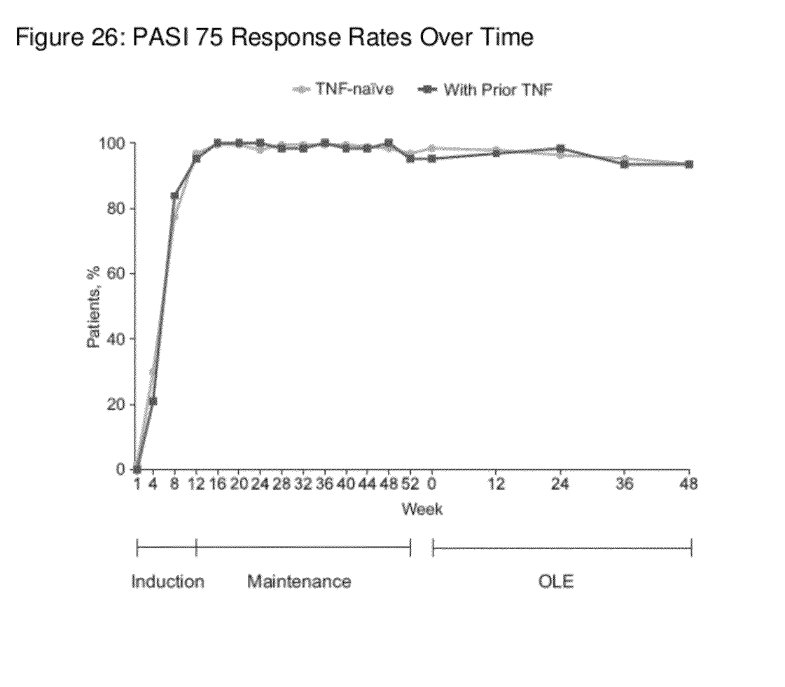

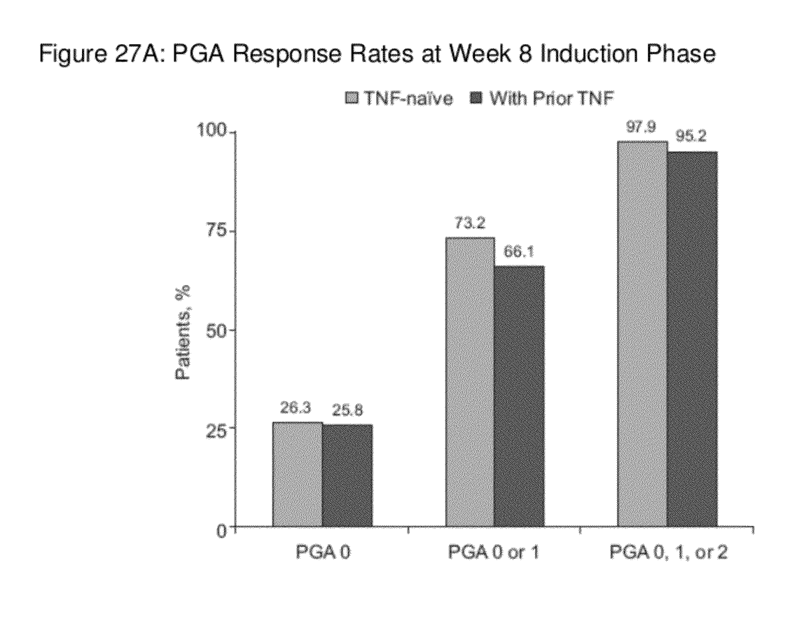

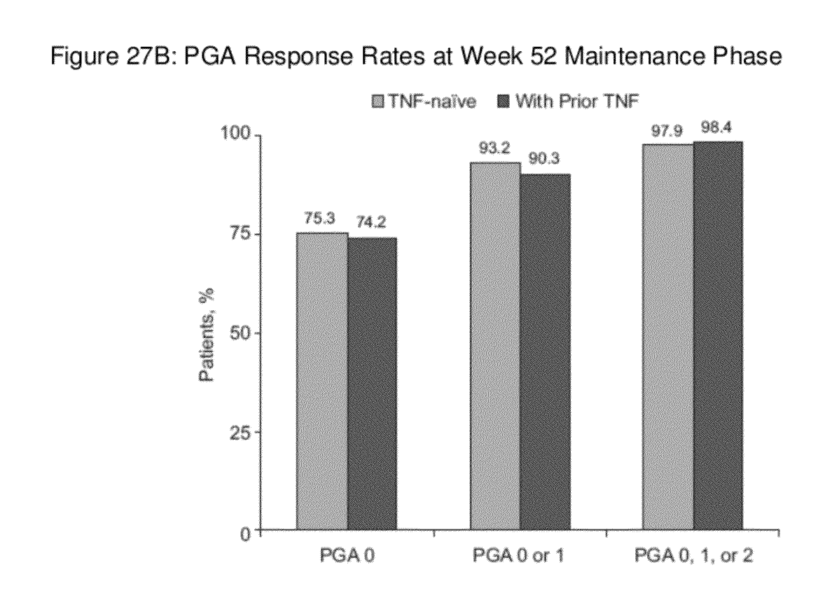

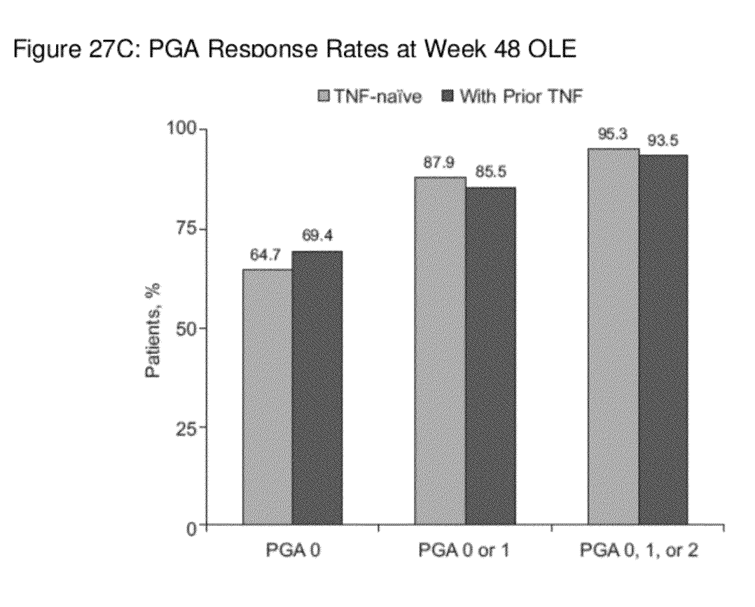

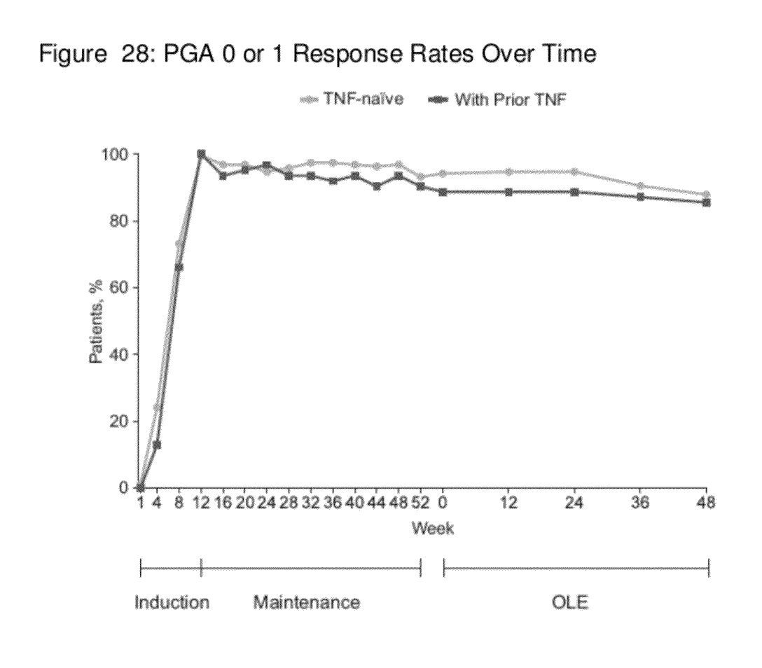

PASI 75, PASI 90 and PASI 100 response rates were similar for anti-TNF naïve vs. prior anti-TNF exposed patients at Week 8 (Induction Phase), Week 52 (Maintenance Phase), and Week 48 (OLE) as shown in FIGS. 25A-25C. The PASI 75 response rates over time were similar for anti-TNF naïve versus prior anti-TNF exposed patients as shown in FIG. 26. Response rates for achieving PGA 1, PGA 0 or 1, and PGA 0, 1, or 2 were similar for anti-TNF naïve versus prior ant-TNF exposed patients for Week 8 (Induction Phase), Week 52 (Maintenance Phase) and Week 48 (OLE) as shown in FIGS. 27A-C. The percentage of patients achieving a PGA 0 or 1 response, over time, was similar for anti-TNF naïve versus prior anti-TNF exposed patients as shown in FIG. 28.

Rates of serious adverse events and adverse events of special interest were similar thorugh OLE Week 48 for anti-TNF naïve versus prior anti-TNF exposed patients (see Table 20).

TABLE 20
Overview of Treatment-emergent Adverse Events

| | Anti-TNF | Prior Anti-TNF |
|---|---|---|
| | Naïve | Use |
| Event | N = 190 | N = 62 |
| Any adverse event | 168 (88.4) | 59 (95.2) |
| Any severe adverse event | 21 (11.1) | 4 (6.5) |
| Any serious adverse event | 8 (4.2) | 2 (3.2) |
| Any adverse event leading to | 6 (3.2) | 0 |
| discontinuation of study drug | | |
| Deaths | 2 (1.1)^{b} | 0 |
| Adverse events of special interest | | |
| Infection | 122 (64.2) | 44 (71.0) |
| Serous infection | 0 | 1 (1.6)^{c} |
| Malignancy | 5 (2.6)^{d} | 3 (4.8) |
| Basal cell carcinoma | 3 (1.6) | 2 (3.2) |
| Squamous cell carcinoma | 2 (1.1) | 1 (1.6) |
| Dysplastic naevus syndrome | 1 (0.5) | 0 |
| Major adverse cardiovascular event | 2 (1.1) | 0 |
| (MACE)^{e} | | |

Values are n (%).
^{a}Adverse events occuring up to 45 days after last dose of study drug.
^{b}One cardiovascular-ralated death; one non-treatment emergent death.
^{c}One patient with pnumonia.
^{d}One patient had dysplastic, naevus syndrome and basal cell carcinoma.
^{e}Included any MACE occuring up to 45 days after last dose of study drug: up to 101 days after last dose of study drug when patient prematurely discontinued and did not have an early termination visit.

### Conclusions

Regardless of prior anti-TNF exposure, a high percentage of briakinumab-treated patients achieved PASI 75 and a PGA of 0 or 1 at Weeks 8 and 52. This level of response was maintained through OLE Week 48. These results show that prior exposure to anti-TNF therapy does not rule out potential benefits from subsequent anti-IL-12/23 therapy for patients with psoriasis. Serious adverse event rates were low and similar across groups.

### Example 10

### Long-term Safety and Efficacy of Briakinumab for the Treatment of Moderate to Severe Psoriasis—Interim Analysis from an Open-label Extension Study

### Introduction

Psoriasis is a chronic immunologic disease, believed to be T-cell mediated. Both IL-12 and Il-23 play prominent roles in T-cell activation. Their cytokine products (IFN-γ, TNF and IL-17) appear to be key to disease mechanisms in psoriasis. Briakinumab is a fully human anti-IL-12p40 antibody, selective for IL-12 and Il-23. High efficacy responses have been reported form Phase II and III clinical studies of briakinumab in moderate to severe psoriasis. The aim of this study was to determine interim safety and efficacy results from an ongoing, open-label extension study (OLE) of the anti-IL-12/23 agent, briakinumab, in moderate to severe psoriasis.

### Study Design

Multi-center, multi-national, open-label extension study (OLE) for long-term assessment of briakinumab's safety and efficacy in psoriasis. Patients eligible for enrollment following completion or loss of response in a preceding phase II or III study (listed in Table 21). Planned duration: 160 weeks. Treatment received: 100 mg briakinumab every 4 weeks, beginning at Week 0 of the OLE.

TABLE 21

| | | Study | OLE |
|---|---|---|---|
| Study | Design | N | N |
| M05-736 | Phase II, 3-period, dose-ranging study; 12-wk | 180 | 84 |
| | DB, 36-wk obs./re-treatment, followed by 60- | | |
| | wk OL re-treatment | | |
| M06-890 | Phase III, 2-period study; 12-wk Induction | 1465 | 1346 |
| | Phase, 40-wk Maintenance Phase | | |
| M10-114 | Phase III, 12-wk, DB, placebo and active | 347 | 308 |
| | comparator (ETN) | | |
| M10-315 | Phase III, 12-wk, DB, placebo and active | 350 | 314 |
| | comparator (ETN) | | |
| M10-255 | Phase III, 52-wk, DB, active-comparator | 317 | 246 |
| | (MTX) | | |

ETN = etanercept; MTX = methotrexate

### Statistical Analyses

Efficacy
Assessed for the Maintenance of Efficacy (ME) population, all patients. Received the loading dose of 200 mg at Weeks 0 and 4, and 100 mg at Week 8 during the preceding study. (Patients enrolled from study M06-890 must have received ≧1 dose of briakinumab during the Induction Phase)
   With a Physician's Global Assessment (PGA) of “clear” or “minimal” (0 or 1) at the last evaluation on or before the first dose in OLE
PGA and Psoriasis Area and Severity Index (PASI) assessed every 12 weeks

Safety
Assessed in all patients with ≧1 dose briakinumab in preceding study or OLE
All AEs recorded from 1st dose of briakinumab (received in preceding study or OLE)
AEs collected throughout preceding studies and up to 45 days from the last dose of study drug

Major Adverse Cardiovascular Events (MACE)
Assessed in all patients with ≧1 dose briakinumab in preceding study or OLE

### Baseline Demographics and Clinical Characteristics

2520 patients (4703.8 PYs drug exposure) received ≧1 dose of briakinumab in the OLE as of October 2010 (Table 22).

TABLE 22

| | AD Briakinumab |
|---|---|
| | (N = 2520) |
| Male, n (%) | 1727 (68.5) |
| White, n (%) | 2301 (91.3) |
| Age (years), mean (SD) | 45.1 ± 13.3 |
| Weight (kg), mean (SD) | 92.9 ± 23.0 |
| Duration of psoriasis (years), mean (SD) | 18.4 ± 12.3 |
| Duration of psoriatic arthritis (years), mean (SD)^{a} | 9.5 ± 8.8 |
| Family history of psoriasis, n (%) | 1225 (48.7) |
| PASI^{b}, mean (SD) | 19.0 ± 7.2 |
| BSA^{b} (%), mean (SD) | 25.0 ± 16.3 |
| PGA^{b}, n (%) | |
| Clear, Minimal or Mild | 0 |
| Moderate | 1302 (51.7) |
| Severe | 1085 (43.1) |
| Very Severe | 133 (5.3) |

^{a}N = 674
^{b}From baseline prior to 1^{st} dose of briakinumab (whether in preceding study or OLE)

### Results

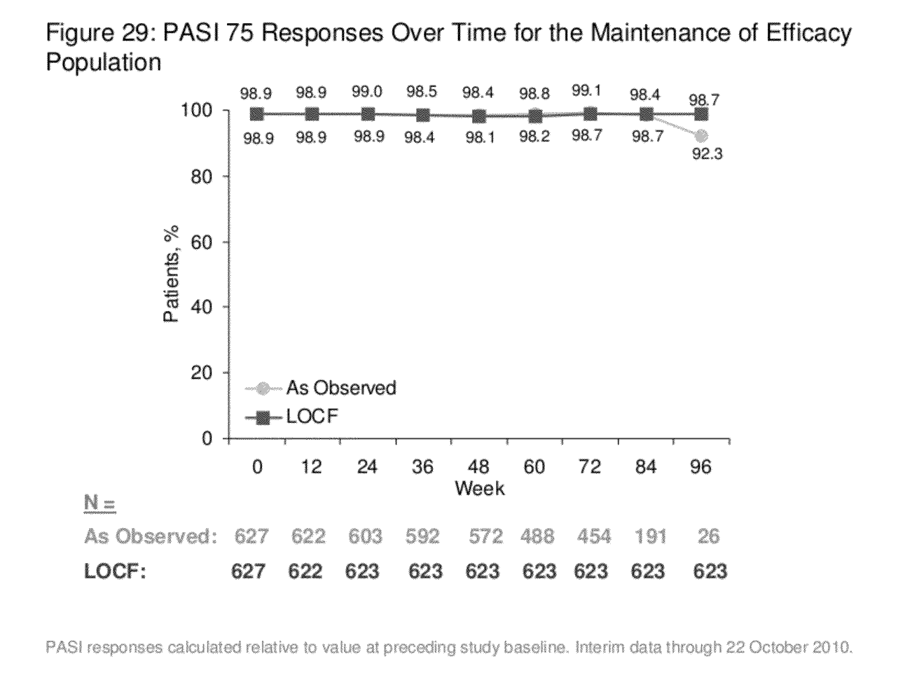

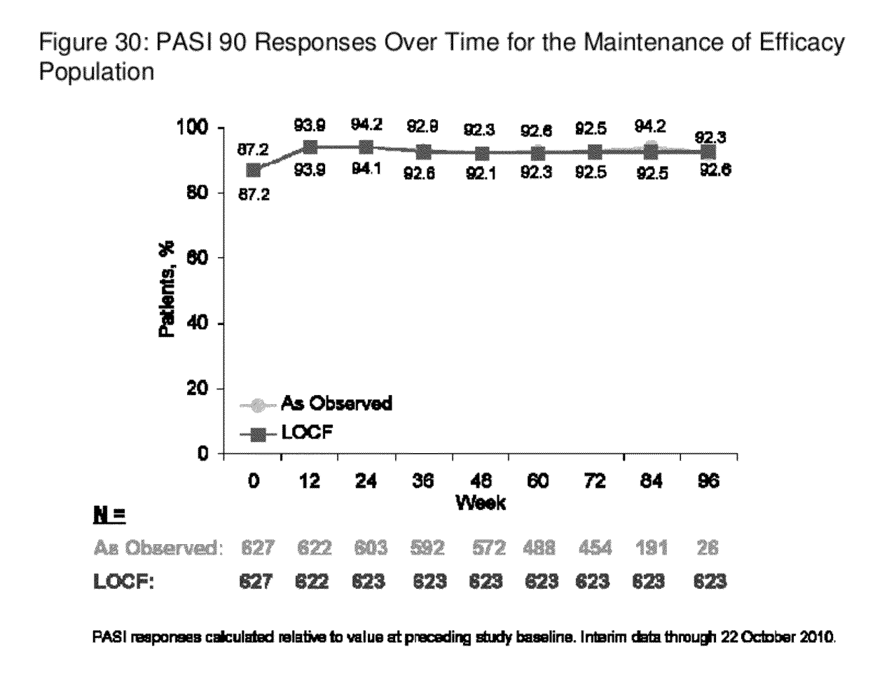

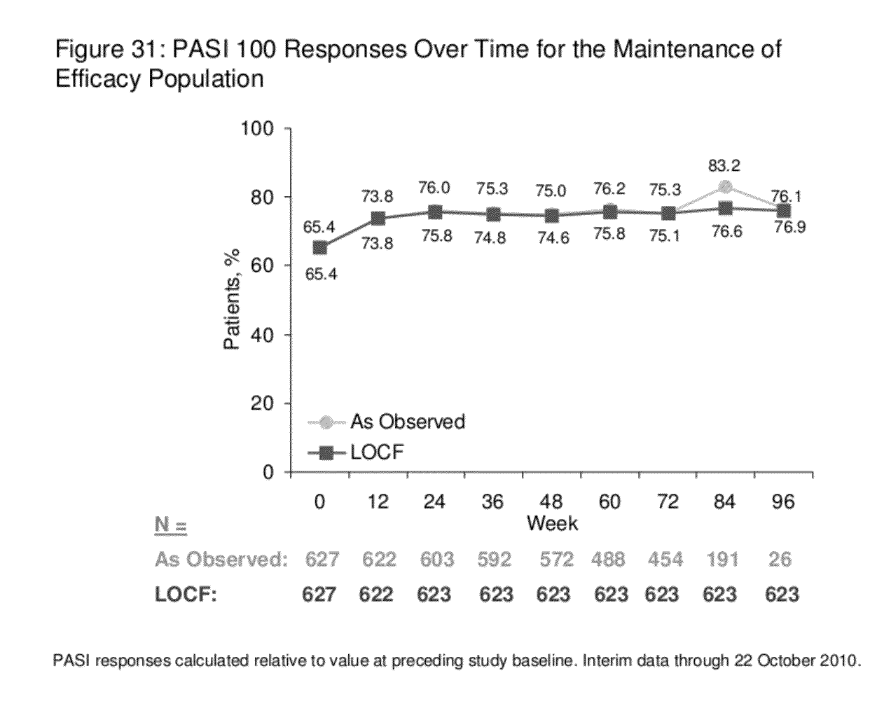

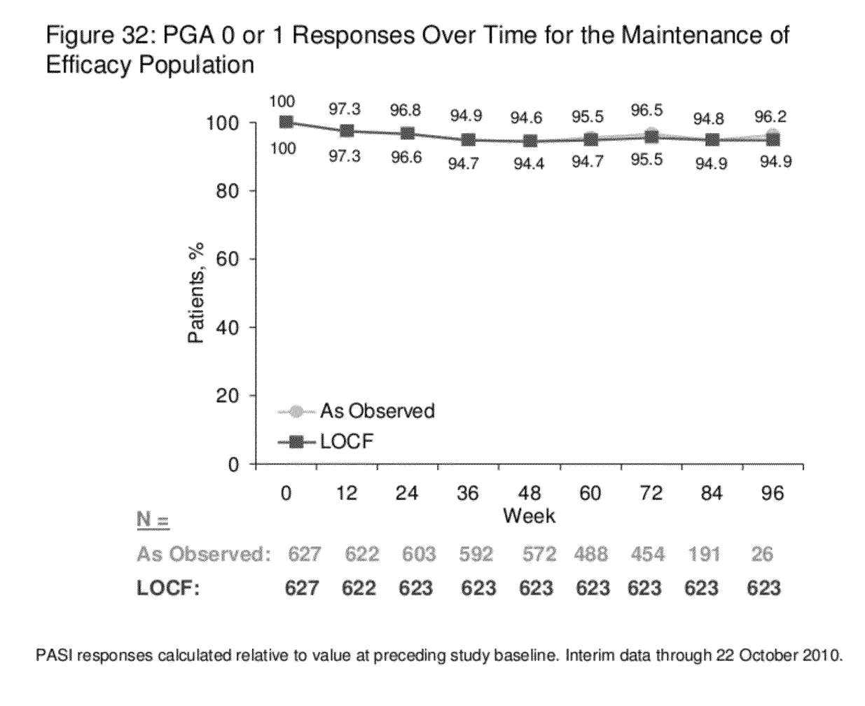

PASI 75, PASI 90 and PASI 100 responses over time for the maintenance of efficacy population are set forth in FIGS. 29-31. PGA 0 or 1 responses over time for the maintenance of efficacy population are set forth in FIG. 32. A summary of adverse events is depicted in the Table 23, below.

TABLE 23

| | Briakinumab Patients | |
|---|---|---|
| | with Events | Events |
| | n (%) | (E/100 PYs) |
| Adverse Event | (N = 2520) | (PYs = 4703.8) |
| Any AE | 2105 (83.5) | 11446 (243.3) |
| Any serious AE | 200 (7.9) | 288 (6.1) |
| Any AE leading to | 150 (6.0) | 179 (3.8) |
| discontinuation | | |
| Any infection | 1382 (54.8) | 3092 (65.7) |
| Any serious infection | 33 (1.3) | 41 (0.9) |
| Opportunistic infections | 16 (0.6) | 17 (0.4) |
| Malignancy | 70 (2.8) | 89 (1.9) |
| Non-melanoma skin cancer | 43 (1.7) | 60 (1.3) |
| Lymphoma | 0 | 0 |
| Most common AEs* | | |
| Nasopharyngitis | 421 (16.7) | 668 (14.2) |
| Upper respiratory tract | 417 (16.5) | 628 (13.4) |
| infection | | |
| Hypertension | 224 (8.9) | 242 (5.1) |
| Headache | 202 (8.0) | 309 (6.6) |
| Arthralgia | 194 (7.7) | 224 (4.8) |
| Back pain | 161 (6.4) | 183 (3.9) |
| Sinusitis | 158 (6.3) | 194 (4.1) |
| Injection site reaction | 142 (5.6) | 394 (8.4) |
| Cough | 125 (5.0) | 140 (3.0) |

*AEs occurring in ≧5% of patients
Interim data through October 2010

A total of 27 MACE were observed. 7 MACE occurred in 1 randomized controlled clinical trial: 5 MACE during initial 12-week placebo-controlled treatment period and 2 MACE between Week 12 and week 52. MACE occurred at an incidence rate of 0.57 E/100 PY (95% CI: 0.38, 0.84).

TABLE 24

| | Briakinumab Patients | |
|---|---|---|
| | with MACE | Events |
| | n (%) | (E/100 PYs) |
| MACE | (N = 2520) | (PYs = 4703.8) |
| Any MACE | 26 (1.0) | 27 (0.57) |
| Any non-fatal myocardial | 18 (0.7) | 19 (0.40) |
| infarction | | |
| Any non-fatal stroke | 3 (0.1) | 3 (0.06) |
| Any cardiovascular death | 5 (0.2) | 5 (0.11) |

The rate of MACE was higher in patients with ≧2 cardiovascular risk factors, compared with patients having 0 or 1 risk factors (Table 25).

TABLE 25

| | Briakinumab Patients | | | |
|---|---|---|---|---|
| Number of Risk | with MACE | | Events Per 100 PYs | |
| Factors | N | n (%) | PYs | E/100 PYs |
| 0 or 1 | 1937 | 10 (0.5) | 3647.6 | 10 (0.27) |
| 2 to 4 | 583 | 16 (2.7) | 1056.2 | 17 (1.61) |
| Overall | 2520 | 26 (1.0) | 4703.8 | 27 (0.57) |

Risk Factors: History of diabetes; BMI ≧30; uncontrolled blood pressure (≧140/90) at baseline; history of CVD.

Cardiovascular (CV) risk factors were analyzed for all patients with ≧1 dose of briakinumab in a prior Phase III or Phase II study, or in the OLE (N=2520). A univariate analysis was conducted and included the following standard CV risk factors: body mass index, triglycerides, HDL/LDL-cholesterol, systolic/diastolic blood pressure, history of hypertension, history of diabetes, history of cardiovascular disease, current cigarette smoking and age. Four specific CV risk factors were identified to be predictive for MACE: history of diabetes mellitus, BMI≧30, inadequate blood pressure control (BP≧140/90), and history of CV disease (defined as ≧1 of the following: myocardial infarction, angina requiring hospitalization, stroke or TIA, peripheral atery disease, coronary artery disease requiring revasculariztation, or congestive heart failure requiring hospitalization).

### Conclusion

Interim results from this OLE study show that high levels of PASI and PGA response are generally maintained with ongoing briakinumab 100 mg (every 4 weeks) treatment. Results support the need for further evaluation of infection, NMSC and cardiovascular events. MACE occurred more frequently in patients with a greater number of CV risk factors. During previous studies, patients with ≧2 CV risk factors were excluded, unless they previously failed other systemic therapies, including TNF inhibitors.

### Example 11

### Efficacy and Safety of Briakinumab for Treatment of Moderate to Severe Psoriasis in Patients Previously Receiving Etanercept—Results from an Open-label Extension

### Introduction

Psoriasis is a chronic immunologic disease characterized by marked inflammation and thickening of the epidermis, currently affecting 1% to 3% of the general population (Greaves and Weinstein, New England J. Med., 1995, 332(9):581-588). Biologic agents have emerged as promising treatments for psoriasis, however, some patients demonstrate loss of response with long-term treatment and further information is needed that demonstrates how these patients respond to their next biologic therapy. Briakinumab, a fully-human, anti-IL-12/23p40 monoclonal antibody, has been shown to be effective and well tolerated for the treatment of psoriasis in a Phase II trial (Kimball et al., Arch. Dermatol., 2008, 2:200-207). The aim of this study was to determine the efficacy and safety of briakinumab in patients with moderate to severe psoriasis who previously received etanercept from 2 briakinumab Phase III psoriasis trials, and upon study completion or loss of response to etanercept enrolled into this ongoing open-label extension (OLE).

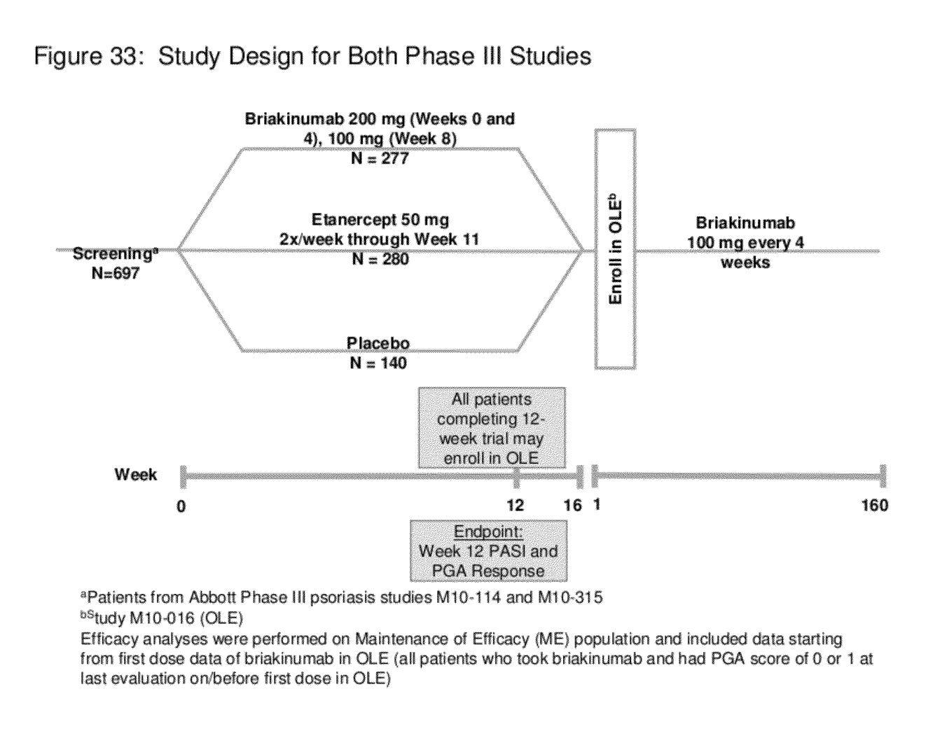

The study design is shown in FIG. 33, and baseline demographics and clinical characteristics are shown in Table 26, below.

TABLE 26

| | Received Etanercept in Previous Studies^{a} | |
|---|---|---|
| | Achieved | Did not achieve |
| | PGA 0/1 | PGA 0/1 |
| Characteristic | N = 94 | N = 159 |
| Age (years), mean (SD) | 41.5 ± 13.6 | 45.6 ± 13.5 |
| Male | 52 (55.3) | 112 (70.4) |
| White | 88 (93.6) | 143 (89.9) |
| Weight (kg), mean (SD) | 89.0 ± 20.4 | 100.1 ± 22.1 |
| BSA affected by psoriasis | 23.4 ± 14.4 | 24.6 ± 14.6 |
| (%), mean (SD) | | |
| PASI score, mean (SD) | 18.5 ± 7.8 | 19.0 ± 6.9 |
| PGA | | |
| Clear, Minimal, or Mild | 0 | 0 |
| Moderate | 59 (62.8) | 70 (44.0) |
| Severe | 33 (35.1) | 79 (49.7) |
| Very Severe | 2 (2.1) | 10 (6.3) |
| History of psoriasis | 50 (53.2) | 77 (48.4) |
| Duration of psoriasis | 15.3 ± 11.9 | 17.2 ± 13.0 |
| (years), mean (SD) | | |
| Duration of psoriatic | 9.5 ± 8.3 | 7.4 ± 6.0 |
| arthritis (years), mean | | |
| (SD)^{b} | | |
| Currently having swollen, | 15 (16.0) | 42 (26.4) |
| tender, stiff joints | | |

Values are n (%) unless otherwise noted
^{a}Patients randomized to etanercept in M10-114 and M10-315 and either achieved or did not achieve PGA 0/1 at Week 12, then enrolled in OLE
^{b}N = 20 for patients who achieved PGA 0/1; N = 51 for patients who did not achieve PGA 0/1
BSA = body surface area; PASI = Psoriasis Area and Severity Index; PGA = Physician's Global Assessment

### Results

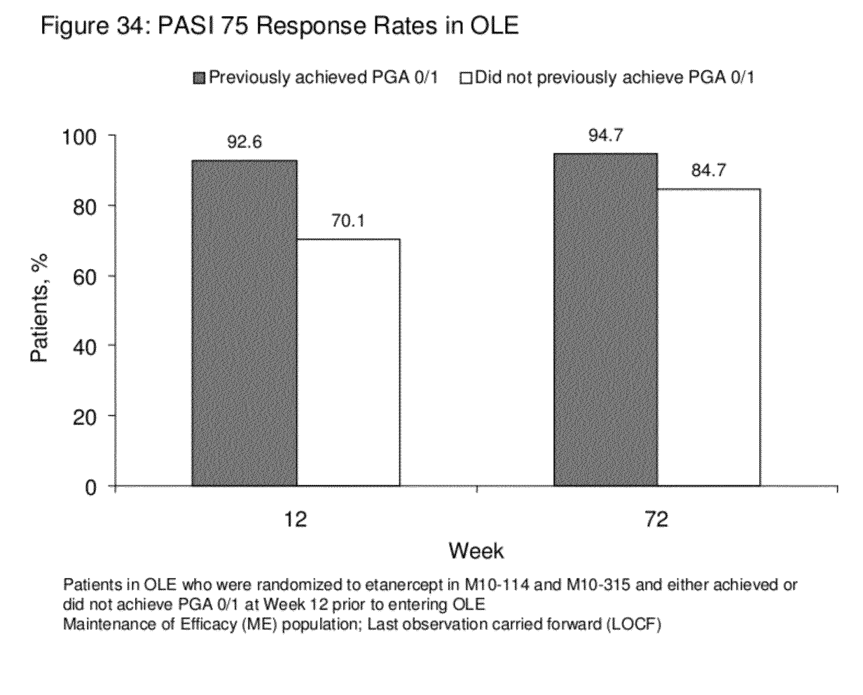

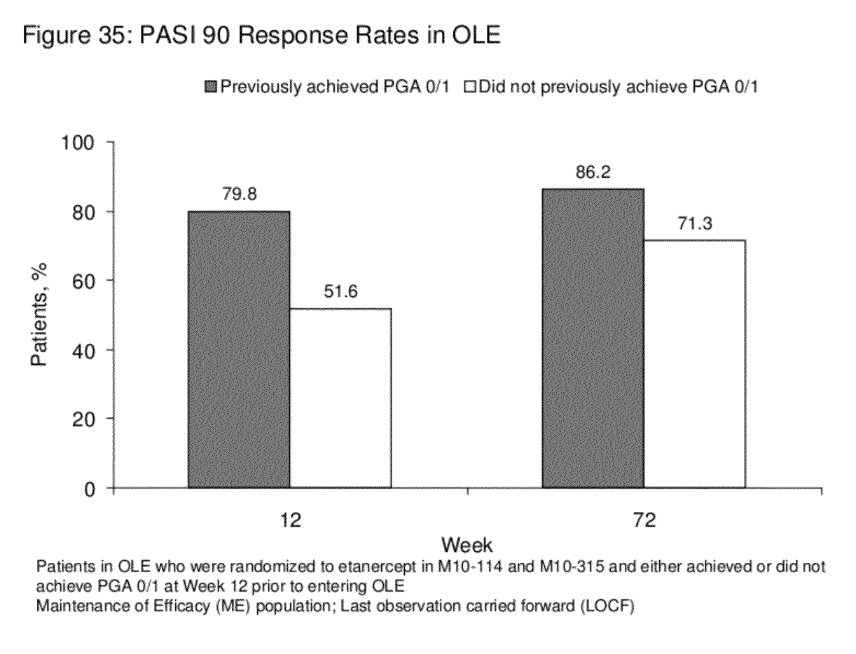

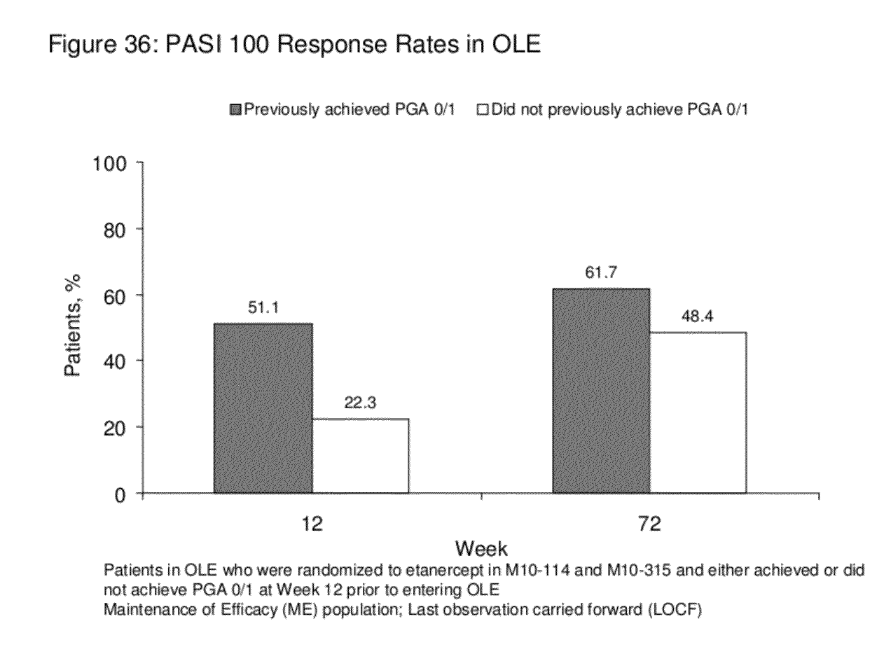

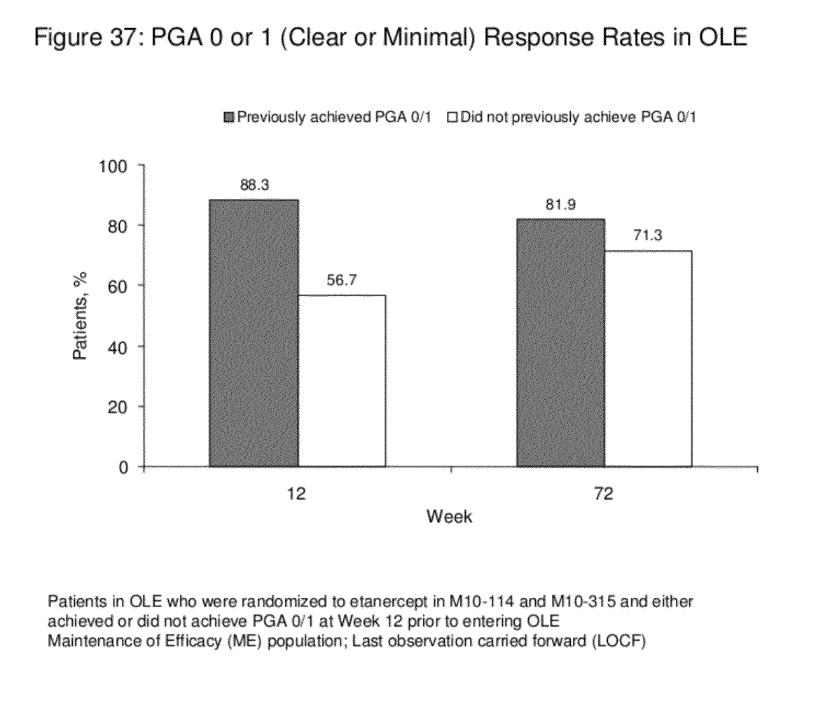

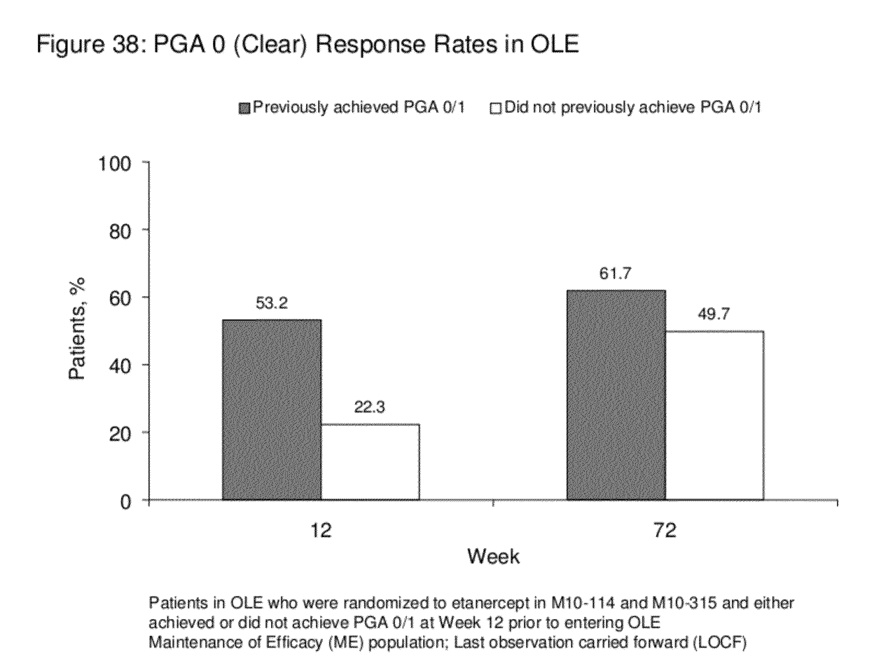

PASI 75, PASI 90 and PASI 100 response rates in OLE are shown in FIGS. 34-36. PGA 0 or 1 (clear or minimal) response rates in OLE are shown in FIG. 37. PGA 0 (clear) response rates in OLE are shown in FIG. 38. Safety data is presented in the Table 27, below.

TABLE 27

| | Received Etanercept in Previous Studies^{a} | |
|---|---|---|
| | Achieved | Did not achieve |
| | PGA 0/1 | PGA 0/1 |
| Adverse Event | N = 94 | N = 159 |
| Any Adverse Event (AE) | 70 (74.5) | 124 (78.0) |
| Any serious AE | 3 (3.2) | 11 (6.9) |
| AEs leading to | 4 (4.3) | 6 (3.8) |
| discontinuation | | |
| Infections | 37 (39.4) | 67 (42.1) |
| Serious infections | 2 (2.1) | 2 (1.3) |
| Opportunistic infections | 0 | 2 (1.3) |
| Malignancies | 1 (1.1) | 2 (1.3) |
| Skin malignancies | 0 | 0 |
| Non-melanoma skin cancer | 0 | 2 (1.3) |
| Injection site reaction | 6 (6.4) | 10 (6.3) |
| Allergic reaction | 13 (13.8) | 23 (14.5) |
| Hepatic events | 0 | 7 (4.4) |
| Ischemic heart disease | 1 (1.1) | 5 (3.1) |
| Major adverse cardiac | 0 | 2 (1.3) |
| event (MACE) | | |
| Congestive heart failure | 0 | 0 |
| Deaths | 0 | 2 (1.3) |

^{a}Patients randomized to etanercept in M10-114 and M10-315 and either achieved or did not achieve PGA 0/1 at Week 12, then enrolled in OLE
Values are n (%)
Interim analysis up to 22 Oct. 2010 of OLE

### Conclusions

In this study, moderate to severe psoriasis patients who were unsuccessful with etanercept therapy were able to achieve an adequate clinical response to treatment with briakinumab, though patients who were less responsive to etanercept tended to show diminished responses to briakinumab as well. Other than MACE (1.3%) and ischemic heart disease (1.1%-3.1%) no other clinically important safety concerns were reported during the study. This trial adds to our understanding of the efficacy of briakinumab by providing efficacy and safety data up to 72 weeks.

### Example 12

### ABT-874 Versus Methotrexate in Moderate to Severe Psoriasis: Effects on Health-Related Quality-of-Life Outcomes

### Introduction:

Psoriasis is a chronic systemic illness characterized by exacerbations and remissions of erythematous plaques on the body surface (Menter et al., J. Am. Acad Dermatol., 2008, 58:826-50 and Emer J J et al., J. Clin. Aesthet. Dermatol., 2010, 3:20-6) and substantial impairment of health-related quality of life (HRQOL) (de Korte J et al., J. Investig. Dermatol. Symp. Proc., 2004, 9:140-147; Krueger G et al., Arch. Dermatol., 2001, 137:280-284; and Finaly A Y et al., Br. J. Dermatol., 1995, 132:236-244). ABT-874 is an anti-IL-12/23 monoclonal antibody that has been shown to be superior to methotrexate (MTX), a conventional systemic agent, for the reduction of skin symptoms in moderate to severe psoriasis. The effects of ABT-874 compared with MTX on HRQOL have not previously been reported.

### Purpose:

To assess and compare short- and long-term health-related quality-of-life (HRQOL) in psoriasis patients treated with ABT-874 vs. methotrexate (MTX) in the treatment of moderate to severe psoriasis.

### Methods:

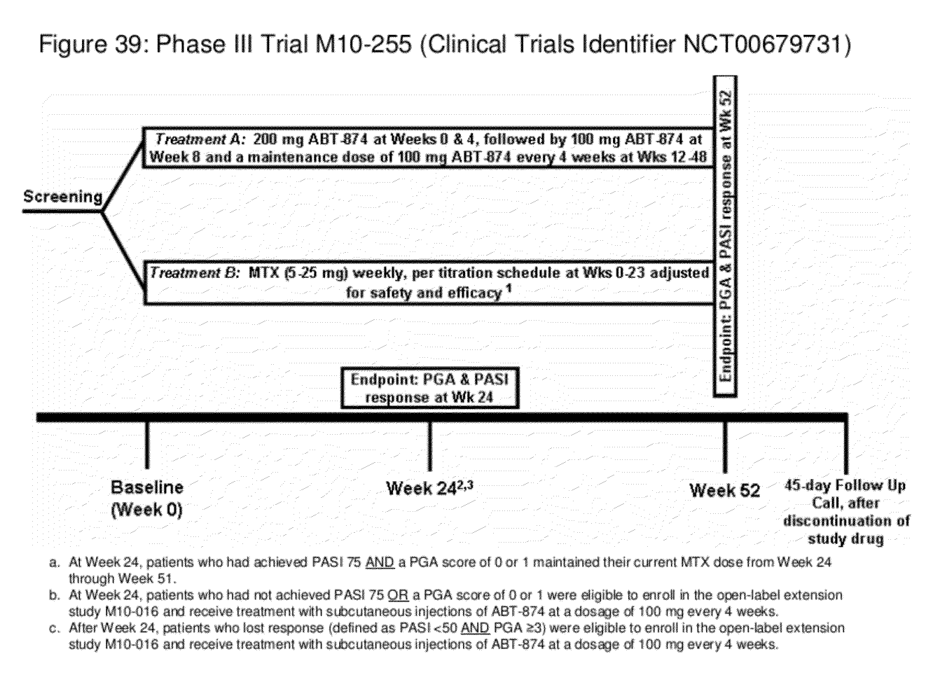

In this 52-week trial, patients were randomized to ABT-874 (200 mg at Weeks 0/4, then 100 mg q4 weeks) or MTX (5-25 mg weekly). The study design is shown in FIG. 39.

Data from study M10-255, a Phase III, 52-week, double-blind, randomized, multicenter, active controlled trial of patients with moderate to severe Ps (ClinicalTrials.gov identifier: NCT00679731). At baseline, patients were randomized 1:1 to double-blind treatment with ABT-874 or MTX. The primary endpoint was achievement of ≧75% improvement in the Psoriasis Area and Severity Index (PASI 75) and a Physician's Global Assessment (PGA) score of 0 or 1 at Week 24. At and after Week 24, non-responding patients were eligible to enroll in an open-label extension study (ClinicalTrials.gov identifier: NCT00626002) and receive treatment with ABT-874 at a dosage of 100 mg every 4 weeks. Patients were followed for a maximum of 52 weeks.

Non-response was defined as <75% improvement in the Psoriasis Area and Severity Index (PASI) and a Physician's Global Assessment (PGA) of “Mild” or worse at Week 24 or PASI<50 improvement and a PGA of “Severe” or worse after Week 24. HRQOL outcomes included Dermatology Life Quality Index (DLQI), visual analog scales for psoriasis-related pain (VAS-Ps), and EuroQOL-5D-Index (EQ-5D) scores. The DLQI was used to assess impact of skin disease on HRQOL and ranges from 0 (no impact) to 30 (worst impact). Greater DLQI scores indicate greater HRQOL impairment. VAS-Ps scores range from 0 (no pain) to 100 (very severe pain). EQ-5D scores are based on the EQ-5D Descriptive System Health Questionnaire (EQ-5D_{index}) and include five dimensions of HRQOL: anxiety/depression, mobility, self care, usual activities and pain/discomfort. The scoring algorithm is based on the social preferences of the UK population, and scores range from −0.594 to 1.0, with 1.0 being the best possible score.

Mean improvements from baseline to Weeks 12 and 52 were compared using analyses of covariance. Percentages of patients with improvement≧the minimum clinically important difference (MCID) and achieving DLQI≦1 were compared using chi-squared tests. For the assessment of mean score improvement, for each HRQOL outcome, mean changes in score from baseline to Week 24 and Week 52 were estimated and compared between treatment groups by analysis of covariance (ANCOVA), adjusting for baseline score. For assessment of treatment response rate, for each HRQOL outcome, minimum clinically important difference (MCID) response rates (defined as the percentage of patients with score improvement≧MCID) were compared between treatment groups according to the following criteria (Table 28):

| DLQI Score¹ | VAS Score¹ | EQ-5D Score² |
|---|---|---|
| ≧5 points | ≧½ SD from baseline score | EQ-5D_{index} ≧0.09 |
| | | EQ-5D-VAS ≧4.59 |

SD, standard deviation. EQ-5D-VAS,
¹Revicki D A, et al. J Dermatolog Treat. 2007; 18: 341-50.
²Shikiar R, et al. Health Qual Life Outcomes. 2006; 4: 71.

Missing values were imputed by last observation carried forward (LOCF) for analysis at Week 24 and Week 52.

### Results:

Baseline demographics, medical history, and HRQOL were similar between groups, except for a greater proportion with very severe PGA in the ABT-874 group. See Table 29.

TABLE 29
Baseline Characteristics

| | Treatment Group | | |
|---|---|---|---|
| | ABT-874 | MTX | |
| Baseline Characteristic | (n = 154) | (n = 163) | P-Value^{a} |
| Demographics and medical | | | |
| history | | | |
| Age (years), mean ± SD | 45.0 ± 13.14 | 43.1 ± 12.87 | 0.1876 |
| Sex (female), n (%) | 43 (27.9) | 52 (31.9) | 0.4390 |
| Race (white), n (%) | 149 (96.8) | 158 (96.9) | 0.9270 |
| Ps history | | | |
| Duration of Ps (years), | 18.6 ± 11.91 | 19.08 ± 11.30 | 0.5917 |
| mean ± SD | | | |
| Concomitant PsA, n (%) | 25 (16.2) | 28 (17.2) | 0.8220 |
| Ps baseline assessments | | | |
| Affected body surface area | 26.1 ± 16.70 | 26.1 ± 16.28 | 0.6756 |
| (%), mean ± SD | | | |
| PASI score, mean ± SD | 18.4 ± 6.69 | 17.8 ± 6.06 | 0.4508 |
| PGA score | | | |
| “Moderate,” n (%) | 75 (48.7) | 87 (53.4) | 0.4050 |
| “Severe,” n (%) | 65 (42.2) | 72 (44.2) | 0.7240 |
| “Very Severe,” n (%) | 14 (9.1) | 4 (2.5) | 0.0110 |
| HRQOL | | | |
| DLQI, mean ± SD | 11.1 ± 6.55 | 11.3 ± 7.47 | 0.9508 |
| EQ5D scores, mean ± SD | | | |
| EQ-5D_{index} | 0.67 ± 0.30 | 0.71 ± 0.29 | 0.3600 |
| EQ-5D-VAS | 60.0 ± 25.22 | 65.4 ± 22.87 | 0.0716 |
| VAS-Ps, mean ± SD | 34.0 ± 31.00 | 29.4 ± 29.06 | 0.2121 |

^{a}Calculated using chi-square test for categorical variables and Kruskal-Wallis test for continuous variables.

ABT-874 (N=154) vs. MTX (N=163) was associated with significantly (p<0.05) greater MCID response rates at Weeks 12 (DLQI: 70.1% vs. 50.9%; VAS-Ps: 49.4% vs. 35.0%; EuroQOL-5D-Index: 57.1% vs. 43.6%) and 52 (DLQI: 56.5% vs. 18.4%; VAS-Ps: 38.3% vs. 11.0%; EuroQOL-5D-Index: 48.7% vs. 17.2%) and with significantly greater mean improvements at Weeks 12 (DLQI: −8.88 vs. −6.00; VAS-Ps: −23.38 vs. −17.84; EuroQOL-5D-Index: 0.20 vs. 0.14) and 52 (DLQI: −9.62 vs. −6.54; VAS-Ps: −24.30 vs. −17.81; EuroQOL-5D-Index: 0.24 vs. 0.15).

Significantly more ABT-874-treated patients achieved DLQI≦1 at Weeks 24 (70.8% vs. 34.4%) and 52 (61.7% vs. 17.8%). At week 24, the ABT-874 group experienced significantly greater improvement compared with the MTX group in DLQI, EQ-5D_{index}, EQ-5D-VAS and VAS-Ps (Table 30).

TABLE 30
Mean Change in HRQOL Outcome Measures at Week 24

| | Within-Group Change | | Between-Group Change | |
|---|---|---|---|---|
| | ABT-874 | MTX | Difference | P-Value |
| Outcome | Change in Score, | Change in Score, | ABT-874 − MTX, | ABT-874 vs. |
| Measure | Mean^{a} (95% CI) | Mean^{a} (95% CI) | Mean^{a} (95% CI) | MTX^{b} |
| DLQI | −9.53 (−10.20, −8.87) | −6.53 (−7.17, −5.89) | −3.00 (−3.93, −2.08) | <0.0001 |
| EQ-5D_{index} | 0.20 (0.17, 0.23) | 0.15 (0.12, 0.18) | 0.05 (0.01, 0.10) | 0.0085 |
| EQ-5D-VAS | 19.49 (16.21,22.77) | 11.90 (8.74, 15.07) | 7.58 (3.02, 12.15) | 0.0012 |
| VAS-Ps | −24.41 (−27.21, −21.60) | −17.11 (−19.83, −14.39) | −7.30 (−11.21, −3.39) | 0.0003 |

CI, confidence interval.
^{a}Least-squares mean.
^{b}Highlighted cells indicate a statistically significant difference at the 5% level.

At week 24, more than 50% of patients in the ABT-874 group achieved score improvements≧MCID for DLQI, EQ-5D_{index}, EQ-5D-VAS and VAS-Ps. ABT-874-treated patients experienced significantly greater MCID response rates compared with MTX-treated patients for DLQI, EQ-5D_{index}, EQ-5D-VAS and VAS-Ps (Table 31).

TABLE 31
MCID Response Rates at Week 24

| | Treatment Group | | |
|---|---|---|---|
| | ABT-874 | MTX | P-Value^{b} |
| Outcome | MCID Responder,^{a} | MCID Responder,^{a} | ABT-874 vs. |
| Measure | n (%) | n (%) | MTX |
| DLQI | 106 (70.7) | 85 (53.1) | 0.0015 |
| EQ-5D_{index} | 90 (61.6) | 77 (49.7) | 0.0368 |
| EQ-5D-VAS | 106 (71.6) | 89 (56.0) | 0.0044 |
| VAS-Ps | 81 (53.3) | 58 (35.8) | 0.0018 |

^{a}Defined as the percentage of patients with score improvement ≧MCID.
^{b}Highlighted cells indicate a statistically significant difference at the 5% level.

At Week 52, the ABT-874 group experienced significantly greater improvement compared with the MTX grouping DLQI, EQ-5D_{index}, EQ-5D-VAS and VAS-Ps (Table 32).

TABLE 32
Mean Change in HRQL Outcome Measures by Week 52

| | Within-Group Change From Baseline to Week 52 | | Between-Group Change | |
|---|---|---|---|---|
| | ABT-874 Change | MTX Change in | Difference | P-Value |
| Outcome | in Score, | Score, | ABT-874 − MTX, | ABT-874 vs. |
| Measure | Mean^{a} (95% CI) | Mean^{a} (95% CI) | Mean^{a} (95% CI) | MTX^{b} |
| DLQI | −9.62 (−10.28, −8.96) | −6.54 (−7.18, −5.89) | −3.08 (−4.01, −2.16) | <0.0001 |
| EQ-5D_{index} | 0.24 (0.21, 0.27) | 0.15 (0.12, 0.18) | 0.09 (0.05, 0.12) | <0.0001 |
| EQ-5D-VAS | 21.38 (18.00, 24.76) | 12.26 (9.00, 15.52) | 9.12 (4.41, 13.83) | 0.0002 |
| VAS-Ps | −24.30 (−27.24, −21.37) | −17.81 (−20.65,−14.97) | −6.50 (−10.59, −2.41) | 0.0019 |

^{a}Least-squares mean.
^{b}Highlighted cells indicate a statistically significant difference at the 5% level.

MCID response rates for ABT-874-treated patients at Week 52 were significantly greater compared with rates for MTX-treated patients for DLQI, EQ-5D_{index}, EQ-5D-VAS and VAS-Ps (Table 33).

TABLE 33
MCID Response Rates at Week 52

| | Treatment Group | | |
|---|---|---|---|
| | ABT-874 | MTX | P-Value^{b} |
| Outcome | MCID Responder,^{a} | MCID Responder,^{a} | ABT-874 vs. |
| Measure | n (%) | n (%) | MTX |
| DLQI | 107 (71.3) | 85 (53.1) | 0.001 |
| EQ-5D_{index} | 98 (67.1) | 76 (49.0) | 0.0015 |
| EQ-5D-VAS | 112 (75.7%) | 89 (56.0%) | 0.0003 |
| VAS-Ps | 80 (52.6) | 57 (35.2) | 0.0018 |

^{a}Defined as the percentage of patients with score improvement ≧MCID.
^{b}Highlighted cells indicate a statistically significant difference at the 5% level.

### Conclusions:

ABT-874 treatment for moderate to severe psoriasis was associated with significantly greater and clinically meaningful improvements in HRQOL outcomes compared with MTX at both 24 and 52 weeks of treatment. Compared with MTX, ABT-874 demonstrated significantly greater mean improvements and percentages of patients with clinically meaningful improvements in DLQI, EQ-5D_{index}, EQ-5D-VAS and VAS-Ps. These data demonstrate the treatment benefits of ABT-874 for patients with psoriasis beyond the previously described clinical efficacy in significantly reducing psoriasis symptoms versus MTX.

1. A method of treating psoriasis in a subject or population of subjects, comprising
selecting a subject or population of subjects who would benefit from an improvement in a Short Form 36 Health Survey domain score selected from the group consisting of a Physical Function score, a Role-Physical score, a Bodily Pain score, a General Health score, a Vitality score, a Social Function score, a Role-Emotional score, and a Mental Health score, and
administering to the subject or population of subjects an antibody, or antigen-binding portion thereof, which is capable of binding to the p40 subunit of IL-12 and/or IL-23,
wherein the subject or population of subjects upon treatment achieves an improvement or mean improvement in a Short Form 36 Health Survey domain score selected from the group consisting of a Physical Function score, a Role-Physical score, a Bodily Pain score, a General Health score, a Vitality score, a Social Function score, a Role-Emotional score, and a Mental Health score.

2. The method of claim 1,
wherein the subject or population of subjects achieves an improvement or mean improvement in a Short Form 36 Health Survey Physical Function score of at least about 3,
wherein the subject or population of subjects achieves an improvement or mean improvement in a Short Form 36 Health Survey Role-Physical score of at least about 2.5,
wherein the subject or population of subjects achieves an improvement or mean improvement in a Short Form 36 Health Survey Bodily Pain score of at least about 6,
wherein the subject or population of subjects achieves an improvement or mean improvement in a Short Form 36 Health Survey General Health score of at least about 2.5,
wherein the subject or population of subjects achieves an improvement or mean improvement in a Short Form 36 Health Survey Vitality score of at least about 2.5,
wherein the subject or population of subjects achieves an improvement or mean improvement in a Short Form 36 Health Survey Social Function score of at least about 5,
wherein the subject or population of subjects achieves an improvement or mean improvement in a Short Form 36 Health Survey Role-Emotional score of at least about 4.5, or
wherein the subject or population of subjects achieves an improvement or mean improvement in a Short Form 36 Health Survey Mental Health score of at least about 2.5.

3. The method of claim 1,
wherein at least 30% of the population of subjects achieves an improvement at or exceeding a minimum clinically important difference (MCID) response for a Short Form 36 Health Survey Physical Function score,
wherein at least 20% of the population of subjects achieves an improvement at or exceeding a minimum clinically important difference (MCID) response for a Short Form 36 Health Survey Role-Physical score,
wherein at least 40% of the population of subjects achieves an improvement at or exceeding a minimum clinically important difference (MCID) response for a Short Form 36 Health Survey Bodily Pain score,
wherein at least 20% of the population of subjects achieves an improvement at or exceeding a minimum clinically important difference (MCID) response for a Short Form 36 Health Survey General Health score,
wherein at least 35% of the population of subjects achieves an improvement at or exceeding a minimum clinically important difference (MCID) response for a Short Form 36 Health Survey Vitality score,
wherein at least 20% of the population of subjects achieves an improvement at or exceeding a minimum clinically important difference (MCID) response for a Short Form 36 Health Survey Social Function score,
wherein at least 5% of the population of subjects achieves an improvement at or exceeding a minimum clinically important difference (MCID) response for a Short Form 36 Health Survey Role Emotional score, or
wherein at least 40% of the population of subjects achieves an improvement at or exceeding a minimum clinically important difference (MCID) response for a Short Form 36 Health Survey Mental Health score.

4. A method of treating psoriasis in a subject or population of subjects, comprising
selecting a subject or population of subjects who would benefit from an improvement or mean improvement in an HRQOL outcome selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP), and
administering to the subject or population of subjects an antibody, or antigen-binding portion thereof, which is capable of binding to the p40 subunit of IL-12 and/or IL-23,
wherein the subject or population of subjects upon treatment achieves an improvement or mean improvement in an HRQOL outcome selected from the group consisting of Dermatology Life Quality Index (DLQI), psoriasis-related pain (VAS-Ps), psoriatic arthritis-related pain (VAS-PsA), and Work Productivity and Activity Impairment-Specific Health Problem for psoriasis (WPAI-SHP).

5. The method of claim 4,
wherein the subject or population of subjects achieves an improvement or mean improvement in a Dermatology Life Quality Index (DLQI) score by at least about −8,
wherein the subject or population of subjects achieves an improvement or mean improvement in a psoriasis-related pain (VAS-Ps) score by at least about −25, or
wherein the subject or population of subjects achieves an improvement or mean improvement in a psoriatic arthritis-related pain (VAS-PsA) score by at least about −15.

6. The method of claim 4,
wherein the subject or population of subjects achieves an improvement or mean improvement in a work productivity and activity impairment-specific health problem for psoriasis (WPAI-SHP) score by at least about −2 for % work time missed,
wherein the subject or population of subjects achieves an improvement or mean improvement in a work productivity and activity impairment-specific health problem for psoriasis (WPAI-SHP) score by at least about −13 for % impairment while working,
wherein the subject or population of subjects achieves an improvement or mean improvement in a work productivity and activity impairment-specific health problem for psoriasis (WPAI-SHP) score by at least about −13 for % overall work impairment, or
wherein the subject or population of subjects achieves an improvement or mean improvement in a work productivity and activity impairment-specific health problem for psoriasis (WPAI-SHP) score by at least about −18 for % overall activity impairment.

7. The method of claim 4,
wherein at least about 60% of the population of subjects achieves an improvement at or exceeding a minimum clinically important difference (MCID) response for psoriasis-related pain (VAS-Ps) by about week 12 or 52,
wherein at least 50% of the population of subjects achieves an improvement at or exceeding a minimum clinically important difference (MCID) response for psoriatic arthritis-related pain (VAS-PsA) by about week 12 or 52,
wherein at least 6% of the population of subjects achieves an improvement at or exceeding a minimum clinically important difference (MCID) response for work productivity and activity impairment-specific health problem for psoriasis (WPAI-SHP) for % work time missed by about week 12 or 52,
wherein at least 35% of the population of subjects achieves an improvement at or exceeding a minimum clinically important difference (MCID) response for work productivity and activity impairment-specific health problem for psoriasis (WPAI-SHP) for % impairment while working,
wherein at least 35% of the population of subjects achieves an improvement at or exceeding a minimum clinically important difference (MCID) response for work productivity and activity impairment-specific health problem for psoriasis (WPAI-SHP) for % overall work impairment, or
wherein at least 45% of the population of subjects achieves an improvement at or exceeding a minimum clinically important difference (MCID) response for work productivity and activity impairment-specific health problem for psoriasis (WPAI-SHP) for % overall activity impairment.

8. The method of claim 1 or claim 4, wherein the improvement is achieved by about week 12, or by about week 52.

9. A method of treating psoriasis in a subject or population of subjects, comprising
selecting a subject or population of subjects who would benefit from an improvement in PGA score, and
administering to the subject or each subject in the population of subjects an antibody, or antigen-binding portion thereof, which is capable of binding to the p40 subunit of IL-12 and/or IL-23,
wherein the subject or the population of subjects upon treatment achieves a PGA score of 0 or 1 at a time or a median time of less than about 60 days.

10. A method of treating psoriasis in a subject or population of subjects, comprising
selecting a subject or population of subjects who would benefit from an improvement in a Psoriasis Area Severity Index (PASI) score, and
administering to the subject or each subject in the population of subjects an antibody, or antigen-binding portion thereof, which is capable of binding to the p40 subunit of IL-12 and/or IL-23,
wherein the subject or the population of subjects upon treatment achieves a Psoriasis Area and Severity Index (PASI) 75 response in a time or a median time of less than about 70 days.

11. A method of treating psoriasis in a subject or population of subjects, comprising
selecting a subject or population of subjects who would benefit from an improvement in a Dermatology Life Quality Index (DLQI) score, and
administering to the subject or each subject in the population of subjects an antibody, or antigen-binding portion thereof, which is capable of binding to the p40 subunit of IL-12 and/or IL-23,
wherein the subject or at least 20% of the population of subjects upon treatment achieves a Dermatology Life Quality Index (DLQI) score of 0 by about week 12.

12. A method of treating psoriasis in a subject or a population of subjects, comprising
selecting a subject or a population of subjects who would benefit from an improvement in a PGA score or a PASI score, and
administering to the subject or each subject in the population an antibody, or antigen-binding portion thereof, which is capable of binding to the p40 subunit of IL-12 and/or IL-23,
wherein the subject or at least about 15% of the population of subjects upon treatment achieves at least a PGA score of 0 or 1 by about week 4,
wherein the subject or at least about 18% of the population of subjects upon treatment achieves a PGA score of 0 or 1 by about week 8,
wherein the subject or at least about 20% of the population of subjects upon treatment achieves at least a PASI 75 response by about week 4,
wherein the subject or at least about 25% of the population of subjects upon treatment achieves at least a PASI 75 response by about week 8,
wherein the subject or at least about 40% of the population of subjects upon treatment achieves at least a PASI 75 response by about week 12,
wherein the subject or at least about 35% of the population of subjects upon treatment achieves at least a PASI 90 response by about week 8, or
wherein the subject or at least about 15% of the population of subjects upon treatment achieves at least a PASI 90 response by about week 12,
wherein the subject or at least about 10% of the population of subjects upon treatment achieves a PASI 100 response by about week 8, or
wherein the subject or at least about 5% of the population of subjects upon treatment achieves a PASI 100 response by about week 12.

13.-14. (canceled)

15. A method of treating psoriasis in subject or a population of subjects, comprising
selecting a subject or population of subjects who would benefit from an improvement in a PASI score, wherein the subject or each subject in the population of subjects was treated with a biologic previously, and
administering to the subject or to each subject in the population an antibody, or antigen-binding portion thereof, which is capable of binding to the p40 subunit of IL-12 and/or IL-23,
wherein the subject or at least 80% of the population of subjects achieves at least a PASI 75 response by about week 52.

16. A method of treating psoriasis in a subject or a population of subjects, comprising
selecting a subject or a population of subjects who would benefit from an improvement in a PASI score, wherein neither the subject nor any subject in the population of subjects was treated with a biologic previously, and
administering to the subject or to each subject in the population an antibody, or antigen-binding portion thereof, which is capable of binding to the p40 subunit of IL-12 and/or IL-23,
wherein the subject or at least 80% of the population of subjects achieves at least a PASI 75 response by about week 52.

17. A method of treating psoriasis in a subject or a population of subjects, comprising
selecting a subject or a population of subjects who would benefit from an improvement in a PGA score or a PASI score, wherein the subject or each subject in the population of subject was treated with a biologic previously and showed no response, and
administering to the subject or each subject in the population an antibody, or antigen-binding portion thereof, which is capable of binding to the p40 subunit of IL-12 and/or IL-23,
wherein the subject or at least 65% of the population of subjects achieves a PGA score of 0 or 1 by about week 12,
wherein the subject or at least 70% of the population of subjects achieves a PGA score of 0 or 1 by about week 52,
wherein the subject or at least 70% of the population of subjects achieves at least a PASI 75 response by about week 12, or
wherein the subject or at least 75% of the population of subjects achieves at least a PASI 75 response by about week 52.

18. A method of treating psoriasis in a subject or a population of subjects, comprising
selecting a subject or a population of subjects who would benefit from an improvement in a PGA score or a PASI score, wherein the subject or each subject in the population of subject was treated with a biologic previously and showed improvement in the PGA score or PASI score, and
administering to the subject or each subject in the population an antibody, or antigen-binding portion thereof, which is capable of binding to the p40 subunit of IL-12 and/or IL-23,
wherein the subject or at least 75% of the population of subjects achieves a PGA score of 0 or 1 by about week 12,
wherein the subject or at least 75% of the population of subjects achieves a PGA score of 0 or 1 by about week 52,
wherein the subject or at least 75% of the population of subjects achieves at least a PASI 75 response by about week 12, or
wherein the subject or at least 75% of the population of subjects achieves at least a PASI 75 response by about week 52.

19.-20. (canceled)

21. A method of treating psoriasis in a subject or a population of subjects, comprising
selecting a subject or a population of subjects who would benefit from an improvement in a PASI score, and
administering to the subject or each subject in the population an antibody, or antigen-binding portion thereof, which is capable of binding to the p40 subunit of IL-12 and/or IL-23,
wherein the subject or at least 80% of the population of subjects achieves at least a PASI 75 response by about week 52, wherein each subject has a prior history of psoriatic arthritis, or
wherein the subject or at least 80% of the population of subjects achieves at least a PASI 75 response by about week 52, wherein none of the subjects has a prior history of psoriatic arthritis.

22. A method of treating psoriasis in a subject or a population of subjects, comprising
selecting a subject or a population of subjects who would benefit from an improvement in a PGA score or a PASI score, wherein the subject or each subject in the population of subjects had a baseline weight of less than 100 kilograms, and
administering to the subject or each subject in the population an antibody, or antigen-binding portion thereof, which is capable of binding to the p40 subunit of IL-12 and/or IL-23,
wherein the subject or at least 80% of the population of subjects achieves a PGA score of 0 or 1 by about week 52, or
wherein the subject or at least 80% of the population of subjects achieves at least a PASI 75 response by about week 52.

23. A method of treating psoriasis in a subject or a population of subjects, comprising
selecting a subject or a population of subjects who would benefit from an improvement in a PGA score or a PASI score, wherein the subject or each subject in the population of subjects had a baseline weight of greater than or equal to 100 kilograms, and
administering to the subject or each subject in the population an antibody, or antigen-binding portion thereof, which is capable of binding to the p40 subunit of IL-12 and/or IL-23,
wherein the subject or at least 70% of the population of subjects achieves a PGA score of 0 or 1 by about week 52, or
wherein the subject or at least 75% of the population of subjects achieves at least a PASI 75 response by about week 52.

24. A method of treating psoriasis in a subject or a population of subjects, comprising
selecting a subject or population of subjects who would benefit from an improvement in a PGA score or a PASI score, wherein the subject or each subject within the population of subjects had a baseline PASI score of less than or equal to 20, and
administering to each subject in the population an antibody, or antigen-binding portion thereof, which is capable of binding to the p40 subunit of IL-12 and/or IL-23,
wherein the subject or at least 80% of the population of subjects achieves a PGA score of 0 or 1 by about week 52, or
wherein the subject or at least 80% of the population of subjects achieves at least a PASI 75 response by about week 52.

25. A method of treating psoriasis in a subject or a population of subjects, comprising
selecting a subject or population of subjects who would benefit from an improvement in a PGA score or PASI score, wherein the subject or each subject within the population of subjects had a baseline PASI score of greater than 20, and
administering to the subject or each subject in the population an antibody, or antigen-binding portion thereof, which is capable of binding to the p40 subunit of IL-12 and/or IL-23,
wherein the subject or at least 70% of the population of subjects achieves a PGA score of 0 or 1 by about week 52, or
wherein the subject or at least 75% of the population of subjects achieves at least a PASI 75 response by about week 52.

26. A method of treating psoriasis in a subject or a population of subjects, comprising
selecting a subject or population of subjects who would benefit from an improvement in a PGA score or a PASI score, wherein the subject or each subject within the population of subjects had less than or equal to 20% body surface area (BSA) affected by psoriasis, and
administering to the subject or each subject in the population an antibody, or antigen-binding portion thereof, which is capable of binding to the p40 subunit of IL-12 and/or IL-23,
wherein the subject or at least 80% of the population of subjects achieves a PGA score of 0 or 1 by about week 12,
wherein the subject or at least 80% of the population of subjects achieves a PGA score of 0 or 1 by about week 52,
wherein the subject or at least 80% of the population of subjects achieves at least a PASI 75 response by about week 12, or
wherein the subject or at least 85% of the population of subjects achieves at least a PASI 75 response by about week 52.

27. A method of treating psoriasis in a subject or a population of subjects, comprising
selecting a subject or population of subjects who would benefit from an improvement in a PGA score or a PASI score, wherein the subject or each subject within the population of subjects had greater than 20% body surface area (BSA) affected by psoriasis, and
administering to the subject or each subject in the population an antibody, or antigen-binding portion thereof, which is capable of binding to the p40 subunit of IL-12 and/or IL-23,
wherein the subject or at least 70% of the population of subjects achieves a PGA score of 0 or 1 by about week 12,
wherein the subject or at least 70% of the population of subjects achieves a PGA score of 0 or 1 by about week 52,
wherein the subject or at least 75% of the population of subjects achieves at least a PASI 75 response by about week 12, or
wherein the subject or at least 75% of the population of subjects achieves at least a PASI 75 response by about week 52.

28.-29. (canceled)

30. A method of treating psoriasis in a subject or a population of subjects, comprising
selecting a subject or population of subjects who would benefit from an improvement in a PASI score or a PGA score, wherein the subject or each subject within the population of subjects has previously been exposed to a tumor necrosis factor- (TNF-) antagonist, and
administering to the subject or each subject in the population an antibody, or antigen binding portion thereof, which is capable of binding to the p40 subunit of IL-12 and/or IL-23,
wherein the subject or at least 70% of the population of subjects achieves at least a PASI 75 at about week 8,
wherein the subject or at least 80% of the population of subjects achieves at least a PASI 75 at about week 52,
wherein the subject or at least 80% of the population of subjects achieves at least a PASI 75 at about week 100,
wherein the subject or at least 50% of the population of subjects achieves a PGA score of 0 or 1 at about week 8,
wherein the subject or at least 75% of the population of subjects achieves a PGA score of 0 or 1 at about week 52, or
wherein the subject or at least 75% of the population of subjects achieves a PGA score of 0 or 1 at about week 100.

31.-32. (canceled)

33. The method of claim 30, wherein the subject or each subject in the population failed to respond to treatment with a TNF-antagonist.

34. The method of claim 30, wherein the TNF antagonist is selected from the group consisting of anti-TNF antibodies, anti-TNF antibody fragments, soluble p55 or p75 TNF receptors and derivatives thereof, soluble IL-13 receptor (sIL-13), and TNFα converting enzyme (TACE) inhibitors.

35. A method of treating psoriasis in a subject or a population of subjects, comprising
selecting a subject or a population of subjects who would benefit from an improvement in a PASI score, and
administering to the subject or each subject in the population an antibody, or antigen binding portion thereof, which is capable of binding to the p40 subunit of IL-12 and/or IL-23,
wherein the subject or at least 90% of the subjects in the population maintains at least a PASI 75 through about week 84, or
wherein the subject or at least 90% of the subjects in the population maintains at least a PASI 75 through about week 124.

36. (canceled)

37. The method of claim 35, wherein the subject or subjects in the population do not suffer an adverse event during treatment with the antibody, or antigen binding portion thereof, which is capable of binding to the p40 subunit of IL-12 and/or IL-23.

38. A method of treating psoriasis in a subject or a population of subjects, comprising
selecting a subject or a population of subjects who would benefit from an improvement in a PASI score, wherein the subject or all of the subjects in the population were previously exposed to a tumor necrosis factor (TNF) antagonist, and
administering to the subject or each subject in the population an antibody, or antigen binding portion thereof, which is capable of binding to the p40 subunit of IL-12 and/or IL-23,
wherein the subject or at least 40% of the population of subjects achieves at least a PASI 90 at about week 8,
wherein the subject or at least 70% of the population of subjects achieves at least a PASI 90 at about week 52,
wherein the subject or at least 70% of the population of subjects achieves at least a PASI 90 at about week 100,
wherein the subject or at least 10% of the population of subjects achieves at least a PASI 100 at about week 8,
wherein the subject or at least 60% of the population of subjects achieves at least a PASI 100 at about week 52,
wherein the subject or at least 50% of the population of subjects achieves at least a PASI 100 at about week 100,
wherein the subject or at least 15% of the population of subjects achieves a PGA score of 0 at about week 8,
wherein the subject or at least 65% of the population of subjects achieves a PGA score of 0 at about week 52, or
wherein the subject or at least 55% of the population of subjects achieves a PGA score of 0 at about week 100.

39.-40. (canceled)

41. The method of claim 38, wherein the subject or each of the subjects in the population failed to respond to treatment with a TNF-antagonist.

42. The method of claim 38, wherein the TNF antagonist is selected from the group consisting of anti-TNF antibodies, anti-TNF antibody fragments, soluble p55 or p75 TNF receptors and derivatives thereof, soluble IL-13 receptor (sIL-13), and TNFα converting enzyme (TACE) inhibitors.

43. (canceled)

44. A method of treating psoriasis in a subject or a population of subjects, comprising selecting a subject or a population of subjects who would benefit from an improvement in a PASI score or a PGA score, and
administering to the subject or each subject in the population an antibody, or antigen binding portion thereof, which is capable of binding to the p40 subunit of IL-12 and/or IL-23,
wherein the subject or at least 90% of the population of subjects maintains at least a PASI 75 through about week 96,
wherein the subject or at least 80% of the population of subjects maintains at least a PASI 90 through about week 96,
wherein the subject or at least 65% of the population of subjects maintains at least a PASI 100 through about week 96,
wherein the subject or each subject in the population maintains a PGA score of 0 or 1 through about week 12, or
wherein the subject or at least 90% of the population of subjects maintains at least a PGA 0 or 1 score through about week 96.

45. The method of claim 43, wherein the subject or each of the subjects in the population do not suffer an adverse event during treatment with the antibody, or antigen binding portion thereof, which is capable of binding to the p40 subunit of IL-12 and/or IL-23.

46. A method of treating psoriasis in a subject or a population of subjects, comprising
selecting a subject or a population of subjects who would benefit from an improvement in a PASI score or a PGA score, wherein the subject or each of the subjects in the population were previously exposed to etanercept, and
administering to the subject or each subject in the population an antibody, or antigen binding portion thereof, which is capable of binding to the p40 subunit of IL-12 and/or IL-23,
wherein the subject or at least 70% of the population of subjects achieves at least a PASI 75 response at about week 28,
wherein the subject or at least 75% of the population of subjects achieves at least a PASI 75 at about week 88,
wherein the subject or at least 50% of the population of subjects achieves at least a PASI 90 at about week 28,
wherein the subject or at least 70% of the population of subjects achieves at least a PASI 90 at about week 88,
wherein the subject or at least 20% of the population of subjects achieves at least a PASI 100 at about week 28,
wherein the subject or at least 45% of the population of subjects achieves at least a PASI 100 at about week 88,
wherein the subject or at least 20% of the population of subjects achieves at least a PGA 0 at about week 28,
wherein the subject or at least 55% of the population of subjects achieves at least a PGA 0 or 1 at about week 28,
wherein the subject or at least 70% of the population of subjects achieves at least a PGA 0 or 1 at about week 88, or
wherein the subject or at least 45% of the population of subjects achieves at least a PGA 0 at about week 88.

47.-50. (canceled)

51. The method of claim 46, wherein the subject or each of the subjects in the population did not previously achieve a PGA response of 0 or 1.

52. The method of claim 46, wherein the subject or each of the subjects in the population previously achieved a PGA response of 0 or 1.

53. The method of claim 1, wherein the antibody, or antigen-binding portion thereof, is administered according to a periodicity of about once every 4 week, thereby treating psoriasis in the subject or the population of subjects.

54. The method of claim 1, wherein the antibody, or antigen-binding portion thereof, is administered according to a periodicity of about once every 12 weeks, thereby treating psoriasis in the subject or the population of subjects.

55. The method of claim 1, wherein the antibody, or antigen-binding portion thereof, is administered in a
a) a first dose amount according to a first periodicity of about once every 4 weeks; and
b) a second dose amount that is about 40-60% of the first dose amount of the antibody, or antigen-binding portion thereof, according to a second periodicity of about once every 4 weeks,
thereby treating psoriasis in the subject or the population of subjects.

56. The method of claim 1, wherein the antibody, or antigen-binding portion thereof, is administered in a
a) a first dose amount of an antibody, or antigen-binding portion thereof, which is capable of binding to the p40 subunit of IL-12 and/or IL-23, according to a first periodicity of about once every 4 weeks; and
b) a second dose amount that is about 40-60% of the first dose amount of the antibody, or antigen-binding portion thereof, according to a second periodicity of about once every 4 weeks; and
c) the second dose amount of the antibody, or antigen-binding portion thereof, according to a third periodicity of about once every 12 weeks,
thereby treating psoriasis in the subject or the population of subjects.

57. The method of claim 55, wherein the first dose amount is at least about 200 mg, or wherein the second dose amount is at least about 100 mg.

58. The method of claim 55, wherein the first dose amount is at least about 200 mg, or wherein the second dose amount is at least about 100 mg.

59. The method of claim 1, wherein the antibody is a human antibody.

60. The method of claim 1, wherein the antibody is ABT-874.

61. The method of claim 1, method comprises administering to the subject or to each subjects in the population:
a) about 200 mg of ABT-874 once every four weeks for two doses; and
b) about 100 mg of ABT-874 every four weeks thereafter.

62. The method of claim 1, wherein the method comprises administering to the subject or to each subjects in the population:
a) about 200 mg of ABT-874 at weeks 0 and 4; and
b) about 100 mg of ABT-874 at week 8 and every 4 weeks thereafter.

63. The method of claim 1, wherein the antibody is administered subcutaneously.

64. The method of claim 1, wherein the psoriasis is moderate to severe or chronic psoriasis, or wherein the psoriasis is plaque psoriasis.


SEQUENCE LISTING (32 sequences)

SEQ ID NO: 1 - protein, 6 aa, Homo sapiens
  MISC_FEATURE (1)..(1): Xaa at position 1 could be either His or Ser
XGSXDX

SEQ ID NO: 2 - protein, 12 aa, Homo sapiens
  MISC_FEATURE (2)..(2): Xaa at position 2 could be either Ser or Thr
QXYXXXXXXXXX

SEQ ID NO: 3 - protein, 17 aa, Homo sapiens
FIRYDGSNKYYADSVKG

SEQ ID NO: 4 - protein, 7 aa, Homo sapiens
  MISC_FEATURE (1)..(1): Xaa at position 1 could be either Gly or Tyr
XNXXRPS

SEQ ID NO: 5 - protein, 9 aa, Homo sapiens
  MISC_FEATURE (5)..(5): Xaa represents either Ser or Glu
FTFSXYGMH

SEQ ID NO: 6 - protein, 13 aa, Homo sapiens
  MISC_FEATURE (1)..(1): Xaa at position 1 could be either Ser or Thr
XGXXSNIXXXXVX

SEQ ID NO: 7 - protein, 115 aa, Homo sapiens
  MISC_FEATURE (6)..(6): Xaa at position 6 could be either Gln or Glu
QVQLVXSGGGVVQPGXSLRLSCAASGFTFSXYGMHWVRQAPGKGLEWVAFIRYDGSNKYYADSBKGRFTISRDNSKNTLYLQMXSLRAEDTAVYYCXXXGSXDXWGQGTMVTVSS

SEQ ID NO: 8 - protein, 112 aa, Homo sapiens
  MISC_FEATURE (1)..(1): Xaa at position 1 could be either Ser or Gln
XXVLTQPPSVSGXPGQRVTISCXGXXSNIXXXXVXWYQQLPGTAPKLLIYXNXXRPSGVPDRFSGSKSGTSASLAITGXQAEDEADYYCQXYXXXXXXXXXFGTGTKVTVLG

SEQ ID NO: 9 - protein, 6 aa, Homo sapiens
  MISC_FEATURE (2)..(2): Xaa at position 2 could be either Gly, Val, Cys or His
HXXXXX

SEQ ID NO: 10 - protein, 12 aa, Homo sapiens
  MISC_FEATURE (4)..(4): Xaa at position 4 could be either Asp or Ser
QSYXXXTHPALL

SEQ ID NO: 11 - protein, 17 aa, Homo sapiens
  MISC_FEATURE (1)..(1): Xaa at position 1 could be either Phe, Thr or Tyr
XIXYXXSXKXYADSVKG

SEQ ID NO: 12 - protein, 7 aa, Homo sapiens
  MISC_FEATURE (1)..(1): Xaa at position 1 could be either Gly, Tyr, Ser, Thr, Asn or Gln
XNDQRPS

SEQ ID NO: 13 - protein, 9 aa, Homo sapiens
  MISC_FEATURE (4)..(5): Xaa at position 4 and 5 represents any amino acid
FTFXXXXMH

SEQ ID NO: 14 - protein, 13 aa, Homo sapiens
  MISC_FEATURE (9)..(9): Xaa at position 9 could be either Ser, Cys, Arg, Asn, Asp or Thr
SGGRSNIGXXXVK

SEQ ID NO: 15 - protein, 114 aa, Homo sapiens
  MISC_FEATURE (5)..(5): Xaa at position 5 could be either Gln or Glu
QVQVXSGGGVVQPGRSLRLSCAASGFTFSXYGMHWVRQAPGKGLEWVAFIRYDGSNKYYADSVKGRFTISRDNSKNTLYLQMXSLRAEDTAVYYCKTHGSHDNWGQGTMVTVSS

SEQ ID NO: 16 - protein, 112 aa, Homo sapiens
  MISC_FEATURE (1)..(1): Xaa at position 1  could be either Ser or Gln
XXVLTQPPSVSGXPGQRVTISCSGXRSNIGSNTVKWYQQLPGTAPKLLIYXNDQRPSGVPDRFSGSKSGTSASLAITGXQAEDEADYYCQSYDRXTHPALLFGTGTKVTVLG

SEQ ID NO: 17 - protein, 6 aa, Homo sapiens
HGSHDN

SEQ ID NO: 18 - protein, 12 aa, Homo sapiens
QSYDRGTHPALL

SEQ ID NO: 19 - protein, 17 aa, Homo sapiens
FIRYDGSNKYYADSVKG

SEQ ID NO: 20 - protein, 7 aa, Homo sapiens
GNDQRPS

SEQ ID NO: 21 - protein, 9 aa, Homo sapiens
FTFSSYGMH

SEQ ID NO: 22 - protein, 13 aa, Homo sapiens
SGGRSNIGSNTVK

SEQ ID NO: 23 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCKTHGSHDNWGQGTMVTVSS

SEQ ID NO: 24 - protein, 112 aa, Homo sapiens
SYVLTQPPSVSGTPGQRVTISCSGGRSWIGSNTVKWYQQLPGTAPKLLIYGNDQRPSGVPDRFSGSKSGTSASLAITGVQAEDEADYYCQSYDRGTHPALLFGTGTKVTVLG

SEQ ID NO: 25 - protein, 6 aa, Homo sapiens
HGSHDN

SEQ ID NO: 26 - protein, 12 aa, Homo sapiens
QSYDRYTHPALL

SEQ ID NO: 27 - protein, 17 aa, Homo sapiens
FIRYDGSNKYYADSVKG

SEQ ID NO: 28 - protein, 7 aa, Homo sapiens
YNDQRPS

SEQ ID NO: 29 - protein, 9 aa, Homo sapiens
FTFSSYGMH

SEQ ID NO: 30 - protein, 13 aa, Homo sapiens
SGSRSNIGSNTVK

SEQ ID NO: 31 - protein, 115 aa, Homo sapiens [sequence omitted]

SEQ ID NO: 32 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG